(19) United States
(12) Patent Application Publication
(10) Pub. No.: US 2021/0177823 A1
(43) Pub. Date: Jun. 17, 2021

(54) NEUROMODULATING COMPOSITIONS AND RELATED THERAPEUTIC METHODS FOR THE TREATMENT OF CANCER

(71) Applicant: Flagship Pioneering Innovations V, Inc., Cambridge, MA (US)

(72) Inventors: Erica WEINSTEIN, Boston, MA (US); Jordi MATA-FINK, Happy Valley, OR (US); Avak KAHVEJIAN, Lexington, MA (US); Noubar B. AFEYAN, Lexington, MA (US); Laura Kristina JEANBART, Zurich (CH); Alexandra LANTERMANN, Boston, MA (US); Jonathan Barry HUROV, Bedford, MA (US)

(21) Appl. No.: 17/023,778

(22) Filed: Sep. 17, 2020

Related U.S. Application Data
(63) Continuation of application No. 16/320,327, filed on Jan. 24, 2019, now abandoned.
(60) Provisional application No. 62/366,773, filed on Jul. 26, 2016.

(51) Int. Cl. A61K 31/4515; A61K 31/7068; A61K 31/519; A61P 35/00

(52) U.S. Cl. CPC A61K 31/4515; A61P 35/00; A61K 31/519; A61K 31/7068

(57) ABSTRACT

Described herein are methods for treating a subject having or at risk of developing cancer by administering a neuromodulating agent.

### BACKGROUND

Cancer is still one of the deadliest threats to human health. In 2012, there were 14 million new cases of cancer worldwide and 8.2 million cancer-related deaths. The number of new cancer cases is expected to rise to 22 million by 2030, and worldwide cancer deaths are project to increase by 60%. Thus, there remains a need in the field for treatments for cancer.

### SUMMARY OF THE INVENTION

The invention relates to the discovery that modulation of neurological signaling pathways can modulate cellular responses in cancer cells and, e.g., can be used to treat cancer. Accordingly, therapeutic and pharmaceutical compositions (as well as veterinary compositions) comprising neuromodulating agents and related methods are disclosed herein for treatment of cancer. The invention also features methods of profiling, categorizing, and selecting treatment for a subject based on neurome gene expression in a tumor or cancer cell.

In one aspect, the invention provides a method of treating a subject with cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject with cancer by contacting a tumor or cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having cancer by contacting a tumor or cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject at risk of developing cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as at risk of developing cancer by administering to the subject an effective amount of a neuromodulating agent selected from the group including a neurotransmission modulator, a neuropeptide signaling modulator, a neuronal growth factor modulator, and a neurome gene expression modulator.

In some embodiments of any of the above aspects, the cancer is pancreatic cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is small cell lung cancer (SCLC) and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is non-small cell lung cancer (NSCLC) and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is melanoma and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is prostate cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is breast cancer and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is glioma and the method includes administering to the subject an effective amount of a neuromodulating agent selected from the group including a neurotransmission modulator, a neuropeptide signaling modulator, a neuronal growth factor modulator, and a neurome gene expression modulator. In some embodiments, the cancer is gastric cancer and the method includes administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing tumor growth by contacting a tumor with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing tumor growth by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing tumor volume by contacting a tumor with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing tumor volume by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing the number or activity of nerve fibers in a tumor or tumor microenvironment by contacting a tumor or tumor microenvironment with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing the number or activity of nerve fibers in a tumor or tumor microenvironment by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the tumor is highly innervated.

In another aspect, the invention provides a method of decreasing cancer cell proliferation by contacting a cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing cancer cell proliferation by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing cancer cell metastasis by contacting a cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing cancer cell metastasis by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing cancer cell invasion by contacting a cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of decreasing cancer cell invasion by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the cancer cell invasion or metastasis occurs along a nerve.

In another aspect, the invention provides a method of increasing cancer cell death by contacting the cancer cell with an effective amount of a neuromodulating agent selected from the group including a neurotransmission modulator, a neuropeptide signaling modulator, a neuronal growth factor modulator, and a neurome gene expression modulator.

In another aspect, the invention provides a method of increasing cancer cell death by administering an effective amount of a neuromodulating agent selected from the group including a neurotransmission modulator, a neuropeptide signaling modulator, a neuronal growth factor modulator, and a neurome gene expression modulator.

In another aspect, the invention provides a method of preventing tumor initiation by contacting a tumor or a tissue at risk of developing a tumor with an effective amount of a neuromodulating agent selected from the group including a neurotransmission modulator, a neuropeptide signaling modulator, a neuronal growth factor modulator, and a neurome gene expression modulator.

In another aspect, the invention provides a method of preventing tumor initiation by administering an effective amount of a neuromodulating agent selected from the group including a neurotransmission modulator, a neuropeptide signaling modulator, a neuronal growth factor modulator, and a neurome gene expression modulator.

In some embodiments of any of the above aspects, the cancer or tumor is a neuro-dependent cancer or tumor.

In some embodiments of any of the above aspects, the method includes administering to a subject that has or is at risk of developing a cancer listed in column 2 of Table 10 an effective amount of a neuromodulating agent that modulates a corresponding gene listed in column 1 of Table 10.

In another aspect, the invention provides a method of decreasing the growth or volume of a neuro-dependent cancer by contacting the tumor with an effective amount of an agent selected from a neuromodulating agent selected from the group including a neurotransmission modulator, a neuropeptide signaling modulator, a neuronal growth factor modulator, and a neurome gene expression modulator.

In another aspect, the invention provides a method of decreasing the growth or volume of a neuro-dependent cancer by administering an effective amount of an agent selected from a neuromodulating agent selected from the group including a neurotransmission modulator, a neuropeptide signaling modulator, a neuronal growth factor modulator, and a neurome gene expression modulator.

In another aspect, the invention provides a method of treating a subject with neuro-dependent cancer by administering to the subject an effective amount of a neuromodulating agent selected from the group including a neurotransmission modulator, a neuropeptide signaling modulator, a neuronal growth factor modulator, and a neurome gene expression modulator.

In another aspect, the invention provides a method of treating a subject identified as having neuro-dependent cancer by administering to the subject an effective amount of a neuromodulating agent selected from the group including a neurotransmission modulator, a neuropeptide signaling modulator, a neuronal growth factor modulator, and a neurome gene expression modulator.

In some embodiments of any of the above aspects, the neuro-dependent cancer overexpresses one or more neurome genes. In some embodiments, the neuromodulating agent reduces the expression of the one or more overexpressed genes or the activity of the protein encoded by the one or more overexpressed genes.

In some embodiments of any of the above aspects, the neuro-dependent cancer under-expresses one or more neurome genes. In some embodiments, the neuromodulating agent increases the expression the one or more under-expressed genes or the activity of the protein encoded by the one or more under-expressed genes.

In some embodiments of any of the above aspects, the one or more neurome genes is a neurome gene listed in Table 7.

In another aspect, the invention provides a method of treating a subject with a neuro-dependent cancer in listed Table 12 by administering to the subject an effective amount of a neuromodulating agent that decreases the expression of a corresponding neurome gene in Table 12 or the activity of the protein encoded by said neurome gene.

In another aspect, the invention provides a method of treating a subject identified as having a neuro-dependent cancer in listed Table 12 by administering to the subject an effective amount of a neuromodulating agent that decreases the expression of a corresponding neurome gene in Table 12 or the activity of the protein encoded by said neurome gene.

In another aspect, the invention provides a method of treating a subject with a neuro-dependent cancer in listed Table 12 by contacting the tumor or cancer cell with an effective amount of a neuromodulating agent that decreases the expression of a corresponding neurome gene in Table 12 or the activity of the protein encoded by said neurome gene.

In another aspect, the invention provides a method of treating a subject identified as having a neuro-dependent cancer in listed Table 12 by contacting the tumor or cancer cell with an effective amount of a neuromodulating agent that decreases the expression of a corresponding neurome gene in Table 12 or the activity of the protein encoded by said neurome gene.

In another aspect, the invention provides a method of treating a subject with a neuro-dependent cancer in listed Table 13 by administering to the subject an effective amount of a neuromodulating agent that increases the expression of a corresponding neurome gene in Table 13 or the activity of the protein encoded by said neurome gene.

In another aspect, the invention provides a method of treating a subject identified as having a neuro-dependent cancer in listed Table 13 by administering to the subject an effective amount of a neuromodulating agent that increases the expression of a corresponding neurome gene in Table 13 or the activity of the protein encoded by said neurome gene.

In another aspect, the invention provides a method of treating a subject with a neuro-dependent cancer in listed Table 13 by contacting the tumor or cancer cell with an effective amount of a neuromodulating agent that increases the expression of a corresponding neurome gene in Table 13 or the activity of the protein encoded by said neurome gene.

In another aspect, the invention provides a method of treating a subject identified as having a neuro-dependent cancer in listed Table 13 by contacting the tumor or cancer cell with an effective amount of a neuromodulating agent that increases the expression of a corresponding neurome gene in Table 13 or the activity of the protein encoded by said neurome gene.

In another aspect, the invention provides a method of treating a subject with a neuro-dependent cancer in listed Tables 14A-14C by administering to the subject an effective amount of a neuromodulating agent that modulates the corresponding biosynthetic enzyme or receptor expressed in the neuro-dependent cancer listed in Tables 14A-14C.

In another aspect, the invention provides a method of treating a subject identified as having a neuro-dependent cancer in listed Tables 14A-14C by administering to the subject an effective amount of a neuromodulating agent that modulates the corresponding biosynthetic enzyme or receptor expressed in the neuro-dependent cancer listed in Tables 14A-14C.

In another aspect, the invention provides a method of treating a subject with a neuro-dependent cancer in listed Tables 14A-14C by contacting the tumor or cancer cell with an effective amount of a neuromodulating agent that modulates the corresponding biosynthetic enzyme or receptor expressed in the neuro-dependent cancer listed in Tables 14A-14C.

In another aspect, the invention provides a method of treating a subject identified as having a neuro-dependent cancer in listed Tables 14A-14C by contacting the tumor or cancer cell with an effective amount of a neuromodulating agent that modulates the corresponding biosynthetic enzyme or receptor expressed in the neuro-dependent cancer listed in Tables 14A-14C.

In some embodiments of any of the above aspects, the method further includes profiling the cancer cell or tumor for expression of one or more neurome genes in Table 7 before administering the neuromodulating agent.

In one aspect, the invention provides a method of identifying a subject as having neuro-dependent cancer by profiling a tumor sample from the subject for expression of one or more neurome genes in Table 7.

In some embodiments of any of the above aspects, the neuromodulating agent is selected based on the cancer cell or tumor expression profile of one or more neurome genes in Table 7.

In some embodiments of any of the above aspects, the neuromodulating agent increases the expression of one or more neurome genes in Table 7 or the activity of the protein encoded by said one or more neurome genes.

In some embodiments of any of the above aspects, the neuromodulating agent decreases the expression of one or more neurome genes in Table 7 or the activity of the protein encoded by said one or more neurome genes.

In another aspect, the invention provides a method of treating a subject with cancer, the method including: a) profiling a tumor sample from the subject for expression of one or more neurome genes in Table 7; b) selecting a neuromodulating agent to administer to the subject based on the neurome gene expression profile of the tumor sample; and c) administering an effective amount of the neuromodulating agent to the subject, wherein the neuromodulating agent is selected from the group including a neurotransmission modulator, a neuropeptide signaling modulator, a neuronal growth factor modulator, and a neurome gene expression modulator.

In another aspect, the invention provides a method of treating a subject with small cell lung cancer by administering to the subject an effective amount of a muscarinic receptor antagonist.

In another aspect, the invention provides a method of treating a subject identified as having small cell lung cancer that overexpresses CHRM4 by administering to the subject an effective amount of a muscarinic receptor antagonist.

In another aspect, the invention provides a method of treating a subject with small cell lung cancer, the method including: a) profiling a tumor sample from the subject for expression of CHRM4; b) identifying the subject as having a small cell lung cancer that overexpresses CHRM4 if CHRM4 expression is at least 1.5 fold higher than a housekeeping gene; and c) administering to the subject an effective amount of a muscarinic receptor antagonist.

In some embodiments of any of the above aspects, the muscarinic receptor antagonist is an antagonist listed in Table 2A or 2E. In some embodiments, the muscarinic receptor antagonist is a CHRM4 antagonist selected from the group including AFDX-384, dicycloverine, himbacine, mamba toxin 3, PD-102,807, PD-0298029, and tropicamide.

In some embodiments of any of the above aspects, the cancer is gastrointestinal cancer, gastric cancer, melanoma, pancreatic cancer, urogenital cancer, prostate cancer, gynecological cancer, ovarian cancer, lung cancer, small cell lung cancer, non-small cell lung cancer, head and neck cancer, esophageal cancer, CNS cancer, glioma, malignant mesothelioma, non-metastatic or metastatic breast cancer, skin cancer, thyroid cancer, bone or soft tissue sarcoma, paraneoplastic cancer, or a hematologic neoplasia. In some embodiments of any of the above aspects, the cancer is pancreatic cancer. In some embodiments of any of the above aspects, the cancer is glioma. In some embodiments of any of the above aspects, the cancer is SCLC. In some embodiments of any of the above aspects, the cancer is NSCLC. In some embodiments of any of the above aspects, the cancer is breast cancer. In some embodiments of any of the above aspects, the cancer is prostate cancer. In some embodiments of any of the above aspects, the cancer is gastric cancer. In some embodiments of any of the above aspects, the cancer is melanoma.

In some embodiments of any of the above aspects, the neuromodulating agent is a dopamine agonist, adrenergic agonist, nicotinic agonist, muscarinic agonist, serotonin agonist, glutamate receptor agonist, histamine agonist, cannabinoid receptor agonist, purinergic receptor agonist, GABA agonist, neuropeptide Y receptor agonist, somatostatin receptor agonist, CGRP receptor agonist, tachykinin receptor agonist, VIP receptor agonist, opioid agonist, oxytocin receptor agonist, or vasopressin receptor agonist. In some embodiments, the agonist is selected from an agonist listed in Tables 2A-2L.

In some embodiments of any of the above aspects, the neuromodulating agent is a dopamine antagonist, adrenergic antagonist, nicotinic antagonist, muscarinic antagonist, serotonin antagonist, glutamate receptor antagonist, histamine antagonist, cannabinoid receptor antagonist, purinergic receptor antagonist, GABA antagonist, neuropeptide Y receptor antagonist, somatostatin receptor antagonist, CGRP receptor antagonist, tachykinin receptor antagonist, VIP receptor antagonist, opioid antagonist, oxytocin receptor antagonist, or vasopressin receptor antagonist. In some embodiments, the antagonist is selected from an antagonist listed in Tables 2A-2L. In some embodiments, the antagonist is a purinergic receptor antagonist listed in Tables 2A or 2K. In some embodiments, the purinergic receptor antagonist is an adenosine receptor antagonist. In some embodiments, the adenosine receptor antagonist is MRS-1220 or KW3902. In some embodiments, the antagonist is a dopamine antagonist listed in Table 2A or 2C. In some embodiments, the dopamine antagonist is haloperidol or L-741,626. In some embodiments, the cancer or tumor expresses a dopamine receptor. In some embodiments, the antagonist is a histamine antagonist listed in Table 2A or 2I. In some embodiments, the histamine antagonist is acrivastine, azelastine, astemizole, bilastine, bromodiphenhydramine, brompheniramine, buclizine, carbinoxamine, or cetirizine.

In some embodiments of any of the above aspects, the neuromodulating agent is neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide.

In some embodiments of any of the above aspects, the neuromodulating agent is a neuropeptide Y, CGRP, somatostatin, bombesin, cholecystokinin, dynorphin, enkephalin, endorphin, gastrin glucagon, melatonin, motilin, neurokinin A, neurokinin B, orexin, oxytocin, pancreatic peptide, peptide YY, substance P, or vasoactive intestinal peptide blocking antibody.

In some embodiments of any of the above aspects, the neuromodulating agent is a blocking or neutralizing antibody against BDNF, NGF, LIF, GDNF, sortilin, artemin, neurturin, CNTF, IGF, TGFβ1, TGFβ2, TGFβ3, NTF3, NTF4, persephin, or VEGFA. In some embodiments, the neuromodulating agent is a human anti-NGF antibody.

In some embodiments of any of the above aspects, the neuromodulating agent is a neurotransmission modulator. In some embodiments, the neurotransmission modulator is a neurotransmitter listed in Tables 1A-1B a neurotransmitter encoded by a gene in Table 7, an agonist or an antagonist of a neurotransmitter of neurotransmitter receptor listed in Tables 1A-1B or encoded by a gene in Table 7, a neurotransmission modulator listed in Table 2M, a modulator of a biosynthesis, channel, ligand receptor, signaling, structural, synaptic, vesicular, or transporter protein encoded by a gene in Table 7, a channel or transporter protein encoded by a gene in Table 8, or a neurotoxin listed in Table 3. In some embodiments, the neurotransmission modulator is a neurotoxin listed in Table 3. In some embodiments, the neurotoxin is tetanospasmin or botulinum toxin. In some embodiments, the agonist or antagonist is an agonist or antagonist listed in Tables 2A-2K.

In some embodiments of any of the above aspects, the neuromodulating agent is a neuropeptide signaling modulator. In some embodiments, the neuropeptide signaling modulator is a neuropeptide listed in Tables 1A-1B or encoded by a gene in Table 7 or analog thereof, an agonist or antagonist of a neuropeptide or neuropeptide receptor listed in in Tables 1A-1B or encoded by a gene in Table 7, or a modulator of a biosynthesis, ligand, receptor, or signaling protein encoded by a gene in Table 7. In some embodiments, the neuropeptide has at least 70%, 75%, 80%, 85%, 90%, 95%, 98%, or 99% identity to the neuropeptide sequence referenced by accession number or Entrez Gene ID in Table 1A-1B or Table 7. In some embodiments, the agonist or antagonist is an agonist or antagonist listed in Table 2A or 2L.

In some embodiments of any of the above aspects, the neuromodulating agent is a neuronal growth factor modulator. In some embodiments, the neuronal growth factor modulator is a neuronal growth factor listed in Table 1C or encoded by a gene in Table 7 or an analog thereof, or a modulator of a ligand, receptor, structural, synaptic, or signaling protein encoded by a gene in Table 7. In some embodiments, the neuronal growth factor has at least 70%, 75%, 80%, 85%, 90%, 90%, 98%, or 99% identity to the neuronal growth factor sequence referenced by accession number or Entrez Gene ID in Table 1C or Table 7. In some embodiments, the neuronal growth factor modulator is an antibody listed in Table 5. In some embodiments, the neuronal growth factor modulator is an agonist or antagonist listed in Table 6. In some embodiments, the neuronal growth factor modulator is etanercept, thalidomide, lenalidomide, pomalidomide, pentoxifylline, bupropion, DOI, disitertide, or trabedersen.

In some embodiments of any of the above aspects, the neuromodulating agent is a neurome gene expression modulator. In some embodiments, the neurome gene expression modulator increases or decreases the expression of a neurome gene in Table 7.

In some embodiments of any of the above aspects, the neuromodulating agent modulates the expression of a neurome gene in Table 7 or the activity of a protein encoded by a neurome gene in Table 7.

In some embodiments of any of the above aspects, the neuromodulating agent is selected from the group including a neurotransmitter, a neuropeptide, an antibody, a small molecule, a DNA molecule, a RNA molecule, a gRNA, and a viral vector. In some embodiments, the antibody is a blocking antibody. In some embodiments, the RNA molecule is an mRNA or an inhibitory RNA. In some embodiments, the viral vector is selected from the group including an adeno-associated virus (AAV), an adenovirus, a parvovirus, a coronavirus, a rhabdovirus, a paramyxovirus, a picornavirus, an alphavirus, a herpes virus, a poxvirus, and a lentivirus. In some embodiments, the herpes virus is a replication deficient herpes virus.

In some embodiments of any of the above aspects, the neuromodulating agent does not cross the blood brain barrier. In some embodiments, the neuromodulating agent has been modified to prevent blood brain barrier crossing by conjugation to a targeting moiety, formulation in a particulate delivery system, addition of a molecular adduct, or through modulation of its size, polarity, flexibility, or lipophilicity.

In some embodiments of any of the above aspects, the neuromodulating agent does not have a direct effect on the central nervous system or gut.

In some embodiments of any of the above aspects, the neuromodulating agent is administered locally. In some embodiments, the neuromodulating agent is administered intratumorally.

In some embodiments of any of the above aspects, the method further includes administering a second therapeutic agent. In some embodiments, the second therapeutic agent is a checkpoint inhibitor, a chemotherapeutic agent, a biologic cancer agent, an anti-angiogenic drug, a drug that targets cancer metabolism, an antibody that marks a cancer cell surface for destruction, an antibody-drug conjugate, a cell therapy, a commonly used anti-neoplastic agent, or a non-drug therapy. In some embodiments, the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands. In some embodiments, the biologic cancer agent is an antibody listed in Table 9.

In some embodiments of any of the above aspects, the neuromodulating agent decreases tumor volume, tumor growth, tumor innervation, cancer cell proliferation, cancer cell invasion, or cancer cell metastasis, or increases cancer cell death.

In some embodiments of any of the above aspects, the method further includes measuring one or more of tumor volume, tumor growth, tumor innervation, cancer cell proliferation, cancer cell invasion, cancer cell metastasis, or tumor neurome gene expression before administration of the neuromodulating agent.

In some embodiments of any of the above aspects, the method further includes measuring one or more of tumor volume, tumor growth, tumor innervation, cancer cell proliferation, cancer cell invasion, cancer cell metastasis, or tumor neurome gene expression after administration of the neuromodulating agent.

In some embodiments of any of the above aspects, the method further includes measuring the expression of one or more neurome genes in Table 7 after administration of the neuromodulating agent.

In some embodiments of any of the above aspects, the one or more neurome genes in Table 7 is a channel, transporter, neurotransmitter, neuropeptide, neurotrophic, signaling, synaptic, structural, ligand, receptor, biosynthesis, other, or vesicular gene.

In some embodiments of any of the above aspects, the subject is not diagnosed as having a neuropsychiatric disorder.

In some embodiments of any of the above aspects, the subject is not diagnosed as having high blood pressure or a cardiac condition.

In some embodiments of any of the above aspects, the neuromodulating agent is administered in an amount sufficient to increase or decrease tumor innervation, decrease nerve activity in a tumor, treat the cancer or tumor, cause remission, reduce tumor growth, reduce tumor volume, reduce tumor metastasis, reduce tumor invasion, reduce tumor proliferation, reduce tumor number, increase cancer cell death, increase time to recurrence, or improve survival.

### Definitions

As used herein, “administration” refers to providing or giving a subject a therapeutic agent (e.g., a neuromodulating agent), by any effective route. Exemplary routes of administration are described herein below.

As used herein, the term “agonist” refers to an agent (e.g., a neurotransmitter, neuropeptide, small molecule, or antibody) that increases receptor activity. An agonist may activate a receptor by directly binding to the receptor, by acting as a cofactor, by modulating receptor conformation (e.g., maintaining a receptor in an open or active state). An agonist may increase receptor activity by 10%, 20%, 30%, 40%, 50%, 60%, 70%, 80%, 90%, 95%, 98% or more. An agonist may induce maximal receptor activation or partial activation depending on the concentration of the agonist and its mechanism of action.

As used herein, the term “analog” refers to a protein of similar nucleotide or amino acid composition or sequence to any of the proteins or peptides of the invention, allowing for variations that do not have an adverse effect on the ability of the protein or peptide to carry out its normal function (e.g., bind to a receptor or initiate neurotransmitter or neuropeptide signaling). Analogs may be the same length, shorter, or longer than their corresponding protein or polypeptide. Analogs may have about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of the naturally occurring protein or peptide. An analog can be a naturally occurring protein or polypeptide sequence that is modified by deletion, addition, mutation, or substitution of one or more amino acid residues.

As used herein, the term “antagonist” refers to an agent (e.g., a neurotransmitter, neuropeptide, small molecule, or antibody) that reduces or inhibits receptor activity. An antagonist may reduce receptor activity by directly binding to the receptor, by blocking the receptor binding site, by modulating receptor conformation (e.g., maintaining a receptor in a closed or inactive state). An antagonist may reduce receptor activity by 10%, 20%, 30%, 40%, 50%, 60%, 70%, 80%, 90%, 95%, 98% or more. An antagonist may also completely block or inhibit receptor activity. Antagonist activity may be concentration-dependent or -independent.

As used herein, the term “antibody” comprises at least the variable domain of a heavy chain, and normally comprises at least the variable domains of a heavy chain and of a light chain of an immunoglobulin, which bind to an antigen of interest. Antibodies and antigen-binding fragments, variants, or derivatives thereof include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “cancer” refers to a condition characterized by unregulated or abnormal cell growth. The terms “cancer cell,” “tumor cell,” and “tumor” refer to an abnormal cell, mass or population of cells that result from excessive division that may be malignant or benign and all pre-cancerous and cancerous cells and tissues.

As used herein, the term “cardiac condition” refers to a medical condition directly affecting the heart or circulatory system. Cardiac conditions include abdominal aortic aneurysm, arrhythmia (e.g., supraventricular tachycardia, inappropriate sinus tachycardia, atrial flutter, atrial fibrillation, ventricular tachycardia, and ventricular fibrillation), angina, atherosclerosis, brugada syndrome, cardiac arrest, cardiomyopathy, cardiovascular disease, congenital heart disease, coronary heart disease, catecholaminergic polymorphic ventricular tachycardia (CVPT), familial hypercholesterolaemia, heart attack, heart failure, heart block, heart valve disease (e.g., heart murmur, valve stenosis, mitral valve prolapse, and heart valve regurgitation), inherited heart conditions, long QT syndrome, progressive cardiac conduction deficit (PCCD), pericarditis, venous thromboembolism, peripheral artery disease, and stroke.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, an agent that “does not cross the blood brain barrier” is an agent that does not significantly cross the barrier between the peripheral circulation and the brain and spinal cord. This can also be referred to as “blood brain barrier impermeable” agent. Agents will have a limited ability to cross the blood brain barrier if they are not lipid soluble or have a molecular weight of over 600 Daltons. Agents that typically cross the blood brain barrier can be modified to become blood brain barrier impermeable based on chemical modifications that increase the size or alter the hydrophobicity of the agent, packaging modifications that reduce diffusion (e.g., packaging an agent within a microparticle or nanoparticle), and conjugation to biologics that direct the agent away from the blood brain barrier (e.g., conjugation to a pancreas-specific antibody). An agent that does not cross the blood brain barrier is an agent for which 30% or less (e.g., 30%, 25%, 20%, 15%, 10%, 5%, 2% or less) of the administered agent crosses the blood brain barrier.

As used herein, an agent that “does not have a direct effect on the central nervous system (CNS) or gut” is an agent that does not directly alter neurotransmission, neuronal numbers, or neuronal morphology in the CNS or gut when administered according to the methods described herein. This may be assessed by administering the agents to animal models and performing electrophysiological recordings or immunohistochemical analysis. An agent will be considered not to have a direct effect on the CNS or gut if administration according to the methods described herein has an effect on neurotransmission, neuronal numbers, or neuronal morphology in the CNS or gut that is 50% or less (e.g., 50%, 45%, 40%, 35%, 30%, 25%, 20%, 15%, 10%, 5%, or less) of the effect observed if the same agent is administered directly to the CNS or gut.

As used herein, the terms “effective amount,” “therapeutically effective amount,” and a “sufficient amount” of composition, vector construct, viral vector or cell described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating cancer it is an amount of the composition, vector construct, viral vector or cell sufficient to achieve a treatment response as compared to the response obtained without administration of the composition, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, viral vector or cell of the present disclosure is an amount which results in a beneficial or desired result in a subject as compared to a control. As defined herein, a therapeutically effective amount of a composition, vector construct, viral vector or cell of the present disclosure may be readily determined by one of ordinary skill by routine methods known in the art. Dosage regime may be adjusted to provide the optimum therapeutic response.

As used herein, the term “high blood pressure” refers to a chronic medical condition in which the systemic arterial blood pressure is elevated. It is classified as blood pressure above 140/90 mmHg.

As used herein, the terms “increasing” and “decreasing” refer to modulating resulting in, respectively, greater or lesser amounts, of function, expression, or activity of a metric relative to a reference. For example, subsequent to administration of an neuromodulating agent in a method described herein, the amount of a marker of a metric (e.g., T cell polarization) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “innervated” refers to a tissue (e.g., a tumor) that contains nerves. “Innervation” refers to the process of nerves entering a tissue.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, the terms “neuro-dependent cancer” and “neuro-dependent tumor” refer to cancer or tumor cells that are characterized in that they express one or more neurome genes, e.g., the genes listed in Tables 1A-1C, Table 7, and Table 8. Neuro-dependent cancers or tumors are responsive to neuromodulating agents as described herein. Cancer or tumor cells can be identified as neuro-dependent using standard techniques known in the art (e.g., quantitative PCR, RNA sequencing, and immunohistochemistry on cancer or tumor cell samples). Neuro-dependent cancers can overexpress or under-express neurome genes, and neuro-dependent cancers that express neuronal growth factors can promote tumor innervation. Some neuro-dependent cancers express both neurotransmitter or neuropeptide-related genes and genes for the corresponding receptors, making them capable of autocrine signaling. Patients with neuro-dependent cancers can be treated using the compositions and methods described herein to target the one or more neurome genes expressed by the cancer, and can be treated with a neuromodulating agent alone or in combination with existing anti-cancer therapies.

As used herein, a “neuromodulating agent” is an agent that affects a nerve impulse, a nerve function, one or more components of a neural pathway, neural structure, function, or activity in a neuron or a cell of an innervated tissue, e.g., in the peripheral nervous system. A neuromodulating agent may, e.g., increase or decrease neurogenesis; potentiate or inhibit the transmission of a nerve impulse; increase or decrease innervation of a tissue or tumor; or increase or decrease adrenergic, dopaminergic, cholinergic, serotonergic, glutamatergic, purinergic, GABAergic, or neuropetidergic signaling in a nerve or cell of an innervated tissue. A neuromodulating agent may be a neuropeptide, a neurotoxin, or a neurotransmitter, and may be any type of agent such as a small molecule (e.g. a neuropeptide or neurotransmitter agonist or antagonist), a peptide, a protein (e.g., an antibody or receptor fusion protein) or a nucleic acid (e.g., a therapeutic mRNA). Neuromodulating agents include neurotransmission modulators, neuropeptide signaling modulators, neuronal growth factor modulators, and neurome gene expression modulators.

As used herein, the term “neurome gene” refers to a gene expressed by a cell or tissue of the nervous system. A list of exemplary neurome genes is provided in Tables 1A-1C, Table 7, and Table 8. Non-nervous system cells and tissues (e.g., tumors) can also express neurome genes, and the invention includes methods of profiling non-nervous system cells and tissues for neurome gene expression, modulating neurome gene expression in in non-nervous system cells and tissues, and treating cancer based on neurome gene expression in in non-nervous system cells and tissues.

As used herein, the term “neurome gene expression modulator” refers to a neuromodulating agent that affects gene expression (e.g., gene transcription, gene translation, or protein levels) of one or more neurome genes. A neurome gene expression modulator may increase or decrease gene expression by 10%, 20%, 30%, 40%, 50%, 60%, 70%, 80%, 90%, 95%, 98%, or more. Neurome gene expression modulators may increase gene expression through epigenetic modifications (e.g., demethylation or acetylation), post-translational modifications (e.g., reducing ubiquitination, or altering sumoylation or phosphorylation), by increasing mRNA translation and stability, or through delivery of exogenous genetic material (e.g., a viral vector expressing a gene of interest). Neurome gene expression modulators may decrease gene expression through epigenetic modifications (e.g., methylation or deacetylation), post-translational modifications (e.g., increasing ubiquitination, or altering sumoylation or phosphorylation), or by decreasing mRNA translation and stability (e.g., using miRNA, siRNA, shRNA, or other therapeutic RNAs).

As used herein, the term “neuronal growth factor modulator” refers to a neuromodulating agent that regulates neuronal growth, development, or survival. Neuronal growth factors include proteins that promote neurogenesis, neuronal growth, and neuronal differentiation (e.g., neurotrophic factors NGF, NT3, BDNF, CNTF, and GDNF), proteins that promote neurite outgrowth (e.g., axon or dendrite outgrowth or stabilization), or proteins that promote synapse formation (e.g., synaptogenesis, synapse assembly, synaptic adhesion, synaptic maturation, synaptic refinement, or synaptic stabilization). These processes lead to innervation of tissue, including neural tissue, muscle, and tumors, and the formation of synaptic connections between two or more neurons and between neurons and non-neural cells (e.g., tumor cells). A neuronal growth factor modulator may block one or more of these processes (e.g., through the use of antibodies that block neuronal growth factors or their receptors) or promote one or more of these processes (e.g., through the use of these proteins or analogs or peptide fragments thereof). Exemplary neuronal growth factors are listed in Table 1C.

As used herein, the term “neuropeptide signaling modulator” refers to a neuromodulating agent that either induces or increases neuropeptide signaling, or decreases or blocks neuropeptide signaling. Neuropeptide signaling modulators can increase or decrease neuropeptide signaling by 10%, 20%, 30%, 40%, 50%, 60%, 70%, 80%, 90%, 95%, 98% or more. Exemplary neuropeptides and neuropeptide receptors are listed in Tables 1A-1B. Neuropeptide signaling modulators that induce or increase neuropeptide signaling include neuropeptides and analogs and fragments thereof, agents that increase neuropeptide receptor activity (e.g., neuropeptide agonists), and agents that reduce neuropeptide degradation or reuptake. Neuropeptide signaling modulators that decrease or block neuropeptide signaling include agents that reduce or inhibit neuropeptide receptor activity (e.g., neuropeptide antagonists), agents that bind to neuropeptides or block their interaction with receptors (e.g., neuropeptide blocking antibodies), or agents that increase neuropeptide degradation or clearance. Exemplary neuropeptide agonists and antagonists are listed in Table 2A and 2L.

As used herein, the term “neuropsychiatric disorder” refers to a psychiatric or mental disorder that may cause suffering or an impaired ability to function. A neuropsychiatric disorder is a syndrome characterized by clinically significant disturbance in an individual's cognition, emotion regulation, or behavior that reflects a dysfunction in the psychological, biological, or developmental processes underlying mental functioning. Neuropsychiatric disorders may be diagnosed by psychiatrists, psychologists, neurologists, or physicians. Neuropsychiatric disorders include mood disorders (e.g., depression, bipolar depression, major depressive disorder), psychotic disorders (e.g., schizophrenia, schizoaffective disorder), personality disorders (e.g., borderline personality disorder, obsessive compulsive personality disorder, narcissistic personality disorder), eating disorders, sleep disorders, sexual disorders, anxiety disorders (e.g., generalized anxiety disorder, social anxiety disorder, post-traumatic stress disorder), developmental disorders (e.g., autism, attention deficit disorder, attention deficit hyperactivity disorder), benign forgetfulness, childhood learning disorders, Alzheimer's disease, addiction, and others listed in the Diagnostic and Statistical Manual of Mental Disorders (DSM).

As used herein, the term “neurotransmission modulator” refers to a neuromodulating agent that either induces or increases neurotransmission or decreases or blocks neurotransmission. Neurotransmission modulators can increase or decrease neurotransmission by 10%, 20%, 30%, 40%, 50%, 60%, 70%, 80%, 90%, 95%, 98% or more. Exemplary neurotransmitters and neurotransmitter receptors are listed in Tables 1A-1B. Neurotransmission modulators may increase neurotransmission by increasing neurotransmitter synthesis or release, preventing neurotransmitter reuptake or degradation, increasing neurotransmitter receptor activity, increasing neurotransmitter receptor synthesis or membrane insertion, decreasing neurotransmitter degradation, and regulating neurotransmitter receptor conformation. Neurotransmission modulators that increase neurotransmission include neurotransmitters and analogs thereof and neurotransmitter receptor agonists. Neurotransmission modulators may decrease neurotransmission by decreasing neurotransmitter synthesis or release, increasing neurotransmitter reuptake or degradation, decreasing neurotransmitter receptor activity, decreasing neurotransmitter receptor synthesis or membrane insertion, increasing neurotransmitter degradation, regulating neurotransmitter receptor conformation, and disrupting the pre- or postsynaptic machinery. Neurotransmission modulators that decrease or block neurotransmission include antibodies that bind to or block the function of neurotransmitters, neurotransmitter receptor antagonists, and toxins that disrupt synaptic release.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, a “pharmaceutical composition” or “pharmaceutical preparation” is a composition or preparation, having pharmacological activity or other direct effect in the mitigation, treatment, or prevention of disease, and/or a finished dosage form or formulation thereof and which is indicated for human use.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “proliferation” refers to an increase in cell numbers through growth and division of cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

### BRIEF DESCRIPTION OF THE DRAWINGS

FIGS. 1A-1C are a series of graphs showing that adenosine receptor antagonists inhibit cancer cell growth. Adenosine receptor antagonists were applied to cancer cell lines in vitro. Adenosine Receptor 1 (A1R) antagonist KW3902 inhibited the growth of BXPC3 pancreatic cancer cells and NCI-H82 small cell lung cancer cells in vitro at low micromolar concentrations (Figs. P.1 A-B). Additionally, Adenosine Receptor 3 (A3R) antagonist MRS-1220 inhibited the growth of NCI-H82 small cell lung cancer cells in vitro at low micromolar concentrations (Fig. P.1C).

FIG. 2 is a graph showing that dopamine antagonist haloperidol decreases tumor weight in mice. Mice with orthotopic MIAPACA2 pancreatic cancer were treated with intraperitoneal (IP) injection of vehicle, haloperidol, chemotherapeutic agent gemcitabine, or a combination of haloperidol and gemcitabine. Mice that received Haldol saw a ˜50% reduction in tumor weight at the end of study (150 mg vs 300 mg for the vehicle treatment group). This reduction was equivalent to the reduction observed in the gemcitabine treatment group. The combination treatment group showed a reduction of tumor weight by about 67% compared to the vehicle treatment control group (200 mg vs 300 mg).

FIG. 3 is a series of images showing that dopamine antagonist L-741,626 induces cancer cell killing in a mouse xenograft model. Small cell lung cancer cell tumors in mice treated with dopamine antagonist in microcassette. Microdose drug delivery devices containing dopamine (DRD2) antagonist L-741,626 or chemotherapeutic agent cisplatin were implanted into patient-derived xenograft tumors in mice. Patient-derived xenograft tumors from patient 5197 showed a dose-dependent cell killing in response to L-741,626, as measured by CC3 positive tissue (left). The same sample showed a negative gradient for phospho-AKT indicating that L-741,626 inhibited dopamine receptor signaling (second from left). The same tumor showed minimal cell killing in response to cisplatin, suggesting that dopamine antagonism is killing the cells through an independent mechanism (second from right). Patient-derived xenograft tumors from patient 5217 that expressed ˜10× lower levels of DRD2 mRNA compared to patient 5197 were at all not impacted by treatment with the L-741,626 dopaminergic antagonist, suggesting that the effect is mediated through dopamine receptor signaling (right).

FIGS. 4A-4B is a series of histograms showing that dopamine antagonists inhibit the growth of cultured glioma cells. DRD2 antagonists haloperidol (FIG. 4A) and L741,626 (FIG. 4B) were added to wells containing glioma cell lines A172, LN-18, T98G, LN 299, U87, and U118 at a range of concentrations between 1 μM and 20 μM. Cell growth was assessed after 9 days in culture. Haloperidol and L741,626 were able to reduce the number of glioblastoma cells growing in culture compared to DMSO control in a dose-dependent fashion in the cell lines (FIGS. 4A-4B). The effect was strongest for both agents in A172, LN-18, LN 229, and U118 cell lines.

FIGS. 5A-5D are a heatmap of a tumor neurome gene signature. 1152 primary tumors (columns) were classified by their RNA expression of 60 different neurotransmitter receptors (rows). The tissue of origin for each tumor is denoted by the colored identifier bar at the top of each column. These data show that the neurological gene signature of a tumor did not always correspond with the tissue type of origin.

FIG. 6 is a plot of a principal components analysis of the neurome gene signature data. Neurotransmitter receptor gene expression enabled tumors to be clustered into five distinct taxa, represented by different shapes in the graph.

FIG. 7 is a series of bar charts showing that tissue of origin does not correlate to neurological taxonomy. Representation from most of all of the taxa is observed for each tumor type. These data demonstrate that the neurological taxonomy does not correspond to the tissue of origin and thus represents a new taxonomy for classifying tumors.

FIGS. 8A-8F are a series of graphs showing the classification of lung cancers based on neurome gene expression. 276 lung cancers were analyzed using RNA sequencing. Distinct clusters of tumor types were identified based on their (1) co-expression of muscarinic receptor CHRM4 and dopamine receptor DRD2, (2) expression of adrenergic receptor ADRB2, or (3) low expression of all three. Neurome gene expression clustering does not correspond to common histological taxonomy, some small cell lung cancer (SCLC) samples appear to be CHRM4 and DRD2 high (Group 1, FIG. 8D), while others are negative (Group 3), and non-small cell lung cancers (NSCLC) samples are distributed into all three groups (FIGS. 8A-8C).

FIGS. 9A-9B are a pair of graphs showing 5 year survival in patients with low or high expression of Chrna6 in intratumoral T regulatory cells (Tregs). Analysis of gene expression of tumor infiltrating Tregs in was performed in 275 non-small cell lung cancer (NSCLC) patients (FIG. 9A) and 177 colorectal cancer (CRC) patients (FIG. 9B) and normalized to CD3G in order to account for differential immune infiltration. Data analysis revealed that patients with low intratumoral Treg expression of Chrna6 had a significantly better 5 year survival than patients with high intratumoral Treg expression of Chrna6. The relationship between Chrna6 expression and survival was observed in both NSCLC and CRC.

### DETAILED DESCRIPTION

Neuromodulating agents described herein can surprisingly have effects on cancer cells, such as effects on cancer cell proliferation, cancer cell death, tumor growth, tumor initiation, tumor innervation, cancer cell metastasis, and cancer cell invasion. It has been found that neuromodulating agents thus can have a therapeutic effect on cancer.

I. Neuromodulating Agents

Neuromodulating agents described herein can agonize or inhibit genes or proteins in neuromodulatory signaling pathways, in order to treat cancer. Neuromodulatory signaling pathway genes are listed in Tables 1A-C (column 1). Additional neurome genes (e.g., genes expressed by a nervous system cell or tissue) are listed in Table 7 and Table 8. The level, activity and/or function of such genes and the proteins they encode can be modulated by pharmaceutical compositions comprising agents described herein. Neuromodulating agents also include neurotransmitter and neuropeptide ligands listed in Table 1B and neuronal growth factors listed in Table 1C.

Neuromodulating agents can be divided into four major categories: 1) neurotransmission modulators (e.g., agents that increase or decrease neurotransmission, such as neurotransmitter agonists or antagonists or neurotoxins), 2) neuropeptide signaling modulators (e.g., neuropeptides and neuropeptide agonists or antagonists), 3) neuronal growth factor modulators (e.g., neuronal growth factors or agents that agonize or antagonize neuronal growth factor signaling), and 4) neurome gene expression modulators (e.g., agents that modulate expression of a gene listed in Table 7 or Table 8). These classes of neuromodulating are described in more detail herein below.

TABLE 1A
NEUROTRANSMITTER & NEUROPEPTIDE GENES & PATHWAYS

| | | | Accession | Entrez |
|---|---|---|---|---|
| Gene | Pathway | Type | Number | Gene ID |
| ABAT | Neurotransmitter | Biosynthesis | P80404 | 18 |
| ACHE | Neurotransmitter | Biosynthesis | P22303 | 43 |
| ADCYAP1 | Neuropeptide | Ligand | P18509 | 116 |
| ADCYAP1R1 | Neuropeptide | Receptor | P41586 | 117 |
| ADIPOQ | Neuropeptide | Ligand | Q15848 | 9370 |
| ADM | Neuropeptide | Ligand | P35318 | 133 |
| ADM2 | Neuropeptide | Ligand | Q7Z4H4 | 79924 |
| ADORA2A | Neurotransmitter | Receptor | P29274 | 135 |
| ADORA2B | Neurotransmitter | Receptor | P29275 | 136 |
| Adra1a | Adrenergic/ | Receptor | P35348 | 148 |
| | Neurotransmitter | | | |
| Adra1b | Adrenergic/ | Receptor | P35368 | 147 |
| | Neurotransmitter | | | |
| Adra1d | Adrenergic/ | Receptor | P25100 | 146 |
| | Neurotransmitter | | | |
| Adra2a | Adrenergic/ | Receptor | P08913 | 150 |
| | Neurotransmitter | | | |
| Adra2b | Adrenergic/ | Receptor | P18089 | 151 |
| | Neurotransmitter | | | |
| Adra2c | Adrenergic/ | Receptor | P18825 | 152 |
| | Neurotransmitter | | | |
| Adrb1 | Adrenergic/ | Receptor | P08588 | 153 |
| | Neurotransmitter | | | |
| Adrb2 | Adrenergic/ | Receptor | P07550 | 154 |
| | Neurotransmitter | | | |
| Adrb3 | Adrenergic/ | Receptor | P13945 | 155 |
| | Neurotransmitter | | | |
| Adrbk1 | Adrenergic | Kinase | P25098 | 156 |
| Adrbk2 | Adrenergic | Kinase | P35626 | 157 |
| AGRN | Neuropeptide | Ligand | O00468 | 375790 |
| AGRP | Neuropeptide | Ligand | O00253 | 181 |
| AGT | Neuropeptide | Ligand | P01019 | 183 |
| AGTR1 | Neuropeptide | Receptor | P30556 | 185 |
| APLN | Neuropeptide | Ligand | Q9ULZ1 | 8862 |
| ASIP | Neuropeptide | Ligand | P42127 | 434 |
| AVP | Neuropeptide | Ligand | P01185 | 551 |
| AVPR1A | Neuropeptide | Receptor | P30560 | 552 |
| AVPR1B | Neuropeptide | Receptor | P47901 | 553 |
| AVPR2 | Neuropeptide | Receptor | P30518 | 554 |
| BACE1 | Neurotransmitter | Biosynthesis | P56817 | 23621 |
| BCHE | Neurotransmitter | Biosynthesis | P06276 | 590 |
| BDKRB2 | Neuropeptide | Receptor | P30411 | 624 |
| BRS3 | Neuromodulator | Receptor | P32247 | P32247 |
| C1QBP | Neuropeptide | Receptor | Q07021 | 708 |
| C4orf48 | Neuropeptide | Ligand | Q5BLP8 | 401115 |
| C6orf89 | Neuromodulator | Receptor | Q6UWU4 | 221477 |
| CALCA | Neuropeptide | Ligand | P06881 | 796 |
| CALCB | Neuropeptide | Ligand | P10092 | 797 |
| CALCR | Neuropeptide | Receptor | P30988 | 799 |
| CALCRL | Neuropeptide | Receptor | Q16602 | 10203 |
| CARTPT | Neuropeptide | Ligand | Q16568 | 9607 |
| CASR | Neuropeptide | Biosynthesis | P41180 | 846 |
| CCK | Neuropeptide | Ligand | P06307 | 885 |
| CCKAR | Neuropeptide | Receptor | P32238 | 886 |
| CCKBR | Neuropeptide | Receptor | P32239 | 887 |
| CCL2 | Neuropeptide | Ligand | P13500 | 6347 |
| CHAT | Neurotransmitter | Biosynthesis | P28329 | 1103 |
| CHGA | Neuropeptide | Ligand | P10645 | 1113 |
| CHGB | Neuropeptide | Ligand | P05060 | 1114 |
| CHRFAM7A | Neurotransmitter | Receptor | Q494W8 | 89832 |
| Chrm1 | Cholinergic/ | Receptor | P11229 | 1128 |
| | Neurotransmitter | | | |
| Chrm2 | Cholinergic/ | Receptor | P08172 | 1129 |
| | Neurotransmitter | | | |
| Chrm3 | Cholinergic/ | Receptor | P20309 | 1131 |
| | Neurotransmitter | | | |
| Chrm4 | Cholinergic/ | Receptor | P08173 | 1132 |
| | Neurotransmitter | | | |
| Chrm5 | Cholinergic/ | Receptor | P08912 | 1133 |
| | Neurotransmitter | | | |
| Chrna1 | Cholinergic/ | Receptor | P02708 | 1134 |
| | Neurotransmitter | | | |
| Chrna10 | Cholinergic/ | Receptor | Q9GZZ6 | 57053 |
| | Neurotransmitter | | | |
| Chrna2 | Cholinergic/ | Receptor | Q15822 | 1135 |
| | Neurotransmitter | | | |
| Chrna3 | Cholinergic/ | Receptor | P32297 | 1136 |
| | Neurotransmitter | | | |
| Chrna4 | Cholinergic/ | Receptor | P43681 | 1137 |
| | Neurotransmitter | | | |
| Chrna5 | Cholinergic/ | Receptor | P30532 | 1138 |
| | Neurotransmitter | | | |
| Chrna6 | Cholinergic/ | Receptor | Q15825 | 8973 |
| | Neurotransmitter | | | |
| Chrna7 | Cholinergic/ | Receptor | P36544 | 1139 |
| | Neurotransmitter | | | |
| Chrna9 | Cholinergic/ | Receptor | Q9UGM1 | 55584 |
| | Neurotransmitter | | | |
| Chrnb1 | Cholinergic/ | Receptor | P11230 | 1140 |
| | Neurotransmitter | | | |
| Chrnb2 | Cholinergic/ | Receptor | P17787 | 1141 |
| | Neurotransmitter | | | |
| Chrnb3 | Cholinergic/ | Receptor | Q05901 | 1142 |
| | Neurotransmitter | | | |
| Chrnb4 | Cholinergic/ | Receptor | P30926 | 1143 |
| | Neurotransmitter | | | |
| Chrnd | Cholinergic/ | Receptor | Q07001 | 1144 |
| | Neurotransmitter | | | |
| Chrne | Cholinergic/ | Receptor | Q04844 | 1145 |
| | Neurotransmitter | | | |
| Chrng | Cholinergic/ | Receptor | P07510 | 1146 |
| | Neurotransmitter | | | |
| CLCF1 | Neuropeptide | Ligand | Q9UBD9 | 23529 |
| CNR1 | Cannabinoid/ | Receptor | P21554 | 1268 |
| | Neurotransmitter | | | |
| CNR2 | Cannabinoid/ | Receptor | P34972 | 1269 |
| | Neurotransmitter | | | |
| CNRIP1 | Neurotransmitter | Receptor | Q96F85 | 25927 |
| COMT | Neurotransmitter | Biosynthesis | P21964 | 1312 |
| CORT | Neuropeptide | Ligand | O00230 | 1325 |
| CPA4 | Neurotransmitter | Biosynthesis | Q9UI42 | 51200 |
| CPE | Neuropeptide/ | Biosynthesis | P16870 | 1363 |
| | Neurotransmitter | | | |
| CRCP | Neuropeptide | Receptor | O75575 | 27297 |
| CREM | Neurotransmitter | Signaling | Q03060 | 1390 |
| CRH | Neuropeptide | Ligand | Q13324 | 1392 |
| CRHBP | Neuropeptide | Receptor | P24387 | 1393 |
| CRHR1 | Neuropeptide | Receptor | P34998 | 1394 |
| CRHR2 | Neuropeptide | Receptor | Q13324 | 1395 |
| CTSH | Neuropeptide | Biosynthesis | P09668 | 1512 |
| CTSV | Neuropeptide | Biosynthesis | O60911 | 1515 |
| CYSLTR1 | Neuropeptide | Receptor | Q9Y271 | 10800 |
| CYSLTR2 | Neuropeptide | Receptor | Q9NS75 | 57105 |
| DAGLA | Neurotransmitter | Biosynthesis | Q9Y4D2 | 747 |
| | (Cannabinoid) | | | |
| DAGLB | Neurotransmitter | Biosynthesis | Q8NCG7 | 221955 |
| | (Cannabinoid) | | | |
| DBH | Neurotransmitter | Biosynthesis | P09172 | 1621 |
| DBI | Neuropeptide | Ligand | P07108 | 1622 |
| DDC | Neurotransmitter | Biosynthesis | P20711 | 1644 |
| DGKI | Neurotransmitter | Biosynthesis | O75912 | 9162 |
| DOPO | Dopaminergic | Receptor | P09172 | 1621 |
| DPP4 | Neurotransmitter | Biosynthesis | P27487 | 1803 |
| Drd1 | Dopaminergic/ | Receptor | P21728 | 1812 |
| | Neurotransmitter | | | |
| Drd2 | Dopaminergic/ | Receptor | P14416 | 1813 |
| | Neurotransmitter | | | |
| Drd3 | Dopaminergic/ | Receptor | P35462 | 1814 |
| | Neurotransmitter | | | |
| Drd4 | Dopaminergic/ | Receptor | P21917 | 1815 |
| | Neurotransmitter | | | |
| Drd5 | Dopaminergic/ | Receptor | P21918 | 1816 |
| | Neurotransmitter | | | |
| ECEL1 | Neurotransmitter | Biosynthesis | O95672 | 9427 |
| EDN1 | Neuropeptide | Ligand | P05305 | 1906 |
| EDN2 | Neuropeptide | Ligand | P20800 | 1907 |
| EDN3 | Neuropeptide | Ligand | P14138 | 1908 |
| EDNRA | Neuropeptide | Receptor | P25101 | 1909 |
| EDNRB | Neuropeptide | Receptor | P24530 | 1910 |
| FAAH | Neurotransmitter | Biosynthesis | O00519 | 2166 |
| FAP | Neuropeptide | Biosynthesis | Q12884 | 2191 |
| FNTA | Neurotransmitter | Signaling | P49354 | 2339 |
| FOLH1 | Neuropeptide | Biosynthesis | Q04609 | 2346 |
| FSHR | Neuropeptide | Receptor | P23945 | 2492 |
| GABARAP | Amine | Receptor | O95166 | 11337 |
| | Neuromodulator/ | | | |
| | Neurotransmitter | | | |
| GABARAPL1 | Amine | Receptor | Q9H0R8 | 23710 |
| | Neuromodulator | | | |
| GABARAPL2 | Amine | Receptor | P60520 | 11345 |
| | Neuromodulator | | | |
| GABBR1 | Amine | Receptor | Q9UBS5 | 2550 |
| | Neuromodulator/ | | | |
| | Neurotransmitter | | | |
| GABBR2 | Amine | Receptor | O75899 | 9568 |
| | Neuromodulator | | | |
| GABRA1 | Amine | Receptor | P14867 | 2554 |
| | Neuromodulator/ | | | |
| | Neurotransmitter | | | |
| GABRA2 | Amine | Receptor | P47869 | 2555 |
| | Neuromodulator/ | | | |
| | Neurotransmitter | | | |
| GABRA3 | Neurotransmitter | Receptor | P34903 | 2556 |
| GABRA4 | Neurotransmitter | Receptor | P48169 | 2557 |
| GABRA5 | Amine | Receptor | P31644 | 2558 |
| | Neuromodulator/NT | | | |
| GABRA6 | Neurotransmitter | Receptor | Q16445 | 2559 |
| GABRB1 | Neurotransmitter | Receptor | P18505 | 2560 |
| GABRB2 | Amine | Receptor | P47870 | 2561 |
| | Neuromodulator/ | | | |
| | Neurotransmitter | | | |
| GABRB3 | Amine | Receptor | P28472 | 2562 |
| | Neuromodulator/ | | | |
| | Neurotransmitter | | | |
| GABRD | Amine | Receptor | O14764 | 2563 |
| | Neuromodulator/ | | | |
| | Neurotransmitter | | | |
| GABRE | Neurotransmitter | Receptor | P78334 | 2564 |
| GABRG1 | Neurotransmitter | Receptor | Q8N1C3 | 2565 |
| GABRG2 | Amine | Receptor | P18507 | 2566 |
| | Neuromodulator/ | | | |
| | Neurotransmitter | | | |
| GABRG3 | Neurotransmitter | Receptor | Q99928 | 2567 |
| GABRP | Neurotransmitter | Receptor | O00591 | 2568 |
| GABRQ | Neurotransmitter | Receptor | Q9UN88 | 55879 |
| GABRR1 | Amine | Receptor | P24046 | 2569 |
| | Neuromodulator/ | | | |
| | Neurotransmitter | | | |
| GABRR2 | Amine | Receptor | P28476 | 2570 |
| | Neuromodulator/ | | | |
| | Neurotransmitter | | | |
| GABRR3 | Neurotransmitter | Receptor | A8MPY1 | 200959 |
| GAD1 | Neurotransmitter | Biosynthesis | Q99259 | 2571 |
| GAD2 | Neurotransmitter | Biosynthesis | Q05329 | 2572 |
| GAL | Neuropeptide | Ligand | P22466 | 51083 |
| GALP | Neuropeptide | Ligand | Q810H5 | 85569 |
| GALR1 | Neuropeptide | Receptor | P47211 | 2587 |
| GALR2 | Neuropeptide | Receptor | O43603 | 8811 |
| GALR3 | Neuropeptide | Receptor | O60755 | 8484 |
| GAST | Neuropeptide | Ligand | P01350 | 2520 |
| GCGR | Secretin | Receptor | P47871 | 2642 |
| GCHFR | Neurotransmitter | Biosynthesis | P30047 | 2644 |
| GH1 | Neuropeptide | Ligand | P01241 | 2688 |
| GHRH | Neuropeptide | Ligand | P01286 | 2691 |
| GHRHR | Neuropeptide | Receptor | Q02643 | 2692 |
| GHRL | Neuropeptide | Ligand | Q9UBU3 | 51738 |
| GIP | Neuropeptide | Ligand | P09681 | 2695 |
| GLRA1 | Neurotransmitter | Receptor | P23415 | 2741 |
| GLRA2 | Neurotransmitter | Receptor | P23416 | 2742 |
| GLRA3 | Neurotransmitter | Receptor | O75311 | 8001 |
| GLRA4 | Neurotransmitter | Receptor | Q5JXX5 | 441509 |
| GLRB | Neurotransmitter | Receptor | P48167 | 2743 |
| GLS | Neurotransmitter | Biosynthesis | O94925 | 2744 |
| GLS2 | Neurotransmitter | Biosynthesis | Q9UI32 | 27165 |
| GluA1 (GluR1) | Amine | Receptor | P42261 | 2890 |
| | Neuromodulator | | | |
| GluK1 (GluR5) | Amine | Receptor | P39086 | 2897 |
| | Neuromodulator | | | |
| GLUL | Neurotransmitter | Biosynthesis | P15104 | 2752 |
| GluN1(NR1) | Amine | Receptor | Q05586 | 2902 |
| | Neuromodulator | | | |
| GNMT | Neurotransmitter | Biosynthesis | Q14749 | 27232 |
| GNRH1 | Neuropeptide | Ligand | P01148 | 2796 |
| GNRH2 | Neuropeptide | Ligand | O43555 | 2797 |
| GPHN | Neuropeptide | Ligand | Q9NQX3 | 10243 |
| GPER1 | Neurotransmitter | Receptor | Q99527 | 2852 |
| GPR1 | Neurotransmitter | Receptor | P46091 | 2825 |
| GPR139 | Neurotransmitter | Receptor | Q6DWJ6 | 124274 |
| GPR143 | Neurotransmitter | Receptor | P51810 | 4935 |
| GPR149 | Neurotransmitter | Receptor | Q86SP6 | 344758 |
| GPR18 | Neurotransmitter | Receptor | Q14330 | 2841 |
| GPR21 | Neurotransmitter | Receptor | Q99679 | 2844 |
| GPR26 | Neurotransmitter | Receptor | Q8NDV2 | 2849 |
| GPR3 | Neurotransmitter | Receptor | P46089 | 2827 |
| GPR35 | Neurotransmitter | Receptor | Q9HC97 | 2859 |
| GPR52 | Neurotransmitter | Receptor | Q9Y2T5 | 9293 |
| GPR55 | Neurotransmitter | Receptor | Q9Y2T6 | 9290 |
| GPR78 | Neurotransmitter | Receptor | Q96P69 | 27201 |
| GPR83 | Neurotransmitter | Receptor | Q9NYM4 | 10888 |
| GPR84 | Neurotransmitter | Receptor | Q9NQS5 | 53831 |
| GPRASP1 | Neurotransmitter | Receptor | Q5JY77 | 9737 |
| GPR50 | Amine | Receptor | Q13585 | 9248 |
| | Neuromodulator | | | |
| GRIA1 | Amine | Receptor | P42261 | 2890 |
| | Neuromodulator/ | | | |
| | Neurotransmitter | | | |
| GRIA2 | Amine | Receptor | P42262 | 2891 |
| | Neuromodulator/ | | | |
| | Neurotransmitter | | | |
| GRIA3 | Amine | Receptor | P42263 | 2892 |
| | Neuromodulator/ | | | |
| | Neurotransmitter | | | |
| GRIA4 | Amine | Receptor | P48058 | 2893 |
| | Neuromodulator/ | | | |
| | Neurotransmitter | | | |
| GRID1 | Neurotransmitter | Receptor | Q9ULK0 | 2894 |
| GRID2 | Neurotransmitter | Receptor | O43424 | 2895 |
| GRIK1 | Amine | Receptor | P39086 | 2897 |
| | Neuromodulator/ | | | |
| | Neurotransmitter | | | |
| GRIK2 | Amine | Receptor | Q13002 | 2898 |
| | Neuromodulator/ | | | |
| | Neurotransmitter | | | |
| GRIK3 | Amine | Receptor | Q13003 | 2899 |
| | Neuromodulator/ | | | |
| | Neurotransmitter | | | |
| GRIK4 | Amine | Receptor | Q16099 | 2900 |
| | Neuromodulator/ | | | |
| | Neurotransmitter | | | |
| GRIK5 | Amine | Receptor | Q16478 | 2901 |
| | Neuromodulator/ | | | |
| | Neurotransmitter | | | |
| GRIN1 | Amine | Receptor | Q05586 | 2902 |
| | Neuromodulator/ | | | |
| | Neurotransmitter | | | |
| GRIN2A | Amine | Receptor | Q12879 | 2903 |
| | Neuromodulator/ | | | |
| | Neurotransmitter | | | |
| GRIN2B | Amine | Receptor | Q13224 | 2904 |
| | Neuromodulator | | | |
| GRIN2C | Amine | Receptor | Q14957 | 2905 |
| | Neuromodulator/ | | | |
| | Neurotransmitter | | | |
| GRIN2D | Amine | Receptor | O15399 | 2906 |
| | Neuromodulator/ | | | |
| | Neurotransmitter | | | |
| GRIN3A | Amine | Receptor | Q8TCU5 | 116443 |
| | Neuromodulator/ | | | |
| | Neurotransmitter | | | |
| GRIN3B | Amine | Receptor | O60391 | 116444 |
| | Neuromodulator/ | | | |
| | Neurotransmitter | | | |
| GRK2 | Neurotransmitter | Receptor | P25098 | 156 |
| GRK3 | Neurotransmitter | Receptor | P35626 | 157 |
| GRM1 | Neurotransmitter | Receptor | Q13255 | 2911 |
| GRM2 | Neurotransmitter | Receptor | Q14416 | 2912 |
| GRM3 | Neurotransmitter | Receptor | Q14832 | 2913 |
| GRM4 | Neurotransmitter | Receptor | Q14833 | 2914 |
| GRM5 | Neurotransmitter | Receptor | P41594 | 2915 |
| GRM6 | Neurotransmitter | Receptor | O15303 | 2916 |
| GRM7 | Neurotransmitter | Receptor | Q14831 | 2917 |
| GRM8 | Neurotransmitter | Receptor | O00222 | 2918 |
| GRP | Neuropeptide | Ligand | P07492 | 2922 |
| GRPR | Neuropeptide | Receptor | P30550 | 2925 |
| HCRT | Neuropeptide | Ligand | O43612 | 3060 |
| HCRTR1 | Neuropeptide/ | Receptor | O43613 | 3061 |
| | Orexin | | | |
| HCRTR2 | Neuropeptide/ | Receptor | O43614 | 3062 |
| | Orexin | | | |
| HNMT | Neurotransmitter | Biosynthesis | P50135 | 3176 |
| HOMER1 | Neurotransmitter | Receptor | Q86YM7 | 9456 |
| HRH1 | Amine | Receptor | P35367 | 3269 |
| | Neuromodulator/ | | | |
| | Neurotransmitter | | | |
| HRH2 | Amine | Receptor | P25021 | 3274 |
| | Neuromodulator/ | | | |
| | Neurotransmitter | | | |
| HRH3 | Amine | Receptor | Q9Y5N1 | 11255 |
| | Neuromodulator/ | | | |
| | Neurotransmitter | | | |
| HRH4 | Amine | Receptor | Q9H3N8 | 59340 |
| | Neuromodulator/ | | | |
| | Neurotransmitter | | | |
| Htr1a | Serotonin/ | Receptor | P08908 | 3350 |
| | Neurotransmitter | | | |
| Htr1b | Serotonin/ | Receptor | P28222 | 3351 |
| | Neurotransmitter | | | |
| Htr1c | Serotonin | Receptor | P28335 | |
| Htr1d | Serotonin/ | Receptor | P28221 | 3352 |
| | Neurotransmitter | | | |
| Htr1e | Serotonin/ | Receptor | P28566 | 3354 |
| | Neurotransmitter | | | |
| Htr1f | Serotonin/ | Receptor | P30939 | 3355 |
| | Neurotransmitter | | | |
| Htr2a | Serotonin/ | Receptor | P28223 | 3356 |
| | Neurotransmitter | | | |
| Htr2b | Serotonin/ | Receptor | P41595 | 3357 |
| | Neurotransmitter | | | |
| Htr2c | Serotonin/ | Receptor | P28335 | 3358 |
| | Neurotransmitter | | | |
| Htr3a | Serotonin/ | Receptor | P46098 | 3359 |
| | Neurotransmitter | | | |
| Htr3b | Serotonin/ | Receptor | O95264 | 9177 |
| | Neurotransmitter | | | |
| Htr3c | Serotonin/ | Receptor | Q8WXA8 | 170572 |
| | Neurotransmitter | | | |
| Htr3d | Serotonin/ | Receptor | Q70Z44 | 200909 |
| | Neurotransmitter | | | |
| HTR3E | Neurotransmitter | Receptor | A5X5Y0 | 285242 |
| Htr4 | Serotonin/ | Receptor | Q13639 | 3360 |
| | Neurotransmitter | | | |
| Htr5a | Serotonin/ | Receptor | P47898 | 3361 |
| | Neurotransmitter | | | |
| Htr5b | Serotonin | Receptor | P35365 | 79247 |
| HTR5BP | Neurotransmitter | Receptor | | 645694 |
| Htr6 | Serotonin/ | Receptor | P50406 | 3362 |
| | Neurotransmitter | | | |
| Htr7 | Serotonin/ | Receptor | P32305 | 3363 |
| | Neurotransmitter | | | |
| IAPP | Neuropeptide | Ligand | P10997 | 3375 |
| ITPR1 | Neurotransmitter | Signaling | Q14643 | 3708 |
| ITPR2 | Neurotransmitter | Signaling | Q14571 | 3709 |
| ITPR3 | Neurotransmitter | Signaling | Q14573 | 3710 |
| KISS1 | Neuropeptide | Ligand | Q15726 | 3814 |
| KISS1R | Neuropeptide | Receptor | Q969F8 | 84634 |
| LEP | Neuropeptide | Ligand | P41159 | 3952 |
| LHCGR | Neuropeptide | Receptor | P22888 | 3973 |
| LIF | Neuropeptide | Ligand | P15018 | 3976 |
| LTB4R | Neuropeptide | Receptor | Q15722 | 1241 |
| LTB4R2 | Neuropeptide | Receptor | Q9NPC1 | 56413 |
| LYNX1 | Neurotransmitter | Receptor | Q9BZG9 | 66004 |
| MAOA | Neurotransmitter | Biosynthesis | P21397 | 4128 |
| MAOB | Neurotransmitter | Biosynthesis | P27338 | 4129 |
| MC1R | Neuropeptide | Receptor | Q01726 | 4157 |
| MC2R | Neuropeptide | Receptor | Q01718 | 4158 |
| MC3R | Neuropeptide | Receptor | P41968 | 4159 |
| MC4R | Neuropeptide | Receptor | P32245 | 4160 |
| MC5R | Neuropeptide | Receptor | P33032 | 4161 |
| MCHR1 | Neuropeptide | Receptor | Q99705 | 2847 |
| MCHR2 | Neuropeptide | Receptor | Q969V1 | 84539 |
| MLN | Neuropeptide | Ligand | P12872 | 4295 |
| MME | Neuropeptide | Biosynthesis | P08473 | 4311 |
| MRAP | Neuropeptide | Receptor | Q8TCY5 | 56246 |
| MRAP2 | Neuropeptide | Receptor | Q96G30 | 112609 |
| MRGPRF | Neurotransmitter | Receptor | Q96AM1 | 116535 |
| MRGPRX1 | Neuropeptide | Receptor | Q96LB2 | 259249 |
| MRGPRX2 | Neurotransmitter | Receptor | Q96LB1 | 117194 |
| MRGPRX3 | Neuropeptide | Receptor | Q96LB0 | 117195 |
| MRGPRX4 | Neuropeptide | Receptor | Q96LA9 | 117196 |
| MTNR1A | Amine | Receptor | P48039 | 4543 |
| | Neuromodulator/ | | | |
| | Neuropeptide | | | |
| MTNR1B | Amine | Receptor | P49286 | 4544 |
| | Neuromodulator/ | | | |
| | Neuropeptide | | | |
| NAALAD2 | Neuropeptide | Biosynthesis | Q9Y3Q0 | 10003 |
| NAMPT | NT | Biosynthesis | P43490 | 10135 |
| NGF | Neuropeptide | Ligand | P01138 | 4803 |
| NISCH | Amine | Receptor | Q9Y2I1 | 11188 |
| | Neuromodulator/ | | | |
| | Neurotransmitter | | | |
| NMB | Neuropeptide | Ligand | P08949 | 4828 |
| NMBR | Neuropeptide | Receptor | P28336 | 4829 |
| NMS | Neuropeptide | Ligand | Q5H8A3 | 129521 |
| NMU | Neuropeptide | Ligand | P48645 | 10874 |
| NMUR1 | Neuropeptide | Receptor | Q9HB89 | 10316 |
| NMUR2 | Neuropeptide | Receptor | Q9GZQ4 | 56923 |
| NOS1 | Neurotransmitter | Biosynthesis | P29475 | 4842 |
| NPB | Neuropeptide | Ligand | Q8NG41 | 256933 |
| NPBWR1 | Neuropeptide | Receptor | P48145 | 2831 |
| NPBWR2 | Neuropeptide | Receptor | P48146 | 2832 |
| NPFF | Neuropeptide | Ligand | O15130 | 8620 |
| NPFFR1 | Neuropeptide | Receptor | Q9GZQ6 | 64106 |
| NPFFR2 | Neuropeptide | Receptor | Q9Y5X5 | 10886 |
| NPPA | Neuropeptide | Ligand | P01160 | 4878 |
| NPPB | Neuropeptide | Ligand | P16860 | 4879 |
| NPPC | Neuropeptide | Ligand | P23582 | 4880 |
| NPS | Neuropeptide | Ligand | P0C0P6 | 594857 |
| NPSR1 | Neuropeptide | Receptor | Q6W5P4 | 387129 |
| NPTN | Neurotransmitter | Receptor | Q9Y639 | 27020 |
| NPVF | Neuropeptide | Ligand | Q9HCQ7 | 64111 |
| NPW | Neuropeptide | Ligand | Q8N729 | 283869 |
| NPY | Neuropeptide | Ligand | P01303 | 4852 |
| NPY1R | Neuropeptide | Receptor | P25929 | 4886 |
| NPY2R | Neuropeptide | Receptor | P49146 | 4887 |
| NPY4R | Neuropeptide | Receptor | P50391 | 5540 |
| NPY5R | Neuropeptide | Receptor | Q15761 | 4889 |
| NPY6R | Neuropeptide | Receptor | Q61212 | 4888 |
| NTS | Neuropeptide | Ligand | Q6FH20 | 4922 |
| NTSR1 | Neuropeptide | Receptor | P30989 | 4923 |
| NTSR2 | Neuropeptide | Receptor | Q63384 | 23620 |
| NXPH1 | Neuropeptide | Ligand | P58417 | 30010 |
| NXPH2 | Neuropeptide | Ligand | O95156 | 11249 |
| NXPH3 | Neuropeptide | Ligand | O95157 | 11248 |
| NXPH4 | Neuropeptide | Ligand | O95158 | 11247 |
| OGFR | Neuropeptide | Receptor | Q9NZT2 | 11054 |
| OPRD1 | Neuropeptide/ | Receptor | P41143 | 4985 |
| | Opioid | | | |
| OPRK1 | Neuropeptide/ | Receptor | P41145 | 4986 |
| | Opioid | | | |
| OPRL1 | Neuropeptide | Receptor | P41146 | 4987 |
| OPRM1 | Neuropeptide/ | Receptor | P35372 | 4988 |
| | Opioid | | | |
| OXT | Neuropeptide | Ligand | P01178 | 5020 |
| OXTR | Neuropeptide | Receptor | P30559 | 5021 |
| P2RX1 | Neurotransmitter | Receptor | P51575 | 5023 |
| P2RX2 | Neurotransmitter | Receptor | Q9UBL9 | 22953 |
| P2RX3 | Neurotransmitter | Receptor | P56373 | 5024 |
| P2RX4 | Neurotransmitter | Receptor | Q99571 | 5025 |
| P2RX5 | Neurotransmitter | Receptor | Q93086 | 5026 |
| P2RX6 | Neurotransmitter | Receptor | O15547 | 9127 |
| P2RX7 | Neurotransmitter | Receptor | Q99572 | 5027 |
| P2RY11 | Neurotransmitter | Receptor | Q96G91 | 5032 |
| PAH | Neurotransmitter | Biosynthesis | P00439 | 5053 |
| PC | Neurotransmitter | Biosynthesis | P11498 | 5091 |
| PCSK1 | Neuropeptide | Biosynthesis | P29120 | 5122 |
| PCSK1N | Neuropeptide | Ligand/ | Q9UHG2 | 27344 |
| | | Biosynthesis | | |
| PDE1B | Neurotransmitter | Signaling | Q01064 | 5153 |
| PDE4A | Neurotransmitter | Signaling | P27815 | 5141 |
| PDE4D | Neurotransmitter | Signaling | Q08499 | 5144 |
| PDYN | Neuropeptide | Ligand | P01213 | 5173 |
| PENK | Neuropeptide | Ligand | P01211 | 5179 |
| PHOX2A | Neurotransmitter | Biosynthesis | O14813 | 401 |
| PHOX2B | Neurotransmitter | Biosynthesis | Q99453 | 8929 |
| PIK3CA | Neurotransmitter | Signaling | P42336 | 5290 |
| PIK3CB | Neurotransmitter | Signaling | P42338 | 5291 |
| PIK3CG | Neurotransmitter | Signaling | P48736 | 5294 |
| PLCB1 | Neurotransmitter | Signaling | Q9NQ66 | 23236 |
| PLCB2 | Neurotransmitter | Signaling | Q00722 | 5330 |
| PLCB3 | Neurotransmitter | Signaling | Q01970 | 5331 |
| PLCB4 | Neurotransmitter | Signaling | Q15147 | 5332 |
| PLCD1 | Neurotransmitter | Signaling | P51178 | 5333 |
| PLCE1 | Neurotransmitter | Signaling | Q9P212 | 51196 |
| PLCG1 | Neurotransmitter | Signaling | P19174 | 5335 |
| PLCL1 | Neurotransmitter | Signaling | Q15111 | 5334 |
| PLCL2 | Neurotransmitter | Signaling | Q9UPR0 | 23228 |
| PMCH | Neuropeptide | Ligand | P20382 | 5367 |
| PNOC | Neuropeptide | Ligand | Q13519 | 5368 |
| POMC | Neuropeptide | Ligand | P01189 | 5443 |
| PPP1CB | Neurotransmitter | Signaling | P62140 | 5500 |
| PPP1CC | Neurotransmitter | Signaling | P36873 | 5501 |
| PPY | Neuropeptide | Ligand | P01298 | 5539 |
| PPY2P | Neuropeptide | Ligand | Q9NRI7 | 23614 |
| PRIMA1 | Neurotransmitter | Biosynthesis | Q86XR5 | 145270 |
| PRKACG | Neurotransmitter | Signaling | P22612 | 5568 |
| PRKAR2B | Neurotransmitter | Signaling | P31323 | 5577 |
| PRKCG | Neurotransmitter | Signaling | P05129 | 5582 |
| PRKX | Neurotransmitter | Signaling | P51817 | 5613 |
| PRL | Neuropeptide | Ligand | P01236 | 5617 |
| PRLH | Neuropeptide | Ligand | P81277 | 51052 |
| PRLHR | Neuropeptide | Receptor | P49683 | 2834 |
| PRLR | Neuropeptide | Receptor | P16471 | 5618 |
| PROK1 | Neuropeptide | Ligand | P58294 | 84432 |
| PROK2 | Neuropeptide | Ligand | Q9HC23 | 60675 |
| PROKR1 | Neuropeptide | Receptor | Q8TCW9 | 10887 |
| PROKR2 | Neuropeptide | Receptor | Q8NFJ6 | 128674 |
| PTGDR | Neuropeptide | Receptor | Q13258 | 5729 |
| PTGDR2 | Neuropeptide | Receptor | Q9Y5Y4 | 11251 |
| PTGER1 | Neuropeptide | Receptor | P34995 | 5731 |
| PTGER2 | Neuropeptide | Receptor | P43116 | 5732 |
| PTGER3 | Neuropeptide | Receptor | P43115 | 5733 |
| PTGER4 | Neuropeptide | Receptor | P35408 | 5734 |
| PTGFR | Neuropeptide | Receptor | P43088 | 5737 |
| PTGIR | Neuropeptide | Receptor | P43119 | 5739 |
| PTGS2 | Neuropeptide | Biosynthesis | P35354 | 5743 |
| PTH | Neuropeptide | Ligand | P01270 | 5741 |
| PTH1R | Neuropeptide | Receptor | Q03431 | 5745 |
| PTH2 | Neuropeptide | Ligand | Q9Y3E5 | 113091 |
| PTH2R | Neuropeptide | Receptor | P49190 | 5746 |
| PTHLH | Neuropeptide | Ligand | P12272 | 5744 |
| PTK2 | Neuropeptide | Signaling | Q05397 | 5747 |
| PTK2B | Neuropeptide | Signaling | Q14289 | 2185 |
| PYY | Neuropeptide | Ligand | P10082 | 5697 |
| PYY2 | Neuropeptide | Ligand | Q9NRI6 | 23615 |
| PYY3 | Neuropeptide | Ligand | Q5JQD4 | 644059 |
| QRFP | Neuropeptide | Ligand | P83859 | 347148 |
| QRFPR | Neuropeptide | Receptor | Q96P65 | 84109 |
| RAMP1 | Neuropeptide | Receptor | O60894 | 10267 |
| RAMP2 | Neuropeptide | Receptor | O60895 | 10266 |
| RAMP3 | Neuropeptide | Receptor | O60896 | 10268 |
| RIC3 | Neurotransmitter | Receptor | Q7Z5B4 | 79608 |
| RLN1 | Neuropeptide | Ligand | P04808 | 6013 |
| RLN2 | Neuropeptide | Ligand | P04090 | 6019 |
| RLN3 | Neuropeptide | Ligand | Q8WXF3 | 117579 |
| RXFP1 | Neuropeptide | Receptor | Q9HBX9 | 59350 |
| RXFP2 | Neuropeptide | Receptor | Q8WXD0 | 122042 |
| RXFP3 | Neuropeptide | Receptor | Q9NSD7 | 51289 |
| RXFP4 | Neuropeptide | Receptor | Q8TDU9 | 339403 |
| S1PR4 | Neuropeptide | Receptor | O95977 | 8698 |
| SCG2 | Neuropeptide | Ligand/ | P13521 | 7857 |
| | | Vesicles | | |
| SCG3 | Neuropeptide | Ligand/ | Q8WXD2 | 29106 |
| | | Vesicles | | |
| SCG5 | Neuropeptide | Ligand/ | P05408 | 6447 |
| | | Vesicles | | |
| SCT | Neuropeptide | Ligand | P09683 | 6343 |
| SCTR | Secretin | Receptor | P47872 | 6344 |
| SHANK3 | Neurotransmitter | Signaling | Q9BYB0 | 85358 |
| SLC6A1 | Amine | Transferase | P30531 | 6529 |
| | Neuromodulator | | | |
| SLC6A13 | Amine | Transferase | Q9NSD5 | 6540 |
| | Neuromodulator | | | |
| Slc6a4 | Serotonin | Transporter | P31645 | 6532 |
| SNX13 | Neurotransmitter | Signaling | Q9Y5W8 | 23161 |
| SPX | Neuropeptide | Ligand | Q9BT56 | 80763 |
| SST | Neuropeptide | Ligand | P61278 | 6750 |
| SSTR1 | Neuropeptide | Receptor | P30872 | 6751 |
| SSTR2 | Neuropeptide | Receptor | P30874 | 6752 |
| SSTR3 | Neuropeptide | Receptor | P32745 | 6753 |
| SSTR4 | Neuropeptide | Receptor | P31391 | 6754 |
| SSTR5 | Neuropeptide | Receptor | P35346 | 6755 |
| TAAR1 | Amine | Receptor | Q96RJ0 | 134864 |
| | Neuromodulator | | | |
| TAAR2 | Amine | Receptor | Q9P1P5 | 9287 |
| | Neuromodulator | | | |
| TAAR5 | Neurotransmitter | Receptor | O14804 | 9038 |
| TAC1 | Neuropeptide | Ligand | P20366 | 6863 |
| TAC3 | Neuropeptide | Ligand | Q9UHF0 | 6866 |
| TAC4 | Neuropeptide | Ligand | Q86UU9 | 255061 |
| TACR1 | Neuropeptide | Receptor | P25103 | 6869 |
| TACR2 | Neuropeptide | Receptor | P21452 | 6865 |
| TACR3 | Neuropeptide | Receptor | P29371 | 6870 |
| TBXA2R | Neuropeptide | Receptor | P21731 | 6915 |
| TH | Neurotransmitter | Biosynthesis | P07101 | 7054 |
| TPH1 | Neurotransmitter | Biosynthesis | P17752 | 7166 |
| TPH2 | Neurotransmitter | Biosynthesis | Q8IWU9 | 121278 |
| TRHDE | Neurotransmitter | Biosynthesis | Q9UKU6 | 29953 |
| TRH | Neuropeptide | Ligand | P20396 | 7200 |
| TRHR | Neuropeptide | Receptor | P34981 | 7201 |
| TSHR | Neuropeptide | Receptor | P16473 | 7253 |
| UCN | Neuropeptide | Ligand | P55089 | 7349 |
| UCN2 | Neuropeptide | Ligand | Q96RP3 | 90226 |
| UCN3 | Neuropeptide | Ligand | Q969E3 | 114131 |
| UTS2 | Neuropeptide | Ligand | O95399 | 10911 |
| UTS2B | Neuropeptide | Ligand | Q756I0 | 257313 |
| UTS2R | Neuropeptide | Receptor | Q9UKP6 | 2837 |
| VIP | Neuropeptide | Ligand | P01282 | 7432 |
| VIPR1 | Neuropeptide | Receptor | P32241 | 7433 |
| VIPR2 | Neuropeptide | Receptor | P41587 | 7434 |

TABLE 1B
NEUROTRANSMITTERS & NEUROPEPTIDE LIGANDS

| | | | Accession |
|---|---|---|---|
| Ligand | Pathway | Type | Number |
| 2-Arachidonoylglycerol | Endocannabinoid | Ligand | |
| 2-Arachidonyl glyceryl ether | Endocannabinoid | Ligand | |
| 3-methoxytyramine | Amines | Ligand | |
| Acetylcholine | Amino Acids | Ligand | |
| Adenosine | Purine | Ligand | |
| Adenosine triphosphate | Purine | Ligand | |
| Agmatine | Amino Acids | Ligand | |
| Anandamide | Endocannabinoid | Ligand | |
| Aspartate | Amino Acids | Ligand | |
| Bombesin | Other | Ligand | |
| Carbon monoxide | Gas | Ligand | |
| Cholecystokinin | Gastrins | Ligand | P06307 |
| Cocaine | Other | Ligand | |
| Corticotropin | Opioids | Ligand | |
| D-serine | Amino Acids | Ligand | |
| Dopamine | Monoamines | Ligand | |
| Dynorphin | Opioids | Ligand | |
| Endorphin | Opioids | Ligand | |
| Enkephaline | Opioids | Ligand | |
| Epinephrine | Monoamines | Ligand | |
| Gamma-aminobutyric acid | Amino Acids | Ligand | |
| Gastrin | Gastrins | Ligand | P01350 |
| Gastrin releasing peptide | Other | Ligand | P07492 |
| Glucagon | Secretins | Ligand | |
| Glutamate | Amino Acids | Ligand | |
| Glycine | Amino Acids | Ligand | |
| Growth hormone-releasing | Secretins | Ligand | Q9UBU3 |
| factor | | | |
| Histamine | Monoamines | Ligand | |
| Melatonin | Monoamines | Ligand | |
| Motilin | Secretins | Ligand | P12872 |
| N-Acetylaspartylglutamate | Neuropeptides | Ligand | |
| N-Arachidonoyl dopamine | Endocannabinoid | Ligand | |
| N-methylphenethylamine | Amines | Ligand | |
| N-methyltryptamine | Amines | Ligand | |
| Neurokinin A | Tachykinins | Ligand | P20366 |
| Neurokinin B | Tachykinins | Ligand | Q334E7 |
| Neuropeptide Y | Neuropeptides | Ligand | P01303 |
| Neurophysin I | Neurohypophyseals | Ligand | P01178 |
| Neurophysin II | Neurohypophyseals | Ligand | P01185 |
| Nitric oxide | Gas | Ligand | |
| Norepinephrine | Monoamines | Ligand | |
| Octopamine | Amines | Ligand | |
| Orexin A | Orexins | Ligand | O43612 |
| Orexin B | Orexins | Ligand | O43613 |
| Oxytocin | Neurohypophyseals | Ligand | |
| Pancreatic polypeptide | Neuropeptides | Ligand | P01298 |
| Peptide YY | Neuropeptides | Ligand | P10082 |
| Phenethylamine | Amines | Ligand | |
| Serotonin | Monoamines | Ligand | |
| Somatostatin | Somatostatins | Ligand | P61278 |
| Substance P | Neuropeptides | Ligand | |
| Synephrine | Amines | Ligand | |
| Tryptamine | Amines | Ligand | |
| Tyramine | Amines | Ligand | |
| Vasoactive intestinal | Secretins | Ligand | P01282 |
| peptide | | | |
| Vasopressin | Neurohypophyseals | Ligand | |
| Virodhamine | Endocannabinoid | Ligand | |

TABLE 1C
NEURONAL GROWTH FACTORS

| | | Accession | Entrez |
|---|---|---|---|
| Gene | Type | Number | Gene ID |
| ARTN | Ligand | Q5T4W7 | 9048 |
| BDNF | Ligand | P23560 | 627 |
| BDNF-AS | Ligand | | 497258 |
| BEX1 | Signaling | Q9HBH7 | 55859 |
| BEX3 | Signaling | Q00994 | 27018 |
| CD34 | Receptor | P28906 | 947 |
| CDNF | Ligand | Q49AH0 | 441549 |
| CNTF | Ligand | P26441 | 1270 |
| CNTFR | Receptor | P26992 | 1271 |
| CRLF1 | Receptor | O75462 | 9244 |
| CSPG5 | Ligand | O95196 | 10675 |
| DCLK1 | Signaling | O15075 | 9201 |
| DISC1 | Signaling | Q9NRI5 | 27185 |
| DNAJC5 | Signaling | Q9H3Z4 | 80331 |
| DPYSL2 | Signaling | Q16555 | 1808 |
| DVL1 | Signaling | O14640 | 1855 |
| EFNA5 | Ligand | P52803 | 1946 |
| EGR3 | Signaling | Q06889 | 1960 |
| ENO2 | Signaling | P09104 | 2026 |
| EphA1 | Receptor | P21709 | 2041 |
| EphA10 | Receptor | Q5JZY3 | 284656 |
| EphA2 | Receptor | P29317 | 1969 |
| EphA3 | Receptor | P29320 | 2042 |
| EphA4 | Receptor | P29317 | 2043 |
| EphA5 | Receptor | P54756 | 2044 |
| EphA6 | Receptor | Q9UF33 | 285220 |
| EphA7 | Receptor | Q15375 | 2045 |
| EphA8 | Receptor | P29322 | 2046 |
| EphB1 | Receptor | P54762 | 2047 |
| EphB2 | Receptor | P29323 | 2048 |
| EphB3 | Receptor | P54753 | 2049 |
| EphB4 | Receptor | P54760 | 2050 |
| EphB6 | Receptor | O15197 | 2051 |
| ETBR2 | Receptor | O60883 | 9283 |
| FSTL4 | Receptor | Q6MZW2 | 23105 |
| GDNF | Ligand | P39905 | 2668 |
| GFRA1 | Receptor | P56159 | 2674 |
| GFRA2 | Receptor | O00451 | 2675 |
| GFRA3 | Receptor | O60609 | 2676 |
| GFRA4 | Receptor | Q9GZZ7 | 64096 |
| GPR37 | Receptor | O15354 | 2861 |
| GPRIN1 | Signaling | Q7Z2K8 | 114787 |
| GPRIN2 | Signaling | O60269 | 9721 |
| GPRIN3 | Signaling | Q6ZVF9 | 285513 |
| GRB2 | Signaling | P62993 | 2885 |
| GZF1 | Signaling | Q9H116 | 64412 |
| IFNA1 | Ligand | P01562 | 3439 |
| IGF1 | Ligand | P05019 | 3479 |
| IGF2 | Ligand | P01344 | 3481 |
| IL11RA | Receptor | Q14626 | 3590 |
| IL1B | Ligand | P01584 | 3553 |
| IL3 | Ligand | P08700 | 3562 |
| IL4 | Ligand | P05112 | 3565 |
| IL6 | Ligand | P05231 | 3569 |
| IL6R | Receptor | P08887 | 3570 |
| IL6ST | Signaling | P40189 | 3572 |
| INS | Ligand | P01308 | 3630 |
| L1CAM | Signaling | P32004 | 3897 |
| LIF | Ligand | P15018 | 3976 |
| LIFR | Receptor | P42702 | 3977 |
| MAGED1 | Signaling | Q9Y5V3 | 9500 |
| MANF | Ligand | P55145 | 7873 |
| NDNF | Ligand | Q8TB73 | 79625 |
| NENF | Ligand | Q9UMX5 | 29937 |
| NENFP1 | Ligand | | 106480294 |
| NENFP2 | Ligand | | 100129880 |
| NENFP3 | Ligand | | 106481703 |
| NGF | Ligand | P01138 | 4803 |
| NGFR | Receptor | P08138 | 4804 |
| NRG1 | Ligand | Q02297 | 3084 |
| NRP1 | Receptor | O14786 | 8829 |
| NRTN | Ligand | Q99748 | 902 |
| NTF3 | Ligand | P20783 | 4908 |
| NTF4 | Ligand | P34130 | 4909 |
| NTRK1 | Receptor | P04629 | 4914 |
| NTRK2 | Receptor | Q16620 | 4915 |
| NTRK3 | Receptor | Q16288 | 4916 |
| PDPK1 | Signaling | O15530 | 5170 |
| PEDF | Ligand | P36955 | 5176 |
| PLEKHH3 | Signaling | Q7Z736 | 79990 |
| PSAP | Ligand | P07602 | 5660 |
| PSEN1 | Signaling | P49768 | 5663 |
| PSPN | Ligand | O70300 | 5623 |
| PTN | Ligand | P21246 | 5764 |
| RELN | Ligand | P78509 | 5649 |
| RET | Signaling | P07949 | 5979 |
| ROR1 | Receptor | Q01973 | 4919 |
| ROR2 | Receptor | Q01974 | 4920 |
| RPS6KA3 | Signaling | P51812 | 6197 |
| SDC3 | Receptor | O75056 | 9672 |
| SEMA3E | Ligand | O15041 | 9723 |
| SERPINE2 | Ligand | P07093 | 5270 |
| SERPINF1 | Ligand | P36955 | 5176 |
| SHC1 | Signaling | P51812 | 6464 |
| SNTG1 | Biosynthesis | P07602 | 54212 |
| SORCS1 | Receptor | O75056 | 114815 |
| SORCS2 | Receptor | O15041 | 57537 |
| SORCS3 | Receptor | P07093 | 22986 |
| SORT1 | Receptor | Q99523 | 6272 |
| SULF1 | Signaling | Q8IWU6 | 23213 |
| SULF2 | Signaling | Q8IWU5 | 55959 |
| TGFB1 | Ligand | P01137 | 7040 |
| TGFB2 | Ligand | P61812 | 7042 |
| TGFB3 | Ligand | P10600 | 7043 |
| TMEM158 | Receptor | Q8WZ71 | 25907 |
| TNF | Ligand | P01375 | 7124 |
| TPM3 | Receptor | P06753 | 7170 |
| VEGFA | Ligand | P15692 | 7422 |
| VEGFB | Ligand | P49765 | 7423 |
| VGF | Ligand | O15240 | 7425 |
| XCR1 | Receptor | P46094 | 2829 |
| ZN274 | Signaling | Q96GC6 | 10782 |

Neurotransmission Modulators

In some embodiments, the neuromodulating agent is a neurotransmission modulator (e.g., an agent that increases or decreases neurotransmission). For example, in some embodiments, the neuromodulating agent is a neurotransmitter or neurotransmitter receptor listed in Table 1A, 1B, Table 7, or Table 8, a modulator of a channel or transporter encoded by a gene in Table 7, or an agonist or antagonist listed in Tables 2A-2K for a corresponding neurotransmitter pathway member. In some embodiments, the neurotransmission modulator is a neurotransmission modulator listed in Table 2M. Neuromodulating agents that increase neurotransmission include neurotransmitters and neurotransmitter receptors listed in Tables 1A, 1B, Table 7, and Table 8 and analogs thereof, and neurotransmitter agonists (e.g., small molecules that agonize a neurotransmitter receptor listed in Table 1A or encoded by a gene in Table 7 or Table 8). Exemplary agonists are listed in Tables 2A-2K. In some embodiments, neurotransmission is increased via administration, local delivery, or stabilization of neurotransmitters (e.g., ligands listed in Tables 1A, 1B, and Table 7). Neurotransmission modulators that increase neurotransmission also include agents that increase neurotransmitter synthesis or release (e.g., agents that increase the activity of a biosynthetic protein encoded by a gene in Table 1A or Table 7 via stabilization, overexpression, or upregulation, or agents that increase the activity of a synaptic or vesicular protein encoded by a gene in Table 7 via stabilization, overexpression, or upregulation), prevent neurotransmitter reuptake or degradation (e.g., agents that block or antagonize transporters encoded by a gene in Table 7 or Table 8 that remove neurotransmitter from the synaptic cleft), increase neurotransmitter receptor activity (e.g., agents that increase the activity of a signaling protein encoded by a gene in Table 1A or Table 7 via stabilization, overexpression, agonism, or upregulation, or agents that upregulate, agonize, or stabilize a neurotransmitter receptor listed in Table 1A or encoded by a gene in Table 7 or Table 8), increase neurotransmitter receptor synthesis or membrane insertion, decrease neurotransmitter degradation, and regulate neurotransmitter receptor conformation (e.g., agents that bind to a receptor and keep it in an “open” or “primed” conformation). In some embodiments, the neurotransmitter receptor is a channel (e.g., a ligand or voltage gated ion channel listed in Table 7 or Table 8), the activity of which can be increased by agonizing, opening, stabilizing, or overexpressing the channel. Neurotransmission modulators that increase neurotransmission further include agents that stabilize a structural protein encoded by a gene in Table 7. Neurotransmission modulators can increase neurotransmission by 10%, 20%, 30%, 40%, 50%, 60%, 70%, 80%, 90%, 95%, 98% or more. Exemplary neurotransmission modulators are listed in Table 2M.

Neuromodulating agents that decrease neurotransmission include neurotransmitter antagonists (e.g., small molecules that antagonize a neurotransmitter receptor listed in Table 1A or Table 7 or Table 8). Exemplary antagonists are listed herein below and in Tables 2A-2K. Neurotransmission modulators that decrease neurotransmission also include agents that decrease neurotransmitter synthesis or release (e.g., agents that decrease the activity of a biosynthetic protein encoded by a gene in Table 1A or Table 7 via inhibition or downregulation, or agents that decrease the activity of a synaptic or vesicular protein encoded by a gene in Table 7 via blocking, disrupting, or downregulating, or antagonizing the protein), increase neurotransmitter reuptake or degradation (e.g., agents that agonize, open, or stabilize transporters encoded by a gene in Table 7 or Table 8 that remove neurotransmitter from the synaptic cleft), decrease neurotransmitter receptor activity (e.g., agents that decrease the activity of a signaling protein encoded by a gene in Table 1A or Table 7 via blocking or antagonizing the protein, or agents that block, antagonize, or downregulate a neurotransmitter receptor listed in Table 1A or encoded by a gene in Table 7 or Table 8), decrease neurotransmitter receptor synthesis or membrane insertion, increase neurotransmitter degradation, regulate neurotransmitter receptor conformation (e.g., agents that bind to a receptor and keep it in a “closed” or “inactive” conformation), and disrupt the pre- or postsynaptic machinery (e.g., agents that block or disrupt a structural protein encoded by a gene in Table 7, or agents that block, disrupt, downregulate, or antagonize a synaptic or vesicular protein encoded by a gene in Table 7). In some embodiments, the neurotransmitter receptor is a channel (e.g., a ligand or voltage gated ion channel listed in Table X or Table X.1), the activity of which can be decreased by blockade, antagonism, or inverse agonism of the channel. Neurotransmission modulators that decrease neurotransmission further include agents that sequester, block, antagonize, or degrade a neurotransmitter listed in Tables 1A, 1B, or encoded by a gene in Table 7. Neurotransmission modulators that decrease or block neurotransmission include antibodies that bind to or block the function of neurotransmitters, neurotransmitter receptor antagonists, and toxins that disrupt synaptic release. Neurotransmission modulators can decrease neurotransmission by 10%, 20%, 30%, 40%, 50%, 60%, 70%, 80%, 90%, 95%, 98% or more.

In some embodiments, the neuromodulating agent is an adrenergic receptor pathway modulator (e.g., a blocker or agonist of an adrenergic receptor listed in Table 1A or Table 7, e.g., an adrenergic blocker or agonist listed in Table 2A or Table 2B); a cholinergic receptor pathway modulator (e.g., a blocker or agonist of a cholinergic receptor listed in Table 1A or Table 7, e.g., a cholinergic blocker or agonist listed in Table 2A, 2E, or 2F); a dopamine receptor pathway modulator (e.g., a blocker or agonist of a dopamine receptor listed in Table 1A or Table 7, e.g., a dopamine blocker or agonist listed in Table 2A or 2C); a serotonin receptor pathway modulator (e.g., a blocker or agonist of a serotonin receptor listed in Table 1A, Table 7, or Table 8, e.g., a serotonin blocker or agonist listed in Table 2A or 2G); a GABA receptor pathway modulator (e.g., a blocker or agonist of a GABA receptor listed in Table 1A, Table 7, or Table 8, e.g., a GABA blocker or agonist listed in Table 2A or 2D); a glutamate receptor pathway modulator (e.g., a blocker or agonist of a glutamate receptor listed in Table 1A, Table 7, or Table 8, e.g., a glutamate blocker or agonist listed in Table 2A or 2H).

TABLE 2A
AGONIST AND ANTAGONIST AGENTS

| Gene | Agonist | Antagonist |
|---|---|---|
| Adrb2 | NCX 950 | Alprenolol |
| Accession Number: | Bitolterol | Carvedilol |
| P07550 | Isoetarine | Desipramine |
| | Norepinephrine | Nadolol |
| | Phenylpropanolamine | Levobunolol |
| | Dipivefrin | Metipranolol |
| | Epinephrine | Bevantolol |
| | Orciprenaline | Oxprenolol |
| | Dobutamine | Nebivolol |
| | Ritodrine | Asenapine |
| | Terbutaline | Bupranolol |
| | Salmeterol | Penbutolol |
| | Formoterol | Celiprolol |
| | Salbutamol | Pindolol |
| | Isoprenaline | Acebutolol |
| | Arbutamine | Bopindolol |
| | Arformoterol | |
| | Fenoterol | |
| | Pirbuterol | |
| | Ephedra | |
| | Procaterol | |
| | Clenbuterol | |
| | Bambuterol | |
| | Indacaterol | |
| | Droxidopa | |
| | Olodaterol | |
| | Vilanterol | |
| | Pseudoephedrine | |
| | Cabergoline | |
| | Mirtazepine | |
| Adra1d | Midodrine | Dapiprazole |
| Accession Number: | Norepinephrine | Amitriptyline |
| P25100 | Clonidine | Alfuzosin |
| | Oxymetazoline | Promazine |
| | Pergolide | Prazosin |
| | Bromocriptine | Imipramine |
| | Droxidopa | Nortriptyline |
| | Xylometazoline | Doxazosin |
| | Ergotamine | Nicardipine |
| | Cirazoline | Dronedarone |
| | Cabergoline | Tamsulosin |
| | Methoxamine | Propiomazine |
| | Epinephrine | Phenoxybenzamine |
| | | Carvedilol |
| | | Doxepin |
| | | Terazosin |
| | | Quetiapine |
| | | Methotrimeprazine |
| | | Silodosin |
| Adrb1 | Isoetarine | Esmolol |
| Accession Number: | Norepinephrine | Betaxolol |
| P08588 | Phenylpropanolamine | Metoprolol |
| | Epinephrine | Atenolol |
| | Dobutamine | Timolol |
| | Salbutamol | Sotalol |
| | Isoprenaline | Propranolol |
| | Arbutamine | Labetalol |
| | Fenoterol | Bisoprolol |
| | Pirbuterol | Alprenolol |
| | Ephedra | Amiodarone |
| | Clenbuterol | Carvedilol |
| | Droxidopa | Nadolol |
| | Pseudoephedrine | Levobunolol |
| | Carteolol | Metipranolol |
| | Cabergoline | Bevantolol |
| | Mirtazapine | Practolol |
| | Loxapine | Oxprenolol |
| | Vortioxetine | Celiprolol |
| | Desipramine | Nebivolol |
| | | Asenapine |
| | | Bupranolol |
| | | Penbutolol |
| | | Pindolol |
| | | Acebutolol |
| | | Bopindolol |
| | | Cartelol |
| Adrb3 | SR 58611 | Bopindolol |
| Accession Number: | Norepinephrine | Propranolol |
| P13945 | Epinephrine | Bupranolol |
| | Isoprenaline | |
| | Arbutamine | |
| | Fenoterol | |
| | Ephedra | |
| | Clenbuterol | |
| | Droxidopa | |
| | Mirabegron | |
| Adrbk1 | ATP | Alprenolol |
| Accession Number: | Carbachol | Heparin |
| P25098 | Dopamine | |
| | Isoproterenol | |
| | Morphine | |
| | DAMGO | |
| | histamine | |
| | Acetylcholine | |
| | Etorphine | |
| | NMDA | |
| | Dopamine | |
| Adrbk2 | Isoproterenol | Propranolol |
| Accession Number: | DAMGO | |
| P26819 | ATP | |
| Chrm3 | cgmp | MT3 |
| Accession Number: | ATP | hexocyclium |
| P20309 | Cevimeline | himbacine |
| | arecoline | Biperiden |
| | oxotremorine-M | lithocholylcholine |
| | NNC 11-1314 | AFDX384 |
| | xanomeline | 4-DAMP |
| | oxotremorine | hexahydrodifenidol |
| | pentylthio-TZTP | VU0255035 |
| | arecaidine propargyl ester | N-methyl scopolamine |
| | NNC 11-1607 | darifenacin |
| | furmethide | Thiethylperazine |
| | NNC 11-1585 | methoctramine |
| | Acetylcholine | silahexocyclium |
| | methylfurmethide | strychnine |
| | Bethanechol | MT7 |
| | Carbachol | Heparin |
| | Succinylcholine | Olanzapine |
| | ALKS 27 | Pirenzepine |
| | itopride | Clidinium |
| | methacholine | Ipratropium |
| | Meperidine | Propantheline |
| | Cinnarizine | Dicyclomine |
| | Trimipramine | Darifenacin |
| | | Tiotropium |
| | | Atropine |
| | | Scopolamine |
| | | Amitriptyline |
| | | Doxepin |
| | | Lidocaine |
| | | Nortriptyline |
| | | Tropicamide |
| | | Metixene |
| | | Homatropine Methylbromide |
| | | Solifenacin |
| | | Glycopyrrolate |
| | | Propiomazine |
| | | Diphemanil Methylsulfate |
| | | Promethazine |
| | | Diphenidol |
| | | Pancuronium |
| | | Ziprasidone |
| | | Quetiapine |
| | | Imipramine |
| | | Clozapine |
| | | Cyproheptadine |
| | | Aripiprazole |
| | | Nicardipine |
| | | Amoxapine |
| | | Loxapine |
| | | Promazine |
| | | Oxyphencyclimine |
| | | Anisotropine Methylbromide |
| | | Tridihexethyl |
| | | Chlorpromazine |
| | | Ketamine |
| | | Cyclosporin A |
| | | Paroxetine |
| | | Benzquinamide |
| | | Tolterodine |
| | | Oxybutynin |
| | | alcuronium |
| | | WIN 62,577 |
| | | Tramadol |
| | | Chlorprothixene |
| | | Aclidinium |
| | | Methotrimeprazine |
| | | Umeclidinium |
| | | Cryptenamine |
| | | Mepenzolate |
| | | Maprotiline |
| | | Brompheniramine |
| | | Isopropamide |
| | | Trihexyphenidyl |
| | | Ipratropium bromide |
| | | Hyoscyamine |
| | | Procyclidine |
| | | Pipecuronium |
| | | Fesoterodine |
| | | Disopyramide |
| | | Desipramine |
| | | Mivacurium |
| Chrna3 | Nicotine | A-867744 |
| Accession Number: | Varenicline | NS1738 |
| P32297 | Acetylcholine | Hexamethonium |
| | Ethanol | Mecamylamine |
| | Cytisine | Dextromethorphan |
| | Levamisole | Pentolinium |
| | Galantamine | Levomethadyl Acetate |
| | | Bupropion |
| Chrna6 | Nicotine | Hexamethonium |
| Accession Number: | Cytisine | Mecamylamine |
| Q15825 | Varenicline | |
| | Galantamine | |
| Chrna9 | Nicotine | Hexamethonium |
| Accession Number: | Galantamine | Mecamylamine |
| Q9UGM1 | Ethanol | Tetraethylammonium |
| | | Muscarine |
| | ATG003 | Strychnine |
| | Lobeline | |
| | RPI-78M | |
| Chrnb1 | Galantamine | |
| Accession Number: | | |
| P11230 | | |
| Chrnb4 | Nicotine | Atropine |
| Accession Number: | Varenicline | Oxybutynin |
| P30926 | PNU-120596 | Pentolinium |
| | Ethanol | Dextromethorphan |
| | Galantamine | |
| Chrng | Galantamine | |
| Accession Number: | | |
| P07510 | | |
| Adcyap1 | Nicotine | Atropine |
| Accession Number: | CGMP | PPADS |
| P18509 | Apomorphine | Onapristone |
| | Suramin | Muscarine |
| | Nifedipine | Haloperidol |
| | ATP | Astressin |
| | Dihydrotestosterone | Melatonin |
| | Maxadilan | Scopolamine |
| | Dexamethasone | Tetrodotoxin |
| | Acetylcholine | Apamin |
| | Histamine | Hexamethonium |
| | Carbachol | Indomethacin |
| | NMDA | Propranolol |
| | Dopamine | Bumetanide |
| | Isoproterenol | Progesterone |
| | Salbutamol | Charybdotoxin |
| | Morphine | Prazosin |
| | Clonidine | |
| | Nimodipine | |
| | 2,6-Diamino-Hexanoic Acid Amide | |
| AVPR1B | dVDAVP | YM 218 |
| Accession Number: | LVP | Tolvaptan |
| P47901 | dAVP | Atosiban |
| | Oxytocin | SSR149415 |
| | Desmopressin | YM 471 |
| | Vasopressin | OH-LVA |
| | Terlipressin | Satavaptan |
| | SR 121463 | |
| | PH-284 | |
| CALCB | Amylin | MK-0974 |
| Accession Number: | | |
| P10092 | | |
| CCK | Omeprazole | devazepide |
| Accession Number: | Caffeine | Methysergide |
| P06307 | Hydrocortisone | Cyproheptadine |
| | cholesterol | Naloxone |
| | lauric acid | lorglumide |
| | Dexamethasone | Atropine |
| | NAADP | Rimonabant |
| | Octreotide | Raclopride |
| | decanoic acid | Nicardipine |
| | Dopamine | Pirenzepine |
| | Vapreotide | Ranitidine |
| | Acetylcholine | chenodeoxycholic |
| | histamine | Dexloxiglumide |
| | Carbachol | Tetrodotoxin |
| | Bethanechol | Diltiazem |
| | Tegaserod | Aspirin |
| | Cisapride | Fenfluramine |
| | Morphine | Cyclosporin A |
| CPE | Dopamine | |
| Accession Number: | Insulin, porcine | |
| P16870 | Insulin Regular | |
| CRHBP | Hydrocortisone | Progesterone |
| Accession Number: | Dexamethasone | Astressin |
| P24387 | Sauvagine | |
| CYSLTR1 | Salbutamol | Montelukast |
| Accession Number: | Dexamethasone | Zafirlukast |
| Q9Y271 | Arachidonic acid | Cinalukast |
| | Histamine | Pranlukast |
| | | Nedocromil |
| | | Theophylline |
| | | Indomethacin |
| | | Zileuton |
| | | iralukast |
| | | pobilukast |
| | | sulukast |
| | | verlukast |
| GAL | CGMP | Colchicine |
| Accession Number: | ATP | Naloxone |
| P22466 | Capsaicin | Atropine |
| | Galnon | Melatonin |
| | Dexamethasone | Tetrodotoxin |
| | Levodopa | Reserpine |
| | Acetylcholine | Glyburide |
| | Hexarelin | tetraethylammonium |
| | Histamine | |
| | Carbachol | |
| | Octreotide | |
| | Dopamine | |
| | Salbutamol | |
| | Morphine | |
| | Clonidine | |
| | Bromocriptine | |
| GALR3 | galanin | C7 |
| Accession Number: | galanin-like peptide | M15 |
| O60755 | galnon | M32 |
| | J18 (galanin analogue) | M35 |
| | J20 (galanin analogue) | M40 |
| | M1145 | M871 |
| | M1151 | SNAP 37889 |
| | M1152 | SNAP 398299 |
| | M1153 | |
| | M1160 | |
| | M617 | |
| GRP | Nicotine | Tamoxifen |
| Accession Number: | Diazoxide | Atropine |
| P11021 | Genistein | Pirenzepine |
| | Nifedipine | Cetrorelix |
| | Capsaicin | BIM 23127 |
| | Caffeine | Tetrodotoxin |
| | Hydrocortisone | Aspirin |
| | Dexamethasone | Glyburide |
| | NAADP | Propranolol |
| | Isoproterenol | Indomethacin |
| | NMDA | |
| | ranatensin | |
| | Bombesin | |
| | Bethanechol | |
| | Octreotide | |
| | Acetylcholine | |
| | phyllolitorin | |
| | Carbachol | |
| | Dopamine | |
| | histamine | |
| | arachidonic acid | |
| LTB4R | LTB | U75302 |
| Accession Number: | ATP | CP105696 |
| Q15722 | Dexamethasone | CP-195543 |
| | cholesterol | etalocib |
| | 20-hydroxy-LTB< | SC-41930 |
| | 12R-HETE | LY255283 |
| | arachidonic acid | Zafirlukast |
| | | ONO-4057 |
| | | RO5101576 |
| | | BILL 260 |
| NMU | EUK2010 | R-PSOP |
| Accession Number: | EUK2011 | |
| Q9GZQ4 | EUK2012 | |
| NPM | Ornithine | Dipyridamole |
| Accession Number: | ATP | |
| P06748 | | |
| NPBWR1 | NPW-23 | CYM50769 |
| Accession Number: | des-Br-NPB-23 | |
| P48145 | Ava3 | |
| | Ava5 | |
| | des-Br-neuropeptide B-23 | |
| | des-Br-neuropeptide B-29 | |
| | neuropeptide B-29 | |
| | neuropeptide W-23 | |
| | neuropeptide W-30 | |
| NPW | Neuropeptide W-23 | |
| Accession Number: | | |
| Q8N729 | | |
| NPY | Nicotine | BIIE0246 |
| Accession Number: | Nifedipine | Heparin |
| P01303 | Capsaicin | Theophylline |
| | ATP | Sulpiride |
| | Prednisolone | BIBO3304 |
| | Bethanechol | BIBP3226 |
| | Phenylephrine | Atropine |
| | Acetylcholine | Naltrexone |
| | histamine | Yohimbine |
| | Octreotide | Phentolamine |
| | kainate | Haloperidol |
| | Muscimol | Tetrodotoxin |
| | NMDA | phencyclidine |
| | Carbachol | Fenfluramine |
| | Methoxamine | Indomethacin |
| | Isoproterenol | Reserpine |
| | arpromidine | Prazosin |
| | Dopamine | |
| | Clonidine | |
| | Pilocarpine | |
| | L-Tyrosinamide | |
| | 2-Aminoisobutyric acid | |
| NPY5R | PYY3-36 | L-152,804 |
| Accession Number: | NPY-(18-36) | BIBP3226 |
| Q15761 | PYY | Velneperit |
| | NPY | FMS586 |
| | histamine | BIIE0246 |
| | PYY-(3-36) | |
| | PYY-(3-36) | |
| | pancreatic polypeptide | |
| | Acetylcholine | |
| | AC162352 | |
| NTS | Apomorphine | Olanzapine |
| Accession Number: | oleic acid | Melatonin |
| Q6FH20 | Nicotine | Yohimbine |
| | Nifedipine | Thioridazine |
| | Capsaicin | Diphenhydramine |
| | ATP | Haloperidol |
| | Dexamethasone | SR142948A |
| | Levodopa | Sulpiride |
| | triamcinolone acetonide | Naloxone |
| | neurotensin | Heparin |
| | QUINPIROLE | Atropine |
| | kainate | Clozapine |
| | histamine | apamin |
| | Carbachol | Tetrodotoxin |
| | NMDA | Anandamide |
| | Isoproterenol | Chlorpromazine |
| | Acetylcholine | phencyclidine |
| | Dopamine | Reserpine |
| | Dexmedetomidine | Indomethacin |
| | Octreotide | Bumetanide |
| | Levocabastine | |
| | Clonidine | |
| | Morphine | |
| | arachidonic acid | |
| NTSR1 | JMV2004 | Meclinertant |
| Accession Number: | JMV431 | SR142948A |
| P30989 | JMV457 | |
| | JMV458 | |
| | Levocabastine | |
| | large neuromedin N | |
| | large neurotensin | |
| | Dopamine | |
| | neurotensin | |
| | contulakin-G | |
| | KH28 | |
| PENK | Dopamine | Naltrexone |
| Accession Number: | kainate | Naloxone |
| P01210 | NMDA | Progesterone |
| | DAMGO | |
| | Morphine | |
| PNOC | Capsaicin | Atropine |
| Accession Number: | ACONITINE | Naloxone |
| Q13519 | Etorphine | Naltrexone |
| | histamine | Buprenorphine |
| | NMDA | naltrindole |
| | Acetylcholine | hexamethonium |
| | Dopamine | Anandamide |
| | DAMGO | DPDPE |
| | Morphine | Progesterone |
| PTH2R | Ostabolin-C | PTH-(7-34) |
| Accession Number: | Teriparatide | PTHrP-(5-36) |
| P49190 | Preotact | TIP39 |
| SCG2 | ATP | |
| Accession Number: | Capsaicin | |
| P13521 | Dexamethasone | |
| | histamine | |
| | Acetylcholine | |
| | Dopamine | |
| SCG5 | ATP | Farnesyl diphosphate |
| Accession Number: | Cholesterol | Heparin |
| P05408 | Dexamethasone | |
| | NMDA | |
| | histamine | |
| SSTR1 | CST-14 | |
| Accession Number: | Octreotide | |
| P30872 | Pasireotide | |
| SSTR2 | Octreotide | Progesterone |
| Accession Number: | Dopamine | Tamoxifen |
| P30874 | | Pasireotide |
| UCN2 | Dopamine | |
| Accession Number: | Acetylcholine | |
| Q96RP3 | histamine | |
| | Sauvagine | |
| UTS2 | lysophosphatidylcholine | palosuran |
| Accession Number: | cholesterol | Atropine |
| O95399 | Acetylcholine | Tetrodotoxin |
| | Phenylephrine | Indomethacin |
| | | Verapamil |
| VIP | cgmp | Melatonin |
| Accession Number: | Suramin | Sumatriptan |
| P01282 | Capsaicin | Phentolamine |
| | Capsaicin | Naloxone |
| | Caffeine | Haloperidol |
| | Nifedipine | Astressin |
| | cromakalim | Atropine |
| | maxadilan | Tetrodotoxin |
| | Dexamethasone | apamin |
| | Prednisolone | hexamethonium |
| | BAY 55-9837 | Amiloride |
| | Dihydrotestosterone | Thiorphan |
| | carbacyclin | Indomethacin |
| | Citrulline | Cyclosporin |
| | Carbachol | Verapamil |
| | histamine | Propranolol |
| | Dopamine | Bumetanide |
| | Phenylephrine | Captopril |
| | Acetylcholine | Sildenafil |
| | Octreotide | Glyburide |
| | Methoxamine | tetraethylammonium |
| | Isoproterenol | |
| | Ephedrine | |
| | Salbutamol | |
| | Bromocriptine | |
| | Morphine | |
| VIPR1 | PHM | Secretin |
| Accession Number: | BAY 55-9837 | neurotensin-(6-11)/VIP-(7-28) |
| P32241 | helodermin | |
| | PACAP-27 | |
| | VIP | |
| | PACAP-38 | |
| | PG 99-465 | |
| | PHI | |
| | PHI | |
| | PHV | |
| | Ro 25-1392 | |
| Htr2c | Apomorphine | Melatonin |
| Accession Number: | Bifeprunox | SB 224289 |
| P28335 | Tramadol | LY334362 |
| | AL-37350A | FR260010 |
| | 5-MeO-DMT | Sulpiride |
| | BW723C86 | Thiethylperazine |
| | CGS-12066 | cyamemazine |
| | DOI | mesulergine |
| | 5-CT | SB 221284 |
| | YM348 | zotepine |
| | LSD | Metergoline |
| | xanomeline | methiothepin |
| | WAY-163909 | spiperone |
| | Dopamine | SB 215505 |
| | LY344864 | tiospirone |
| | VER-3323 | SB 228357 |
| | TFMPP | pizotifen |
| | 8-OH-DPAT | SB 206553 |
| | MK-212 | SB 204741 |
| | NMDA | SDZ SER-082 |
| | org 12962 | Ritanserin |
| | 5-MeOT | SB 242084 |
| | RU 24969 | S33084 |
| | Acetylcholine | roxindole |
| | QUINPIROLE | RS-127445 |
| | quipazine | terguride |
| | tryptamine | EGIS-7625 |
| | Ro 60-0175 | SB 243213 |
| | Oxymetazoline | RS-102221 |
| | Ergotamine | Olanzapine |
| | Cabergoline | Aripiprazole |
| | Lorcaserin | Agomelatine |
| | Pergolide | Ziprasidone |
| | Methylergonovine | Quetiapine |
| | Renzapride | Sarpogrelate |
| | Pramipexole | Perphenazine |
| | GR-127935 | Thioridazine |
| | BRL-15572 | Sertindole |
| | ipsapirone | Loxapine |
| | SB 216641 | Methysergide |
| | SL65.0155 | Risperidone |
| | S 16924 | Asenapine |
| | Bromocriptine | Mianserin |
| | Lisuride | Clozapine |
| | Tegaserod | Trifluoperazine |
| | Epicept NP-1 | Trazodone |
| | dapoxetine | Doxepin |
| | Dexfenfluramine | Nortriptyline |
| | 3,4-Methylenedioxymethamphetamine | Chlorprothixene |
| | Ropinirole | Minaprine |
| | Maprotiline | Propiomazine |
| | Desipramine | Mirtazapine |
| | | Amoxapine |
| | | Yohimbine |
| | | Cyproheptadine |
| | | Imipramine |
| | | Amitriptyline |
| | | Promazine |
| | | Chlorpromazine |
| | | Ketamine |
| | | Propranolol |
| | | Fluoxetine |
| | | Ketanserin |
| | | mesulergine |
| | | AC-90179 |
| | | Ergoloid mesylate 2 |
| | | Methotrimeprazine |
| | | Paliperidone |
| | | Clomipramine |
| | | Trimipramine |
| | | Captodiame |
| | | Nefazodone |
| GABA Receptor | Bamaluzole | bicuculline |
| Accession Numbers | GABA | metrazol |
| (Q9UBS5, O95166, | Gabamide | flumazenil |
| O75899, P28472, P18507, | GABOB | thiothixine |
| P47870, P47869, O14764) | Gaboxadol | bupropion |
| | Ibotenic acid | caffeine |
| | Isoguvacine | |
| | Isonipecotic acid | |
| | Muscimol | |
| | Phenibut | |
| | Picamilon | |
| | Progabide | |
| | Quisqualamine | |
| | SL 75102 | |
| | Thiomuscimol | |
| | Alcohols (e.g., ethanol, isopropanol) | |
| | Avermectins (e.g., ivermectin) | |
| | Barbiturates (e.g., phenobarbital) | |
| | Benzodiazepines | |
| | Bromides (e.g., potassium bromide | |
| | Carbamates (e.g., meprobamate, | |
| | carisoprodol) | |
| | Chloralose | |
| | Chlormezanone | |
| | Clomethiazole | |
| | Dihydroergolines (e.g., ergoloid | |
| | (dihydroergotoxine)) | |
| | Etazepine | |
| | Etifoxine | |
| | Imidazoles (e.g., etomidate) | |
| | Kavalactones (found in kava) | |
| | Loreclezole | |
| | Neuroactive steroids (e.g., | |
| | allopregnanolone, ganaxolone) | |
| | Nonbenzodiazepines (e.g., | |
| | zaleplon, zolpidem, zopiclone, | |
| | eszopiclone) | |
| | Petrichloral | |
| | Phenols (e.g., propofol) | |
| | Piperidinediones (e.g., glutethimide, | |
| | methyprylon) | |
| | Propanidid | |
| | Pyrazolopyridines (e.g., etazolate) | |
| | Quinazolinones (e.g., | |
| | methaqualone) | |
| | Skullcap constituents | |
| | Stiripentol | |
| | Sulfonylalkanes (e.g., | |
| | sulfonmethane, tetronal, trional) | |
| | Valerian constituents (e.g., valeric | |
| | acid, valerenic acid) | |
| | Volatiles/gases (e.g., chloral | |
| | hydrate, chloroform, diethyl ether, | |
| | sevoflurane) | |
| Glutamate Receptor | 3,5-dihydroxyphenylglycine | APICA |
| Accession Number: | eglumegad | EGLU |
| (P42261, P39086, | Biphenylindanone A | LY-341,495 |
| P39086, Q13585, P42261, | DCG-IV | |
| P42262, P42263, P48058, | L-AP4 | |
| P39086, Q13002, | | |
| Q13003, Q13003, | | |
| Q16478, Q12879, | | |
| Q14957, Q13224, | | |
| Q14957, O15399, | | |
| Q8TCU5, O60391) | | |
| CNR1/CNR2 | N-Arachidonoylethanolamine | SR 141716A |
| Accession Number: | 2-Arachidonoyl-glycerol | LY-320135 |
| (P21554, P34972) | 2-Arachidonoyl-glycerylether | AM251 |
| | N-Arachidonoyl-dopamine | AM281 |
| | O-Arachidonoyl-ethanolamine | SR 144528 |
| | N-Arachidonoylethanolamine | AM630 |
| | 2-Arachidonoyl-glycerol | |
| | 2-Arachidonoyl-glycerylether | |
| | N-Arachidonoyl-dopamine | |
| | O-Arachidonoyl-ethanolamine | |
| | Δ 9-THC | |
| | CP-55,940 | |
| | R(+)-WIN 55,212-2 | |
| | HU-210 | |
| | Levonantradol | |
| | Nabilone | |
| | Methanandamide | |
| | ACEA | |
| | O-1812 | |
| | Δ9-THC | |
| | CP-55,940 | |
| | R(+)-WIN 55,212-2 | |
| | HU-210 | |
| | Levonantradol | |
| | Nabilone | |
| | Methanandamide | |
| | JWH-015 | |
| | JWH-133 | |

TABLE 2B
ADRENERGIC AGONISTS AND ANTAGONISTS

| Receptor | Agonist | Antagonist |
|---|---|---|
| Non-selective | Adrenaline (epinephrine), | Carvedilol, arotinolol, and |
| | Noradrenaline (norepinephrine), | labetalol |
| | Isoprenaline (isoproterenol), | |
| | dopamine, caffeine, nicotine, | |
| | tyramine, methylphenidate, | |
| | ephedrine and pseudophedrine. | |
| α1 selective | Phenylephrine, methoxamine, | Acepromazine, alfuzosin, |
| (ADRA1A, ADRA1B, | midodrine, cirazoline, | doxazosin, labetalol, |
| ADRA1D) | Xylometazoline, metaraminol | phenoxybenzamine, KW3902, |
| | Chloroehtylclonidine, | phentolamine, prazosin, |
| | oxymetazoline | tamsulosin, terazosin, tolazoline, |
| | | trazodone, amitriptyline, |
| | | silodosin, clomipramine, doxepin, |
| | | trimipramine, typical and atypical |
| | | antipsychotics, and |
| | | antihistamines, such as |
| | | hyroxyzine |
| α2 selective | A-methyl dopa, clonidine, | Phentolamine, |
| (ADRA2A, ADRA2B, | Brimonidine, agmatine, | phenoxybenzamine, yohimbine, |
| ADRA2C) | Dexmedetomidine, | idazoxan, atipamezole, |
| | Medetomidine, romifidine | mirtazapine, tolazoline, |
| | Chloroethylclonidine, | trazodone, and typical and |
| | Detomidine, lofexidine, xylazine, | atypical antipsychotics |
| | Tizanidine, guanfacine, and | |
| | amitraz | |
| β1 selective | Dobutamine | Metroprolol, atenolol, acebutolol, |
| (ADRB1) | | bisoprolol, betaxolol, |
| | | levobetaxolol, esmolol, celiprolol, |
| | | carteolol, landiolol, oxprenolol, |
| | | propanolol, practolol, penbutolol, |
| | | timolol, labetalol, nebivolol, |
| | | levobunolol, nadolol, pindolol, |
| | | sotalol, metipranolol, tertatolol, |
| | | vortioxene |
| β2 selective | Salbutamol, albuterol, bitolterol | Butaxamine, acebutolol, timolol, |
| (ADRB2) | mesylate, levabuterol, ritodrine, | propanolol, levobunolol, |
| | metaproterenol, terbutaline, | carteolol, labetalol, pindolol, |
| | salmeterol, formoterol, and | oxprenolol, nadolol, metipranolol, |
| | pirbuterol | penbutolol, tertatolol, sotalol |
| β3 selective | L-796568, amibegron, | SR 59230A, arotinolol |
| (ADRB3) | solabegron, mirabegron | |

TABLE 2C
DOPAMINE AGONISTS AND ANTAGONISTS

| Receptor | Agonist | Antagonist |
|---|---|---|
| Non-selective | Pramipexole, ropinirole, | Haloperidol, paliperidone, |
| | rotigotine, apomorphine, | clozapine, risperidone, |
| | propylnorapomorphine, | olanzapine, quetiapine, |
| | bromocriptine, cabergoline, | ziprasidone, metoclopramide, |
| | ciladopa, dihydrexidine, | droperidol, dromperidone, |
| | dinapsoline, doxamthrine, | amoxapine, clomipramine, |
| | epicriptine, lisuride, pergolide, | trimipramine, choline, melatonin, |
| | piribedil, quinagolide, roxindole, | acepromazine, amisulpride, |
| | dopamine | asenapine, azaperone, |
| | | benperidol, bromopride, |
| | | butaclamol, chlorpromazine, |
| | | chlorprothixene, clopenthixol, |
| | | eticlopride, flupenthixol, |
| | | fluphenazine, fluspirilene, |
| | | hydroxyzine, iodobenzamide, |
| | | levomepromazine, loxapine, |
| | | mesoridazine, nafadotride, |
| | | nemonapride, penfluridol, |
| | | perazine, perphenazine, |
| | | pimozide, prochlorperazine, |
| | | promazine, raclopride, |
| | | remoxipride, spiperone, |
| | | spiroxatrine, stepholidine, |
| | | sulpiride, suitopride, |
| | | tetrahydropalmatine, |
| | | thiethylperazine, thioridazine, |
| | | thiothixene, tiapride, |
| | | trifluoperazine, trifluperidol, |
| | | triflupromazine, and ziprasidone |
| D1 (DRD1) | Fenoldopam, A-86929, | Sch-23,390, skf-83,959, |
| | dihydrexidine, dinapsoline, | ecopipam |
| | dinoxyline, doxanthrine, SKF- | |
| | 81297, SKF-82958, SKF-38393, | |
| | G-BR-APB, dopexamine | |
| D2 (DRD2) | Cabergoline, pergolide, | Chloroethylnorapomorphine, |
| | quinelorane, sumanirole, | desmethoxyfallypride, |
| | talipexole, piribedil, quinpirole, | domperidone, eticlopride, |
| | quinelorane, dinoxyline, | fallypride, hydroxyzine, itopride, |
| | dopexamine | L-741,626, SV 293, yohimbine, |
| | | raclopride, sulpiride, |
| D3 (DRD3) | Piribedil, quinpirole, captodiame, | Domperidone, FAUC 365, |
| | compound R, R-16, FAUC 54, | nafadotride, raclopride, PNU- |
| | FAUC 73, PD-128,907, PF- | 99,194, SB-277011-A, sulpiride, |
| | 219,061, PF-592,379, CJ-1037, | risperidone, YQA14, U99194, SR |
| | FAUC 460, FAUC 346, | 21502 |
| | cariprazine | |
| D4 (DRD4) | Way-100635, a-412,997, abt- | A-381393, FAUC 213, L- |
| | 724, abt-670, fauc 316, pd-168, | 745,870, L-570,667, ML-398, |
| | 077, cp-226,269 | fananserin, clozapine |
| D5 (DRD5) | Dihydrexidine, rotigotine, SKF- | Sch 23390 |
| | 83,959, fenoldopam, | |
| Partial | Aplindore, brexpiprazole, | |
| | aripiprazole, CY-208,243, | |
| | pardoprunox, phencyclidine, and | |
| | salvinorin A | |

TABLE 2D
GABA AGONISTS AND ANTAGONISTS

| Receptor | Agonist | Antagonist |
|---|---|---|
| GABA_{A} | Barbiturates (e.g., allobarbital, | Bicuculline, gabazine, hydrastine, |
| | amobarbital, aprobarbital, | pitrazepin, sinomenine, tutin, |
| | alphenal, barbital, brallobarbital, | thiocolchicoside, metrazol, |
| | phenobarbital, secobarbital, | securinine, gabazine |
| | thiopental), bamaluzole, gaba, | |
| | gabob, gaboxadol, ibotenic acid, | |
| | isoguvacine, isonipecotic acid, | |
| | muscimol, phenibut, picamilon, | |
| | progabide, quisqualamine, sl | |
| | 75102, thiomuscimol, positive | |
| | allosteric modulators (pams) | |
| | (e.g., alcohols, such as ethanol | |
| | and isopropanol; avermectins, | |
| | such as ivermectin; | |
| | benzodiazepines, such as | |
| | diazepam, alprazolam, | |
| | chlordiazepoxide, clonazepam, | |
| | flunitrazepam, lorazepam, | |
| | midazolam, oxazepam, | |
| | prazepam, brotizolam, triazolam, | |
| | estazolam, lormetazepam, | |
| | nitrazepam, temazepam, | |
| | flurazepam, clorazepate | |
| | halazepam, prazepam, | |
| | nimetazapem, adinazolam, and | |
| | climazolam; bromides, such as | |
| | potassium bromide; carbamates, | |
| | such as meprobamate and | |
| | carisoprodol; chloralose; | |
| | chlormezanone; chlomethiazole; | |
| | dihydroergolines, such as | |
| | ergoloid; etazepine; etifoxine; | |
| | imidazoles, such as etomidate; | |
| | imidazopyridines, such as | |
| | alpidem and necopdiem; | |
| | kavalactones; loreclezole; | |
| | neuroactive steroids, such as | |
| | allogregnanolone, pregnanolone, | |
| | dihydrodeoxycorticosterone, | |
| | tetrahydrodeoxycortisosterone, | |
| | androstenol, androsterone, | |
| | etiocholanolone, 3α- | |
| | androstanediol, 5α, 5β, or 3α- | |
| | dihydroprogesterone, and | |
| | ganaxolone; | |
| | nonbenzodiazepines, such as | |
| | zalepon, zolpidem, zopiclone, | |
| | and eszopiclone; petrichloral; | |
| | phenols, such as propofol; | |
| | piperidinediones, such as | |
| | glutethimide and methyprylon; | |
| | propanidid; pyrazolopyridines, | |
| | such as etazolate; | |
| | pyrazolopyrimidines, such as | |
| | divaplon and fasiplon; | |
| | cyclopyrrolones, sush as | |
| | pagoclone and suproclone; β- | |
| | cabolines, such as abecarnil and | |
| | geodecarnil; quinazolinones, | |
| | such as methaqualone; | |
| | scutellaria constituents; | |
| | stiripentol; sulfonylalkanes, such | |
| | as sulfonomethane, teronal, and | |
| | trional; valerian constituents, | |
| | such as valeric acid and | |
| | valerenic acid; and gases, such | |
| | as chloral hydrate, chloroform, | |
| | homotaurine, diethyl ether, and | |
| | sevoflurane. | |
| GABA_{B} | 1,4-butanediol, baclofen, GABA, | CGP-35348, homotaurine, |
| | Gabamide, GABOB, gamma- | phaclofen, saclofen, and SCH- |
| | butyrolactone, gamma- | 50911 |
| | hydroxybutyric acid, gamma- | |
| | hyrdoxyvaleric acid, gamma- | |
| | valerolactone, isovaline, | |
| | lesogaberan, phenibut, | |
| | picamilon, progabide, | |
| | homotaurine, SL-75102, | |
| | tolgabide | |
| GABA_{A}-ρ | CACA, CAMP, GABA, GABOB, | Gabazine, gaboxadol, |
| | N4-chloroacetylcytosine | isonipecotic acid, SKF-97,541, |
| | arabinoside, picamilon, | and (1,2,5,6-Tetrahydropyridin-4- |
| | progabide, tolgabide, and | yl)methylphosphinic acid |
| | neuroactive steroids, such as | |
| | allopregnanolone, THDOC, and | |
| | alphaxolone | |

TABLE 2E
MUSCARINC AGONISTS AND ANTAGONISTS

| Receptor | Agonist | Antagonist |
|---|---|---|
| Chrm1 | AF102B, AF150(S), AF267B, | Atropine, dicycloverine, |
| | acetylcholine, carbachol, | hyoscyamine, ipratropium, |
| | cevimeline, muscarine, | mamba toxin muscarinic toxin 7 |
| | oxotremorine, pilocarpine, | (MT7)^{,} olanzapine, oxybutynin, |
| | vedaclidine, 77-LH-28-1, CDD- | pirenzepine, telenzepine, and |
| | 0097, mcn-A-343, L689,660, and | tolterodine |
| | xanomeline | |
| Chrm2 | Acetylcholine, methacholine, | Atropine, dicycloverine, |
| | iper-8-naph, berbine, and | hyoscyamine, otenzepad, aqra- |
| | (2S,2′R,3′S,5′R)-1-methyl-2-(2- | 741, afdx-384, thorazine, |
| | methyl-1,3-oxathiolan-5- | diphenhydramine, |
| | yl)pyrrolidine 3-sulfoxide methyl | dimenhydrinate, ipratropium, |
| | iodide | oxybutynin, pirenzepine, |
| | | methoctramine, tripitramine, |
| | | gallamine, and tolterodine |
| Chrm3 | Acetylcholine, bethanechol, | Atropine, dicycloverine, |
| | carbachol, L689, 660, | hyoscyamine, alcidium bromide, |
| | oxotremorine, pilocarpine, | 4-damp, darifenacin, dau-5884, |
| | aceclidine, arecoline, and | hl-031,120, ipratropium, j- |
| | cevimeline | 104,129, oxybutynin, tiotropium, |
| | | zamifenacin, and tolterodine |
| Chrm4 | Acetylcholine, carbachol, and | Afdx-384, dicycloverine, |
| | oxotremorine), and Chrm5 | himbacine, mamba toxin 3, pd- |
| | agonists (e.g., acetylcholine, | 102,807, pd-0298029, and |
| | milameline, sabcomeline | tropicamide |
| Chrm5 | Acetylcholine, milameline, | Vu-0488130, xanomeline |
| | sabcomeline | |
| Non-selective | | Scopolamine, hydroxyzine, |
| | | doxylamine, dicyclomine, |
| | | flavoxate, cyclopentolate, |
| | | atropine methonitrate, |
| | | trihexyphenidyl/benzhexol, |
| | | solifenacin, benzatropine, |
| | | mebeverine, and procyclidine |

TABLE 2F
NICOTINIC AGONISTS AND ANTAGONISTS

| Receptor | Agonist | Antagonist |
|---|---|---|
| Chrna receptors | Choline, acetylcholine, | Turbocurarine, bupropion, |
| | carbachol, methacholine, | mecamylamine, 18- |
| | nicotine, varenicline tartrate, | methozycoronaridine, |
| | galantamine hydrobromide, | hexamethonium, trimethaphan, |
| | suxamethonium chloride | atraciurium, doxacurium, |
| | (succinylcholine chloride), | mivacurium, pancuronium, |
| | epibatidine, iobeline, | vecuronium, succinylcholine, |
| | decamethonium, | dextromethorphan, neramexane, |
| | isopronicline/TC-1734/AZD3480 | dextrophan, and 3- |
| | (TC-1734), AZD1446 (TC-6683), | methoxymorphinan |
| | TC-5619, TC-5214, MEM 3454 | |
| | (RG3487), ABT-894, ABT-560, | |
| | EVP-6124, EVP-4473, PNU- | |
| | 282987, AR-R17779, SSR | |
| | 189711, JN403, ABBF, PHA- | |
| | 543613, SEN12333, GTS- | |
| | 21/DMXB-A, AZD0328, A- | |
| | 582941, ABT-418, 5-iodo-A- | |
| | 85380, SIB-1765F, ABT-089, and | |
| | ABT-594 | |

TABLE 2G
SEROTONIN AGONISTS AND ANTAGONISTS

| Receptor | Agonist | Antagonist |
|---|---|---|
| 5-HT_{1A} | Azapirones, such as alnespirone, | Pindolol, tertatolol, alprenolol, |
| | binosperone, buspirone, | AV-965, BMY-7,378, |
| | enilospirone, etapirone, geprione, | cyanopindolol, dotarizine, |
| | ipsaprione, revospirone, | flopropione, GR-46,611, |
| | zalospirone, perospirone, | iodocyanopindolol, isamoltane, |
| | tiosperone, umespirone, and | lecozotan, mefway, methiothepin, |
| | tandospirone; 8-OH-DPAT, | methysergide, MPPF, NAN-190, |
| | befiradol, F-15,599, lesopitron, | oxprenolol, pindobind, |
| | MKC-242, LY-283,284, | propanolol, risperidone, |
| | osemozotan, repinotan, U- | robalzotan, SB-649,915, SDZ- |
| | 92,016-A, RU-24969, 2C-B, 2C- | 216,525, spiperone, spiramide, |
| | E, 2C-T-2, aripiprazole, | spiroxatrine, UH-301, WAY- |
| | asenapine, bacoside, befiradol, | 100,135, WAY-100,635, and |
| | brexpiprazole, bufotenin, | xylamidine |
| | cannabidiol, and fibanserin | |
| 5-HT_{1B} | Triptans, such as sumatriptan, | Methiothepin, yohimbine, |
| | rizatriptan, eletriptan, donitripatn, | metergoline, aripiprazole, |
| | almotriptan, frovatriptan, | isamoltane, AR-A000002, SB- |
| | avitriptan, zolmitriptan, and | 216,641, SB-224,289, GR- |
| | naratriptan; ergotamine, 5- | 127,935, SB-236,057 |
| | carboxamidotryptamine, CGS- | |
| | 12066A, CP-93,129, CP-94,253, | |
| | CP-122,288, CP-135,807, RU- | |
| | 24969, vortioxetine, ziprasidone, | |
| | and asenapine | |
| 5-HT_{1D} | Triptans, such as sumatriptan, | Ziprasidone, methiothepin, |
| | rizatriptan, and naratriptan; | yohimbine, metergoline, |
| | ergotamine, 5- | ergotamine, BRL-15572, |
| | (nonyloxy)tryptaime, 5-(t-butyl)- | vortioxetine, GR-127,935, LY- |
| | N-methyltryptamine, CP-286,601, | 310,762, LY-367,642, LY- |
| | PNU-109,291, PNU-142,633, | 456,219, and LY-456,220 |
| | GR-46611, L-694,247, L- | |
| | 772,405, CP-122,288, and CP- | |
| | 135,807 | |
| 5-HT_{1E} | BRL-54443, eletriptan | |
| 5-HT_{1F} | LY-334,370, 5-n-butyryloxy-DMT, | |
| | BRL-54443, eletriptan, LY- | |
| | 344,864, naratriptan, and | |
| | lasmiditan | |
| 5-HT_{2A} | 25I-NBOH, 25I-nbome, (R)-DOI, | Cyproheptadine, methysergide, |
| | TCB-2, mexamine, O-4310, | quetiapine, nefazodone, |
| | PHA-57378, OSU-6162, 25CN- | olanzapine, asenapine, pizotifen, |
| | NBOH, juncosamine, efavirenz, | LY-367,265, AMDA, hydroxyzine, |
| | mefloquine, lisuride, and 2C-B | 5-meo-nbpbrt, and niaprazine |
| 5-HT_{2B} | Fenfluramine, pergolide, | Agomelatine, aripiprazole, |
| | cabergoline, mefloquine, BW- | sarpogrelate, lisuride, tegaserod, |
| | 723C86, Ro60-0175, VER-3323, | metadoxine, RS-127,445, SDZ |
| | 6-APB, guanfacine, | SER-082, EGIS-7625, PRX- |
| | norfenfluramine, 5-meo-DMT, | 08066, SB-200,646, SB-204,741, |
| | DMT, mcpp, aminorex, | SB-206,553, SB-215,505, SB- |
| | chlorphentermine, MEM, MDA, | 228,357, LY-266,097, and LY- |
| | LSD, psilocin, MDMA | 272,015 |
| 5-HT_{2C} | Lorcaserin, lisuride, A-372,159, | Agomelatine, CPC, eltoprazine, |
| | AL-38022A, CP-809,101, | etoperidone, fluoxetine, FR- |
| | fenfluramine, mesulergine, MK- | 260,010, LU AA24530, |
| | 212, naphthyllisopropylamine, | methysergide, nefazodone, |
| | norfenfluramine, ORG-12,962, | norfluoxetine, O- |
| | ORG-37,684, oxaflozane, PNU- | desmethyltramadol, RS-102,221, |
| | 22395, PNU-181731, | SB-200,646, SB-221,284, SB- |
| | lysergamides, phenethylamines, | 242,084, SDZ SER-082, |
| | piperazines, tryptamines, Ro60- | tramadol, and trazodone |
| | 0175, vabicaserin, WAY-629, | |
| | WAY-161,503, WAY-163,909, | |
| | and YM-348 | |
| 5-HT_{2A/2C} | | Ketanserin, risperidone, |
| | | trazodone, mirtazapine, |
| | | clozapine |
| 5-HT₃ | 2-methyl-5-HT, alpha- | Dolasetron, granisetron, |
| | methyltryptamine, bufotenin, | ondansetron, palonosetron, |
| | chlorophenylbiguanide, ethanol, | tropisetron, alosetron, |
| | ibogaine, phenylbiguanide, | cilanosetron, mirtazapine, AS- |
| | quipazine, RS-56812, SR-57227, | 8112, bantopride, |
| | varenicline, and YM-31636 | metroclopramide, renzapride, |
| | | zacopride, mianserin, |
| | | vortioxetine, clozapine, |
| | | olanzapine, quetiapine, menthol, |
| | | thujone, lamotigrine, and 3- |
| | | tropanyl indole-3-carboxylate |
| 5-HT₄ | Cisapride, tegaserod, | Piboserod, GR-113,808, GR- |
| | prucalopride, BIMU-8, CJ- | 125,487, RS-39604, SB-203,186, |
| | 033,466, ML-10302, mosapride, | SB-204,070, and chamomile |
| | renzapride, RS-67506, RS- | |
| | 67333, SL65.1055, zacopride, | |
| | metoclopramide, and sulpride | |
| 5-HT_{5A} | Valeronic acid | ASP-5736, AS-2030680, AS- |
| | | 2674723, latrepiridine, |
| | | risperidone, and SB-699,551 |
| 5-HT₆ | EMDT, WAY-181,187, WAY- | ALX-1161, AVN-211, BVT-5182, |
| | 208,466, N-(inden-5- | BVT-74316, cerlapiridine, EGIS- |
| | yl)imidazothiazole-5-sulfonamide, | 12233, idalopiridine, interpridine, |
| | E-6837, E-6801, and EMD- | latrepiridine, MS-245, PRX- |
| | 386,088 | 07034, SB-258,585, SB-271,046, |
| | | SB-357,134, SB-339,885, Ro 04- |
| | | 6790, Ro-4368554, sertindole, |
| | | olanzapine, asenapine, |
| | | clozapine, rosa rugosa extract, |
| | | and WAY-255315 |
| 5-HT₇ | AS-19, 5-CT, 5-meot, 8-OH- | Amisulpride, amitriptyline, |
| | DAPT, aripiprazole, E-55888, E- | amoxapine, clomipramine, |
| | 57431, LP-12, LP-44, MSD-5a, | clozapine, DR-4485, |
| | RA-7, and N,N- | fluphenazine, fluperlapine, ICI |
| | Dimethyltryptamine | 169,369, imipramine, |
| | | ketanserine, JNJ-18038683, |
| | | loxapine, lurasidone, LY- |
| | | 215,840, maprotiline, |
| | | methysergide, mesulergine, |
| | | mianserin, olanzepine, pimozide, |
| | | ritanserin, SB-258,719, SB- |
| | | 258,741, SB-269,970, SB- |
| | | 656,104-A, SB-691,673, |
| | | sertindole, spiperone, tenilapine, |
| | | TFMPP, vortioxetine, |
| | | trifluoperazine, ziprasidone, and |
| | | zotepine |
| Non-selective | | Chlorpromazine, cyproheptadine, |
| 5-HT antagonists | | pizotifen, oxetorone, spiperone, |
| | | ritanserin, |
| | | parachlorophenylalanine, |
| | | metergoline, propranolol, |
| | | mianserin, carbinoxamine, |
| | | methdilazine, promethazine, |
| | | pizotifen, oxatomide, feverfew, |
| | | fenclonin, and reserpine |

TABLE 2H
GLUATAMATE RECEPTOR AGONISTS AND ANTAGONISTS

| Receptor | Agonist | Antagonist |
|---|---|---|
| Ionotropic | AMPA, glutamic acid, ibotenic | AP5, AP7, cppene, selfotel, HU- |
| (GRIA-14, GRIK1-5, | acid, kainic acid, NMDA, | 211, Huperzine A, gabapentin, |
| and GRIN1-3B) | quisqualic acid | remacemide, amantadine, |
| | | atomoxetine, AZD6765, |
| | | agmatine, chloroform, |
| | | dextrallorphan, |
| | | dextromethorphan, dextrorphan, |
| | | diphenidine, dizocilpine (MK- |
| | | 801), ethanol, eticyclidine, |
| | | gacyclidine, ibogaine, ifenprodil, |
| | | ketamine, kynurenic acid, |
| | | memantine, magnesium, |
| | | methoxetamine, nitromemantine, |
| | | nitrous oxide, PD-137889, |
| | | perampanel, phencyclidine, |
| | | rolicyclidine, tenocyclidine, |
| | | methoxydine, tiletamine, |
| | | neramexane, eliprodil, etoxadrol, |
| | | dexoxadrol, WMS-2539, NEFA, |
| | | delucemine, 8A-PDHQ, |
| | | aptiganel, rhynchophylline |
| Metabotropic | L-ap4, acpd, l-qa, chpg, ly- | AIDA, fenobam, MPEP, LY- |
| (GRM1-8) | 379,268, ly-354,740, acpt, vu | 367,385, EGLU, CPPG, MAP4, |
| | 0155041 | MSOP, LY-341,495 |
| Glycine | | Rapastinel, NRX-1074, 7- |
| antagonists | | chlorokynurenic acid, 4- |
| | | chlorokynurenine, 5,7- |
| | | dichlorokynurenic acid, kynurenic |
| | | acid, TK-40, 1- |
| | | aminocyclopropanecarboxylic |
| | | acid (ACPC), L-phenylalanine, |
| | | and xenon |

TABLE 2I
HISTAMINE AGONISTS AND ANTAGONISTS

| Receptor | Agonist | Antagonist |
|---|---|---|
| Non-selective | Histamine dihydrochloride, HTMT | |
| | dimaleate, 2-pyridylethlyamine | |
| | dihydrochloride | |
| H₁ | | Acrivastine, azelastine, |
| | | astemizole, bilastine, |
| | | bromodiphenhydramine, |
| | | brompheniramine, buclizine, |
| | | carbinoxamine, cetirizine, |
| | | cetirizine dihydrochloride, |
| | | clemastine fumarate, clemizole |
| | | hydrochloride, |
| | | chlorodiphenhydramine, |
| | | chlorphenamine, chlorpromazine, |
| | | clemastine, cyclizine, |
| | | cyproheptadine, |
| | | dexbrompheniramine, |
| | | dexchlorpheniramine, |
| | | dimenhydrinate, dimethindene |
| | | maleate, dimetindene, |
| | | diphenhydramine, |
| | | diphenhydramine hydrochloride, |
| | | doxepin hydrochloride, |
| | | doxylamine, ebastine, |
| | | embramine, fexofenadine, |
| | | fexofenadine hydrochloride, |
| | | hydroxyzine, ketotifen fumarate, |
| | | loratadine, meclizine, meclizine |
| | | dihydrochloride, mepyramine |
| | | maleate, mirtazapine, |
| | | olopatadine, olopatadine |
| | | hydrochloride, orphenadrine, |
| | | phenindamine, pheniramine, |
| | | phenyltoloxamine, promethazine, |
| | | quetiapine, rupatadine, |
| | | terfenadine, tripelennamine, |
| | | zotepine, trans- triprolidine |
| | | hydrochloride, and triprolidine |
| H₁ inverse agonists | | Cetirizine, levocetirizine, |
| | | desloratadine, and pyrilamine |
| H₂ | Betazole, impromidine, dimaprit | Aminopotentidine, cimetidine, |
| | dihydrochloride, and amthamine | famotidine, ICI 162,846, |
| | dihyrdobromide | lafutidine, nizatidine, ranitidine, |
| | | ranitidine hyrdochloride, |
| | | roxatidine, zolantadine |
| | | dimaleate, and toitidine |
| H₃ | Imetit dihydropbromide, immepip | Clobenpropit, clobenpropit |
| | dihyrdrobromide, immethridine | dihydrobromide, A 3314440 |
| | dihydrobromide, α- | dihyrdochloride, BF 2649 |
| | Methylhistamine dihydrobromide, | hydrochloride, carcinine |
| | N-methylhistamine | ditrifluoroacetate, ABT-239, |
| | dihydrochloride, proxyfan | ciprofaxin, conessine, GT 2016, |
| | oxalate, and betahistine | A-349,821, impentamine |
| | | dihydrobromide, iodophenpropit |
| | | dihydrobromide, JNJ 10181457 |
| | | dihydrochloride, JNJ 5207852 |
| | | dihydrochloride, ROS 234 |
| | | dioxalate, SEN 12333, VUF 5681 |
| | | dihydrobromide, and |
| | | thioperamide |
| H₄ | Imetit dihydropbromide, immepip | Thioperamide, JNJ 7777120, A |
| | dihyrdrobromide, 4- | 943931 dihydrochloride, A |
| | methylhistamine dihydrochloride, | 987306, JNJ 10191584 maleate, |
| | clobenpropit dihydrobromide, | and VUF-6002 |
| | VUF 10460, and VUF 8430 | |
| | dihydrobromide | |

TABLE 2J
CANNABINOID AGONISTS AND ANTAGONISTS

| Receptor | Agonist | Antagonist |
|---|---|---|
| Cannabinoid receptor (non- | Anandamide, N-Arachidonoyl | |
| selective) | dopamine, 2- | |
| | Arachidonoylglycerol (2-AG), 2- | |
| | Arachidonyl glyceryl ether, Δ-9- | |
| | Tetrahydrocannabinol, EGCG, | |
| | Yangonin, AM-1221, AM-1235, | |
| | AM-2232, UR-144, JWH-007, | |
| | JWH-015, JWH-018, ACEA, | |
| | ACPA, arvanil, CP 47497, DEA, | |
| | leelamine, methanandamide, | |
| | NADA, noladin ether, oleamide, | |
| | CB 65, GP-1a, GP-2a, GW | |
| | 405833, HU 308, JWH-133, L- | |
| | 759,633, L-759,656, LEI 101, | |
| | MDA 19, and SER 601 | |
| CB₁ receptor | ACEA, ACPA, RVD-Hpα, (R)-(+)- | Rimonabant, cannabidiol, Δ⁹- |
| | methanandamide | tetrahydrocannabivarin (THCV), |
| | | taranabant, otenabant, |
| | | surinabant, rosonabant, SLV- |
| | | 319, AVE1625, V24343, AM 251, |
| | | AM 281, AM 6545, hemopressin, |
| | | LY 320135, MJ 15, CP 945598, |
| | | NIDA 41020, PF 514273, SLV |
| | | 319, SR 1141716A, and TC-C |
| | | 14G |
| CB₂ receptor | CB 65, GP 1a, GP 2a, GW | Cannabidiol, Δ⁹- |
| | 405833, HU 308, JWH 133, L- | tetrahydrocannabivarin (THCV), |
| | 759,656, L-759,633, SER 601, | AM 630, COR 170, JTE 907, and |
| | LEI 101 | SR 144528 |

TABLE 2K
PURINERGIC RECEPTOR AGONISTS AND ANTAGONISTS

| Receptor | Agonist | Antagonist |
|---|---|---|
| ADORA1 (P1 adenosine | Adenosine, N6- | Caffeine, theophylline, 8- |
| receptor) | Cyclopentyladenosine, N6-3- | Cyclopentyl-1,3-dimethylxanthine |
| | methoxyl-4-hydroxybenzyl | (CPX), 8-Cyclopentyl-1,3- |
| | adenine riboside (B2), CCPA, | dipropylxanthine (DPCPX), 8- |
| | tecadenoson, selodenoson, | Phenyl-1,3-dipropylxanthine, |
| | Certain Benzodiazepines and | bamifylline, BG-9719, BG09928, |
| | Barbiturates, 2′-meccpa, GR | FK-453, FK838, rolofylline, N- |
| | 79236, and SDZ WAG 994 | 0861, and PSB 36 |
| ADORA2A (P1 adenosine | Adenosine, N6-3-methoxyl-4- | Caffeine, theophylline, |
| receptor) | hydroxybenzyl adenine riboside | istradefylline, SCH-58261, SCH- |
| | (B2), YT-146, DPMA, UK- | 442,416, ATL-444, MSX-3, |
| | 423,097, limonene, NECA, CV- | preladenant, SCH-412,348, VER- |
| | 3146, binodenoson, ATL-146e, | 6623, VER-6947, VER-7835, |
| | CGS-21680, and Regadenoson | vipadenant, and ZM-241,385 |
| ADORA2B (P1 adenosine | Adenosine, 5′-N- | Caffeine, theophylline, CVT- |
| receptor) | ethylcarboxamidoadenosine, | 6883, ATL-801, compound 38, |
| | BAY 60-6583, LUF-5835, NECA, | MRS-1706, MRS-1754, OSIP- |
| | (S)-PHPNECA, and LUF-5845 | 339,391, PSB-603, PSB-0788, |
| | | and PSB-1115 |
| ADORA3 (P1 adenosine | Adenosine, 2-(1-Hexynyl)-N- | Caffeine, theophylline, MRS- |
| receptor) | methyladenosine, CF-101 (IB- | 1191, MRS-1220, MRS-1334, |
| | MECA), CF-102, 2-Cl-IB-MECA, | MRS-1523, MRS-3777, |
| | CP-532,903, inosine, LUF-6000, | MRE3008F20, MRE3005F20, |
| | and MRS-3558 | OT-7999, SSR161421, KF- |
| | | 26777, PSB-10, PSB-11, and |
| | | VUF-5574 |
| P2Y receptor | ATP, ADP, UTP, UDP, UDP- | Clopidogrel, elinogrel, prasugrel, |
| | glucose, 2-methylthioladenosine | ticlopidine, ticagrelor, AR-C |
| | 5′ diphosphate (2-mesadp), | 118925XX, AR-C 66096, AR-C |
| | lysophosphatidic acid, PSB 1114, | 69931, AZD 1283, MRS 2179, |
| | PSB 0474, NF 546, MRS 2365, | MRS 2211, MRS 2279, MRS |
| | MRS 2690, MRS 2693, MRS | 2500, MRS 2578, NF 157, NF |
| | 2768, MRS 2905, MRS 2957, | 340, PPADS, PPTN |
| | MRS 4062, and denufosol (P2Y₂ | hydrochloride, PSD 0739, SAR |
| | agonist) | 216471, and suramin |
| P2X receptor | Atp | A 438079, A 740003, A 804598, |
| | | A 839977, AZ 10606120, AZ |
| | | 11645373, 5-BDBD, BX 430, |
| | | Evans Blue, JNJ 47965567, KN- |
| | | 62, NF 023, NF 110, NF 157, NF |
| | | 279, NF 449, PPADS, iso- |
| | | PPADS, PPNDS, Ro 0437626, |
| | | Ro 51, RO-3, TC-P 262, suramin, |
| | | TNP-ATP, and P2X₇ antagonists |
| | | NF279, calmidazolium, and KN- |
| | | 62 |

TABLE 2L
NEUROPEPTIDE AGONISTS AND ANTAGONISTS

| Gene | Agonist | Antagonist |
|---|---|---|
| Neuropeptide Y receptor (non- | Neuropeptide Y, pancreatic | |
| selective) | polypeptide, BWX-46, and | |
| | Peptide YY | |
| Neuropeptide Y₁ receptor | | BVD-10, GR-231,118, BIBO- |
| | | 3304, BIBP-3226, PD-160,170, |
| | | and BMS 193885 |
| Neuropeptide Y₂ receptor | | BIIE-0246, CYM 9484, JNJ |
| | | 5207787, and SF 11 |
| Neuropeptide Y₄ receptor | | UR-AK49 and GR-231,118 |
| Neuropeptide Y₅ receptor | | Lu AA-33810, CGP 71683 |
| | | hydrochloride, GW 438014A, L- |
| | | 152,804, NPY 5RA972, NTNCB |
| | | hydrochloride, velneperit, and S |
| | | 25585 |
| Somatostatin receptor | Somatostatin, cortistatin, | Cyclomastatin and CYN 154806 |
| | octreotide, lanreotide CH 275, TT | |
| | 232, TC-G 1003, seglitide, RC | |
| | 160, NNC 26-9100, L-817,818, L- | |
| | 803,087 trifluoroacetate, and | |
| | (1R,1′S,3′R/1R,1′R,3′S)-L- | |
| | 054,264 | |
| CGRP receptor (calcitonin gene- | A-CGRP, β-CGRP, calcitonin, | Telcagepant, sumatriptan |
| related peptide receptor) | PHM 27, amylin, pramlintide, | (decreases CGRP promoter |
| | CRSP-1, and SUN-B 8155 | activity), MK-3207, and BIBN |
| | | 4096 BS, MK-0974 |
| Tachykinin 1 receptor (NK₁ | Substance P, GR-73632, and | Aprepitant, Casopitant, |
| receptor) | C14TKL-1 | Ezlopitant, Fosaprepitant, |
| | | Lanepitant, Maropitant, |
| | | Vestipitant, L-733,060, L- |
| | | 741,671, L-742,694, RP-67580, |
| | | RPR-100,893, CP-96345, CP- |
| | | 99994, GR-205,171, TAK-637, |
| | | FK 888, GR 82334, L-760,735, L- |
| | | 732,138, L-733,060, SDZ NKT |
| | | 343, Spantide 1, SR 140333, and |
| | | T-2328 |
| Tachykinin 2 receptor (NK₂ | Neurokinin A and GR-64349 | Ibodutant, saredutant, GR- |
| receptor) | | 159,897, GR 94800, MDL |
| | | 29,913, and MEN 11420, MEN- |
| | | 10376 |
| Tachykinin 3 receptor (NK₃ | Neurokinin B and senktide | Fezolinetant, MLE-4901, |
| receptor) | | Osanetant, Talnetant, SB- |
| | | 222,200, SSR 146977, and SB- |
| | | 218,795 |
| Vasoactive intestinal polypeptide | VIP, PACAP, PACAP-38, | VIP (6-28), [D-p-Cl-Phe⁶,Leu¹²]- |
| receptor 1 (VIPR1/VPAC1) and | PACAP-27, peptide histidine | VIP, and AC-Tyr¹,D-Phe²]GRF 1- |
| Vasoactive intestinal polypeptide | isoleucineamide (PHI), peptide | 29 amide |
| receptor 2 (VIPR2/VPAC2) | histidine methionineamide | |
| | (PHM), peptide histidine valine | |
| | (PHV), and Bay 55-9837 | |
| Opioid receptor (non-selective) | Dynorphins, enkephalins, | |
| | endorphins, endomorphins, and | |
| | nociceptin | |
| μ-opioid receptor | DAMAGO, endomorphin-1, | Naloxone, naltrexone, |
| | endomorphin-2, fentanyl, | nalmefene, diprenorphine, |
| | loperamide, meptazinol, | nalorphine, nalorphine |
| | oxycodone, PL 017, sinomenine, | dinicotinate, levallorphan, |
| | and buprenorphine (partial) | samidorphan, nalodeine, |
| | | alvimopan, methylnaltrexone, |
| | | naloxegol, 6β-naltrexol, |
| | | axelopran, bevenopran, |
| | | methylsamidorphan, |
| | | naldemedine, buprenorphine, |
| | | dezocine, eptazocine, CTAP, |
| | | CTOP, cyprodime, clocinnamox |
| | | mesylate, naloxonazine, |
| | | funaltrexamine, and cyprodamine |
| κ-opioid receptor | Alazocine, Bremazocine, 8- | 5′-Acetamidinoethylnaltrindole, |
| | Carboxamidocyclazocine, | 5′-Guanidinonaltrindole, 6′- |
| | Cyclazocine, Ketazocine, | Guanidinonaltrindole, |
| | Metazocine, Pentazocine, | Amentoflavone, AT-076, |
| | Phenazocine, Morphinans (e.g., | Binaltorphimine, BU09059, |
| | 6′-Guanidinonaltrindole, | Buprenorphine, CERC-501, |
| | Butorphan, Butorphanol, | Dezocine, DIPPA, jdtic, LY- |
| | Cyclorphan, Diprenorphine, | 255582, LY-2459989, LY- |
| | Etorphine, Levallorphan, | 2795050, Methylnaltrexone, |
| | Levomethorphan, Levorphanol, | ML190, ML350, MR-2266, |
| | Morphine, Nalbuphine, | Naloxone, Naltrexone, |
| | Nalfurafine, Nalmefene, | Noribogaine, Norbinaltorphimine, |
| | Nalodeine, Nalorphine, | Pawhuskin A, PF-4455242, |
| | Norbuprenorphine, | Quadazocine, RB-64, and |
| | Norbuprenorphine-3-glucuronide, | Zyklophin |
| | Oxilorphan, Oxycodone, | |
| | Proxorphan, Samidorphan, and | |
| | Xorphanol), Arylacetamides (e.g., | |
| | Asimadoline, BRL-52537, | |
| | Eluxadoline, Enadoline, GR- | |
| | 89696, ICI-204,448, ICI-199,441, | |
| | LPK-26, MB-1C-OH, Niravoline, | |
| | N-MPPP, Spiradoline, U-50,488, | |
| | U-54,494A, and U-69,593), | |
| | CR665, Difelikefalin (CR845), | |
| | Dynorphins (dynorphin A, | |
| | dynorphin B, big dynorphin), | |
| | Terpenoids (e.g., Collybolide, | |
| | Erinacine E, Mentholm RB-64, | |
| | Salvinorin A, and 2- | |
| | Methoxymethyl salvinorin B), | |
| | Apadoline, HS665, HZ-2, | |
| | Ibogaine, Ketamine, Noribogaine, | |
| | Pentazocine, Tifluadom, and | |
| | Nalfurafine | |
| δ-opioid receptor | Leu-enkephalin, Met-enkephalin, | Buprenorphine, naltriben, |
| | Deltorphins I and II, DADLE, AR- | naltrindole, SDM25N, ICI- |
| | M 100390, 7- | 174,864, ICI-154,129, BNTX, and |
| | Spiroindanyloxymorphone, N- | benzylnatrindole |
| | Phenethyl-14-ethoxymetopon, | |
| | ADL-5859, BU-48, SNC-80, | |
| | SNC-162, FIT, 6′-GTI, DPDPE, | |
| | BW373U86, DPI-221, DPI-287, | |
| | DPI-3290, TAN-67, RWJ- | |
| | 394674, Norbuprenorphine, | |
| | Cannabidiol, | |
| | Tetrahydrocannabinol, | |
| | Xorphanol, Mitragynine, and | |
| | Mitragynine pseudoindoxyl | |
| NOP receptor (opioid) | Orphanin, SCH-221510, NNC | UFP-101, Trap 101, nocistatin, |
| | 63-0532, MCOPPB, and Ac- | BAN ORL 24, J 113397, JTC |
| | RYYRWK-NH₂ | 801, and SB 612111 |
| Oxytocin receptor | Carbetocin, demoxytocin, lipo- | Atosiban, barusiban, epelsiban, |
| | oxytocin-1, merotocin, oxytocin, | L-368,899, L-371,257, L-372- |
| | TC OT 39, and WAY-267,464 | 662, retosiban, SSR-126,768, |
| | | and WAY-162,720 |
| Vasopressin receptor | Vasopressin and desmopressin | TC OT 39, OPC 21268, SR |
| | | 49059, TASP 0390325, |
| | | conivaptan, tolvaptan, |
| | | mozavaptan, lixivaptan, |
| | | satavaptan, relcovaptan, |
| | | nelivaptan, demeclocycline, and |
| | | lithium |

TABLE 2M
NEUROTRANSMISSION MODULATORS

| Type | Modulators |
|---|---|
| Norepinephrine reuptake inhibitors | Amedalin, atomoxetine, CP-39,332, daledalin, |
| (increase adrenergic neurotransmission) | edivoxetine, esreboxetine, lortalamine, nisoxetine, |
| | reboxetine, talopram, talsupram, tandamine, |
| | viloxazine, bupropion, ciclazindol, manifaxine, |
| | maprotiline, radafaxine, tapentadol, teniloxazine, |
| | protriptyline, nortriptyline, and desipramine |
| Norepineprhine-dopamine reuptake inhibitors | Amineptine, bupropion, desoxypipradrol, |
| (increase adrenergic and dopamine | dexmethylphenidate, difemetorex, diphenylprolinol, |
| neurotransmission) | ethylphenidate, fencamfamine, fencamine, |
| | lefetamine, methylenedioxypyrovalerone, |
| | methylphenidate, nomifensine, O-2172, oxolinic |
| | acid, pipradrol, prolintane, pyrovalerone, |
| | tametraline, and WY-46824 |
| Serotonin-norepinephrine-dopamine reuptake | Mazindol, nefazodone, sibutramine, venlafaxine, |
| inhibitors (SNDRIs) and serotonin-norepinephrine | esketamine, duloxetine, ketamine, phencyclidine, |
| reuptake inhibitors (SNRIs) | tripelennamine, mepiprazole, amitifadine, AN788, |
| (increase adrengergic, dopamine, and serotonin | ansofaxine, centanafadine, atomoxetine, |
| neurotransmission) | desvenlafaxine, milnacipran, levomilnacipran, |
| | dasotraline, Lu AA34893, Lu AA37096, NS-2360, |
| | tedatioxetine, tesofensine, bicifadine, BMS- |
| | 866,949, brasofensine, diclofensine, DOV-216,303, |
| | EXP-561, liafensine, NS-2359, RG-7166, SEP- |
| | 227,162, SEP-228,425, SEP-228,432, naphyrone, |
| | 3,3-Diphenylcyclobutanamine, 3,4- |
| | Dichlorotametraline, D-161, desmethylsertraline, |
| | DMNPC, DOV-102,677, fezolamine, |
| | GSK1360707F, indatraline, JNJ-7925476, JZ-IV- |
| | 10, JZAD-IV-22, LR-5182, methylnaphthidate, MI-4, |
| | PRC200-SS, PRC050, PRC025, SKF-83,959, TP1, |
| | phenyltropanes (e.g., WF-23, dichloropane, and |
| | RTI-55), Ginkgo biloba extract, St John's Wort, |
| | hyperforin, adhyperforin, and uliginosin B |
| Dopamine reuptake inhibitors | Dopamine reuptake inhbiitors (e.g., altropane, |
| (increase dopamine neurotransmission) | amfonelic acid, amineptine, BTCP, 3C-PEP, DBL- |
| | 583, difluoropine, GBR-12783, GBR-12935, GBR- |
| | 13069, GBR-13098, GYKI-52895, lometopane, |
| | methylphenidate, ethylphenidate, modafinil, |
| | armodafinil, RTI-229, vanoxerine, adrafinil, |
| | benztropine, bupropion, fluorenol, medifoxamine, |
| | metaphit, rimcazole, venlafaxine, Chaenomeles |
| | speciosa, and oroxylin A), dopamine releasing |
| | agents (e.g., p-Tyramine), dextroamphetamine, |
| | lisdexamfetamine, dexmethylphenidate, and |
| | cathinone |
| Dopamine prodrugs | Levopoda, docarpamine |
| (increase dopamine neurotransmission) | |
| GABA reuptake inhibitors | CL-996, deramciclane, gabaculine, guvacine, |
| (increase GABA neurotransmission) | nipecotic acid, NNC-711, NNC 05-2090, SKF- |
| | 89976A, SNAP-5114, tiagabine, and hyperforin |
| GABA analogs | Gabapentin, butyric acid, valproic acid, valpromide, |
| (increase GABA neurotransmission) | valnoctamide, 3-hydroxybutanal, GHB, sodium, |
| | oxybate, aceburic acid, GBL, GHBAL, GHV, GVL, |
| | GHC, GCL, HOCPCA, UMB68, pregabalin, tolibut, |
| | phaclofen, sacolfen, arecaidine, gaboxadol, |
| | isonipecotic acid, 3-Methyl-GABA, AABA, BABA, |
| | DAVA, GAVA, Glutamic acid, hopantenic acid, |
| | piracetam, and vigabatrin |
| GABA prodrugs | L-Glutamine, N-Isonicotinoyl-GABA, picamilon, |
| (increase GABA neurotransmission) | progabide, tolgabide |
| Acetylcholinesterase inhibitors | Carbamates, physostigmine, neostigmine, |
| (increase nicotinic and muscarinic | pyridostigmine, ambenonium, demecarium, |
| neurotransmission) | rivastigmine, phenanthrene derivatives, |
| | galantamine, caffeine, rosmarinic acid, alpha- |
| | pinene, piperidines, donepezil, tacrine, |
| | edrophonium, Huperzine A, ladostigil, ungeremine, |
| | lactucopicrin, dyflos, echothiophate, parathion, and |
| | quasi-irreversible acetylcholinesterase inhibitors |
| Serotonin reuptake inhibitors | Alaproclate, cericlamine, citalopram, dapoxetine, |
| (increase serotonin neurotransmission) | escitalopram, femoxetine, fluoxetine, fluvoxamine, |
| | ifoxetine, indalpine, omiloxetine, panuramine, |
| | paroxetine, pirandamine, RTI-353, sertraline, |
| | zimelidine, desmethylcitalopram, |
| | didesmethylcitalopram, seproxetine ((S)- |
| | norfluoxetine), desvenlafaxine, cianopramine, |
| | litoxetine, lubazodone, SB-649,915, trazodone, |
| | vilazodone, vortioxetine, dextromethorphan, |
| | dextropropoxyphene, dimenhydrinate, |
| | diphenhydramine, mepyramine (pyrilamine), |
| | mifepristone, delucemine, mesembrenone, |
| | mesembrine, roxindole, duloxetine, |
| | levomilnacipran, milnacipran, dapoxetine, |
| | sibutramine, chlorpheniramine, |
| | dextropmethorphan, and methadone |
| Serotonin releasing agents | Chlorphentermine, cloforex, dexfenfluramine, |
| (increase serotonin neurotransmission) | etolorex, fenfluramine, flucetorex, indeloxazine, |
| | levofenfluramine, tramadol, carbamazepine, |
| | amiflamine (FLA-336), viqualine (PK-5078), 2- |
| | Methyl-3,4-methylenedioxyamphetamine (2-Methyl- |
| | MDA), 3-Methoxy-4-methylamphetamine (MMA), 3- |
| | Methyl-4,5-methylenedioxyamphetamine (5-Methyl- |
| | MDA), 3,4-Ethylenedioxy-N-methylamphetamine |
| | (EDMA), 4-Methoxyamphetamine (PMA), 4- |
| | Methoxy-N-ethylamphetamine (PMEA), 4-Methoxy- |
| | N-methylamphetamine (PMMA), 4- |
| | Methylthioamphetamine (4-MTA), 5-(2- |
| | Aminopropyl)-2,3-dihydrobenzofuran (5-APDB), 5- |
| | Indanyl-2-aminopropane (IAP), 5-Methoxy-6- |
| | methylaminoindane (MMAI), 5-Trifluoromethyl-2- |
| | aminoindane (TAI), 5,6-Methylenedioxy-2- |
| | aminoindane (MDAI), 5,6-Methylenedioxy-N- |
| | methyl-2-aminoindane (MDMAI), 6-Chloro-2- |
| | aminotetralin (6-CAT), 6-Tetralinyl-2-aminopropane |
| | (TAP), 6,7-Methylenedioxy-2-aminotetralin (MDAT), |
| | 6,7-Methylenedioxy-N-methyl-2-aminotetralin |
| | (MDMAT), N-Ethyl-5-trifluoromethyl-2-aminoindane |
| | (ETAI), N-Methyl-5-indanyl-2-aminopropane, |
| | aminorex, MDMA, MDEA, MDA, MBDB, and |
| | tryptamines, such as DMT, αmt, 5meo-NMT, NMT, |
| | NETP, Dimethyl-Serotonin, 5meo-NET, αet and |
| | αmt |
| Excitatory amino acid reuptake inhibitors | Didydrokanic acid, WAY-213,613, L-trans-2,4-PDC, |
| (increase Glutamate receptor neurotransmission) | amphetamine, and L-Theanine |
| Glycine reuptake inhibitors | Bitopertin, Org 24598, Org 25935, ALX-5407, |
| (increase Glutamate receptor neurotransmission) | sacrosine, Org 25543, and N-arachidonylglycerine |
| Histidine decarboxylase inhibitors | Tritoqualine, catechin |
| (decrease histamine neurotransmission) | |
| Endocannabinoid enhancers | AM404, fatty acid amide hydrolase inhibitors (e.g., |
| (increase cannabinoid neurotransmission) | AM374, ARN2508, BIA 10-2472, BMS-469908, |
| | CAY-10402, JNJ-245, JNJ-1661010, JNJ- |
| | 28833155, JNJ-40413269, JNJ-42119779, JNJ- |
| | 42165279, MK-3168, MK-4409, MM-433593, OL- |
| | 92, OL-135, PF-622, PF-750, PF-3845, PF- |
| | 04457845, PF-04862853, RN-450, SA-47, SA-73, |
| | SSR-411298, ST-4068, TK-25, URB524, URB597, |
| | URB694, URB937, VER-156084, and V-158866 |
| Monoacylglycerol lipase inhibitors | N-arachidonoyl maleimide, JZL184 |
| (increase cannabinoid neurotransmission) | |
| Endocannabinoid transporter inhibitors | Sb-fi-26 |
| (increase cannabinoid neurotransmission) | |
| Endocannabinoid reuptake inhibitors | AM404, AM1172, LY-2183240, O-2093, OMDM-2, |
| (increase cannabinoid neurotransmission) | UCM-707, VDM-11, guineensine, ETI-T-24_B_I, |
| | WOBE437, and RX-055 |
| Adenosine uptake inhibitors | Cilostazol, dilazep, and dipyramidole |
| (increase purinergic neurotransmission) | |
| Nucleoside transporter inhibitors | 8MDP, Decynium 22, 5-iodotubercidin, NBMPR, |
| (increase purinergic neurotransmission) | and TC-T 6000 |

Neurotoxins

In some embodiments, the neurotransmission modulator is a neurotoxin (e.g., a neurotoxin listed in Table 3), or a functional fragment or variant thereof. Neurotoxins include, without limitation, convulsants, nerve agents, parasympathomimetics, and uranyl compounds. Neurotoxins may be bacterial in origin, or fungal in origin, or plant in origin, or derived from a venom or other natural product. Neurotoxins may be synthetic or engineered molecules, derived de novo or from a natural product. Suitable neurotoxins include but are not limited to botulinum toxin and conotoxin. Exemplary neurotoxins are listed in Table 3.

TABLE 3
NEUROTOXINS
NEUROTOXINS

2,4,5-Trihydroxyamphetamine
2,4,5-Trihydroxymethamphetamine
3,4-Dichloroamphetamine
5,7-Dihydroxytryptamine
5-Iodowillardiine
Ablomin
Aconitine
Aconitum
Aconitum anthora
AETX
Agelenin
Agitoxin
Aldrin
Alpha-Methyldopamine
Alpha-neurotoxin
Altitoxin
Anatoxin-a
Androctonus australis hector insect toxin
Anisatin
Anthopleurin
Antillatoxin
Anuroctoxin
Apamin
Arum italicum
Arum maculatum
Babycurus toxin 1
Batrachotoxin
BDS-1
Bestoxin
Beta-Methylamino-L-alanine
BgK
Birtoxin
BmKAEP
BmTx3
BotlT2
BotlT6
Botulinum toxin
Brevetoxin
Bukatoxin
Butantoxin
Calcicludine
Calciseptine
Calitoxin
Caramboxin
Carbon disulfide
CgNa toxin
Charybdotoxin
Cicutoxin
Ciguatoxin
Cll1
Clostridium botulinum
Conantokins
Conhydrine
Coniine
Conotoxin
Contryphan
CssII
CSTX
Curare
Cyanide poisoning
Cylindrospermopsin
Cypermethrin
Delta atracotoxin
Dendrotoxin
Dieldrin
Diisopropyl fluorophosphates
Dimethylmercury
Discrepin
Domoic acid
Dortoxin
DSP-4
Ergtoxin
Falcarinol
Fenpropathrin
Gabaculine
Ginkgotoxin
Grammotoxin
Grayanotoxin
Hainantoxin
Halcurin
Hefutoxin
Helothermine
Heteroscodratoxin-1
Histrionicotoxin
Homoquinolinic acid
Hongotoxin
Huwentoxin
Ibotenic acid
Ikitoxin
Inhibitor cystine knot
Jingzhaotoxin
Kainic acid
Kaliseptine
Kappa-bungarotoxin
Kodaikanal mercury poisoning
Kurtoxin
Latrotoxin
Lq2
Maitotoxin
Margatoxin
Maurotoxin
Mercury (element)
Methanol
Methiocarb
MPP+
MPTP
Nemertelline
Neosaxitoxin
Nicotine
N-Methylconiine
Oenanthotoxin
Oxalyldiaminopropionic acid
Oxidopamine
Oxotoxin
Pahutoxin
Palytoxin
Pandinotoxin
Para-Bromoamphetamine
Para-Chloroamphetamine
Para-Chloromethamphetamine
Para-Iodoamphetamine
Penitrem A
Phaiodotoxin
Phenol
Phoneutria nigriventer toxin-3
Phrixotoxin
Polyacrylamide
Poneratoxin
Psalmotoxin
Pumiliotoxin
Quinolinic acid
Raventoxin
Resiniferatoxin
Samandarin
Saxitoxin
Scyllatoxin
Sea anemone neurotoxin
Slotoxin
SNX-482
Stichodactyla toxin
Taicatoxin
Taipoxin
Tamapin
Tertiapin
Tetanospasmin
Tetraethylammonium
Tetramethylenedisulfotetramine
Tetrodotoxin
Tityustoxin
Tricresyl phosphate
TsIV
Vanillotoxin
Veratridine

Neurotransmission modulators also include antibodies that bind to neurotransmitters or neurotransmitter receptors listed in Tables 1A, 1B, Table 7, and Table 8 and decrease neurotransmission. These antibodies include blocking and neutralizing antibodies. Antibodies to neurotransmitters or neurotransmitter receptors listed in Tables 1A, 1B, Table 7, and Table 8 can be generated by those of skill in the art using well established and routine methods.

Neuropeptide Signaling Modulators

In some embodiments, a neuromodulating agent is a neuropeptide signaling modulator (e.g., an agent that increases or decreases neuropeptide signaling), such as a blocker or agonist of a neuropeptide receptor listed in Table 1A. Neuromodulating agents that increase neuropeptide signaling include neuropeptides and neuropeptide receptors (e.g., a neuropeptide (ligand) listed in Table 1A, Table 1B, or Table 7, e.g., a neuropeptide having the sequence referenced by accession number or Entrez Gene ID of a neuropeptide listed in Table 1A, Table 1B, or Table 7, or an analog thereof, e.g., a sequence having at least 70%, 75%, 80%, 85%, 90%, 95%, 98%, or 99% identity to the sequence referenced by accession number or Entrez Gene ID. The neuromodulating agent can be an endocannabinoid, amine, amino acid, purine, gas, gastrin, opioid, monoamine, secretin, tachykinin, neuropeptide, neurohypophyseal, orexin, or somatostatin, e.g., listed in Table 1B. In some embodiments, neuropeptide signaling is increased by administering, locally delivering, or stabilizing a neuropeptide listed in Tables 1A, 1B, or encoded by a gene in Table 7. Neuromodulating agents that increase neuropeptide signaling also include agents that increase neuropeptide receptor activity (e.g., neuromodulating agents that increase the activity of a neuropeptide receptor or signaling protein listed in Table 1A or encoded by a gene in Table 7 via upregulation, stabilization, agonism, or overexpression). Exemplary neuropeptide agonists are listed in Table 2A and 2L. Neuromodulating agents that increase neuropeptide signaling also include agents that reduce neuropeptide degradation or reuptake, agents that increase neuropeptide synthesis or release (e.g., agents that increase the activity of a biosynthetic protein encoded by a gene in Table 1A or Table 7 via stabilization, overexpression, or upregulation), increase neuropeptide receptor synthesis or membrane insertion, and regulate neuropeptide receptor conformation (e.g., agents that bind to a receptor and keep it in an “open” or “primed” conformation). Neuropeptide signaling modulators can increase neuropeptide signaling by 10%, 20%, 30%, 40%, 50%, 60%, 70%, 80%, 90%, 95%, 98% or more.

Neuromodulating agents that decrease neuropeptide signaling include agents that decrease neuropeptide receptor activity (e.g., neuromodulating agents that decrease the activity of a neuropeptide receptor or signaling protein listed in Table 1A or encoded by a gene in Table 7 via blockade, antagonism, or downregulation). Exemplary neuropeptide antagonists are listed in Table 2A or 2L. Neuromodulating agents that decrease neuropeptide signaling also include agents that bind to neuropeptides or block their interaction with receptors (e.g., neuropeptide blocking or neutralizing antibodies), agents that increase neuropeptide degradation or clearance, agents that decrease neuropeptide synthesis or release (e.g., agents that decrease the activity of a biosynthetic protein encoded by a gene in Table 1A or Table 7 via inhibition or downregulation), decrease neuropeptide receptor synthesis or membrane insertion, and regulate neuropeptide receptor conformation (e.g., agents that bind to a receptor and keep it in a “closed” or “inactive” conformation). In some embodiments, neuropeptide signaling is decreased by sequestering, blocking, antagonizing, or degrading a neuropeptide listed in Tables 1A, 1B, or encoded by a gene in Table 7. Neuropeptide signaling modulators can decrease neuropeptide signaling by 10%, 20%, 30%, 40%, 50%, 60%, 70%, 80%, 90%, 95%, 98% or more.

Neuropeptide signaling modulators also include antibodies that bind to neuropeptides or neuropeptide receptors listed in Tables 1A, 1B, and Table 7 and decrease neuropeptide signaling. These antibodies include blocking and neutralizing antibodies. Exemplary neuropeptide signaling blocking and neutralizing antibodies are listed below in Table 4. Antibodies to neuropeptides and neuropeptide receptors listed in Tables 1A, 1B, and Table 7 can also be generated by those of skill in the art using well established and routine methods.

TABLE 4
NEUROPEPTIDE AND NEUROPEPTIDE
RECEPTOR ANTIBODIES

| Neuropeptide or | | |
|---|---|---|
| Neuropeptide | | |
| Receptor | Antibody | Company |
| CGRP | LY2951742 | Eli Lilly |
| | (Galcanezumab) | |
| CGRP | ALD-403 | Alder Biopharmaceuticals |
| CGRP | TEV-48125 (LBR-101) | Teva Pharmaceuticals |
| CGRP | AMG 334 | Amgen |
| Glucagon | REGN1193 | Regeneron |

### Neuronal Growth Factor Modulators

In some embodiments, a neuromodulating agent is a neuronal growth factor modulator (e.g., an agent that decreases or increases neurogenic/axonogenic signals, e.g., a neuronal growth factor or neuronal growth factor mimic, or an agonist or antagonist of a neuronal growth factor or neuronal growth factor receptor). For example, the neuromodulating agent is a neuronal growth factor listed in Table 1C or Table 7, e.g., a neuronal growth factor having the sequence referenced by accession number or Entrez Gene ID in Table 1C or Table 7, or an analog thereof, e.g., a sequence having at least 75%, 80%, 85%, 90%, 90%, 98%, 99% identity to the sequence referenced by accession number or Entrez Gene ID in Table 1C or Table 7. Neuronal growth factor modulators also include agonists and antagonists of neuronal growth factors and neuronal growth factor receptors listed in Table 1C or Table 7. A neuronal growth factor modulator may increase or decrease neurogenesis, neuronal growth, neuronal differentiation, neurite outgrowth, synapse formation, synaptic maturation, synaptic refinement, or synaptic stabilization. Neuronal growth factor modulators regulate innervation and the formation of synaptic connections between two or more neurons and between neurons and non-neural cells. A neuronal growth factor modulator may block one or more of these processes (e.g., through the use of antibodies that block neuronal growth factors or their receptors) or promote one or more of these processes (e.g., through the use of neuronal growth factors or analogs thereof). Neuronal growth factor modulators can increase or decrease one of the above mentioned processes by 10%, 20%, 30%, 40%, 50%, 60%, 70%, 80%, 90%, 95%, 98%, 200%, 500% or more.

In some embodiments, the neuromodulating agent decreases neurogenic/axonogenic signals, e.g., the method includes administering to the subject or contacting a cell with a neuromodulating agent (e.g., a neuronal growth factor modulator) in an amount and for a time sufficient to decrease neurogenesis or axonogenesis. For example, the neuromodulating agent that leads to a decrease in neurogenesis or axonogenesis is a blocking or neutralizing antibody against a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRα family ligands and receptors (e.g., GFRα1, GFRα2, GFRα3, GFRα4, GDNF), CNTF, LIF, neurturin, artemin, persephin, neurotrophin, chemokines, cytokines, and others listed in Table 1C or Table 7. Receptors for these factors can also be targeted, as well as downstream signaling pathways including Jak-Stat inducers, and cell cycle and MAPK signaling pathways. In some embodiments, the neuronal growth factor modulator decreases neurogenesis, axonogenesis or any of the processes mentioned above by sequestering, blocking, antagonizing, degrading, or downregulating a neuronal growth factor or a neuronal growth factor receptor listed in Table 1C or encoded by a gene in Table 7. In some embodiments, the neuronal growth factor modulator decreases neurogenesis, axonogenesis or any of the processes mentioned above by blocking or antagonizing a signaling protein encoded by a gene in Table 7 that is downstream of a neuronal growth factor. In some embodiments, the neuronal growth factor modulator decreases neurogenesis, axonogenesis or any of the processes mentioned above by blocking, disrupting, or antagonizing a synaptic or structural protein encoded by a gene in Table 7. Neurogenesis, axonogenesis, neuronal growth, neuronal differentiation, neurite outgrowth, synapse formation, synaptic maturation, synaptic refinement, or synaptic stabilization can be decreased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, or synaptic stabilization can be decreased in the subject between 5-20%, between 5-50%, between 10-50%, between 20-80%, between 20-70%.

In some embodiments, the neuromodulating agent is one that increases neurogenic/axonogenic signals, e.g., the method includes administering to the subject or contacting a cell with a neuromodulating agent (e.g., a neuronal growth factor modulator) in an amount and for a time sufficient to increase neurogenesis or axonogenesis. For example, the neuromodulating agent that leads to an increase in neurogenesis or axonogenesis is a neurotrophic factor. Relevant neurotrophic factors include NGF, BDNF, ProNGF, Sortilin, TGFβ and TGFβ family ligands and receptors (e.g., TGFβR1, TGFβR2, TGFβ1, TGFβ2 TGFβ4), GFRα family ligands and receptors (e.g., GFRα1, GFRα2, GFRα3, GFRα4, GDNF), CNTF, LIF, neurturin, artemin, persephin, neurotrophin, chemokines, cytokines, and others listed in Table 1C or Table 7. Receptors for these factors may also be targeted, as well as downstream signaling pathways including Jak-Stat inducers, and cell cycle and MAPK signaling pathways. In some embodiments, the neuronal growth factor modulator increases neurogenesis, axonogenesis or any of the processes mentioned above by administering, locally delivering, or stabilizing a neuronal growth factor listed in Table 1C or encoded by a gene in Table 7, or by upregulating, agonizing, or stabilizing a neuronal growth factor receptor listed in Table 1C or encoded by a gene in Table 7. In some embodiments, the neuronal growth factor modulator increases neurogenesis, axonogenesis or any of the processes mentioned above by stabilizing, agonizing, overexpressing, or upregulating a signaling protein encoded by a gene in Table 7 that is downstream of a neuronal growth factor. In some embodiments, the neuronal growth factor modulator increases neurogenesis, axonogenesis or any of the processes mentioned above by stabilizing, overexpressing, or upregulating a synaptic or structural protein encoded by a gene in Table 7. Neurogenesis, axonogenesis, neuronal growth, neuronal differentiation, neurite outgrowth, synapse formation, synaptic maturation, synaptic refinement, or synaptic stabilization can be increased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, or synaptic stabilization can be increased in the subject between 5-20%, between 5-50%, between 10-50%, between 20-80%, or between 20-70%.

In some embodiments, the neuromodulating agent that increases or decreases the number of nerves in an affected tissue. For example, the subject has cancer (e.g., the subject has a highly innervated tumor). For example, the neuromodulating agent is administered in an amount and for a time sufficient to decrease neurogenesis/axonogenesis. The neuromodulating agent can be, e.g., an inhibitor of neuronal growth factor signaling such as a blocking antibody directed to a neuronal growth factor or neuronal growth factor receptor.

Neuronal growth factor modulators also include antibodies that bind to neuronal growth factors or neuronal growth factor receptors and decrease their signaling (e.g., blocking antibodies). Exemplary neuronal growth factor blocking antibodies are listed below in Table 5. Antibodies to neuronal growth factors listed in Table 1C and Table 7 can also be generated by those of skill in the art using well established and routine methods.

TABLE 5
NEURONAL GROWTH FACTOR ANTIBODIES

| Neuronal | | |
|---|---|---|
| Growth | | |
| Factor | Antibody | Company |
| BDNF | 38B8 (agonist antibody) | Pfizer |
| BDNF | 29D7 (agonist antibody) | Pfizer |
| EphA3 | KB004 | KaloBios Pharmaceuticals, |
| | | Inc. |
| IFNA1 | Faralimomab | Creative Biolabs |
| IFNA1 | Sifalimumab (MEDI-545) | MedImmune |
| IFNA1 | Rontalizumab | Genentech |
| IGF | Figitumumab (CP-751,871) - | Pfizer |
| | an IGR-1R MAb | |
| IGF | SCH717454 (Robatumamab, | Merck |
| | inhibits IGF initiated | |
| | phosphorylation) | |
| IGF | Cixutumumab (IGF-1R | Eli Lilly |
| | antibody) | |
| IGF | Teprotumumab (IGF-1R | Genmab/Roche |
| | blocking antibody) | |
| IGF-2 | Dusigitumab | MedImmune/AstraZeneca |
| IGF-2 | DX-2647 | Dyax/Shire |
| IGF | Xentuzumab | Boehringer Ingelheim/Eli |
| | | Lilly |
| IGF | Dalotuzumab (IGFR1 | Merck & Co. |
| | blocking antibody) | |
| IGF | Figitumumab (IGFR1 | Pfizer |
| | blocking antibody) | |
| IGF | Ganitumab (IGFR1 | Amgen |
| | blocking antibody) | |
| IGF | Robatumumab (IGFR1 | Roche/Schering-Plough |
| | blocking antibody) | |
| IL1B | Canakinumab | Novartis |
| IL1B | APX002 | Apexigen |
| IL1B | Gevokizumab | XOMA |
| IL4 | Pascolizumab | GlaxoSmithKline |
| IL4 | Dupilumab | Regeneraon/Sanofi |
| IL6 | Siltuximab | Janssen Biotech, Inc. |
| IL6 | Olokizumab | UCB/R-Pharm |
| IL6 | Elsilimomab | Orphan Pharma |
| | | International |
| IL6 | Sirukumab | Centocor |
| IL6 | Clazakizumab | Bristol Myers Squib/Alder |
| | | Biopharmaceuticals |
| IL6 | Gerilimzumab (ARGX-109) | arGEN-X/RuiYi |
| IL6 | FE301 | Ferring Pharmaceuticals |
| IL6 | FM101 | Femta Pharmaceuticals |
| IL-6R | Sarilumab (directed against | Regeneron/Sanofi |
| | IL6R) | |
| IL-6R | Tocilizumab | Hoffmann-La Roche/Chugai |
| IL-6R | Sapelizumab | Chugai |
| IL-6R | Vobarilizumab | Ablynx |
| L1CAM | AB417 | Creative biolabs |
| L1CAM | L1-9.3 | Creative biolabs |
| L1CAM | L1-14.10 | Biolegend |
| NGF | Tanezumab | Pfizer |
| NGF | Fulranumab (JNJ-42160443), | Amgen |
| NGF | MNAC13 (anti-TrkA, the | Creative Biolabs |
| | NGF receptor) | |
| NGF | mAb 911 | Rinat/Pfizer |
| NGF | Fasinumab | Regeneron/Teva |
| NRG1 | 538.24 | Hoffman-La Roche |
| NRP1 | Vesencumab | Genentech/Roche |
| ROR1 | Cirmtuzumab | Oncternal Therapeutics |
| SAP | GSK2398852 | GlaxoSmithKline |
| TGFβ | Fresolimumab (pan-TGFβ | Genzyme/Aventis |
| | antibody) | |
| TGFβ | IMC-TR1 (LY3022859) | Eli Lilly |
| | (MAb against TGFβRII) | |
| TGFβ | TβM1 (anti-TGFβ1 MAb) | Eli Lilly |
| TGFβ2 | Lerdelimumab (CAT-152) | Genzyme |
| TGFβ1 | Metelimumab | Genzyme |
| TGFβ1 | LY2382770 | Eli Lilly |
| TGFβ | PF-03446962 (MAb against | Pfizer |
| | TGFβRI) | |
| TNF | Infliximab | Janssen Biotech, Inc. |
| TNF | Adalimumab | AbbVie Inc. |
| TNF | Certolizumab pegol | UCB |
| TNF | Golimumab | Janssen Biotech, Inc. |
| TNF | Afelimomab | |
| TNF | Placulumab | Teva Pharmaceutical |
| | | Industries, Inc. |
| TNF | Nerelimomab | Chiron/Celltech |
| TNF | Ozoralizumab | Pfizer/Ablynx |
| VEGFA | Bevacizumab | Genzyme |
| VEGFA | Ranibizumab | Lucentis |
| VEGF | Alacizumab pegol (anti- | UCB |
| | VEGFR2) | |
| VEGFA | Brolucizumab | Novartis |
| VEGF | Icrucumab (anti-VEGFR1) | Eli Lilly |
| VEGF | Ramucirumab (anti-VEGFR2) | Eli Lilly |

Neuronal growth factor modulators also include agents that agonize or antagonize neuronal growth factors and neuronal growth factor receptors. For example, neuronal growth factor modulators include TNF inhibitors (e.g., etanercept, thalidomide, lenalidomide, pomalidomide, pentoxifylline, bupropion, and DOI), TGFβ1 inhibitors, (e.g., disitertide (P144)), TGFβ2 inhibitors (e.g., trabedersen (AP12009)). Exemplary neuronal growth factor agonists and antagonists are listed in Table 6.

TABLE 6
NEURONAL GROWTH FACTOR AGONISTS AND ANTAGONISTS

| | Agonist | Antagonist |
|---|---|---|
| TrkA | NGF, amitriptyline, and gambogic | Ale-0540 |
| | amide, gambogic acid | |
| TrkB | BDNF, NT3, NT4, 3,7- | ANA-12, cyclotraxin B, and |
| | Dihydroxyflavone, 3,7,8,2′- | gossypetin |
| | Tetrahydroxyflavone, 4′- | |
| | Dimethylamino-7,8- | |
| | dihydroxyflavone, 7,3′- | |
| | Dihydroxyflavone, 7,8- | |
| | Dihydroxyflavone, 7,8,2′- | |
| | Trihydroxyflavone, 7,8,3′- | |
| | Trihydroxyflavone, Amitriptyline^{,} | |
| | Deoxygedunin, Diosmetin, HIOC, | |
| | LM22A-4, N-Acetylserotonin, | |
| | Norwogonin (5,7,8-THF), R7, | |
| | LM22A4, and TDP6 | |
| Pan-Trk receptor | | Entrectinib (RXDX-101), AG 879, |
| | | GNF 5837, GW 441756, and PF |
| | | 06273340 |
| GFRα1R | GDNF and XIB4035 | |
| VEGF receptor | | AEE 788, AG 879, AP 24534, |
| | | axitinib, DMH4, GSK 1363089, Ki |
| | | 8751, RAF 265, SU 4312, SU |
| | | 5402, SU 5416, SU 6668, |
| | | sunitinib, toceranib, vatalanib, XL |
| | | 184, ZM 306416, and ZM 323881 |
| TGFβRI | | Galunisertib (LY2157299), TEW- |
| | | 7197, SB-431542, A 83-01, D |
| | | 4476, GW 788388, LY 364947, R |
| | | 268712, repsox, SB 505124, SB |
| | | 525334, and SD 208 |

Modulators of Gene Expression

In some embodiments, a neuromodulating agent is a neurome gene expression modulator (e.g., an agent that affects the expression of a neurome gene listed in Table 7 or Table 8, e.g., a channel, transporter, neuropeptide, neurotransmitter, neurotrophic, signaling, synaptic, biosynthesis, ligand, receptor, structural, or vesicular gene). A neurome gene expression modulator can affect gene expression through modulation of gene transcription, gene translation, or protein levels. Neurome gene expression modulators may increase gene expression through epigenetic modifications (e.g., demethylation or acetylation), post-translational modifications (e.g., reducing ubiquitination, or altering sumoylation or phosphorylation), by increasing mRNA translation and stability, or through delivery of exogenous genetic material (e.g., a viral vector expressing a gene of interest). In some embodiments, the neurome gene expression modulator increases neurome gene expression by stabilizing, upregulating, or promoting overexpression of a biosynthesis, channel, ligand, receptor, signaling, structural, synaptic, transporter, vesicular, neuropeptide, neurotransmitter, or neurotrophic gene in Table 7 or a channel or transporter gene in Table 8. Neurome gene expression modulators may decrease gene expression through epigenetic modifications (e.g., methylation or deacetylation), post-translational modifications (e.g., increasing ubiquitination, or altering sumoylation or phosphorylation), or by decreasing mRNA translation and stability (e.g., using miRNA, siRNA, shRNA, or other therapeutic RNAs). In some embodiments, the neurome gene expression modulator decreases neurome gene expression by downregulating, inhibiting, or disrupting expression of a biosynthesis, channel, ligand, receptor, signaling, structural, synaptic, transporter, vesicular, neuropeptide, neurotransmitter, or neurotrophic gene in Table 7 or a channel or transporter gene in Table 8. A neurome gene expression modulator may increase or decrease gene expression by 10%, 20%, 30%, 40%, 50%, 60%, 70%, 80%, 90%, 95%, 98%, or more.

In some embodiments, a neurome gene expression modulator increases or decreases the expression of a neurome gene listed in Table 7 to treat a subject with cancer (e.g., a cancer that expresses the neurome gene, e.g., through altering the growth, metastasis, invasion, proliferation, or viability of the cancer cell expressing the modulated gene). In some embodiments, a neurome gene expression modulator increases or decreases the expression of a neurome gene listed in Table 10 or in Table 12, 13, or 14A-14C to treat the corresponding cancer. The neurome gene expression modulator can be introduced systemically (e.g., injected intravenously into blood stream), or administered locally (e.g., administered to a tumor or a tissue at risk of developing a tumor).

TABLE 7
NEUROME GENES

| Approved | | Entrez | Gene type/ | |
|---|---|---|---|---|
| Symbol | Approved name | Gene ID | family | Category |
| ABAT | 4-aminobutyrate | 18 | Neurotransmitter | Biosynthesis |
| | aminotransferase | | | |
| ACHE | Acetylcholinesterase | 43 | Neurotransmitter | Biosynthesis |
| ACTR1A | ARP1 actin-related protein 1 | 10121 | Synaptic | Structural |
| | homolog A, centractin alpha | | | |
| ACTR1B | ARP1 actin-related protein 1 | 10120 | Synaptic | Structural |
| | homolog B, centractin beta | | | |
| ADCY1 | Adenylate cyclase 1 | 107 | Signaling | Signaling |
| ADCY10 | Adenylate cyclase 10, soluble | 55811 | Signaling | Signaling |
| ADCY2 | Adenylate cyclase 2 | 108 | Signaling | Signaling |
| ADCY3 | Adenylate cyclase 3 | 109 | Signaling | Signaling |
| ADCY4 | Adenylate cyclase 4 | 196883 | Signaling | Signaling |
| ADCY5 | Adenylate cyclase 5 | 111 | Signaling | Signaling |
| ADCY6 | Adenylate cyclase 6 | 112 | Signaling | Signaling |
| ADCY7 | Adenylate cyclase 7 | 113 | Signaling | Signaling |
| ADCY8 | Adenylate cyclase 8 | 114 | Signaling | Signaling |
| ADCY9 | Adenylate cyclase 9 | 115 | Signaling | Signaling |
| ADCYAP1 | Adenylate cyclase activating | 116 | Signaling | Signaling |
| | polypeptide 1 | | | |
| ADCYAP1R1 | ADCYAP receptor type I | 117 | Neurotransmitter | Receptor |
| ADIPOQ | Adiponectin, C1Q and | 9370 | Neuropeptide | Ligand |
| | collagen domain containing | | | |
| ADK | Adenosine kinase | 132 | Signaling | Signaling |
| ADM | Adrenomedullin | 133 | Neuropeptide | Ligand |
| ADM2 | Adrenomedullin 2 | 79924 | Neuropeptide | Ligand |
| ADNP | Activity dependent | 23394 | Signaling | Structural |
| | neuroprotector homeobox | | | |
| ADORA2A | Adenosine A2a receptor | 135 | Neurotransmitter | Receptor |
| ADORA2B | Adenosine A2b receptor | 136 | Neurotransmitter | Receptor |
| ADRA1A | Adrenoceptor alpha 1A | 148 | Neurotransmitter | Receptor |
| ADRA1B | Adrenoceptor alpha 1B | 147 | Neurotransmitter | Receptor |
| ADRA1D | Adrenoceptor alpha 1D | 146 | Neurotransmitter | Receptor |
| ADRA2A | Adrenoceptor alpha 2A | 150 | Neurotransmitter | Receptor |
| ADRA2B | Adrenoceptor alpha 2B | 151 | Neurotransmitter | Receptor |
| ADRA2C | Adrenoceptor alpha 2C | 152 | Neurotransmitter | Receptor |
| ADRB1 | Adrenoceptor beta 1 | 153 | Neurotransmitter | Receptor |
| ADRB2 | Adrenoceptor beta 2 | 154 | Neurotransmitter | Receptor |
| ADRB3 | Adrenoceptor beta 3 | 155 | Neurotransmitter | Receptor |
| AGRN | Agrin | 375790 | Neuropeptide | Ligand |
| AGRP | Agouti related neuropeptide | 181 | Neuropeptide | Ligand |
| AGT | Angiotensinogen | 183 | Neuropeptide | Ligand |
| AGTR1 | Angiotensin II receptor type 1 | 185 | Neuropeptide | Receptor |
| AKAP1 | A-kinase anchoring protein 1 | 8165 | Signaling | Signaling |
| AKAP10 | A-kinase anchoring protein 10 | 11216 | Signaling | Signaling |
| AKAP11 | A-kinase anchoring protein 11 | 11215 | Signaling | Signaling |
| AKAP12 | A-kinase anchoring protein 12 | 9590 | Signaling | Signaling |
| AKAP13 | A-kinase anchoring protein 13 | 11214 | Signaling | Signaling |
| AKAP14 | A-kinase anchoring protein 14 | 158798 | Signaling | Signaling |
| AKAP17A | A-kinase anchoring protein | 8227 | Signaling | Signaling |
| | 17A | | | |
| AKAP2 | A-kinase anchoring protein 2 | 11217 | Signaling | Signaling |
| AKAP3 | A-kinase anchoring protein 3 | 10566 | Signaling | Signaling |
| AKAP4 | A-kinase anchoring protein 4 | 8852 | Signaling | Signaling |
| AKAP5 | A-kinase anchoring protein 5 | 9495 | Signaling | Signaling |
| AKAP6 | A-kinase anchoring protein 6 | 9472 | Signaling | Signaling |
| AKAP7 | A-kinase anchoring protein 7 | 9465 | Signaling | Signaling |
| AKAP8 | A-kinase anchoring protein 8 | 10270 | Signaling | Signaling |
| AKAP9 | A-kinase anchoring protein 9 | 10142 | Signaling | Signaling |
| AKT1 | AKT serine/threonine kinase 1 | 207 | Signaling | Signaling |
| AKT2 | AKT serine/threonine kinase 2 | 208 | Signaling | Signaling |
| AKT3 | AKT serine/threonine kinase 3 | 10000 | Signaling | Signaling |
| ALDH5A1 | Aldehyde dehydrogenase 5 | 7915 | Other | Miscelaneous |
| | family member A1 | | | |
| ALDH9A1 | Aldehyde dehydrogenase 9 | 223 | Other | Miscelaneous |
| | family member A1 | | | |
| ALOX15 | Arachidonate 15-lipoxygenase | 246 | Other | Miscelaneous |
| ALPL | Alkaline phosphatase, | 249 | Other | Miscelaneous |
| | liver/bone/kidney | | | |
| ANXA1 | Annexin A1 | 301 | Other | Miscelaneous |
| AP3B2 | Adaptor related protein | 8120 | Vesicular | Vesicles |
| | complex 3 beta 2 subunit | | | |
| AP3D1 | Adaptor related protein | 8943 | Vesicular | Vesicles |
| | complex 3 delta 1 subunit | | | |
| APLN | Apelin | 8862 | Neuropeptide | Ligand |
| APOE | Apolipoprotein E | 348 | Other | Miscelaneous |
| ARID1B | AT-rich interaction domain 1B | 57492 | Other | Miscelaneous |
| ARPP19 | Camp regulated | 10776 | Signaling | Signaling |
| | phosphoprotein 19 | | | |
| ARR3 | Arrestin 3 | 407 | Signaling | Signaling |
| ARRB1 | Arrestin beta 1 | 408 | Signaling | Signaling |
| ARRB2 | Arrestin beta 2 | 409 | Signaling | Signaling |
| ARTN | Artemin | 9048 | Neurotrophic | Ligand |
| ASIC1 | Acid sensing ion channel | 41 | Channel or | Channel |
| | subunit 1 | | transporter | |
| ASIC2 | Acid sensing ion channel | 40 | Channel or | Channel |
| | subunit 2 | | transporter | |
| ASIC3 | Acid sensing ion channel | 9311 | Channel or | Channel |
| | subunit 3 | | transporter | |
| ASIP | Agouti signaling protein | 434 | Neuropeptide | Ligand |
| ATP10A | Atpase phospholipid | 57194 | Channel or | Transporter |
| | transporting 10A (putative) | | transporter | |
| ATP10B | Atpase phospholipid | 23120 | Channel or | Transporter |
| | transporting 10B (putative) | | transporter | |
| ATP10D | Atpase phospholipid | 57205 | Channel or | Transporter |
| | transporting 10D (putative) | | transporter | |
| ATP11A | Atpase phospholipid | 23250 | Channel or | Transporter |
| | transporting 11A | | transporter | |
| ATP11B | Atpase phospholipid | 23200 | Channel or | Transporter |
| | transporting 11B (putative) | | transporter | |
| ATP11C | Atpase phospholipid | 286410 | Channel or | Transporter |
| | transporting 11C | | transporter | |
| ATP12A | Atpase H+/K+ transporting | 479 | Channel or | Transporter |
| | non-gastric alpha2 subunit | | transporter | |
| ATP1A1 | Atpase Na+/K+ transporting | 476 | Channel or | Transporter |
| | subunit alpha 1 | | transporter | |
| ATP1A2 | Atpase Na+/K+ transporting | 477 | Channel or | Transporter |
| | subunit alpha 2 | | transporter | |
| ATP1A3 | Atpase Na+/K+ transporting | 478 | Channel or | Transporter |
| | subunit alpha 3 | | transporter | |
| ATP1A4 | Atpase Na+/K+ transporting | 480 | Channel or | Transporter |
| | subunit alpha 4 | | transporter | |
| ATP1B1 | Atpase Na+/K+ transporting | 481 | Channel or | Transporter |
| | subunit beta 1 | | transporter | |
| ATP1B2 | Atpase Na+/K+ transporting | 482 | Channel or | Transporter |
| | subunit beta 2 | | transporter | |
| ATP1B3 | Atpase Na+/K+ transporting | 483 | Channel or | Transporter |
| | subunit beta 3 | | transporter | |
| ATP2A1 | Atpase | 487 | Channel or | Transporter |
| | sarcoplasmic/endoplasmic | | transporter | |
| | reticulum Ca2+ transporting 1 | | | |
| ATP2A2 | Atpase | 488 | Channel or | Transporter |
| | sarcoplasmic/endoplasmic | | transporter | |
| | reticulum Ca2+ transporting 2 | | | |
| ATP2A3 | Atpase | 489 | Channel or | Transporter |
| | sarcoplasmic/endoplasmic | | transporter | |
| | reticulum Ca2+ transporting 3 | | | |
| ATP2B1 | Atpase plasma membrane | 490 | Channel or | Transporter |
| | Ca2+ transporting 1 | | transporter | |
| ATP2B2 | Atpase plasma membrane | 491 | Channel or | Transporter |
| | Ca2+ transporting 2 | | transporter | |
| ATP2B3 | Atpase plasma membrane | 492 | Channel or | Transporter |
| | Ca2+ transporting 3 | | transporter | |
| ATP2B4 | Atpase plasma membrane | 493 | Channel or | Transporter |
| | Ca2+ transporting 4 | | transporter | |
| ATP2C1 | Atpase secretory pathway | 27032 | Channel or | Transporter |
| | Ca2+ transporting 1 | | transporter | |
| ATP2C2 | Atpase secretory pathway | 9914 | Channel or | Transporter |
| | Ca2+ transporting 2 | | transporter | |
| ATP4A | Atpase H+/K+ transporting | 495 | Channel or | Transporter |
| | alpha subunit | | transporter | |
| ATP4B | Atpase H+/K+ transporting | 496 | Channel or | Transporter |
| | beta subunit | | transporter | |
| ATP5A1 | ATP synthase, H+ | 498 | Channel or | Transporter |
| | transporting, mitochondrial F1 | | transporter | |
| | complex, alpha subunit 1, | | | |
| | cardiac muscle | | | |
| ATP5B | ATP synthase, H+ | 506 | Channel or | Transporter |
| | transporting, mitochondrial F1 | | transporter | |
| | complex, beta polypeptide | | | |
| ATP5C1 | ATP synthase, H+ | 509 | Channel or | Transporter |
| | transporting, mitochondrial F1 | | transporter | |
| | complex, gamma polypeptide | | | |
| | 1 | | | |
| ATP5D | ATP synthase, H+ | 513 | Channel or | Transporter |
| | transporting, mitochondrial F1 | | transporter | |
| | complex, delta subunit | | | |
| ATP5E | ATP synthase, H+ | 514 | Channel or | Transporter |
| | transporting, mitochondrial F1 | | transporter | |
| | complex, epsilon subunit | | | |
| ATP5F1 | ATP synthase, H+ | 515 | Channel or | Transporter |
| | transporting, mitochondrial Fo | | transporter | |
| | complex subunit B1 | | | |
| ATP5H | ATP synthase, H+ | 10476 | Channel or | Transporter |
| | transporting, mitochondrial Fo | | transporter | |
| | complex subunit D | | | |
| ATP5I | ATP synthase, H+ | 521 | Channel or | Transporter |
| | transporting, mitochondrial Fo | | transporter | |
| | complex subunit E | | | |
| ATP5J | ATP synthase, H+ | 522 | Channel or | Transporter |
| | transporting, mitochondrial Fo | | transporter | |
| | complex subunit F6 | | | |
| ATP5J2 | ATP synthase, H+ | 9551 | Channel or | Transporter |
| | transporting, mitochondrial Fo | | transporter | |
| | complex subunit F2 | | | |
| ATP5L2 | ATP synthase, H+ | 267020 | Channel or | Transporter |
| | transporting, mitochondrial Fo | | transporter | |
| | complex subunit G2 | | | |
| ATP6V0A1 | Atpase H+ transporting V0 | 535 | Channel or | Transporter |
| | subunit a1 | | transporter | |
| ATP6V0A2 | Atpase H+ transporting V0 | 23545 | Channel or | Transporter |
| | subunit a2 | | transporter | |
| ATP6V0A4 | Atpase H+ transporting V0 | 50617 | Channel or | Transporter |
| | subunit a4 | | transporter | |
| ATP6V0B | Atpase H+ transporting V0 | 533 | Channel or | Transporter |
| | subunit b | | transporter | |
| ATP6V0C | Atpase H+ transporting V0 | 527 | Channel or | Transporter |
| | subunit c | | transporter | |
| ATP6V0D1 | Atpase H+ transporting V0 | 9114 | Channel or | Transporter |
| | subunit d1 | | transporter | |
| ATP6V0D2 | Atpase H+ transporting V0 | 245972 | Channel or | Transporter |
| | subunit d2 | | transporter | |
| ATP6V0E1 | Atpase H+ transporting V0 | 8992 | Channel or | Transporter |
| | subunit e1 | | transporter | |
| ATP6V0E2 | Atpase H+ transporting V0 | 155066 | Channel or | Transporter |
| | subunit e2 | | transporter | |
| ATP6V1A | Atpase H+ transporting V1 | 523 | Channel or | Transporter |
| | subunit A | | transporter | |
| ATP6V1B1 | Atpase H+ transporting V1 | 525 | Channel or | Transporter |
| | subunit B1 | | transporter | |
| ATP6V1B2 | Atpase H+ transporting V1 | 526 | Channel or | Transporter |
| | subunit B2 | | transporter | |
| ATP6V1C1 | Atpase H+ transporting V1 | 528 | Channel or | Transporter |
| | subunit C1 | | transporter | |
| ATP6V1C2 | Atpase H+ transporting V1 | 245973 | Channel or | Transporter |
| | subunit C2 | | transporter | |
| ATP6V1D | Atpase H+ transporting V1 | 51382 | Channel or | Transporter |
| | subunit D | | transporter | |
| ATP6V1E1 | Atpase H+ transporting V1 | 529 | Channel or | Transporter |
| | subunit E1 | | transporter | |
| ATP6V1E2 | Atpase H+ transporting V1 | 90423 | Channel or | Transporter |
| | subunit E2 | | transporter | |
| ATP6V1F | Atpase H+ transporting V1 | 9296 | Channel or | Transporter |
| | subunit F | | transporter | |
| ATP6V1G1 | Atpase H+ transporting V1 | 9550 | Channel or | Transporter |
| | subunit G1 | | transporter | |
| ATP6V1G2 | Atpase H+ transporting V1 | 534 | Channel or | Transporter |
| | subunit G2 | | transporter | |
| ATP6V1G3 | Atpase H+ transporting V1 | 127124 | Channel or | Transporter |
| | subunit G3 | | transporter | |
| ATP6V1H | Atpase H+ transporting V1 | 51606 | Channel or | Transporter |
| | subunit H | | transporter | |
| ATP7A | Atpase copper transporting | 538 | Channel or | Transporter |
| | alpha | | transporter | |
| ATP7B | Atpase copper transporting | 540 | Channel or | Transporter |
| | beta | | transporter | |
| ATP8A1 | Atpase phospholipid | 10396 | Channel or | Transporter |
| | transporting 8A1 | | transporter | |
| ATP8A2 | Atpase phospholipid | 51761 | Channel or | Transporter |
| | transporting 8A2 | | transporter | |
| ATP8B1 | Atpase phospholipid | 5205 | Channel or | Transporter |
| | transporting 8B1 | | transporter | |
| ATP8B2 | Atpase phospholipid | 57198 | Channel or | Transporter |
| | transporting 8B2 | | transporter | |
| ATP8B3 | Atpase phospholipid | 148229 | Channel or | Transporter |
| | transporting 8B3 | | transporter | |
| ATP8B4 | Atpase phospholipid | 79895 | Channel or | Transporter |
| | transporting 8B4 (putative) | | transporter | |
| ATP9A | Atpase phospholipid | 10079 | Channel or | Transporter |
| | transporting 9A (putative) | | transporter | |
| ATP9B | Atpase phospholipid | 374868 | Channel or | Transporter |
| | transporting 9B (putative) | | transporter | |
| AVP | Arginine vasopressin | 551 | Neuropeptide | Ligand |
| AVPR1A | Arginine vasopressin receptor | 552 | Neuropeptide | Receptor |
| | 1A | | | |
| AVPR1B | Arginine vasopressin receptor | 553 | Neuropeptide | Receptor |
| | 1B | | | |
| AVPR2 | Arginine vasopressin receptor | 554 | Neuropeptide | Receptor |
| | 2 | | | |
| BACE1 | Beta-secretase 1 | 23621 | Neurotransmitter | Biosynthesis |
| BAG2 | BCL2 associated athanogene | 9532 | Signaling | Signaling |
| | 2 | | | |
| BAIAP3 | BAI1 associated protein 3 | 8938 | Signaling | Signaling |
| BCHE | Butyrylcholinesterase | 590 | Neurotransmitter | Biosynthesis |
| BCL2 | BCL2, apoptosis regulator | 596 | Signaling | Signaling |
| BDKRB2 | Bradykinin receptor B2 | 624 | Neuropeptide | Receptor |
| BDNF | Brain derived neurotrophic | 627 | Neurotrophic | Ligand |
| | factor | | | |
| BDNF-AS | BDNF antisense RNA | 497258 | Neurotrophic | Ligand |
| BEX1 | Protein BEX1 (Brain- | 55859 | Neurotrophic | Signaling |
| | expressed X-linked protein 1) | | | |
| BEX3 | Protein BEX3 (Brain- | 27018 | Neurotrophic | Signaling |
| | expressed X-linked protein 3) | | | |
| BRAF | B-Raf proto-oncogene, | 673 | Signaling | Signaling |
| | serine/threonine kinase | | | |
| BRSK1 | BR serine/threonine kinase 1 | 84446 | Vesicular | Vesicles |
| BSN | Bassoon presynaptic | 8927 | Other | Miscelaneous |
| | cytomatrix protein | | | |
| BTBD9 | BTB domain containing 9 | 114781 | Vesicular | Vesicles |
| C1QBP | Complement C1q binding | 708 | Neuropeptide | Receptor |
| | protein | | | |
| C2CD4A | C2 calcium dependent domain | 145741 | Other | Miscelaneous |
| | containing 4A | | | |
| C2CD4B | C2 calcium dependent domain | 388125 | Other | Miscelaneous |
| | containing 4B | | | |
| C2CD4C | C2 calcium dependent domain | 126567 | Other | Miscelaneous |
| | containing 4C | | | |
| C2CD4D | C2 calcium dependent domain | 100191040 | Other | Miscelaneous |
| | containing 4D | | | |
| C4orf48 | Chromosome 4 open reading | 401115 | Neuropeptide | Ligand |
| | frame 48 | | | |
| CACNA1A | Calcium voltage-gated | 773 | Channel or | Channel |
| | channel subunit alpha1 A | | transporter | |
| CACNA1B | Calcium voltage-gated | 774 | Channel or | Channel |
| | channel subunit alpha1 B | | transporter | |
| CACNA1C | Calcium voltage-gated | 775 | Channel or | Channel |
| | channel subunit alpha1 C | | transporter | |
| CACNA1D | Calcium voltage-gated | 776 | Channel or | Channel |
| | channel subunit alpha1 D | | transporter | |
| CACNA1E | Calcium voltage-gated | 777 | Channel or | Channel |
| | channel subunit alpha1 E | | transporter | |
| CACNA1F | Calcium voltage-gated | 778 | Channel or | Channel |
| | channel subunit alpha1 F | | transporter | |
| CACNA1G | Calcium voltage-gated | 8913 | Channel or | Channel |
| | channel subunit alpha1 G | | transporter | |
| CACNA1H | Calcium voltage-gated | 8912 | Channel or | Channel |
| | channel subunit alpha1 H | | transporter | |
| CACNA1I | Calcium voltage-gated | 8911 | Channel or | Channel |
| | channel subunit alpha1 I | | transporter | |
| CACNA1S | Calcium voltage-gated | 779 | Channel or | Channel |
| | channel subunit alpha1 S | | transporter | |
| CACNB1 | Calcium voltage-gated | 782 | Channel or | Channel |
| | channel auxiliary subunit beta | | transporter | |
| | 1 | | | |
| CACNB2 | Calcium voltage-gated | 783 | Channel or | Channel |
| | channel auxiliary subunit beta | | transporter | |
| | 2 | | | |
| CACNB4 | Calcium voltage-gated | 785 | Channel or | Channel |
| | channel auxiliary subunit beta | | transporter | |
| | 4 | | | |
| CALCA | Calcitonin related polypeptide | 796 | Neuropeptide | Ligand |
| | alpha, CGRP | | | |
| CALCB | Calcitonin related polypeptide | 797 | Neuropeptide | Ligand |
| | beta | | | |
| CALCR | Calcitonin receptor | 799 | Neuropeptide | Receptor |
| CALCRL | Calcitonin receptor like | 10203 | Neuropeptide | Receptor |
| | receptor | | | |
| CALM1 | Calmodulin 1 | 801 | Signaling | Signaling |
| CALM2 | Calmodulin 2 | 805 | Signaling | Signaling |
| CALM3 | Calmodulin 3 | 808 | Signaling | Signaling |
| CALY | Calcyon neuron specific | 50632 | Other | Miscelaneous |
| | vesicular protein | | | |
| CAMK2A | Calcium/calmodulin | 815 | Signaling | Signaling |
| | dependent protein kinase II | | | |
| | alpha | | | |
| CAMK2B | Calcium/calmodulin | 816 | Signaling | Signaling |
| | dependent protein kinase II | | | |
| | beta | | | |
| CAMK4 | Calcium/calmodulin | 814 | Signaling | Signaling |
| | dependent protein kinase IV | | | |
| CARF | Calcium responsive | 79800 | Signaling | Signaling |
| | transcription factor | | | |
| CARTPT | CART prepropeptide | 9607 | Neuropeptide | Ligand |
| CASK | Calcium/calmodulin | 8573 | Signaling | Signaling |
| | dependent serine protein | | | |
| | kinase | | | |
| CASR | Calcium sensing receptor | 846 | Neuropeptide | Biosynthesis |
| CATSPER1 | Cation channel sperm | 117144 | Channel or | Channel |
| | associated 1 | | transporter | |
| CATSPER2 | Cation channel sperm | 117155 | Channel or | Channel |
| | associated 2 | | transporter | |
| CATSPER3 | Cation channel sperm | 347732 | Channel or | Channel |
| | associated 3 | | transporter | |
| CATSPER4 | Cation channel sperm | 378807 | Channel or | Channel |
| | associated 4 | | transporter | |
| CBLN1 | Cerebellin 1 precursor | 869 | Other | Miscelaneous |
| CBLN2 | Cerebellin 2 precursor | 147381 | Other | Miscelaneous |
| CBLN3 | Cerebellin 3 precursor | 643866 | Other | Miscelaneous |
| CBLN4 | Cerebellin 4 precursor | 140689 | Other | Miscelaneous |
| CCK | Cholecystokinin | 885 | Neuropeptide | Ligand |
| CCKAR | Cholecystokinin A receptor | 886 | Neuropeptide | Receptor |
| CCKBR | Cholecystokinin B receptor | 887 | Neuropeptide | Receptor |
| CCL2 | C-C motif chemokine ligand 2 | 6347 | Neuropeptide | Ligand |
| CCR1 | C-C motif chemokine receptor | 1230 | Other | Miscelaneous |
| | 1 | | | |
| CD34 | Hematopoietic progenitor cell | 947 | Neurotrophic | Receptor |
| | antigen CD34 (CD antigen | | | |
| | CD34) | | | |
| CD38 | CD38 molecule | 952 | Signaling | Signaling |
| CDH2 | Cadherin 2 | 1000 | Signaling | Signaling |
| CDK5 | Cyclin dependent kinase 5 | 1020 | Signaling | Signaling |
| CDK5R1 | Cyclin dependent kinase 5 | 8851 | Signaling | Signaling |
| | regulatory subunit 1 | | | |
| CDKN1A | Cyclin dependent kinase | 1026 | Signaling | Signaling |
| | inhibitor 1A | | | |
| CDNF | Cerebral dopamine | 441549 | Neurotrophic | Ligand |
| | neurotrophic factor | | | |
| CHAT | Choline O-acetyltransferase | 1103 | Neurotransmitter | Biosynthesis |
| CHGA | Chromogranin A | 1113 | Neuropeptide | Ligand |
| CHGB | Chromogranin B | 1114 | Neuropeptide | Ligand |
| CHMP4A | Charged multivesicular body | 29082 | Vesicular | Vesicles |
| | protein 4A | | | |
| CHMP4B | Charged multivesicular body | 128866 | Vesicular | Vesicles |
| | protein 4B | | | |
| CHRFAM7A | CHRNA7 (exons 5-10) and | 89832 | Neurotransmitter | Receptor |
| | FAM7A (exons A-E) fusion | | | |
| CHRM1 | Cholinergic receptor | 1128 | Neurotransmitter | Receptor |
| | muscarinic 1 | | | |
| CHRM2 | Cholinergic receptor | 1129 | Neurotransmitter | Receptor |
| | muscarinic 2 | | | |
| CHRM3 | Cholinergic receptor | 1131 | Neurotransmitter | Receptor |
| | muscarinic 3 | | | |
| CHRM4 | Cholinergic receptor | 1132 | Neurotransmitter | Receptor |
| | muscarinic 4 | | | |
| CHRM5 | Cholinergic receptor | 1133 | Neurotransmitter | Receptor |
| | muscarinic 5 | | | |
| CHRNA1 | Cholinergic receptor nicotinic | 1134 | Neurotransmitter | Receptor |
| | alpha 1 subunit | | | |
| CHRNA10 | Cholinergic receptor nicotinic | 57053 | Neurotransmitter | Receptor |
| | alpha 10 subunit | | | |
| CHRNA2 | Cholinergic receptor nicotinic | 1135 | Neurotransmitter | Receptor |
| | alpha 2 subunit | | | |
| CHRNA3 | Cholinergic receptor nicotinic | 1136 | Neurotransmitter | Receptor |
| | alpha 3 subunit | | | |
| CHRNA4 | Cholinergic receptor nicotinic | 1137 | Neurotransmitter | Receptor |
| | alpha 4 subunit | | | |
| CHRNA5 | Cholinergic receptor nicotinic | 1138 | Neurotransmitter | Receptor |
| | alpha 5 subunit | | | |
| CHRNA6 | Cholinergic receptor nicotinic | 8973 | Neurotransmitter | Receptor |
| | alpha 6 subunit | | | |
| CHRNA7 | Cholinergic receptor nicotinic | 1139 | Neurotransmitter | Receptor |
| | alpha 7 subunit | | | |
| CHRNA9 | Cholinergic receptor nicotinic | 55584 | Neurotransmitter | Receptor |
| | alpha 9 subunit | | | |
| CHRNB1 | Cholinergic receptor nicotinic | 1140 | Neurotransmitter | Receptor |
| | beta 1 subunit | | | |
| CHRNB2 | Cholinergic receptor nicotinic | 1141 | Neurotransmitter | Receptor |
| | beta 2 subunit | | | |
| CHRNB3 | Cholinergic receptor nicotinic | 1142 | Neurotransmitter | Receptor |
| | beta 3 subunit | | | |
| CHRNB4 | Cholinergic receptor nicotinic | 1143 | Neurotransmitter | Receptor |
| | beta 4 subunit | | | |
| CHRND | Cholinergic receptor nicotinic | 1144 | Neurotransmitter | Receptor |
| | delta subunit | | | |
| CHRNE | Cholinergic receptor nicotinic | 1145 | Neurotransmitter | Receptor |
| | epsilon subunit | | | |
| CHRNG | Cholinergic receptor nicotinic | 1146 | Neurotransmitter | Receptor |
| | gamma subunit | | | |
| CLCF1 | Cardiotrophin-like cytokine | 23529 | Neuropeptide | Ligand |
| | factor 1 | | | |
| CLCN1 | Chloride voltage-gated | 1180 | Channel or | Channel |
| | channel 1 | | transporter | |
| CLCN2 | Chloride voltage-gated | 1181 | Channel or | Channel |
| | channel 2 | | transporter | |
| CLCN3 | Chloride voltage-gated | 1182 | Channel or | Channel |
| | channel 3 | | transporter | |
| CLCN4 | Chloride voltage-gated | 1183 | Channel or | Channel |
| | channel 4 | | transporter | |
| CLCN5 | Chloride voltage-gated | 1184 | Channel or | Channel |
| | channel 5 | | transporter | |
| CLCN6 | Chloride voltage-gated | 1185 | Channel or | Channel |
| | channel 6 | | transporter | |
| CLCN7 | Chloride voltage-gated | 1186 | Channel or | Channel |
| | channel 7 | | transporter | |
| CLCNKA | Chloride voltage-gated | 1187 | Channel or | Channel |
| | channel Ka | | transporter | |
| CLCNKB | Chloride voltage-gated | 1188 | Channel or | Channel |
| | channel Kb | | transporter | |
| CLIC6 | Chloride intracellular channel | 54102 | Channel or | Channel |
| | 6 | | transporter | |
| CLN3 | CLN3, battenin | 1201 | Other | Miscelaneous |
| CNGA1 | Cyclic nucleotide gated | 1259 | Channel or | Channel |
| | channel alpha 1 | | transporter | |
| CNGA2 | Cyclic nucleotide gated | 1260 | Channel or | Channel |
| | channel alpha 2 | | transporter | |
| CNGA3 | Cyclic nucleotide gated | 1261 | Channel or | Channel |
| | channel alpha 3 | | transporter | |
| CNGA4 | Cyclic nucleotide gated | 1262 | Channel or | Channel |
| | channel alpha 4 | | transporter | |
| CNGB1 | Cyclic nucleotide gated | 1258 | Channel or | Channel |
| | channel beta 1 | | transporter | |
| CNGB3 | Cyclic nucleotide gated | 54714 | Channel or | Channel |
| | channel beta 3 | | transporter | |
| CNR1 | Cannabinoid receptor 1 | 1268 | Neurotransmitter | Receptor |
| CNR2 | Cannabinoid receptor 2 | 1269 | Neurotransmitter | Receptor |
| CNRIP1 | Cannabinoid receptor | 25927 | Neurotransmitter | Receptor |
| | interacting protein 1 | | | |
| CNTF | Ciliary neurotrophic factor | 1270 | Neurotrophic | Ligand |
| CNTFR | Ciliary neurotrophic factor | 1271 | Neurotrophic | Receptor |
| | receptor | | | |
| CNTNAP4 | Contactin associated protein | 85445 | Vesicular | Vesicles |
| | like 4 | | | |
| COMT | Catechol-O-methyltransferase | 1312 | Neurotransmitter | Biosynthesis |
| CORT | Cortistatin | 1325 | Neuropeptide | Ligand |
| CPA4 | Carboxypeptidase A4 | 51200 | Neurotransmitter | Biosynthesis |
| CPE | Carboxypeptidase E | 1363 | Neurotransmitter | Biosynthesis |
| CPLX1 | Complexin 1 | 10815 | Vesicular | Vesicles |
| CPLX2 | Complexin 2 | 10814 | Vesicular | Vesicles |
| CPLX3 | Complexin 3 | 594855 | Vesicular | Vesicles |
| CPLX4 | Complexin 4 | 339302 | Vesicular | Vesicles |
| CRCP | CGRP receptor component | 27297 | Neuropeptide | Receptor |
| CREB1 | Camp responsive element | 1385 | Signaling | Signaling |
| | binding protein 1 | | | |
| CREM | Camp responsive element | 1390 | Neurotransmitter | Signaling |
| | modulator | | | |
| CRH | Corticotropin releasing | 1392 | Neuropeptide | Ligand |
| | hormone | | | |
| CRHR1 | Corticotropin releasing | 1394 | Neuropeptide | Receptor |
| | hormone receptor 1 | | | |
| CRHR2 | Corticotropin releasing | 1395 | Neuropeptide | Receptor |
| | hormone receptor 2 | | | |
| CRLF1 | Cytokine receptor like factor 1 | 9244 | Neurotrophic | Receptor |
| CSK | C-src tyrosine kinase | 1445 | Signaling | Signaling |
| CSNK1E | Casein kinase 1 epsilon | 1454 | Signaling | Signaling |
| CSPG5 | Chondroitin sulfate | 10675 | Neurotrophic | Ligand |
| | proteoglycan 5 | | | |
| CST3 | Cystatin C | 1471 | Other | Miscelaneous |
| CTAGE15 | CTAGE family member 15 | 441294 | Other | Miscelaneous |
| CTAGE4 | CTAGE family member 4 | 100128553 | Other | Miscelaneous |
| CTAGE6 | CTAGE family member 6 | 340307 | Other | Miscelaneous |
| CTAGE8 | CTAGE family member 8 | 100142659 | Other | Miscelaneous |
| CTAGE9 | CTAGE family member 9 | 643854 | Other | Miscelaneous |
| CTBP2 | C-terminal binding protein 2 | 1488 | Signaling | Signaling |
| CTSH | Cathepsin H | 1512 | Neuropeptide | Biosynthesis |
| CTSV | Cathepsin V | 1515 | Neuropeptide | Biosynthesis |
| CXCR4 | C-X-C motif chemokine | 7852 | Other | Miscelaneous |
| | receptor 4 | | | |
| CYFIP1 | Cytoplasmic FMR1 interacting | 23191 | Signaling | Signaling |
| | protein 1 | | | |
| CYP19A1 | Cytochrome P450 family 19 | 1588 | Other | Miscelaneous |
| | subfamily A member 1 | | | |
| CYSLTR1 | Cysteinyl leukotriene receptor | 10800 | Neuropeptide | Receptor |
| | 1 | | | |
| CYSLTR2 | Cysteinyl leukotriene receptor | 57105 | Neuropeptide | Receptor |
| | 2 | | | |
| DAGLA | Diacylglycerol lipase alpha | 747 | Neurotransmitter | Biosynthesis |
| DAGLB | Diacylglycerol lipase beta | 221955 | Neurotransmitter | Biosynthesis |
| DBH | Dopamine beta-hydroxylase | 1621 | Neurotransmitter | Biosynthesis |
| DBI | Diazepam binding inhibitor, | 1622 | Neuropeptide | Ligand |
| | acyl-coa binding protein | | | |
| DCLK1 | Doublecortin like kinase 1 | 9201 | Neurotrophic | Signaling |
| DDC | Dopa decarboxylase | 1644 | Neurotransmitter | Biosynthesis |
| DDR1 | Discoidin domain receptor | 780 | Other | Miscelaneous |
| | tyrosine kinase 1 | | | |
| DDR2 | Discoidin domain receptor | 4921 | Other | Miscelaneous |
| | tyrosine kinase 2 | | | |
| DGKI | Diacylglycerol kinase iota | 9162 | Neurotransmitter | Biosynthesis |
| DIRC2 | Disrupted in renal carcinoma 2 | 84925 | Channel or | Transporter |
| | | | transporter | |
| DISC1 | Disrupted in schizophrenia 1 | 27185 | Neurotrophic | Signaling |
| DKK1 | Dickkopf WNT signaling | 22943 | Other | Miscelaneous |
| | pathway inhibitor 1 | | | |
| DLGAP2 | DLG associated protein 2 | 9228 | Vesicular | Vesicles |
| DNAJC5 | Dnaj heat shock protein family | 80331 | Neurotrophic | Signaling |
| | (Hsp40) member C5 | | | |
| DNM1 | Dynamin 1 | 1759 | Vesicular | Vesicles |
| DNM2 | Dynamin 2 | 1785 | Vesicular | Vesicles |
| DOC2A | Double C2 domain alpha | 8448 | Signaling | Signaling |
| DOC2B | Double C2 domain beta | 8447 | Signaling | Signaling |
| DPP4 | Dipeptidyl peptidase 4 | 1803 | Neurotransmitter | Biosynthesis |
| DPYSL2 | Dihydropyrimidinase like 2 | 1808 | Neurotrophic | Signaling |
| DRD1 | Dopamine receptor D1 | 1812 | Neurotransmitter | Receptor |
| DRD2 | Dopamine receptor D2 | 1813 | Neurotransmitter | Receptor |
| DRD3 | Dopamine receptor D3 | 1814 | Neurotransmitter | Receptor |
| DRD4 | Dopamine receptor D4 | 1815 | Neurotransmitter | Receptor |
| DRD5 | Dopamine receptor D5 | 1816 | Neurotransmitter | Receptor |
| DTNA | Dystrobrevin alpha | 1837 | Other | Miscelaneous |
| DTNBP1 | Dystrobrevin binding protein 1 | 84062 | Other | Miscelaneous |
| DVL1 | Dishevelled segment polarity | 1855 | Neurotrophic | Signaling |
| | protein 1 | | | |
| ECEL1 | Endothelin converting enzyme | 9427 | Neurotransmitter | Biosynthesis |
| | like 1 | | | |
| EDN1 | Endothelin 1 | 1906 | Neuropeptide | Ligand |
| EDN2 | Endothelin 2 | 1907 | Neuropeptide | Ligand |
| EDN3 | Endothelin 3 | 1908 | Neuropeptide | Ligand |
| EDNRA | Endothelin receptor type A | 1909 | Neuropeptide | Receptor |
| EDNRB | Endothelin receptor type B | 1910 | Neuropeptide | Receptor |
| EEF2 | Eukaryotic translation | 1938 | Other | Miscelaneous |
| | elongation factor 2 | | | |
| EEF2K | Eukaryotic elongation factor 2 | 29904 | Other | Miscelaneous |
| | kinase | | | |
| EFNA5 | Ephrin-A5 (AL-1) (EPH-related | 1946 | Neurotrophic | Ligand |
| | receptor tyrosine kinase ligand | | | |
| | 7) (LERK-7) | | | |
| EGF | Epidermal growth factor | 1950 | Other | Miscelaneous |
| EGFR | Epidermal growth factor | 1956 | Other | Miscelaneous |
| | receptor | | | |
| EGR3 | Early growth response 3 | 1960 | Neurotrophic | Signaling |
| EIF4A3 | Eukaryotic translation initiation | 9775 | Other | Miscelaneous |
| | factor 4A3 | | | |
| EIF4EBP2 | Eukaryotic translation initiation | 1979 | Other | Miscelaneous |
| | factor 4E binding protein 2 | | | |
| EN1 | Engrailed homeobox 1 | 2019 | Other | Miscelaneous |
| ENO2 | Enolase 2 | 2026 | Neurotrophic | Signaling |
| EphA1 | Ephrin type-A receptor 1 | 2041 | Neurotrophic | Receptor |
| EphA10 | Ephrin type-A receptor 10 | 284656 | Neurotrophic | Receptor |
| EphA2 | Ephrin type-A receptor 2 | 1969 | Neurotrophic | Receptor |
| EphA3 | Ephrin type-A receptor 3 | 2042 | Neurotrophic | Receptor |
| EphA4 | Ephrin type-A receptor 4 | 2043 | Neurotrophic | Receptor |
| EphA5 | Ephrin type-A receptor 5 | 2044 | Neurotrophic | Receptor |
| EphA6 | Ephrin type-A receptor 6 | 285220 | Neurotrophic | Receptor |
| EphA7 | Ephrin type-A receptor 7 | 2045 | Neurotrophic | Receptor |
| EphA8 | Ephrin type-A receptor 8 | 2046 | Neurotrophic | Receptor |
| EphB1 | Ephrin type-B receptor 1 | 2047 | Neurotrophic | Receptor |
| EphB2 | Ephrin type-B receptor 2 | 2048 | Neurotrophic | Receptor |
| EphB3 | Ephrin type-B receptor 3 | 2049 | Neurotrophic | Receptor |
| EphB4 | Ephrin type-B receptor 4 | 2050 | Neurotrophic | Receptor |
| EphB6 | Ephrin type-B receptor 6 | 2051 | Neurotrophic | Receptor |
| EPO | Erythropoietin | 2056 | Other | Miscelaneous |
| ERBB2 | Erb-b2 receptor tyrosine | 2064 | Other | Miscelaneous |
| | kinase 2 | | | |
| ERC1 | ELKS/RAB6-interacting/CAST | 23085 | Signaling | Signaling |
| | family member 1 | | | |
| ERC2 | ELKS/RAB6-interacting/CAST | 26059 | Signaling | Signaling |
| | family member 2 | | | |
| ERG | ERG, ETS transcription factor | 2078 | Other | Miscelaneous |
| ETBR2 | Receptor for prosaposin | 9283 | Neurotrophic | Receptor |
| ETV5 | ETS variant 5 | 2119 | Other | Miscelaneous |
| EXOC3L1 | Exocyst complex component 3 | 283849 | Other | Miscelaneous |
| | like 1 | | | |
| F2RL1 | F2R like trypsin receptor 1 | 2150 | Other | Miscelaneous |
| FAAH | Fatty acid amide hydrolase | 2166 | Neurotransmitter | Biosynthesis |
| FAP | Fibroblast activation protein | 2191 | Neuropeptide | Biosynthesis |
| | alpha | | | |
| FEV | FEV, ETS transcription factor | 54738 | Other | Miscelaneous |
| FFAR3 | Free fatty acid receptor 3 | 2865 | Other | Miscelaneous |
| FGF14 | Fibroblast growth factor 14 | 2259 | Other | Miscelaneous |
| FGF2 | Fibroblast growth factor 2 | 2247 | Other | Miscelaneous |
| FGF20 | Fibroblast growth factor 20 | 26281 | Other | Miscelaneous |
| FKBP5 | FK506 binding protein 5 | 2289 | Other | Miscelaneous |
| FLNA | Filamin A | 2316 | Other | Miscelaneous |
| FLVCR1 | Feline leukemia virus | 28982 | Channel or | Transporter |
| | subgroup C cellular receptor 1 | | transporter | |
| FLVCR2 | Feline leukemia virus | 55640 | Channel or | Transporter |
| | subgroup C cellular receptor | | transporter | |
| | family member 2 | | | |
| FMR1 | Fragile X mental retardation 1 | 2332 | Other | Miscelaneous |
| FNTA | Farnesyltransferase, CAAX | 2339 | Neurotransmitter | Signaling |
| | box, alpha | | | |
| FOLH1 | Folate hydrolase 1 | 2346 | Neuropeptide | Biosynthesis |
| FRS2 | Fibroblast growth factor | 10818 | Other | Miscelaneous |
| | receptor substrate 2 | | | |
| FRS3 | Fibroblast growth factor | 10817 | Other | Miscelaneous |
| | receptor substrate 3 | | | |
| FSHR | Follicle stimulating hormone | 2492 | Neuropeptide | Receptor |
| | receptor | | | |
| FSTL4 | Follistatin like 4 | 23105 | Neurotrophic | Receptor |
| FXYD2 | FXYD domain containing ion | 486 | Channel or | Transporter |
| | transport regulator 2 | | transporter | |
| GAB1 | GRB2 associated binding | 2549 | Signaling | Signaling |
| | protein 1 | | | |
| GABARAP | GABA type A receptor- | 11337 | Neurotransmitter | Receptor |
| | associated protein | | | |
| GABBR1 | Gamma-aminobutyric acid | 2550 | Neurotransmitter | Receptor |
| | type B receptor subunit 1 | | | |
| GABRA1 | Gamma-aminobutyric acid | 2554 | Neurotransmitter | Receptor |
| | type A receptor alpha1 subunit | | | |
| GABRA2 | Gamma-aminobutyric acid | 2555 | Neurotransmitter | Receptor |
| | type A receptor alpha2 subunit | | | |
| GABRA3 | Gamma-aminobutyric acid | 2556 | Neurotransmitter | Receptor |
| | type A receptor alpha3 subunit | | | |
| GABRA4 | Gamma-aminobutyric acid | 2557 | Neurotransmitter | Receptor |
| | type A receptor alpha4 subunit | | | |
| GABRA5 | Gamma-aminobutyric acid | 2558 | Neurotransmitter | Receptor |
| | type A receptor alpha5 subunit | | | |
| GABRA6 | Gamma-aminobutyric acid | 2559 | Neurotransmitter | Receptor |
| | type A receptor alpha6 subunit | | | |
| GABRB1 | Gamma-aminobutyric acid | 2560 | Neurotransmitter | Receptor |
| | type A receptor beta1 subunit | | | |
| GABRB2 | Gamma-aminobutyric acid | 2561 | Neurotransmitter | Receptor |
| | type A receptor beta2 subunit | | | |
| GABRB3 | Gamma-aminobutyric acid | 2562 | Neurotransmitter | Receptor |
| | type A receptor beta3 subunit | | | |
| GABRD | Gamma-aminobutyric acid | 2563 | Neurotransmitter | Receptor |
| | type A receptor delta subunit | | | |
| GABRE | Gamma-aminobutyric acid | 2564 | Neurotransmitter | Receptor |
| | type A receptor epsilon | | | |
| | subunit | | | |
| GABRG1 | Gamma-aminobutyric acid | 2565 | Neurotransmitter | Receptor |
| | type A receptor gamma1 | | | |
| | subunit | | | |
| GABRG2 | Gamma-aminobutyric acid | 2566 | Neurotransmitter | Receptor |
| | type A receptor gamma2 | | | |
| | subunit | | | |
| GABRG3 | Gamma-aminobutyric acid | 2567 | Neurotransmitter | Receptor |
| | type A receptor gamma3 | | | |
| | subunit | | | |
| GABRP | Gamma-aminobutyric acid | 2568 | Neurotransmitter | Receptor |
| | type A receptor pi subunit | | | |
| GABRQ | Gamma-aminobutyric acid | 55879 | Neurotransmitter | Receptor |
| | type A receptor theta subunit | | | |
| GABRR1 | Gamma-aminobutyric acid | 2569 | Neurotransmitter | Receptor |
| | type A receptor rho1 subunit | | | |
| GABRR2 | Gamma-aminobutyric acid | 2570 | Neurotransmitter | Receptor |
| | type A receptor rho2 subunit | | | |
| GABRR3 | Gamma-aminobutyric acid | 200959 | Neurotransmitter | Receptor |
| | type A receptor rho3 subunit | | | |
| | (gene/pseudogene) | | | |
| GAD1 | Glutamate decarboxylase 1 | 2571 | Neurotransmitter | Biosynthesis |
| GAD2 | Glutamate decarboxylase 2 | 2572 | Neurotransmitter | Biosynthesis |
| GAL | Galanin and GMAP | 51083 | Neuropeptide | Ligand |
| | prepropeptide | | | |
| GALP | Galanin like peptide | 85569 | Neuropeptide | Ligand |
| GALR1 | Galanin receptor 1 | 2587 | Neuropeptide | Receptor |
| GALR2 | Galanin receptor 2 | 8811 | Neuropeptide | Receptor |
| GALR3 | Galanin receptor 3 | 8484 | Neuropeptide | Receptor |
| GAST | Gastrin | 2520 | Neuropeptide | Ligand |
| GCGR | Glucagon receptor | 2642 | Other | Miscelaneous |
| GCHFR | GTP cyclohydrolase I | 2644 | Neurotransmitter | Biosynthesis |
| | feedback regulator | | | |
| GDNF | Glial cell derived neurotrophic | 2668 | Neurotrophic | Ligand |
| | factor | | | |
| GFAP | Glial fibrillary acidic protein | 2670 | Other | Miscelaneous |
| GFRA1 | GDNF family receptor alpha 1 | 2674 | Neurotrophic | Receptor |
| GFRA2 | GDNF family receptor alpha 2 | 2675 | Neurotrophic | Receptor |
| GFRA3 | GDNF family receptor alpha 3 | 2676 | Neurotrophic | Receptor |
| GFRA4 | GDNF family receptor alpha 4 | 64096 | Neurotrophic | Receptor |
| GH1 | Growth hormone 1 | 2688 | Neuropeptide | Ligand |
| GHRH | Growth hormone releasing | 2691 | Neuropeptide | Ligand |
| | hormone | | | |
| GHRHR | Growth hormone releasing | 2692 | Neuropeptide | Receptor |
| | hormone receptor | | | |
| GHRL | Ghrelin and obestatin | 51738 | Neuropeptide | Ligand |
| | prepropeptide | | | |
| GIP | Gastric inhibitory polypeptide | 2695 | Neuropeptide | Ligand |
| GJA1 | Gap junction protein alpha 1 | 2697 | Channel or | Channel |
| | | | transporter | |
| GJA10 | Gap junction protein alpha 10 | 84694 | Channel or | Channel |
| | | | transporter | |
| GJA3 | Gap junction protein alpha 3 | 2700 | Channel or | Channel |
| | | | transporter | |
| GJA4 | Gap junction protein alpha 4 | 2701 | Channel or | Channel |
| | | | transporter | |
| GJA5 | Gap junction protein alpha 5 | 2702 | Channel or | Channel |
| | | | transporter | |
| GJA8 | Gap junction protein alpha 8 | 2703 | Channel or | Channel |
| | | | transporter | |
| GJA9 | Gap junction protein alpha 9 | 81025 | Channel or | Channel |
| | | | transporter | |
| GJB1 | Gap junction protein beta 1 | 2705 | Channel or | Channel |
| | | | transporter | |
| GJB2 | Gap junction protein beta 2 | 2706 | Channel or | Channel |
| | | | transporter | |
| GJB3 | Gap junction protein beta 3 | 2707 | Channel or | Channel |
| | | | transporter | |
| GJB4 | Gap junction protein beta 4 | 127534 | Channel or | Channel |
| | | | transporter | |
| GJB5 | Gap junction protein beta 5 | 2709 | Channel or | Channel |
| | | | transporter | |
| GJB6 | Gap junction protein beta 6 | 10804 | Channel or | Channel |
| | | | transporter | |
| GJB7 | Gap junction protein beta 7 | 375519 | Channel or | Channel |
| | | | transporter | |
| GJC1 | Gap junction protein gamma 1 | 10052 | Channel or | Channel |
| | | | transporter | |
| GJC2 | Gap junction protein gamma 2 | 57165 | Channel or | Channel |
| | | | transporter | |
| GJC3 | Gap junction protein gamma 3 | 349149 | Channel or | Channel |
| | | | transporter | |
| GJD2 | Gap junction protein delta 2 | 57369 | Channel or | Channel |
| | | | transporter | |
| GJD3 | Gap junction protein delta 3 | 125111 | Channel or | Channel |
| | | | transporter | |
| GJD4 | Gap junction protein delta 4 | 219770 | Channel or | Channel |
| | | | transporter | |
| GJE1 | Gap junction protein epsilon 1 | 100126572 | Channel or | Channel |
| | | | transporter | |
| GLRA1 | Glycine receptor alpha 1 | 2741 | Neurotransmitter | Receptor |
| GLRA2 | Glycine receptor alpha 2 | 2742 | Neurotransmitter | Receptor |
| GLRA3 | Glycine receptor alpha 3 | 8001 | Neurotransmitter | Receptor |
| GLRA4 | Glycine receptor alpha 4 | 441509 | Neurotransmitter | Receptor |
| GLRB | Glycine receptor beta | 2743 | Neurotransmitter | Receptor |
| GLS | Glutaminase | 2744 | Neurotransmitter | Biosynthesis |
| GLS2 | Glutaminase 2 | 27165 | Neurotransmitter | Biosynthesis |
| GLUL | Glutamate-ammonia ligase | 2752 | Neurotransmitter | Biosynthesis |
| GNA11 | G protein subunit alpha 11 | 2767 | Signaling | Signaling |
| GNA13 | G protein subunit alpha 13 | 10672 | Signaling | Signaling |
| GNA14 | G protein subunit alpha 14 | 9630 | Signaling | Signaling |
| GNA15 | G protein subunit alpha 15 | 2769 | Signaling | Signaling |
| GNAI1 | G protein subunit alpha i1 | 2770 | Signaling | Signaling |
| GNAI2 | G protein subunit alpha i2 | 2771 | Signaling | Signaling |
| GNAI3 | G protein subunit alpha i3 | 2773 | Signaling | Signaling |
| GNAL | G protein subunit alpha L | 2774 | Signaling | Signaling |
| GNAO1 | G protein subunit alpha o1 | 2775 | Signaling | Signaling |
| GNAQ | G protein subunit alpha q | 2776 | Signaling | Signaling |
| GNAS | GNAS complex locus | 2778 | Signaling | Signaling |
| GNAZ | G protein subunit alpha z | 2781 | Signaling | Signaling |
| GNB1 | G protein subunit beta 1 | 2782 | Signaling | Signaling |
| GNB2 | G protein subunit beta 2 | 2783 | Signaling | Signaling |
| GNB3 | G protein subunit beta 3 | 2784 | Signaling | Signaling |
| GNB4 | G protein subunit beta 4 | 59345 | Signaling | Signaling |
| GNB5 | G protein subunit beta 5 | 10681 | Signaling | Signaling |
| GNG10 | G protein subunit gamma 10 | 2790 | Signaling | |
| GNG11 | G protein subunit gamma 11 | 2791 | Signaling | |
| GNG12 | G protein subunit gamma 12 | 55970 | Signaling | Signaling |
| GNG13 | G protein subunit gamma 13 | 51764 | Signaling | Signaling |
| GNG2 | G protein subunit gamma 2 | 54331 | Signaling | Signaling |
| GNG3 | G protein subunit gamma 3 | 2785 | Signaling | Signaling |
| GNG4 | G protein subunit gamma 4 | 2786 | Signaling | |
| GNG5 | G protein subunit gamma 5 | 2787 | Signaling | |
| GNG7 | G protein subunit gamma 7 | 2788 | Signaling | |
| GNG8 | G protein subunit gamma 8 | 94235 | Signaling | Signaling |
| GNGT2 | G protein subunit gamma | 2793 | Signaling | |
| | transducin 2 | | | |
| GNMT | Glycine N-methyltransferase | 27232 | Neurotransmitter | Biosynthesis |
| GNRH1 | Gonadotropin releasing | 2796 | Neuropeptide | Ligand |
| | hormone 1 | | | |
| GNRH2 | Gonadotropin releasing | 2797 | Neuropeptide | Ligand |
| | hormone 2 | | | |
| GPER1 | G protein-coupled estrogen | 2852 | Other | Receptor |
| | receptor 1 | | | |
| GPHN | Gephyrin | 10243 | Neuropeptide | Ligand |
| GPI | Glucose-6-phosphate | 2821 | Signaling | Signaling |
| | isomerase | | | |
| GPR1 | G protein-coupled receptor 1 | 2825 | Other | Receptor |
| GPR139 | G protein-coupled receptor | 124274 | Neurotransmitter | Receptor |
| | 139 | | | |
| GPR143 | G protein-coupled receptor | 4935 | Neurotransmitter | Receptor |
| | 143 | | | |
| GPR149 | G protein-coupled receptor | 344758 | Neurotransmitter | Receptor |
| | 149 | | | |
| GPR18 | G protein-coupled receptor 18 | 2841 | Other | Receptor |
| GPR21 | G protein-coupled receptor 21 | 2844 | Other | Receptor |
| GPR26 | G protein-coupled receptor 26 | 2849 | Other | Receptor |
| GPR35 | G protein-coupled receptor 35 | 2859 | Other | Receptor |
| GPR37 | Receptor for prosaposin | 2861 | Neurotrophic | Receptor |
| GPR52 | G protein-coupled receptor 52 | 9293 | Neurotransmitter | Receptor |
| GPR55 | G protein-coupled receptor 55 | 9290 | Neurotransmitter | Receptor |
| GPR78 | G protein-coupled receptor 78 | 27201 | Neurotransmitter | Receptor |
| GPR83 | G protein-coupled receptor 83 | 10888 | Neurotransmitter | Receptor |
| GPR84 | G protein-coupled receptor 84 | 53831 | Neurotransmitter | Receptor |
| GPRASP1 | G protein-coupled receptor | 9737 | Neurotransmitter | Receptor |
| | associated sorting protein 1 | | | |
| GPRC6A | G protein-coupled receptor | 222545 | Signaling | Signaling |
| | class C group 6 member A | | | |
| GPRIN1 | G protein regulated inducer of | 114787 | Neurotrophic | Signaling |
| | neurite outgrowth 1 | | | |
| GPRIN2 | G protein regulated inducer of | 9721 | Neurotrophic | Signaling |
| | neurite outgrowth 2 | | | |
| GPRIN3 | G protein regulated inducer of | 285513 | Neurotrophic | Signaling |
| | neurite outgrowth 3 | | | |
| GRB2 | Growth factor receptor bound | 2885 | Neurotrophic | Signaling |
| | protein 2 | | | |
| GRIA1 | Glutamate ionotropic receptor | 2890 | Neurotransmitter | Receptor |
| | AMPA type subunit 1 | | | |
| GRIA2 | Glutamate ionotropic receptor | 2891 | Neurotransmitter | Receptor |
| | AMPA type subunit 2 | | | |
| GRIA3 | Glutamate ionotropic receptor | 2892 | Neurotransmitter | Receptor |
| | AMPA type subunit 3 | | | |
| GRIA4 | Glutamate ionotropic receptor | 2893 | Neurotransmitter | Receptor |
| | AMPA type subunit 4 | | | |
| GRID1 | Glutamate ionotropic receptor | 2894 | Neurotransmitter | Receptor |
| | delta type subunit 1 | | | |
| GRID2 | Glutamate ionotropic receptor | 2895 | Neurotransmitter | Receptor |
| | delta type subunit 2 | | | |
| GRIK1 | Glutamate ionotropic receptor | 2897 | Neurotransmitter | Receptor |
| | kainate type subunit 1 | | | |
| GRIK2 | Glutamate ionotropic receptor | 2898 | Neurotransmitter | Receptor |
| | kainate type subunit 2 | | | |
| GRIK3 | Glutamate ionotropic receptor | 2899 | Neurotransmitter | Receptor |
| | kainate type subunit 3 | | | |
| GRIK4 | Glutamate ionotropic receptor | 2900 | Neurotransmitter | Receptor |
| | kainate type subunit 4 | | | |
| GRIK5 | Glutamate ionotropic receptor | 2901 | Neurotransmitter | Receptor |
| | kainate type subunit 5 | | | |
| GRIN1 | Glutamate ionotropic receptor | 2902 | Neurotransmitter | Receptor |
| | NMDA type subunit 1 | | | |
| GRIN2A | Glutamate ionotropic receptor | 2903 | Neurotransmitter | Receptor |
| | NMDA type subunit 2A | | | |
| GRIN2B | Glutamate ionotropic receptor | 2904 | Neurotransmitter | Receptor |
| | NMDA type subunit 2B | | | |
| GRIN2C | Glutamate ionotropic receptor | 2905 | Neurotransmitter | Receptor |
| | NMDA type subunit 2C | | | |
| GRIN2D | Glutamate ionotropic receptor | 2906 | Neurotransmitter | Receptor |
| | NMDA type subunit 2D | | | |
| GRIN3A | Glutamate ionotropic receptor | 116443 | Neurotransmitter | Receptor |
| | NMDA type subunit 3A | | | |
| GRIN3B | Glutamate ionotropic receptor | 116444 | Neurotransmitter | Receptor |
| | NMDA type subunit 3B | | | |
| GRK2 | G protein-coupled receptor | 156 | Neurotransmitter | Receptor |
| | kinase 2 | | | |
| GRK3 | G protein-coupled receptor | 157 | Neurotransmitter | Receptor |
| | kinase 3 | | | |
| GRK4 | G protein-coupled receptor | 2868 | Signaling | Signaling |
| | kinase 4 | | | |
| GRK5 | G protein-coupled receptor | 2869 | Signaling | Signaling |
| | kinase 5 | | | |
| GRM1 | Glutamate metabotropic | 2911 | Neurotransmitter | Receptor |
| | receptor 1 | | | |
| GRM2 | Glutamate metabotropic | 2912 | Neurotransmitter | Receptor |
| | receptor 2 | | | |
| GRM3 | Glutamate metabotropic | 2913 | Neurotransmitter | Receptor |
| | receptor 3 | | | |
| GRM4 | Glutamate metabotropic | 2914 | Neurotransmitter | Receptor |
| | receptor 4 | | | |
| GRM5 | Glutamate metabotropic | 2915 | Neurotransmitter | Receptor |
| | receptor 5 | | | |
| GRM6 | Glutamate metabotropic | 2916 | Neurotransmitter | Receptor |
| | receptor 6 | | | |
| GRM7 | Glutamate metabotropic | 2917 | Neurotransmitter | Receptor |
| | receptor 7 | | | |
| GRM8 | Glutamate metabotropic | 2918 | Neurotransmitter | Receptor |
| | receptor 8 | | | |
| GRP | Gastrin releasing peptide | 2922 | Neuropeptide | Ligand |
| GRPR | Gastrin releasing peptide | 2925 | Neuropeptide | Receptor |
| | receptor | | | |
| GSK3A | Glycogen synthase kinase 3 | 2931 | Signaling | Signaling |
| | alpha | | | |
| GSK3B | Glycogen synthase kinase 3 | 2932 | Signaling | Signaling |
| | beta | | | |
| GTF2H2 | General transcription factor | 2966 | Other | Miscelaneous |
| | IIH subunit 2 | | | |
| GZF1 | GDNF-inducible zinc finger | 64412 | Neurotrophic | Signaling |
| | protein 1 | | | |
| HAP1 | Huntingtin associated protein | 9001 | Other | Miscelaneous |
| | 1 | | | |
| HCN1 | Hyperpolarization activated | 348980 | Channel or | Channel |
| | cyclic nucleotide gated | | transporter | |
| | potassium channel 1 | | | |
| HCN2 | Hyperpolarization activated | 610 | Channel or | Channel |
| | cyclic nucleotide gated | | transporter | |
| | potassium channel 2 | | | |
| HCN3 | Hyperpolarization activated | 57657 | Channel or | Channel |
| | cyclic nucleotide gated | | transporter | |
| | potassium channel 3 | | | |
| HCN4 | Hyperpolarization activated | 10021 | Channel or | Channel |
| | cyclic nucleotide gated | | transporter | |
| | potassium channel 4 | | | |
| HCRT | Hypocretin neuropeptide | 3060 | Neuropeptide | Ligand |
| | precursor | | | |
| HCRTR1 | Hypocretin receptor 1 | 3061 | Neuropeptide | Receptor |
| HCRTR2 | Hypocretin receptor 2 | 3062 | Neuropeptide | Receptor |
| HIP1 | Huntingtin interacting protein 1 | 3092 | Other | Miscelaneous |
| HK2 | Hexokinase 2 | 3099 | Other | Miscelaneous |
| HMOX1 | Heme oxygenase 1 | 3162 | Other | Miscelaneous |
| HMOX2 | Heme oxygenase 2 | 3163 | Other | Miscelaneous |
| HNMT | Histamine N- | 3176 | Neurotransmitter | Biosynthesis |
| | methyltransferase | | | |
| HOMER1 | Homer scaffolding protein 1 | 9456 | Neurotransmitter | Receptor |
| HRAS | Hras proto-oncogene, gtpase | 3265 | Signaling | Signaling |
| HRH1 | Histamine receptor H1 | 3269 | Neurotransmitter | Receptor |
| HRH2 | Histamine receptor H2 | 3274 | Neurotransmitter | Receptor |
| HRH3 | Histamine receptor H3 | 11255 | Neurotransmitter | Receptor |
| HRH4 | Histamine receptor H4 | 59340 | Neurotransmitter | Receptor |
| HSPA8 | Heat shock protein family A | 3312 | Vesicular | Vesicles |
| | (Hsp70) member 8 | | | |
| HTL | High L-leucine transport | 3343 | Channel or | Transporter |
| | | | transporter | |
| HTR1A | 5-hydroxytryptamine receptor | 3350 | Neurotransmitter | Receptor |
| | 1A | | | |
| HTR1B | 5-hydroxytryptamine receptor | 3351 | Neurotransmitter | Receptor |
| | 1B | | | |
| HTR1D | 5-hydroxytryptamine receptor | 3352 | Neurotransmitter | Receptor |
| | 1D | | | |
| HTR1E | 5-hydroxytryptamine receptor | 3354 | Neurotransmitter | Receptor |
| | 1E | | | |
| HTR1F | 5-hydroxytryptamine receptor | 3355 | Neurotransmitter | Receptor |
| | 1F | | | |
| HTR2A | 5-hydroxytryptamine receptor | 3356 | Neurotransmitter | Receptor |
| | 2A | | | |
| HTR2B | 5-hydroxytryptamine receptor | 3357 | Neurotransmitter | Receptor |
| | 2B | | | |
| HTR2C | 5-hydroxytryptamine receptor | 3358 | Neurotransmitter | Receptor |
| | 2C | | | |
| HTR3A | 5-hydroxytryptamine receptor | 3359 | Neurotransmitter | Receptor |
| | 3A | | | |
| HTR3B | 5-hydroxytryptamine receptor | 9177 | Neurotransmitter | Receptor |
| | 3B | | | |
| HTR3C | 5-hydroxytryptamine receptor | 170572 | Neurotransmitter | Receptor |
| | 3C | | | |
| HTR3D | 5-hydroxytryptamine receptor | 200909 | Neurotransmitter | Receptor |
| | 3D | | | |
| HTR3E | 5-hydroxytryptamine receptor | 285242 | Neurotransmitter | Receptor |
| | 3E | | | |
| HTR4 | 5-hydroxytryptamine receptor | 3360 | Neurotransmitter | Receptor |
| | 4 | | | |
| HTR5A | 5-hydroxytryptamine receptor | 3361 | Neurotransmitter | Receptor |
| | 5A | | | |
| HTR5BP | 5-hydroxytryptamine receptor | 645694 | Neurotransmitter | Receptor |
| | 5B, pseudogene | | | |
| HTR6 | 5-hydroxytryptamine receptor | 3362 | Neurotransmitter | Receptor |
| | 6 | | | |
| HTR7 | 5-hydroxytryptamine receptor | 3363 | Neurotransmitter | Receptor |
| | 7 | | | |
| HTT | Huntingtin | 3064 | Other | Miscelaneous |
| HVCN1 | Hydrogen voltage gated | 84329 | Channel or | Channel |
| | channel 1 | | transporter | |
| IAPP | Islet amyloid polypeptide | 3375 | Neuropeptide | Ligand |
| ICA1 | Islet cell autoantigen 1 | 3382 | Other | Miscelaneous |
| IFNA1 | Interferon alpha 1 | 3439 | Neurotrophic | Ligand |
| IGF1 | Insulin like growth factor 1 | 3479 | Neurotrophic | Ligand |
| IGF2 | Insulin like growth factor 2 | 3481 | Neurotrophic | Ligand |
| IL11RA | Interleukin 11 receptor subunit | 3590 | Neurotrophic | Receptor |
| | alpha | | | |
| IL1B | Interleukin 1 beta | 3553 | Neurotrophic | Ligand |
| IL3 | Interleukin 3 | 3562 | Neurotrophic | Ligand |
| IL4 | Interleukin 4 | 3565 | Neurotrophic | Ligand |
| IL6 | Interleukin 6 | 3569 | Neurotrophic | Ligand |
| IL6R | Interleukin 6 receptor | 3570 | Neurotrophic | Receptor |
| IL6ST | Interleukin 6 signal transducer | 3572 | Neurotrophic | Signaling |
| INS | Insulin | 3630 | Neurotrophic | Ligand |
| ITPR1 | Inositol 1,4,5-trisphosphate | 3708 | Neurotransmitter | Signaling |
| | receptor type 1 | | | |
| ITPR2 | Inositol 1,4,5-trisphosphate | 3709 | Neurotransmitter | Signaling |
| | receptor type 2 | | | |
| ITPR3 | Inositol 1,4,5-trisphosphate | 3710 | Neurotransmitter | Signaling |
| | receptor type 3 | | | |
| KALRN | Kalirin, rhogef kinase | 8997 | Vesicular | Vesicles |
| KCNA1 | Potassium voltage-gated | 3736 | Channel or | Channel |
| | channel subfamily A member | | transporter | |
| | 1 | | | |
| KCNA10 | Potassium voltage-gated | 3744 | Channel or | Channel |
| | channel subfamily A member | | transporter | |
| | 10 | | | |
| KCNA2 | Potassium voltage-gated | 3737 | Channel or | Channel |
| | channel subfamily A member | | transporter | |
| | 2 | | | |
| KCNA3 | Potassium voltage-gated | 3738 | Channel or | Channel |
| | channel subfamily A member | | transporter | |
| | 3 | | | |
| KCNA4 | Potassium voltage-gated | 3739 | Channel or | Channel |
| | channel subfamily A member | | transporter | |
| | 4 | | | |
| KCNA5 | Potassium voltage-gated | 3741 | Channel or | Channel |
| | channel subfamily A member | | transporter | |
| | 5 | | | |
| KCNA6 | Potassium voltage-gated | 3742 | Channel or | Channel |
| | channel subfamily A member | | transporter | |
| | 6 | | | |
| KCNA7 | Potassium voltage-gated | 3743 | Channel or | Channel |
| | channel subfamily A member | | transporter | |
| | 7 | | | |
| KCNAB1 | Potassium voltage-gated | 7881 | Channel or | Channel |
| | channel subfamily A member | | transporter | |
| | regulatory beta subunit 1 | | | |
| KCNAB2 | Potassium voltage-gated | 8514 | Channel or | Channel |
| | channel subfamily A | | transporter | |
| | regulatory beta subunit 2 | | | |
| KCNB1 | Potassium voltage-gated | 3745 | Channel or | Channel |
| | channel subfamily B member | | transporter | |
| | 1 | | | |
| KCNB2 | Potassium voltage-gated | 9312 | Channel or | Channel |
| | channel subfamily B member | | transporter | |
| | 2 | | | |
| KCNC1 | Potassium voltage-gated | 3746 | Channel or | Channel |
| | channel subfamily C member | | transporter | |
| | 1 | | | |
| KCNC2 | Potassium voltage-gated | 3747 | Channel or | Channel |
| | channel subfamily C member | | transporter | |
| | 2 | | | |
| KCNC3 | Potassium voltage-gated | 3748 | Channel or | Channel |
| | channel subfamily C member | | transporter | |
| | 3 | | | |
| KCNC4 | Potassium voltage-gated | 3749 | Channel or | Channel |
| | channel subfamily C member | | transporter | |
| | 4 | | | |
| KCND1 | Potassium voltage-gated | 3750 | Channel or | Channel |
| | channel subfamily D member | | transporter | |
| | 1 | | | |
| KCND2 | Potassium voltage-gated | 3751 | Channel or | Channel |
| | channel subfamily D member | | transporter | |
| | 2 | | | |
| KCND3 | Potassium voltage-gated | 3752 | Channel or | Channel |
| | channel subfamily D member | | transporter | |
| | 3 | | | |
| KCNE2 | Potassium voltage-gated | 9992 | Channel or | Channel |
| | channel subfamily E | | transporter | |
| | regulatory subunit 2 | | | |
| KCNE3 | Potassium voltage-gated | 10008 | Channel or | Channel |
| | channel subfamily E | | transporter | |
| | regulatory subunit 3 | | | |
| KCNE4 | Potassium voltage-gated | 23704 | Channel or | Channel |
| | channel subfamily E | | transporter | |
| | regulatory subunit 4 | | | |
| KCNF1 | Potassium voltage-gated | 3754 | Channel or | Channel |
| | channel modifier subfamily F | | transporter | |
| | member 1 | | | |
| KCNG1 | Potassium voltage-gated | 3755 | Channel or | Channel |
| | channel modifier subfamily G | | transporter | |
| | member 1 | | | |
| KCNG2 | Potassium voltage-gated | 26251 | Channel or | Channel |
| | channel modifier subfamily G | | transporter | |
| | member 2 | | | |
| KCNG3 | Potassium voltage-gated | 170850 | Channel or | Channel |
| | channel modifier subfamily G | | transporter | |
| | member 3 | | | |
| KCNG4 | Potassium voltage-gated | 93107 | Channel or | Channel |
| | channel modifier subfamily G | | transporter | |
| | member 4 | | | |
| KCNH1 | Potassium voltage-gated | 3756 | Channel or | Channel |
| | channel subfamily H member | | transporter | |
| | 1 | | | |
| KCNH2 | Potassium voltage-gated | 3757 | Channel or | Channel |
| | channel subfamily H member | | transporter | |
| | 2 | | | |
| KCNH3 | Potassium voltage-gated | 23416 | Channel or | Channel |
| | channel subfamily H member | | transporter | |
| | 3 | | | |
| KCNH4 | Potassium voltage-gated | 23415 | Channel or | Channel |
| | channel subfamily H member | | transporter | |
| | 4 | | | |
| KCNH5 | Potassium voltage-gated | 27133 | Channel or | Channel |
| | channel subfamily H member | | transporter | |
| | 5 | | | |
| KCNH6 | Potassium voltage-gated | 81033 | Channel or | Channel |
| | channel subfamily H member | | transporter | |
| | 6 | | | |
| KCNH7 | Potassium voltage-gated | 90134 | Channel or | Channel |
| | channel subfamily H member | | transporter | |
| | 7 | | | |
| KCNH8 | Potassium voltage-gated | 131096 | Channel or | Channel |
| | channel subfamily H member | | transporter | |
| | 8 | | | |
| KCNJ1 | Potassium voltage-gated | 3758 | Channel or | Channel |
| | channel subfamily J member 1 | | transporter | |
| KCNJ10 | Potassium voltage-gated | 3766 | Channel or | Channel |
| | channel subfamily J member | | transporter | |
| | 10 | | | |
| KCNJ11 | Potassium voltage-gated | 3767 | Channel or | Channel |
| | channel subfamily J member | | transporter | |
| | 11 | | | |
| KCNJ12 | Potassium voltage-gated | 3768 | Channel or | Channel |
| | channel subfamily J member | | transporter | |
| | 12 | | | |
| KCNJ13 | Potassium voltage-gated | 3769 | Channel or | Channel |
| | channel subfamily J member | | transporter | |
| | 13 | | | |
| KCNJ14 | Potassium voltage-gated | 3770 | Channel or | Channel |
| | channel subfamily J member | | transporter | |
| | 14 | | | |
| KCNJ15 | Potassium voltage-gated | 3772 | Channel or | Channel |
| | channel subfamily J member | | transporter | |
| | 15 | | | |
| KCNJ16 | Potassium voltage-gated | 3773 | Channel or | Channel |
| | channel subfamily J member | | transporter | |
| | 16 | | | |
| KCNJ2 | Potassium voltage-gated | 3759 | Channel or | Channel |
| | channel subfamily J member 2 | | transporter | |
| KCNJ3 | Potassium voltage-gated | 3760 | Channel or | Channel |
| | channel subfamily J member 3 | | transporter | |
| KCNJ4 | Potassium voltage-gated | 3761 | Channel or | Channel |
| | channel subfamily J member 4 | | transporter | |
| KCNJ5 | Potassium voltage-gated | 3762 | Channel or | Channel |
| | channel subfamily J member 5 | | transporter | |
| KCNJ6 | Potassium voltage-gated | 3763 | Channel or | Channel |
| | channel subfamily J member 6 | | transporter | |
| KCNJ8 | Potassium voltage-gated | 3764 | Channel or | Channel |
| | channel subfamily J member 8 | | transporter | |
| KCNJ9 | Potassium voltage-gated | 3765 | Channel or | Channel |
| | channel subfamily J member 9 | | transporter | |
| KCNK1 | Potassium two pore domain | 3775 | Channel or | Channel |
| | channel subfamily K member | | transporter | |
| | 1 | | | |
| KCNK10 | Potassium two pore domain | 54207 | Channel or | Channel |
| | channel subfamily K member | | transporter | |
| | 10 | | | |
| KCNK12 | Potassium two pore domain | 56660 | Channel or | Channel |
| | channel subfamily K member | | transporter | |
| | 12 | | | |
| KCNK13 | Potassium two pore domain | 56659 | Channel or | Channel |
| | channel subfamily K member | | transporter | |
| | 13 | | | |
| KCNK15 | Potassium two pore domain | 60598 | Channel or | Channel |
| | channel subfamily K member | | transporter | |
| | 15 | | | |
| KCNK16 | Potassium two pore domain | 83795 | Channel or | Channel |
| | channel subfamily K member | | transporter | |
| | 16 | | | |
| KCNK17 | Potassium two pore domain | 89822 | Channel or | Channel |
| | channel subfamily K member | | transporter | |
| | 17 | | | |
| KCNK18 | Potassium two pore domain | 338567 | Channel or | Channel |
| | channel subfamily K member | | transporter | |
| | 18 | | | |
| KCNK2 | Potassium two pore domain | 3776 | Channel or | Channel |
| | channel subfamily K member | | transporter | |
| | 2 | | | |
| KCNK3 | Potassium two pore domain | 3777 | Channel or | Channel |
| | channel subfamily K member | | transporter | |
| | 3 | | | |
| KCNK4 | Potassium two pore domain | 50801 | Channel or | Channel |
| | channel subfamily K member | | transporter | |
| | 4 | | | |
| KCNK5 | Potassium two pore domain | 8645 | Channel or | Channel |
| | channel subfamily K member | | transporter | |
| | 5 | | | |
| KCNK6 | Potassium two pore domain | 9424 | Channel or | Channel |
| | channel subfamily K member | | transporter | |
| | 6 | | | |
| KCNK7 | Potassium two pore domain | 10089 | Channel or | Channel |
| | channel subfamily K member | | transporter | |
| | 7 | | | |
| KCNK9 | Potassium two pore domain | 51305 | Channel or | Channel |
| | channel subfamily K member | | transporter | |
| | 9 | | | |
| KCNMA1 | Potassium calcium-activated | 3778 | Channel or | Channel |
| | channel subfamily M alpha 1 | | transporter | |
| KCNMB4 | Potassium calcium-activated | 27345 | Channel or | Channel |
| | channel subfamily M | | transporter | |
| | regulatory beta subunit 4 | | | |
| KCNN1 | Potassium calcium-activated | 3780 | Channel or | Channel |
| | channel subfamily N member | | transporter | |
| | 1 | | | |
| KCNN2 | Potassium calcium-activated | 3781 | Channel or | Channel |
| | channel subfamily N member | | transporter | |
| | 2 | | | |
| KCNN3 | Potassium calcium-activated | 3782 | Channel or | Channel |
| | channel subfamily N member | | transporter | |
| | 3 | | | |
| KCNN4 | Potassium calcium-activated | 3783 | Channel or | Channel |
| | channel subfamily N member | | transporter | |
| | 4 | | | |
| KCNQ1 | Potassium voltage-gated | 3784 | Channel or | Channel |
| | channel subfamily Q member | | transporter | |
| | 1 | | | |
| KCNQ2 | Potassium voltage-gated | 3785 | Channel or | Channel |
| | channel subfamily Q member | | transporter | |
| | 2 | | | |
| KCNQ3 | Potassium voltage-gated | 3786 | Channel or | Channel |
| | channel subfamily Q member | | transporter | |
| | 3 | | | |
| KCNQ4 | Potassium voltage-gated | 9132 | Channel or | Channel |
| | channel subfamily Q member | | transporter | |
| | 4 | | | |
| KCNQ5 | Potassium voltage-gated | 56479 | Channel or | Channel |
| | channel subfamily Q member | | transporter | |
| | 5 | | | |
| KCNS1 | Potassium voltage-gated | 3787 | Channel or | Channel |
| | channel modifier subfamily S | | transporter | |
| | member 1 | | | |
| KCNS2 | Potassium voltage-gated | 3788 | Channel or | Channel |
| | channel modifier subfamily S | | transporter | |
| | member 2 | | | |
| KCNS3 | Potassium voltage-gated | 3790 | Channel or | Channel |
| | channel modifier subfamily S | | transporter | |
| | member 3 | | | |
| KCNT1 | Potassium sodium-activated | 57582 | Channel or | Channel |
| | channel subfamily T member | | transporter | |
| | 1 | | | |
| KCNT2 | Potassium sodium-activated | 343450 | Channel or | Channel |
| | channel subfamily T member | | transporter | |
| | 2 | | | |
| KCNU1 | Potassium calcium-activated | 157855 | Channel or | Channel |
| | channel subfamily U member | | transporter | |
| | 1 | | | |
| KCNV1 | Potassium voltage-gated | 27012 | Channel or | Channel |
| | channel modifier subfamily V | | transporter | |
| | member 1 | | | |
| KCNV2 | Potassium voltage-gated | 169522 | Channel or | Channel |
| | channel modifier subfamily V | | transporter | |
| | member 2 | | | |
| KIF1B | Kinesin family member 1B | 23095 | Vesicular | Vesicles |
| KISS1 | Kiss-1 metastasis-suppressor | 3814 | Neuropeptide | Ligand |
| KISS1R | KISS1 receptor | 84634 | Neuropeptide | Receptor |
| KLF16 | Kruppel like factor 16 | 83855 | Other | Miscelaneous |
| KRAS | KRAS proto-oncogene, gtpase | 3845 | Signaling | Signaling |
| L1CAM | L1 cell adhesion molecule | 3897 | Neurotrophic | Signaling |
| LAMTOR3 | Late endosomal/lysosomal | 8649 | Signaling | Signaling |
| | adaptor, MAPK and MTOR | | | |
| | activator 3 | | | |
| LEP | Leptin | 3952 | Neuropeptide | Ligand |
| LHCGR | Luteinizing | 3973 | Neuropeptide | Receptor |
| | hormone/choriogonadotropin | | | |
| | receptor | | | |
| LIF | Leukemia inhibitory factor | 3976 | Neuropeptide | Ligand |
| LIFR | Leukemia inhibitory factor | 3977 | Neurotrophic | Receptor |
| | receptor alpha | | | |
| LIN7A | Lin-7 homolog A, crumbs cell | 8825 | Vesicular | Vesicles |
| | polarity complex component | | | |
| LIN7B | Lin-7 homolog B, crumbs cell | 64130 | Vesicular | Vesicles |
| | polarity complex component | | | |
| LIN7C | Lin-7 homolog C, crumbs cell | 55327 | Vesicular | Vesicles |
| | polarity complex component | | | |
| LPAR3 | Lysophosphatidic acid | 23566 | Other | Miscelaneous |
| | receptor 3 | | | |
| LRP8 | LDL receptor related protein 8 | 7804 | Other | Miscelaneous |
| LRRK2 | Leucine rich repeat kinase 2 | 120892 | Other | Miscelaneous |
| LTB4R | Leukotriene B4 receptor | 1241 | Other | Miscelaneous |
| LTB4R2 | Leukotriene B4 receptor 2 | 56413 | Other | Miscelaneous |
| LYNX1 | Ly6/neurotoxin 1 | 66004 | Neurotransmitter | Receptor |
| MAGED1 | Melanoma-associated antigen | 9500 | Neurotrophic | Signaling |
| | D1 | | | |
| MANF | Mesencephalic astrocyte | 7873 | Neurotrophic | Ligand |
| | derived neurotrophic factor | | | |
| MAOA | Monoamine oxidase A | 4128 | Neurotransmitter | Biosynthesis |
| MAOB | Monoamine oxidase B | 4129 | Neurotransmitter | Biosynthesis |
| MAP2K1 | Mitogen-activated protein | 5604 | Signaling | Signaling |
| | kinase kinase 1 | | | |
| MAP2K2 | Mitogen-activated protein | 5605 | Signaling | Signaling |
| | kinase kinase 2 | | | |
| MAP3K1 | Mitogen-activated protein | 4214 | Signaling | Signaling |
| | kinase kinase kinase 1 | | | |
| MAPK1 | Mitogen-activated protein | 5594 | Signaling | Signaling |
| | kinase 1 | | | |
| MAPK14 | Mitogen-activated protein | 1432 | Signaling | Signaling |
| | kinase 14 | | | |
| MAPK3 | Mitogen-activated protein | 5595 | Signaling | Signaling |
| | kinase 3 | | | |
| MAPK8IP2 | Mitogen-activated protein | 23542 | Signaling | Signaling |
| | kinase 8 interacting protein 2 | | | |
| MC1R | Melanocortin 1 receptor | 4157 | Neuropeptide | Receptor |
| MC2R | Melanocortin 2 receptor | 4158 | Neuropeptide | Receptor |
| MC3R | Melanocortin 3 receptor | 4159 | Neuropeptide | Receptor |
| MC4R | Melanocortin 4 receptor | 4160 | Neuropeptide | Receptor |
| MC5R | Melanocortin 5 receptor | 4161 | Neuropeptide | Receptor |
| MCHR1 | Melanin concentrating | 2847 | Neuropeptide | Receptor |
| | hormone receptor 1 | | | |
| MCHR2 | Melanin concentrating | 84539 | Neuropeptide | Receptor |
| | hormone receptor 2 | | | |
| MCOLN1 | Mucolipin 1 | 57192 | Channel or | Channel |
| | | | transporter | |
| MCOLN2 | Mucolipin 2 | 255231 | Channel or | Channel |
| | | | transporter | |
| MCOLN3 | Mucolipin 3 | 55283 | Channel or | Channel |
| | | | transporter | |
| MEF2C | Myocyte enhancer factor 2C | 4208 | Other | Miscelaneous |
| MFSD7 | Major facilitator superfamily | 84179 | Channel or | Transporter |
| | domain containing 7 | | transporter | |
| MIR204 | Microrna 204 | 406987 | Other | Miscelaneous |
| MLN | Motilin | 4295 | Neuropeptide | Ligand |
| MME | Membrane | 4311 | Neuropeptide | Biosynthesis |
| | metalloendopeptidase | | | |
| MRAP | Melanocortin 2 receptor | 56246 | Neuropeptide | Receptor |
| | accessory protein | | | |
| MRAP2 | Melanocortin 2 receptor | 112609 | Neuropeptide | Receptor |
| | accessory protein 2 | | | |
| MRGPRF | MAS related GPR family | 116535 | Neurotransmitter | Receptor |
| | member F | | | |
| MRGPRX2 | MAS related GPR family | 117194 | Neurotransmitter | Receptor |
| | member X2 | | | |
| MT3 | Metallothionein 3 | 4504 | Other | Miscelaneous |
| MT-ATP6 | Mitochondrially encoded ATP | 4508 | Channel or | Transporter |
| | synthase 6 | | transporter | |
| MT-ATP8 | Mitochondrially encoded ATP | 4509 | Channel or | Transporter |
| | synthase 8 | | transporter | |
| MTCH1 | Mitochondrial carrier 1 | 23787 | Channel or | Transporter |
| | | | transporter | |
| MTCH2 | Mitochondrial carrier 2 | 23788 | Channel or | Transporter |
| | | | transporter | |
| MTNR1A | Melatonin receptor 1A | 4543 | Neuropeptide | Receptor |
| MTNR1B | Melatonin receptor 1B | 4544 | Neuropeptide | Receptor |
| NAAA | N-acylethanolamine acid | 27163 | Vesicular | Vesicles |
| | amidase | | | |
| NAALAD2 | N-acetylated alpha-linked | 10003 | Neuropeptide | Biosynthesis |
| | acidic dipeptidase 2 | | | |
| NALCN | Sodium leak channel, non- | 259232 | Channel or | Channel |
| | selective | | transporter | |
| NAMPT | Nicotinamide | 10135 | Neurotransmitter | Biosynthesis |
| | phosphoribosyltransferase | | | |
| NANOGNB | NANOG neighbor homeobox | 360030 | Vesicular | Vesicles |
| NDNF | Neuron derived neurotrophic | 79625 | Neurotrophic | Ligand |
| | factor | | | |
| NDP | NDP, norrin cystine knot | 4693 | Other | Miscelaneous |
| | growth factor | | | |
| NENF | Neudesin neurotrophic factor | 29937 | Neurotrophic | Ligand |
| NENFP1 | Neudesin neurotrophic factor | 106480294 | Neurotrophic | Ligand |
| | pseudogene 1 | | | |
| NENFP2 | Neudesin neurotrophic factor | 100129880 | Neurotrophic | Ligand |
| | pseudogene 2 | | | |
| NENFP3 | Neudesin neurotrophic factor | 106481703 | Neurotrophic | Ligand |
| | pseudogene 3 | | | |
| NF1 | Neurofibromin 1 | 4763 | Signaling | Signaling |
| NFKB1 | Nuclear factor kappa B | 4790 | Other | Miscelaneous |
| | subunit 1 | | | |
| NGF | Nerve growth factor | 4803 | Neurotrophic | Ligand |
| NGFR | Nerve growth factor receptor | 4804 | Neurotrophic | Receptor |
| NIPSNAP1 | Nipsnap homolog 1 | 8508 | Vesicular | Vesicles |
| | (C. Elegans) | | | |
| NISCH | Nischarin | 11188 | Neurotransmitter | Receptor |
| NLGN1 | Neuroligin 1 | 22871 | Synaptic | Synapse |
| NLGN2 | Neuroligin 2 | 57555 | Synaptic | Synapse |
| NLGN3 | Neuroligin 3 | 54413 | Synaptic | Synapse |
| NLGN4Y | Neuroligin 4, Y-linked | 22829 | Synaptic | Synapse |
| NMB | Neuromedin B | 4828 | Neuropeptide | Ligand |
| NMS | Neuromedin S | 129521 | Neuropeptide | Ligand |
| NMU | Neuromedin U | 10874 | Neuropeptide | Ligand |
| NMUR1 | Neuromedin U receptor 1 | 10316 | Neuropeptide | Receptor |
| NMUR2 | Neuromedin U receptor 2 | 56923 | Neuropeptide | Receptor |
| NOS1 | Nitric oxide synthase 1 | 4842 | Neurotransmitter | Biosynthesis |
| NPB | Neuropeptide B | 256933 | Neuropeptide | Ligand |
| NPBWR1 | Neuropeptides B/W receptor 1 | 2831 | Neuropeptide | Receptor |
| NPBWR2 | Neuropeptides B/W receptor 2 | 2832 | Neuropeptide | Receptor |
| NPC1L1 | NPC1 like intracellular | 29881 | Channel or | Transporter |
| | cholesterol transporter 1 | | transporter | |
| NPFF | Neuropeptide FF-amide | 8620 | Neuropeptide | Ligand |
| | peptide precursor | | | |
| NPFFR1 | Neuropeptide FF receptor 1 | 64106 | Neuropeptide | Receptor |
| NPFFR2 | Neuropeptide FF receptor 2 | 10886 | Neuropeptide | Receptor |
| NPPA | Natriuretic peptide A | 4878 | Neuropeptide | Ligand |
| NPPB | Natriuretic peptide B | 4879 | Neuropeptide | Ligand |
| NPPC | Natriuretic peptide C | 4880 | Neuropeptide | Ligand |
| NPS | Neuropeptide S | 594857 | Neuropeptide | Ligand |
| NPSR1 | Neuropeptide S receptor 1 | 387129 | Neuropeptide | Receptor |
| NPTN | Neuroplastin | 27020 | Neurotransmitter | Receptor |
| NPVF | Neuropeptide VF precursor | 64111 | Neuropeptide | Ligand |
| NPW | Neuropeptide W | 283869 | Neuropeptide | Ligand |
| NPY | Neuropeptide Y | 4852 | Neuropeptide | Ligand |
| NPY1R | Neuropeptide Y receptor Y1 | 4886 | Neuropeptide | Receptor |
| NPY2R | Neuropeptide Y receptor Y2 | 4887 | Neuropeptide | Receptor |
| NPY4R | Neuropeptide Y receptor Y4 | 5540 | Neuropeptide | Receptor |
| NPY5R | Neuropeptide Y receptor Y5 | 4889 | Neuropeptide | Receptor |
| NPY6R | Neuropeptide Y receptor Y6 | 4888 | Neuropeptide | Receptor |
| | (pseudogene) | | | |
| NQO1 | NAD(P)H quinone | 1728 | Other | Miscelaneous |
| | dehydrogenase 1 | | | |
| NR4A2 | Nuclear receptor subfamily 4 | 4929 | Other | Miscelaneous |
| | group A member 2 | | | |
| NRG1 | Neuregulin 1 | 3084 | Neurotrophic | Ligand |
| NRP1 | Neuropilin 1 | 8829 | Neurotrophic | Receptor |
| NRTN | Neurturin | 4902 | Neurotrophic | Ligand |
| NRXN1 | Neurexin 1 | 9378 | Synaptic | Receptor |
| NRXN2 | Neurexin 2 | 9379 | Synaptic | Receptor |
| NRXN3 | Neurexin 3 | 9369 | Synaptic | Receptor |
| NSF | N-ethylmaleimide sensitive | 4905 | Signaling | Signaling |
| | factor, vesicle fusing atpase | | | |
| NTF3 | Neurotrophin 3 | 4908 | Neurotrophic | Ligand |
| NTF4 | Neurotrophin 4 | 4909 | Neurotrophic | Ligand |
| NTRK1 | Neurotrophic receptor tyrosine | 4914 | Neurotrophic | Receptor |
| | kinase 1 | | | |
| NTRK2 | Neurotrophic receptor tyrosine | 4915 | Neurotrophic | Receptor |
| | kinase 2 | | | |
| NTRK3 | Neurotrophic receptor tyrosine | 4916 | Neurotrophic | Receptor |
| | kinase 3 | | | |
| NTS | Neurotensin | 4922 | Neuropeptide | Ligand |
| NTSR1 | Neurotensin receptor 1 | 4923 | Neuropeptide | Receptor |
| NTSR2 | Neurotensin receptor 2 | 23620 | Neuropeptide | Receptor |
| NUCB2 | Nucleobindin 2 | 4925 | Other | Miscelaneous |
| NXPH1 | Neurexophilin 1 | 30010 | Neuropeptide | Ligand |
| NXPH2 | Neurexophilin 2 | 11249 | Neuropeptide | Ligand |
| NXPH3 | Neurexophilin 3 | 11248 | Neuropeptide | Ligand |
| NXPH4 | Neurexophilin 4 | 11247 | Neuropeptide | Ligand |
| OGFR | Opioid growth factor receptor | 11054 | Neuropeptide | Receptor |
| OPHN1 | Oligophrenin 1 | 4983 | Other | Miscelaneous |
| OPRD1 | Opioid receptor delta 1 | 4985 | Neuropeptide | Receptor |
| OPRK1 | Opioid receptor kappa 1 | 4986 | Neuropeptide | Receptor |
| OPRL1 | Opioid related nociceptin | 4987 | Neuropeptide | Receptor |
| | receptor 1 | | | |
| OPRM1 | Opioid receptor mu 1 | 4988 | Neuropeptide | Receptor |
| OTOF | Otoferlin | 9381 | Other | Miscelaneous |
| OXT | Oxytocin/neurophysin I | 5020 | Neuropeptide | Ligand |
| | prepropeptide | | | |
| OXTR | Oxytocin receptor | 5021 | Neuropeptide | Receptor |
| P2RX1 | Purinergic receptor P2X 1 | 5023 | Neurotransmitter | Receptor |
| P2RX2 | Purinergic receptor P2X 2 | 22953 | Neurotransmitter | Receptor |
| P2RX3 | Purinergic receptor P2X 3 | 5024 | Neurotransmitter | Receptor |
| P2RX4 | Purinergic receptor P2X 4 | 5025 | Neurotransmitter | Receptor |
| P2RX5 | Purinergic receptor P2X 5 | 5026 | Neurotransmitter | Receptor |
| P2RX6 | Purinergic receptor P2X 6 | 9127 | Neurotransmitter | Receptor |
| P2RX7 | Purinergic receptor P2X 7 | 5027 | Neurotransmitter | Receptor |
| P2RY11 | Purinergic receptor P2Y11 | 5032 | Neurotransmitter | Receptor |
| PAH | Phenylalanine hydroxylase | 5053 | Neurotransmitter | Biosynthesis |
| PANX1 | Pannexin 1 | 24145 | Channel or | Channel |
| | | | transporter | |
| PANX2 | Pannexin 2 | 56666 | Channel or | Channel |
| | | | transporter | |
| PANX3 | Pannexin 3 | 116337 | Channel or | Channel |
| | | | transporter | |
| PARK2 | Parkin RBR E3 ubiquitin | 5071 | Other | Miscelaneous |
| | protein ligase | | | |
| PARK7 | Parkinsonism associated | 11315 | Other | Miscelaneous |
| | deglycase | | | |
| PATE4 | Prostate and testis expressed | 399968 | Other | Miscelaneous |
| | 4 | | | |
| PC | Pyruvate carboxylase | 5091 | Neurotransmitter | Biosynthesis |
| PCLO | Piccolo presynaptic cytomatrix | 27445 | Other | Miscelaneous |
| | protein | | | |
| PCSK1 | Proprotein convertase | 5122 | Neuropeptide | Biosynthesis |
| | subtilisin/kexin type 1 | | | |
| PCSK1N | Proprotein convertase | 27344 | Neuropeptide | Biosynthesis |
| | subtilisin/kexin type 1 inhibitor | | | |
| PDE1B | Phosphodiesterase 1B | 5153 | Neurotransmitter | Signaling |
| PDE4A | Phosphodiesterase 4A | 5141 | Neurotransmitter | Signaling |
| PDE4D | Phosphodiesterase 4D | 5144 | Neurotransmitter | Signaling |
| PDK1 | Pyruvate dehydrogenase | 5163 | Other | Miscelaneous |
| | kinase 1 | | | |
| PDPK1 | 3-phosphoinositide dependent | 5170 | Neurotrophic | Signaling |
| | protein kinase 1 | | | |
| PDYN | Prodynorphin | 5173 | Other | Miscelaneous |
| PDZD11 | PDZ domain containing 11 | 51248 | Vesicular | Vesicles |
| PENK | Proenkephalin | 5179 | Neuropeptide | Ligand |
| PHOX2A | Paired like homeobox 2a | 401 | Neurotransmitter | Biosynthesis |
| PHOX2B | Paired like homeobox 2b | 8929 | Neurotransmitter | Biosynthesis |
| PIK3CA | Phosphatidylinositol-4,5- | 5290 | Neurotransmitter | Signaling |
| | bisphosphate 3-kinase | | | |
| | catalytic subunit alpha | | | |
| PIK3CB | Phosphatidylinositol-4,5- | 5291 | Neurotransmitter | Signaling |
| | bisphosphate 3-kinase | | | |
| | catalytic subunit beta | | | |
| PIK3CG | Phosphatidylinositol-4,5- | 5294 | Neurotransmitter | Signaling |
| | bisphosphate 3-kinase | | | |
| | catalytic subunit gamma | | | |
| PINK1 | PTEN induced putative kinase | 65018 | Other | Miscelaneous |
| | 1 | | | |
| PITX3 | Paired like homeodomain 3 | 5309 | Other | Miscelaneous |
| PKD2 | Polycystin 2, transient | 5311 | Channel or | Channel |
| | receptor potential cation | | transporter | |
| | channel | | | |
| PKD2L1 | Polycystin 2 like 1, transient | 9033 | Channel or | Channel |
| | receptor potential cation | | transporter | |
| | channel | | | |
| PKD2L2 | Polycystin 2 like 2, transient | 27039 | Channel or | Channel |
| | receptor potential cation | | transporter | |
| | channel | | | |
| PLAT | Plasminogen activator, tissue | 5327 | Other | Miscelaneous |
| | type | | | |
| PLCB1 | Phospholipase C beta 1 | 23236 | Neurotransmitter | Signaling |
| PLCB2 | Phospholipase C beta 2 | 5330 | Neurotransmitter | Signaling |
| PLCB3 | Phospholipase C beta 3 | 5331 | Neurotransmitter | Signaling |
| PLCB4 | Phospholipase C beta 4 | 5332 | Neurotransmitter | Signaling |
| PLCD1 | Phospholipase C delta 1 | 5333 | Neurotransmitter | Signaling |
| PLCE1 | Phospholipase C epsilon 1 | 51196 | Neurotransmitter | Signaling |
| PLCG1 | Phospholipase C gamma 1 | 5335 | Neurotransmitter | Signaling |
| PLCL1 | Phospholipase C like 1 | 5334 | Neurotransmitter | Signaling |
| PLCL2 | Phospholipase C like 2 | 23228 | Neurotransmitter | Signaling |
| PLEKHH3 | Pleckstrin homology, myth4 | 79990 | Neurotrophic | Signaling |
| | and FERM domain containing | | | |
| | H3 | | | |
| PMCH | Pro-melanin concentrating | 5367 | Neuropeptide | Ligand |
| | hormone | | | |
| PNKD | Paroxysmal nonkinesigenic | 25953 | Vesicular | Vesicles |
| | dyskinesia | | | |
| PNOC | Prepronociceptin | 5368 | Neuropeptide | Ligand |
| POMC | Proopiomelanocortin | 5443 | Neuropeptide | Ligand |
| PPARG | Peroxisome proliferator | 5468 | Other | Miscelaneous |
| | activated receptor gamma | | | |
| PPFIA1 | PTPRF interacting protein | 8500 | Vesicular | Vesicles |
| | alpha 1 | | | |
| PPFIA2 | PTPRF interacting protein | 8499 | Vesicular | Vesicles |
| | alpha 2 | | | |
| PPFIA3 | PTPRF interacting protein | 8541 | Vesicular | Vesicles |
| | alpha 3 | | | |
| PPFIA4 | PTPRF interacting protein | 8497 | Vesicular | Vesicles |
| | alpha 4 | | | |
| PPP1CA | Protein phosphatase 1 | 5499 | Signaling | Signaling |
| | catalytic subunit alpha | | | |
| PPP1CB | Protein phosphatase 1 | 5500 | Neurotransmitter | Signaling |
| | catalytic subunit beta | | | |
| PPP1CC | Protein phosphatase 1 | 5501 | Neurotransmitter | Signaling |
| | catalytic subunit gamma | | | |
| PPP1R12A | Protein phosphatase 1 | 4659 | Signaling | Signaling |
| | regulatory subunit 12A | | | |
| PPP1R1B | Protein phosphatase 1 | 84152 | Signaling | Signaling |
| | regulatory inhibitor subunit 1B | | | |
| PPP1R9A | Protein phosphatase 1 | 55607 | Signaling | Signaling |
| | regulatory subunit 9A | | | |
| PPP2CA | Protein phosphatase 2 | 5515 | Signaling | Signaling |
| | catalytic subunit alpha | | | |
| PPP3CA | Protein phosphatase 3 | 5530 | Signaling | Signaling |
| | catalytic subunit alpha | | | |
| PPP3CB | Protein phosphatase 3 | 5532 | Signaling | Signaling |
| | catalytic subunit beta | | | |
| PPP3CC | Protein phosphatase 3 | 5533 | Signaling | Signaling |
| | catalytic subunit gamma | | | |
| PPP3R1 | Protein phosphatase 3 | 5534 | Signaling | Signaling |
| | regulatory subunit B, alpha | | | |
| PPP3R2 | Protein phosphatase 3 | 5535 | Signaling | Signaling |
| | regulatory subunit B, beta | | | |
| PPT1 | Palmitoyl-protein thioesterase | 5538 | Other | Miscelaneous |
| | 1 | | | |
| PPY | Pancreatic polypeptide | 5539 | Neuropeptide | Ligand |
| PPY2P | Pancreatic polypeptide 2, | 23614 | Neuropeptide | Ligand |
| | pseudogene | | | |
| PRIMA1 | Proline rich membrane anchor | 145270 | Neurotransmitter | Biosynthesis |
| | 1 | | | |
| PRKACA | Protein kinase camp-activated | 5566 | Signaling | Signaling |
| | catalytic subunit alpha | | | |
| PRKACB | Protein kinase camp-activated | 5567 | Signaling | Signaling |
| | catalytic subunit beta | | | |
| PRKACG | Protein kinase camp-activated | 5568 | Neurotransmitter | Signaling |
| | catalytic subunit gamma | | | |
| PRKAR1A | Protein kinase camp- | 5573 | Signaling | Signaling |
| | dependent type I regulatory | | | |
| | subunit alpha | | | |
| PRKAR2A | Protein kinase camp- | 5576 | Signaling | Signaling |
| | dependent type II regulatory | | | |
| | subunit alpha | | | |
| PRKAR2B | Protein kinase camp- | 5577 | Neurotransmitter | Signaling |
| | dependent type II regulatory | | | |
| | subunit beta | | | |
| PRKCA | Protein kinase C alpha | 5578 | Signaling | Signaling |
| PRKCD | Protein kinase C delta | 5580 | Signaling | Signaling |
| PRKCE | Protein kinase C epsilon | 5581 | Signaling | Signaling |
| PRKCG | Protein kinase C gamma | 5582 | Neurotransmitter | Signaling |
| PRKX | Protein kinase, X-linked | 5613 | Neurotransmitter | Signaling |
| PRL | Prolactin | 5617 | Neuropeptide | Ligand |
| PRLH | Prolactin releasing hormone | 51052 | Neuropeptide | Ligand |
| PRLHR | Prolactin releasing hormone | 2834 | Neuropeptide | Receptor |
| | receptor | | | |
| PRLR | Prolactin receptor | 5618 | Neuropeptide | Receptor |
| PROK1 | Prokineticin 1 | 84432 | Neuropeptide | Ligand |
| PROK2 | Prokineticin 2 | 60675 | Neuropeptide | Ligand |
| PROKR1 | Prokineticin receptor 1 | 10887 | Neuropeptide | Receptor |
| PROKR2 | Prokineticin receptor 2 | 128674 | Neuropeptide | Receptor |
| PROM1 | Prominin 1 | 8842 | Other | Miscelaneous |
| PSAP | Prosaposin | 5660 | Neurotrophic | Ligand |
| PSEN1 | Presenilin 1 | 5663 | Neurotrophic | Signaling |
| PSPN | Persephin | 5623 | Neurotrophic | Ligand |
| PTEN | Phosphatase and tensin | 5728 | Signaling | Signaling |
| | homolog | | | |
| PTGDR | Prostaglandin D2 receptor | 5729 | Neuropeptide | Receptor |
| PTGDR2 | Prostaglandin D2 receptor 2 | 11251 | Neuropeptide | Receptor |
| PTGER1 | Prostaglandin E receptor 1 | 5731 | Neuropeptide | Receptor |
| PTGER2 | Prostaglandin E receptor 2 | 5732 | Neuropeptide | Receptor |
| PTGER3 | Prostaglandin E receptor 3 | 5733 | Neuropeptide | Receptor |
| PTGER4 | Prostaglandin E receptor 4 | 5734 | Neuropeptide | Receptor |
| PTGFR | Prostaglandin F receptor | 5737 | Neuropeptide | Receptor |
| PTGIR | Prostaglandin I2 (prostacyclin) | 5739 | Neuropeptide | Receptor |
| | receptor (IP) | | | |
| PTGS2 | Prostaglandin-endoperoxide | 5743 | Neuropeptide | Biosynthesis |
| | synthase 2 | | | |
| PTH | Parathyroid hormone | 5741 | Neuropeptide | Ligand |
| PTH1R | Parathyroid hormone 1 | 5745 | Neuropeptide | Receptor |
| | receptor | | | |
| PTH2 | Parathyroid hormone 2 | 113091 | Neuropeptide | Ligand |
| PTH2R | Parathyroid hormone 2 | 5746 | Neuropeptide | Receptor |
| | receptor | | | |
| PTHLH | Parathyroid hormone like | 5744 | Neuropeptide | Ligand |
| | hormone | | | |
| PTK2 | Protein tyrosine kinase 2 | 5747 | Neuropeptide | Signaling |
| PTK2B | Protein tyrosine kinase 2 beta | 2185 | Neuropeptide | Signaling |
| PTN | Pleiotrophin | 5764 | Neurotrophic | Ligand |
| PTPA | Protein phosphatase 2 | 5524 | Signaling | Signaling |
| | phosphatase activator | | | |
| PTPRN2 | Protein tyrosine phosphatase, | 5799 | Other | Miscelaneous |
| | receptor type N2 | | | |
| PXK | PX domain containing | 54899 | Vesicular | Vesicles |
| | serine/threonine kinase like | | | |
| PYY | Peptide YY | 5697 | Neuropeptide | Ligand |
| PYY2 | Peptide YY 2 (pseudogene) | 23615 | Neuropeptide | Ligand |
| PYY3 | Peptide YY 3 (pseudogene) | 644059 | Neuropeptide | Ligand |
| QRFP | Pyroglutamylated rfamide | 347148 | Neuropeptide | Ligand |
| | peptide | | | |
| QRFPR | Pyroglutamylated rfamide | 84109 | Neuropeptide | Receptor |
| | peptide receptor | | | |
| RAB3A | RAB3A, member RAS | 5864 | Signaling | Signaling |
| | oncogene family | | | |
| RAB3GAP1 | RAB3 gtpase activating | 22930 | Vesicular | Vesicles |
| | protein catalytic subunit 1 | | | |
| RAF1 | Raf-1 proto-oncogene, | 5894 | Signaling | Signaling |
| | serine/threonine kinase | | | |
| RAI1 | Retinoic acid induced 1 | 10743 | Other | Miscelaneous |
| RAMP1 | Receptor activity modifying | 10267 | Neuropeptide | Receptor |
| | protein 1 | | | |
| RAMP2 | Receptor activity modifying | 10266 | Neuropeptide | Receptor |
| | protein 2 | | | |
| RAMP3 | Receptor activity modifying | 10268 | Neuropeptide | Receptor |
| | protein 3 | | | |
| RAP1A | RAP1A, member of RAS | 5906 | Signaling | Signaling |
| | oncogene family | | | |
| RAP1GAP | RAP1 gtpase activating | 5909 | Other | Miscelaneous |
| | protein | | | |
| RAPGEF2 | Rap guanine nucleotide | 9693 | Other | Miscelaneous |
| | exchange factor 2 | | | |
| RAPGEF3 | Rap guanine nucleotide | 10411 | Signaling | Signaling |
| | exchange factor 3 | | | |
| RAPSN | Receptor associated protein of | 5913 | Synaptic | Receptor |
| | the synapse | | | |
| RELN | Reelin | 5649 | Neurotrophic | Ligand |
| RET | Ret proto-oncogene | 5979 | Neurotrophic | Signaling |
| RETN | Resistin | 56729 | Other | Miscelaneous |
| RETNLB | Resistin like beta | 84666 | Other | Miscelaneous |
| RGN | Regucalcin | 9104 | Signaling | Signaling |
| RGS10 | Regulator of G-protein | 6001 | Signaling | Signaling |
| | signaling 10 | | | |
| RGS8 | Regulator of G-protein | 85397 | Signaling | Signaling |
| | signaling 8 | | | |
| RGS9 | Regulator of G-protein | 8787 | Signaling | Signaling |
| | signaling 9 | | | |
| RHAG | Rh-associated glycoprotein | 6005 | Channel or | Transporter |
| | | | transporter | |
| RHBG | Rh family B glycoprotein | 57127 | Channel or | Transporter |
| | (gene/pseudogene) | | transporter | |
| RHCG | Rh family C glycoprotein | 51458 | Channel or | Transporter |
| | | | transporter | |
| RHOA | Ras homolog family member | 387 | Signaling | Signaling |
| | A | | | |
| RIC3 | RIC3 acetylcholine receptor | 79608 | Neurotransmitter | Receptor |
| | chaperone | | | |
| RIC8A | RIC8 guanine nucleotide | 60626 | Signaling | Signaling |
| | exchange factor A | | | |
| RIMS1 | Regulating synaptic | 22999 | Vesicular | Vesicles |
| | membrane exocytosis 1 | | | |
| RIMS2 | Regulating synaptic | 9699 | Vesicular | Vesicles |
| | membrane exocytosis 2 | | | |
| RIMS3 | Regulating synaptic | 9783 | Vesicular | Vesicles |
| | membrane exocytosis 3 | | | |
| RIMS4 | Regulating synaptic | 140730 | Vesicular | Vesicles |
| | membrane exocytosis 4 | | | |
| RLN1 | Relaxin 1 | 6013 | Neuropeptide | Ligand |
| RLN2 | Relaxin 2 | 6019 | Neuropeptide | Ligand |
| RLN3 | Relaxin 3 | 117579 | Neuropeptide | Ligand |
| RNF40 | Ring finger protein 40 | 9810 | Other | Miscelaneous |
| ROR1 | Receptor tyrosine kinase like | 4919 | Neurotrophic | Receptor |
| | orphan receptor 1 | | | |
| ROR2 | Receptor tyrosine kinase like | 4920 | Neurotrophic | Receptor |
| | orphan receptor 2 | | | |
| RPH3A | Rabphilin 3A | 22895 | Vesicular | Vesicles |
| RPH3AL | Rabphilin 3A-like (without C2 | 9501 | Vesicular | Vesicles |
| | domains) | | | |
| RPS6KA3 | Ribosomal protein S6 kinase | 6197 | Neurotrophic | Signaling |
| | A3 | | | |
| RPSA | Ribosomal protein SA | 3921 | Other | Miscelaneous |
| RXFP1 | Relaxin/insulin like family | 59350 | Neuropeptide | Receptor |
| | peptide receptor 1 | | | |
| RXFP2 | Relaxin/insulin like family | 122042 | Neuropeptide | Receptor |
| | peptide receptor 2 | | | |
| RXFP3 | Relaxin/insulin like family | 51289 | Neuropeptide | Receptor |
| | peptide receptor 3 | | | |
| RXFP4 | Relaxin/insulin like family | 339403 | Neuropeptide | Receptor |
| | peptide receptor 4 | | | |
| RYR1 | Ryanodine receptor 1 | 6261 | Channel or | Channel |
| | | | transporter | |
| RYR2 | Ryanodine receptor 2 | 6262 | Channel or | Channel |
| | | | transporter | |
| RYR3 | Ryanodine receptor 3 | 6263 | Channel or | Channel |
| | | | transporter | |
| S100B | S100 calcium binding protein | 6285 | Signaling | Signaling |
| | B | | | |
| S1PR4 | Sphingosine-1-phosphate | 8698 | Neuropeptide | Receptor |
| | receptor 4 | | | |
| SCG2 | Secretogranin II | 7857 | Neuropeptide | Vesicles |
| SCG3 | Secretogranin III | 29106 | Neuropeptide | Vesicles |
| SCG5 | Secretogranin V | 6447 | Neuropeptide | Vesicles |
| SCN10A | Sodium voltage-gated channel | 6336 | Channel or | Channel |
| | alpha subunit 10 | | transporter | |
| SCN11A | Sodium voltage-gated channel | 11280 | Channel or | Channel |
| | alpha subunit 11 | | transporter | |
| SCN1A | Sodium voltage-gated channel | 6323 | Channel or | Channel |
| | alpha subunit 1 | | transporter | |
| SCN1B | Sodium voltage-gated channel | 6324 | Channel or | Channel |
| | beta subunit 1 | | transporter | |
| SCN2A | Sodium voltage-gated channel | 6326 | Channel or | Channel |
| | alpha subunit 2 | | transporter | |
| SCN2B | Sodium voltage-gated channel | 6327 | Channel or | Channel |
| | beta subunit 2 | | transporter | |
| SCN3A | Sodium voltage-gated channel | 6328 | Channel or | Channel |
| | alpha subunit 3 | | transporter | |
| SCN3B | Sodium voltage-gated channel | 55800 | Channel or | Channel |
| | beta subunit 3 | | transporter | |
| SCN4A | Sodium voltage-gated channel | 6329 | Channel or | Channel |
| | alpha subunit 4 | | transporter | |
| SCN4B | Sodium voltage-gated channel | 6330 | Channel or | Channel |
| | beta subunit 4 | | transporter | |
| SCN5A | Sodium voltage-gated channel | 6331 | Channel or | Channel |
| | alpha subunit 5 | | transporter | |
| SCN7A | Sodium voltage-gated channel | 6332 | Channel or | Channel |
| | alpha subunit 7 | | transporter | |
| SCN8A | Sodium voltage-gated channel | 6334 | Channel or | Channel |
| | alpha subunit 8 | | transporter | |
| SCN9A | Sodium voltage-gated channel | 6335 | Channel or | Channel |
| | alpha subunit 9 | | transporter | |
| SCNN1A | Sodium channel epithelial 1 | 6337 | Channel or | Channel |
| | alpha subunit | | transporter | |
| SCNN1B | Sodium channel epithelial 1 | 6338 | Channel or | Channel |
| | beta subunit | | transporter | |
| SCNN1D | Sodium channel epithelial 1 | 6339 | Channel or | Channel |
| | delta subunit | | transporter | |
| SCNN1G | Sodium channel epithelial 1 | 6340 | Channel or | Channel |
| | gamma subunit | | transporter | |
| SCT | Secretin | 6343 | Neuropeptide | Ligand |
| SDC3 | Syndecan 3 | 9672 | Neurotrophic | Receptor |
| SEC14L1 | SEC14 like lipid binding 1 | 6397 | Other | Miscelaneous |
| SEMA3E | Semaphorin 3E | 9723 | Neurotrophic | Ligand |
| SERPINE2 | Serpin family E member 2 | 5270 | Neurotrophic | Ligand |
| SERPINF1 | Serpin family F member 1 | 5176 | Neurotrophic | Ligand |
| SHANK3 | SH3 and multiple ankyrin | 85358 | Neurotransmitter | Signaling |
| | repeat domains 3 | | | |
| SHC1 | SHC adaptor protein 1 | 6464 | Neurotrophic | Signaling |
| SHROOM1 | Shroom family member 1 | 134549 | Channel or | Channel |
| | | | transporter | |
| SHROOM2 | Shroom family member 2 | 357 | Channel or | Channel |
| | | | transporter | |
| SHROOM3 | Shroom family member 3 | 57619 | Channel or | Channel |
| | | | transporter | |
| SHROOM4 | Shroom family member 4 | 57477 | Channel or | Channel |
| | | | transporter | |
| SLC10A1 | Solute carrier family 10 | 6554 | Channel or | Transporter |
| | member 1 | | transporter | |
| SLC10A2 | Solute carrier family 10 | 6555 | Channel or | Transporter |
| | member 2 | | transporter | |
| SLC10A3 | Solute carrier family 10 | 8273 | Channel or | Transporter |
| | member 3 | | transporter | |
| SLC10A4 | Solute carrier family 10 | 201780 | Channel or | Transporter |
| | member 4 | | transporter | |
| SLC10A5 | Solute carrier family 10 | 347051 | Channel or | Transporter |
| | member 5 | | transporter | |
| SLC10A6 | Solute carrier family 10 | 345274 | Channel or | Transporter |
| | member 6 | | transporter | |
| SLC10A7 | Solute carrier family 10 | 84068 | Channel or | Transporter |
| | member 7 | | transporter | |
| SLC11A1 | Solute carrier family 11 | 6556 | Channel or | Transporter |
| | member 1 | | transporter | |
| SLC11A2 | Solute carrier family 11 | 4891 | Channel or | Transporter |
| | member 2 | | transporter | |
| SLC12A1 | Solute carrier family 12 | 6557 | Channel or | Transporter |
| | member 1 | | transporter | |
| SLC12A2 | Solute carrier family 12 | 6558 | Channel or | Transporter |
| | member 2 | | transporter | |
| SLC12A3 | Solute carrier family 12 | 6559 | Channel or | Transporter |
| | member 3 | | transporter | |
| SLC12A4 | Solute carrier family 12 | 6560 | Channel or | Transporter |
| | member 4 | | transporter | |
| SLC12A5 | Solute carrier family 12 | 57468 | Channel or | Transporter |
| | member 5 | | transporter | |
| SLC12A6 | Solute carrier family 12 | 9990 | Channel or | Transporter |
| | member 6 | | transporter | |
| SLC12A7 | Solute carrier family 12 | 10723 | Channel or | Transporter |
| | member 7 | | transporter | |
| SLC12A8 | Solute carrier family 12 | 84561 | Channel or | Transporter |
| | member 8 | | transporter | |
| SLC12A9 | Solute carrier family 12 | 56996 | Channel or | Transporter |
| | member 9 | | transporter | |
| SLC13A1 | Solute carrier family 13 | 6561 | Channel or | Transporter |
| | member 1 | | transporter | |
| SLC13A2 | Solute carrier family 13 | 9058 | Channel or | Transporter |
| | member 2 | | transporter | |
| SLC13A3 | Solute carrier family 13 | 64849 | Channel or | Transporter |
| | member 3 | | transporter | |
| SLC13A4 | Solute carrier family 13 | 26266 | Channel or | Transporter |
| | member 4 | | transporter | |
| SLC13A5 | Solute carrier family 13 | 284111 | Channel or | Transporter |
| | member 5 | | transporter | |
| SLC14A1 | Solute carrier family 14 | 6563 | Channel or | Transporter |
| | member 1 (Kidd blood group) | | transporter | |
| SLC14A2 | Solute carrier family 14 | 8170 | Channel or | Transporter |
| | member 2 | | transporter | |
| SLC15A1 | Solute carrier family 15 | 6564 | Channel or | Transporter |
| | member 1 | | transporter | |
| SLC15A2 | Solute carrier family 15 | 6565 | Channel or | Transporter |
| | member 2 | | transporter | |
| SLC15A3 | Solute carrier family 15 | 51296 | Channel or | Transporter |
| | member 3 | | transporter | |
| SLC15A4 | Solute carrier family 15 | 121260 | Channel or | Transporter |
| | member 4 | | transporter | |
| SLC16A1 | Solute carrier family 16 | 6566 | Channel or | Transporter |
| | member 1 | | transporter | |
| SLC16A10 | Solute carrier family 16 | 117247 | Channel or | Transporter |
| | member 10 | | transporter | |
| SLC16A11 | Solute carrier family 16 | 162515 | Channel or | Transporter |
| | member 11 | | transporter | |
| SLC16A12 | Solute carrier family 16 | 387700 | Channel or | Transporter |
| | member 12 | | transporter | |
| SLC16A13 | Solute carrier family 16 | 201232 | Channel or | Transporter |
| | member 13 | | transporter | |
| SLC16A14 | Solute carrier family 16 | 151473 | Channel or | Transporter |
| | member 14 | | transporter | |
| SLC16A2 | Solute carrier family 16 | 6567 | Channel or | Transporter |
| | member 2 | | transporter | |
| SLC16A3 | Solute carrier family 16 | 9123 | Channel or | Transporter |
| | member 3 | | transporter | |
| SLC16A4 | Solute carrier family 16 | 9122 | Channel or | Transporter |
| | member 4 | | transporter | |
| SLC16A5 | Solute carrier family 16 | 9121 | Channel or | Transporter |
| | member 5 | | transporter | |
| SLC16A6 | Solute carrier family 16 | 9120 | Channel or | Transporter |
| | member 6 | | transporter | |
| SLC16A7 | Solute carrier family 16 | 9194 | Channel or | Transporter |
| | member 7 | | transporter | |
| SLC16A8 | Solute carrier family 16 | 23539 | Channel or | Transporter |
| | member 8 | | transporter | |
| SLC16A9 | Solute carrier family 16 | 220963 | Channel or | Transporter |
| | member 9 | | transporter | |
| SLC17A1 | Solute carrier family 17 | 6568 | Channel or | Transporter |
| | member 1 | | transporter | |
| SLC17A2 | Solute carrier family 17 | 10246 | Channel or | Transporter |
| | member 2 | | transporter | |
| SLC17A3 | Solute carrier family 17 | 10786 | Channel or | Transporter |
| | member 3 | | transporter | |
| SLC17A4 | Solute carrier family 17 | 10050 | Channel or | Transporter |
| | member 4 | | transporter | |
| SLC17A5 | Solute carrier family 17 | 26503 | Channel or | Transporter |
| | member 5 | | transporter | |
| SLC17A6 | Solute carrier family 17 | 57084 | Channel or | Transporter |
| | member 6 | | transporter | |
| SLC17A7 | Solute carrier family 17 | 57030 | Channel or | Transporter |
| | member 7 | | transporter | |
| SLC17A8 | Solute carrier family 17 | 246213 | Channel or | Transporter |
| | member 8 | | transporter | |
| SLC17A9 | Solute carrier family 17 | 63910 | Channel or | Transporter |
| | member 9 | | transporter | |
| SLC18A1 | Solute carrier family 18 | 6570 | Channel or | Transporter |
| | member A1 | | transporter | |
| SLC18A2 | Solute carrier family 18 | 6571 | Channel or | Transporter |
| | member A2 | | transporter | |
| SLC18A3 | Solute carrier family 18 | 6572 | Channel or | Transporter |
| | member A3 | | transporter | |
| SLC18B1 | Solute carrier family 18 | 116843 | Channel or | Transporter |
| | member B1 | | transporter | |
| SLC19A1 | Solute carrier family 19 | 6573 | Channel or | Transporter |
| | member 1 | | transporter | |
| SLC19A2 | Solute carrier family 19 | 10560 | Channel or | Transporter |
| | member 2 | | transporter | |
| SLC19A3 | Solute carrier family 19 | 80704 | Channel or | Transporter |
| | member 3 | | transporter | |
| SLC1A1 | Solute carrier family 1 | 6505 | Channel or | Transporter |
| | member 1 | | transporter | |
| SLC1A2 | Solute carrier family 1 | 6506 | Channel or | Transporter |
| | member 2 | | transporter | |
| SLC1A3 | Solute carrier family 1 | 6507 | Channel or | Transporter |
| | member 3 | | transporter | |
| SLC1A6 | Solute carrier family 1 | 6511 | Channel or | Transporter |
| | member 6 | | transporter | |
| SLC1A7 | Solute carrier family 1 | 6512 | Channel or | Transporter |
| | member 7 | | transporter | |
| SLC20A1 | Solute carrier family 20 | 6574 | Channel or | Transporter |
| | member 1 | | transporter | |
| SLC20A2 | Solute carrier family 20 | 6575 | Channel or | Transporter |
| | member 2 | | transporter | |
| SLC22A1 | Solute carrier family 22 | 6580 | Channel or | Transporter |
| | member 1 | | transporter | |
| SLC22A10 | Solute carrier family 22 | 387775 | Channel or | Transporter |
| | member 10 | | transporter | |
| SLC22A11 | Solute carrier family 22 | 55867 | Channel or | Transporter |
| | member 11 | | transporter | |
| SLC22A12 | Solute carrier family 22 | 116085 | Channel or | Transporter |
| | member 12 | | transporter | |
| SLC22A13 | Solute carrier family 22 | 9390 | Channel or | Transporter |
| | member 13 | | transporter | |
| SLC22A14 | Solute carrier family 22 | 9389 | Channel or | Transporter |
| | member 14 | | transporter | |
| SLC22A15 | Solute carrier family 22 | 55356 | Channel or | Transporter |
| | member 15 | | transporter | |
| SLC22A16 | Solute carrier family 22 | 85413 | Channel or | Transporter |
| | member 16 | | transporter | |
| SLC22A17 | Solute carrier family 22 | 51310 | Channel or | Transporter |
| | member 17 | | transporter | |
| SLC22A18 | Solute carrier family 22 | 5002 | Channel or | Transporter |
| | member 18 | | transporter | |
| SLC22A2 | Solute carrier family 22 | 6582 | Channel or | Transporter |
| | member 2 | | transporter | |
| SLC22A20 | Solute carrier family 22 | 440044 | Channel or | Transporter |
| | member 20 | | transporter | |
| SLC22A23 | Solute carrier family 22 | 63027 | Channel or | Transporter |
| | member 23 | | transporter | |
| SLC22A24 | Solute carrier family 22 | 283238 | Channel or | Transporter |
| | member 24 | | transporter | |
| SLC22A25 | Solute carrier family 22 | 387601 | Channel or | Transporter |
| | member 25 | | transporter | |
| SLC22A3 | Solute carrier family 22 | 6581 | Channel or | Transporter |
| | member 3 | | transporter | |
| SLC22A31 | Solute carrier family 22 | 146429 | Channel or | Transporter |
| | member 31 | | transporter | |
| SLC22A4 | Solute carrier family 22 | 6583 | Channel or | Transporter |
| | member 4 | | transporter | |
| SLC22A5 | Solute carrier family 22 | 6584 | Channel or | Transporter |
| | member 5 | | transporter | |
| SLC22A6 | Solute carrier family 22 | 9356 | Channel or | Transporter |
| | member 6 | | transporter | |
| SLC22A7 | Solute carrier family 22 | 10864 | Channel or | Transporter |
| | member 7 | | transporter | |
| SLC22A8 | Solute carrier family 22 | 9376 | Channel or | Transporter |
| | member 8 | | transporter | |
| SLC22A9 | Solute carrier family 22 | 114571 | Channel or | Transporter |
| | member 9 | | transporter | |
| SLC23A1 | Solute carrier family 23 | 9963 | Channel or | Transporter |
| | member 1 | | transporter | |
| SLC23A2 | Solute carrier family 23 | 9962 | Channel or | Transporter |
| | member 2 | | transporter | |
| SLC23A3 | Solute carrier family 23 | 151295 | Channel or | Transporter |
| | member 3 | | transporter | |
| SLC23A4P | Solute carrier family 23 | 641842 | Channel or | Transporter |
| | member 4, pseudogene | | transporter | |
| SLC24A1 | Solute carrier family 24 | 9187 | Channel or | Transporter |
| | member 1 | | transporter | |
| SLC24A2 | Solute carrier family 24 | 25769 | Channel or | Transporter |
| | member 2 | | transporter | |
| SLC24A3 | Solute carrier family 24 | 57419 | Channel or | Transporter |
| | member 3 | | transporter | |
| SLC24A4 | Solute carrier family 24 | 123041 | Channel or | Transporter |
| | member 4 | | transporter | |
| SLC24A5 | Solute carrier family 24 | 283652 | Channel or | Transporter |
| | member 5 | | transporter | |
| SLC25A1 | Solute carrier family 25 | 6576 | Channel or | Transporter |
| | member 1 | | transporter | |
| SLC25A10 | Solute carrier family 25 | 1468 | Channel or | Transporter |
| | member 10 | | transporter | |
| SLC25A11 | Solute carrier family 25 | 8402 | Channel or | Transporter |
| | member 11 | | transporter | |
| SLC25A12 | Solute carrier family 25 | 8604 | Channel or | Transporter |
| | member 12 | | transporter | |
| SLC25A13 | Solute carrier family 25 | 10165 | Channel or | Transporter |
| | member 13 | | transporter | |
| SLC25A14 | Solute carrier family 25 | 9016 | Channel or | Transporter |
| | member 14 | | transporter | |
| SLC25A15 | Solute carrier family 25 | 10166 | Channel or | Transporter |
| | member 15 | | transporter | |
| SLC25A16 | Solute carrier family 25 | 8034 | Channel or | Transporter |
| | member 16 | | transporter | |
| SLC25A17 | Solute carrier family 25 | 10478 | Channel or | Transporter |
| | member 17 | | transporter | |
| SLC25A18 | Solute carrier family 25 | 83733 | Channel or | Transporter |
| | member 18 | | transporter | |
| SLC25A19 | Solute carrier family 25 | 60386 | Channel or | Transporter |
| | member 19 | | transporter | |
| SLC25A2 | Solute carrier family 25 | 83884 | Channel or | Transporter |
| | member 2 | | transporter | |
| SLC25A20 | Solute carrier family 25 | 788 | Channel or | Transporter |
| | member 20 | | transporter | |
| SLC25A21 | Solute carrier family 25 | 89874 | Channel or | Transporter |
| | member 21 | | transporter | |
| SLC25A22 | Solute carrier family 25 | 79751 | Channel or | Transporter |
| | member 22 | | transporter | |
| SLC25A23 | Solute carrier family 25 | 79085 | Channel or | Transporter |
| | member 23 | | transporter | |
| SLC25A24 | Solute carrier family 25 | 29957 | Channel or | Transporter |
| | member 24 | | transporter | |
| SLC25A25 | Solute carrier family 25 | 114789 | Channel or | Transporter |
| | member 25 | | transporter | |
| SLC25A26 | Solute carrier family 25 | 115286 | Channel or | Transporter |
| | member 26 | | transporter | |
| SLC25A27 | Solute carrier family 25 | 9481 | Channel or | Transporter |
| | member 27 | | transporter | |
| SLC25A28 | Solute carrier family 25 | 81894 | Channel or | Transporter |
| | member 28 | | transporter | |
| SLC25A29 | Solute carrier family 25 | 123096 | Channel or | Transporter |
| | member 29 | | transporter | |
| SLC25A3 | Solute carrier family 25 | 5250 | Channel or | Transporter |
| | member 3 | | transporter | |
| SLC25A30 | Solute carrier family 25 | 253512 | Channel or | Transporter |
| | member 30 | | transporter | |
| SLC25A31 | Solute carrier family 25 | 83447 | Channel or | Transporter |
| | member 31 | | transporter | |
| SLC25A32 | Solute carrier family 25 | 81034 | Channel or | Transporter |
| | member 32 | | transporter | |
| SLC25A33 | Solute carrier family 25 | 84275 | Channel or | Transporter |
| | member 33 | | transporter | |
| SLC25A34 | Solute carrier family 25 | 284723 | Channel or | Transporter |
| | member 34 | | transporter | |
| SLC25A35 | Solute carrier family 25 | 399512 | Channel or | Transporter |
| | member 35 | | transporter | |
| SLC25A36 | Solute carrier family 25 | 55186 | Channel or | Transporter |
| | member 36 | | transporter | |
| SLC25A37 | Solute carrier family 25 | 51312 | Channel or | Transporter |
| | member 37 | | transporter | |
| SLC25A38 | Solute carrier family 25 | 54977 | Channel or | Transporter |
| | member 38 | | transporter | |
| SLC25A39 | Solute carrier family 25 | 51629 | Channel or | Transporter |
| | member 39 | | transporter | |
| SLC25A4 | Solute carrier family 25 | 291 | Channel or | Transporter |
| | member 4 | | transporter | |
| SLC25A40 | Solute carrier family 25 | 55972 | Channel or | Transporter |
| | member 40 | | transporter | |
| SLC25A41 | Solute carrier family 25 | 284427 | Channel or | Transporter |
| | member 41 | | transporter | |
| SLC25A42 | Solute carrier family 25 | 284439 | Channel or | Transporter |
| | member 42 | | transporter | |
| SLC25A43 | Solute carrier family 25 | 203427 | Channel or | Transporter |
| | member 43 | | transporter | |
| SLC25A44 | Solute carrier family 25 | 9673 | Channel or | Transporter |
| | member 44 | | transporter | |
| SLC25A45 | Solute carrier family 25 | 283130 | Channel or | Transporter |
| | member 45 | | transporter | |
| SLC25A46 | Solute carrier family 25 | 91137 | Channel or | Transporter |
| | member 46 | | transporter | |
| SLC25A47 | Solute carrier family 25 | 283600 | Channel or | Transporter |
| | member 47 | | transporter | |
| SLC25A48 | Solute carrier family 25 | 153328 | Channel or | Transporter |
| | member 48 | | transporter | |
| SLC25A5 | Solute carrier family 25 | 292 | Channel or | Transporter |
| | member 5 | | transporter | |
| SLC25A51 | Solute carrier family 25 | 92014 | Channel or | Transporter |
| | member 51 | | transporter | |
| SLC25A52 | Solute carrier family 25 | 147407 | Channel or | Transporter |
| | member 52 | | transporter | |
| SLC25A53 | Solute carrier family 25 | 401612 | Channel or | Transporter |
| | member 53 | | transporter | |
| SLC25A6 | Solute carrier family 25 | 293 | Channel or | Transporter |
| | member 6 | | transporter | |
| SLC26A1 | Solute carrier family 26 | 10861 | Channel or | Transporter |
| | member 1 | | transporter | |
| SLC26A10 | Solute carrier family 26 | 65012 | Channel or | Transporter |
| | member 10 | | transporter | |
| SLC26A11 | Solute carrier family 26 | 284129 | Channel or | Transporter |
| | member 11 | | transporter | |
| SLC26A2 | Solute carrier family 26 | 1836 | Channel or | Transporter |
| | member 2 | | transporter | |
| SLC26A3 | Solute carrier family 26 | 1811 | Channel or | Transporter |
| | member 3 | | transporter | |
| SLC26A4 | Solute carrier family 26 | 5172 | Channel or | Transporter |
| | member 4 | | transporter | |
| SLC26A5 | Solute carrier family 26 | 375611 | Channel or | Transporter |
| | member 5 | | transporter | |
| SLC26A6 | Solute carrier family 26 | 65010 | Channel or | Transporter |
| | member 6 | | transporter | |
| SLC26A7 | Solute carrier family 26 | 115111 | Channel or | Transporter |
| | member 7 | | transporter | |
| SLC26A8 | Solute carrier family 26 | 116369 | Channel or | Transporter |
| | member 8 | | transporter | |
| SLC26A9 | Solute carrier family 26 | 115019 | Channel or | Transporter |
| | member 9 | | transporter | |
| SLC27A1 | Solute carrier family 27 | 376497 | Channel or | Transporter |
| | member 1 | | transporter | |
| SLC27A2 | Solute carrier family 27 | 11001 | Channel or | Transporter |
| | member 2 | | transporter | |
| SLC27A3 | Solute carrier family 27 | 11000 | Channel or | Transporter |
| | member 3 | | transporter | |
| SLC27A4 | Solute carrier family 27 | 10999 | Channel or | Transporter |
| | member 4 | | transporter | |
| SLC27A5 | Solute carrier family 27 | 10998 | Channel or | Transporter |
| | member 5 | | transporter | |
| SLC27A6 | Solute carrier family 27 | 28965 | Channel or | Transporter |
| | member 6 | | transporter | |
| SLC28A1 | Solute carrier family 28 | 9154 | Channel or | Transporter |
| | member 1 | | transporter | |
| SLC28A2 | Solute carrier family 28 | 9153 | Channel or | Transporter |
| | member 2 | | transporter | |
| SLC28A3 | Solute carrier family 28 | 64078 | Channel or | Transporter |
| | member 3 | | transporter | |
| SLC29A1 | Solute carrier family 29 | 2030 | Channel or | Transporter |
| | member 1 (Augustine blood | | transporter | |
| | group) | | | |
| SLC29A2 | Solute carrier family 29 | 3177 | Channel or | Transporter |
| | member 2 | | transporter | |
| SLC29A3 | Solute carrier family 29 | 55315 | Channel or | Transporter |
| | member 3 | | transporter | |
| SLC29A4 | Solute carrier family 29 | 222962 | Channel or | Transporter |
| | member 4 | | transporter | |
| SLC2A1 | Solute carrier family 2 | 6513 | Channel or | Transporter |
| | member 1 | | transporter | |
| SLC2A10 | Solute carrier family 2 | 81031 | Channel or | Transporter |
| | member 10 | | transporter | |
| SLC2A11 | Solute carrier family 2 | 66035 | Channel or | Transporter |
| | member 11 | | transporter | |
| SLC2A12 | Solute carrier family 2 | 154091 | Channel or | Transporter |
| | member 12 | | transporter | |
| SLC2A13 | Solute carrier family 2 | 114134 | Channel or | Transporter |
| | member 13 | | transporter | |
| SLC2A14 | Solute carrier family 2 | 144195 | Channel or | Transporter |
| | member 14 | | transporter | |
| SLC2A2 | Solute carrier family 2 | 6514 | Channel or | Transporter |
| | member 2 | | transporter | |
| SLC2A3 | Solute carrier family 2 | 6515 | Channel or | Transporter |
| | member 3 | | transporter | |
| SLC2A4 | Solute carrier family 2 | 6517 | Channel or | Transporter |
| | member 4 | | transporter | |
| SLC2A5 | Solute carrier family 2 | 6518 | Channel or | Transporter |
| | member 5 | | transporter | |
| SLC2A6 | Solute carrier family 2 | 11182 | Channel or | Transporter |
| | member 6 | | transporter | |
| SLC2A7 | Solute carrier family 2 | 155184 | Channel or | Transporter |
| | member 7 | | transporter | |
| SLC2A8 | Solute carrier family 2 | 29988 | Channel or | Transporter |
| | member 8 | | transporter | |
| SLC2A9 | Solute carrier family 2 | 56606 | Channel or | Transporter |
| | member 9 | | transporter | |
| SLC30A1 | Solute carrier family 30 | 7779 | Channel or | Transporter |
| | member 1 | | transporter | |
| SLC30A10 | Solute carrier family 30 | 55532 | Channel or | Transporter |
| | member 10 | | transporter | |
| SLC30A2 | Solute carrier family 30 | 7780 | Channel or | Transporter |
| | member 2 | | transporter | |
| SLC30A3 | Solute carrier family 30 | 7781 | Channel or | Transporter |
| | member 3 | | transporter | |
| SLC30A4 | Solute carrier family 30 | 7782 | Channel or | Transporter |
| | member 4 | | transporter | |
| SLC30A5 | Solute carrier family 30 | 64924 | Channel or | Transporter |
| | member 5 | | transporter | |
| SLC30A6 | Solute carrier family 30 | 55676 | Channel or | Transporter |
| | member 6 | | transporter | |
| SLC30A7 | Solute carrier family 30 | 148867 | Channel or | Transporter |
| | member 7 | | transporter | |
| SLC30A8 | Solute carrier family 30 | 169026 | Channel or | Transporter |
| | member 8 | | transporter | |
| SLC30A9 | Solute carrier family 30 | 10463 | Channel or | Transporter |
| | member 9 | | transporter | |
| SLC31A1 | Solute carrier family 31 | 1317 | Channel or | Transporter |
| | member 1 | | transporter | |
| SLC31A2 | Solute carrier family 31 | 1318 | Channel or | Transporter |
| | member 2 | | transporter | |
| SLC32A1 | Solute carrier family 32 | 140679 | Channel or | Transporter |
| | member 1 | | transporter | |
| SLC33A1 | Solute carrier family 33 | 9197 | Channel or | Transporter |
| | member 1 | | transporter | |
| SLC34A1 | Solute carrier family 34 | 6569 | Channel or | Transporter |
| | member 1 | | transporter | |
| SLC34A2 | Solute carrier family 34 | 10568 | Channel or | Transporter |
| | member 2 | | transporter | |
| SLC34A3 | Solute carrier family 34 | 142680 | Channel or | Transporter |
| | member 3 | | transporter | |
| SLC35A1 | Solute carrier family 35 | 10559 | Channel or | Transporter |
| | member A1 | | transporter | |
| SLC35A2 | Solute carrier family 35 | 7355 | Channel or | Transporter |
| | member A2 | | transporter | |
| SLC35A3 | Solute carrier family 35 | 23443 | Channel or | Transporter |
| | member A3 | | transporter | |
| SLC35A4 | Solute carrier family 35 | 113829 | Channel or | Transporter |
| | member A4 | | transporter | |
| SLC35A5 | Solute carrier family 35 | 55032 | Channel or | Transporter |
| | member A5 | | transporter | |
| SLC35B1 | Solute carrier family 35 | 10237 | Channel or | Transporter |
| | member B1 | | transporter | |
| SLC35B2 | Solute carrier family 35 | 347734 | Channel or | Transporter |
| | member B2 | | transporter | |
| SLC35B3 | Solute carrier family 35 | 51000 | Channel or | Transporter |
| | member B3 | | transporter | |
| SLC35B4 | Solute carrier family 35 | 84912 | Channel or | Transporter |
| | member B4 | | transporter | |
| SLC35C1 | Solute carrier family 35 | 55343 | Channel or | Transporter |
| | member C1 | | transporter | |
| SLC35C2 | Solute carrier family 35 | 51006 | Channel or | Transporter |
| | member C2 | | transporter | |
| SLC35D1 | Solute carrier family 35 | 23169 | Channel or | Transporter |
| | member D1 | | transporter | |
| SLC35D2 | Solute carrier family 35 | 11046 | Channel or | Transporter |
| | member D2 | | transporter | |
| SLC35D3 | Solute carrier family 35 | 340146 | Channel or | Transporter |
| | member D3 | | transporter | |
| SLC35E1 | Solute carrier family 35 | 79939 | Channel or | Transporter |
| | member E1 | | transporter | |
| SLC35E2 | Solute carrier family 35 | 9906 | Channel or | Transporter |
| | member E2 | | transporter | |
| SLC35E2B | Solute carrier family 35 | 728661 | Channel or | Transporter |
| | member E2B | | transporter | |
| SLC35E3 | Solute carrier family 35 | 55508 | Channel or | Transporter |
| | member E3 | | transporter | |
| SLC35E4 | Solute carrier family 35 | 339665 | Channel or | Transporter |
| | member E4 | | transporter | |
| SLC35F1 | Solute carrier family 35 | 222553 | Channel or | Transporter |
| | member F1 | | transporter | |
| SLC35F2 | Solute carrier family 35 | 54733 | Channel or | Transporter |
| | member F2 | | transporter | |
| SLC35F3 | Solute carrier family 35 | 148641 | Channel or | Transporter |
| | member F3 | | transporter | |
| SLC35F4 | Solute carrier family 35 | 341880 | Channel or | Transporter |
| | member F4 | | transporter | |
| SLC35F5 | Solute carrier family 35 | 80255 | Channel or | Transporter |
| | member F5 | | transporter | |
| SLC35F6 | Solute carrier family 35 | 54978 | Channel or | Transporter |
| | member F6 | | transporter | |
| SLC35G1 | Solute carrier family 35 | 159371 | Channel or | Transporter |
| | member G1 | | transporter | |
| SLC35G2 | Solute carrier family 35 | 80723 | Channel or | Transporter |
| | member G2 | | transporter | |
| SLC35G3 | Solute carrier family 35 | 146861 | Channel or | Transporter |
| | member G3 | | transporter | |
| SLC35G4 | Solute carrier family 35 | 646000 | Channel or | Transporter |
| | member G4 | | transporter | |
| SLC35G5 | Solute carrier family 35 | 83650 | Channel or | Transporter |
| | member G5 | | transporter | |
| SLC35G6 | Solute carrier family 35 | 643664 | Channel or | Transporter |
| | member G6 | | transporter | |
| SLC36A1 | Solute carrier family 36 | 206358 | Channel or | Transporter |
| | member 1 | | transporter | |
| SLC36A2 | Solute carrier family 36 | 153201 | Channel or | Transporter |
| | member 2 | | transporter | |
| SLC36A3 | Solute carrier family 36 | 285641 | Channel or | Transporter |
| | member 3 | | transporter | |
| SLC36A4 | Solute carrier family 36 | 120103 | Channel or | Transporter |
| | member 4 | | transporter | |
| SLC37A1 | Solute carrier family 37 | 54020 | Channel or | Transporter |
| | member 1 | | transporter | |
| SLC37A2 | Solute carrier family 37 | 219855 | Channel or | Transporter |
| | member 2 | | transporter | |
| SLC37A3 | Solute carrier family 37 | 84255 | Channel or | Transporter |
| | member 3 | | transporter | |
| SLC37A4 | Solute carrier family 37 | 2542 | Channel or | Transporter |
| | member 4 | | transporter | |
| SLC38A1 | Solute carrier family 38 | 81539 | Channel or | Transporter |
| | member 1 | | transporter | |
| SLC38A10 | Solute carrier family 38 | 124565 | Channel or | Transporter |
| | member 10 | | transporter | |
| SLC38A11 | Solute carrier family 38 | 151258 | Channel or | Transporter |
| | member 11 | | transporter | |
| SLC38A2 | Solute carrier family 38 | 54407 | Channel or | Transporter |
| | member 2 | | transporter | |
| SLC38A3 | Solute carrier family 38 | 10991 | Channel or | Transporter |
| | member 3 | | transporter | |
| SLC38A4 | Solute carrier family 38 | 55089 | Channel or | Transporter |
| | member 4 | | transporter | |
| SLC38A5 | Solute carrier family 38 | 92745 | Channel or | Transporter |
| | member 5 | | transporter | |
| SLC38A6 | Solute carrier family 38 | 145389 | Channel or | Transporter |
| | member 6 | | transporter | |
| SLC38A7 | Solute carrier family 38 | 55238 | Channel or | Transporter |
| | member 7 | | transporter | |
| SLC38A8 | Solute carrier family 38 | 146167 | Channel or | Transporter |
| | member 8 | | transporter | |
| SLC38A9 | Solute carrier family 38 | 153129 | Channel or | Transporter |
| | member 9 | | transporter | |
| SLC39A1 | Solute carrier family 39 | 27173 | Channel or | Transporter |
| | member 1 | | transporter | |
| SLC39A10 | Solute carrier family 39 | 57181 | Channel or | Transporter |
| | member 10 | | transporter | |
| SLC39A11 | Solute carrier family 39 | 201266 | Channel or | Transporter |
| | member 11 | | transporter | |
| SLC39A12 | Solute carrier family 39 | 221074 | Channel or | Transporter |
| | member 12 | | transporter | |
| SLC39A13 | Solute carrier family 39 | 91252 | Channel or | Transporter |
| | member 13 | | transporter | |
| SLC39A14 | Solute carrier family 39 | 23516 | Channel or | Transporter |
| | member 14 | | transporter | |
| SLC39A2 | Solute carrier family 39 | 29986 | Channel or | Transporter |
| | member 2 | | transporter | |
| SLC39A3 | Solute carrier family 39 | 29985 | Channel or | Transporter |
| | member 3 | | transporter | |
| SLC39A4 | Solute carrier family 39 | 55630 | Channel or | Transporter |
| | member 4 | | transporter | |
| SLC39A5 | Solute carrier family 39 | 283375 | Channel or | Transporter |
| | member 5 | | transporter | |
| SLC39A6 | Solute carrier family 39 | 25800 | Channel or | Transporter |
| | member 6 | | transporter | |
| SLC39A7 | Solute carrier family 39 | 7922 | Channel or | Transporter |
| | member 7 | | transporter | |
| SLC39A8 | Solute carrier family 39 | 64116 | Channel or | Transporter |
| | member 8 | | transporter | |
| SLC39A9 | Solute carrier family 39 | 55334 | Channel or | Transporter |
| | member 9 | | transporter | |
| SLC3A1 | Solute carrier family 3 | 6519 | Channel or | Transporter |
| | member 1 | | transporter | |
| SLC3A2 | Solute carrier family 3 | 6520 | Channel or | Transporter |
| | member 2 | | transporter | |
| SLC40A1 | Solute carrier family 40 | 30061 | Channel or | Transporter |
| | member 1 | | transporter | |
| SLC41A1 | Solute carrier family 41 | 254428 | Channel or | Transporter |
| | member 1 | | transporter | |
| SLC41A2 | Solute carrier family 41 | 84102 | Channel or | Transporter |
| | member 2 | | transporter | |
| SLC41A3 | Solute carrier family 41 | 54946 | Channel or | Transporter |
| | member 3 | | transporter | |
| SLC43A1 | Solute carrier family 43 | 8501 | Channel or | Transporter |
| | member 1 | | transporter | |
| SLC43A2 | Solute carrier family 43 | 124935 | Channel or | Transporter |
| | member 2 | | transporter | |
| SLC43A3 | Solute carrier family 43 | 29015 | Channel or | Transporter |
| | member 3 | | transporter | |
| SLC44A1 | Solute carrier family 44 | 23446 | Channel or | Transporter |
| | member 1 | | transporter | |
| SLC44A2 | Solute carrier family 44 | 57153 | Channel or | Transporter |
| | member 2 | | transporter | |
| SLC44A3 | Solute carrier family 44 | 126969 | Channel or | Transporter |
| | member 3 | | transporter | |
| SLC44A4 | Solute carrier family 44 | 80736 | Channel or | Transporter |
| | member 4 | | transporter | |
| SLC44A5 | Solute carrier family 44 | 204962 | Channel or | Transporter |
| | member 5 | | transporter | |
| SLC45A1 | Solute carrier family 45 | 50651 | Channel or | Transporter |
| | member 1 | | transporter | |
| SLC45A2 | Solute carrier family 45 | 51151 | Channel or | Transporter |
| | member 2 | | transporter | |
| SLC45A3 | Solute carrier family 45 | 85414 | Channel or | Transporter |
| | member 3 | | transporter | |
| SLC45A4 | Solute carrier family 45 | 57210 | Channel or | Transporter |
| | member 4 | | transporter | |
| SLC46A1 | Solute carrier family 46 | 113235 | Channel or | Transporter |
| | member 1 | | transporter | |
| SLC46A2 | Solute carrier family 46 | 57864 | Channel or | Transporter |
| | member 2 | | transporter | |
| SLC46A3 | Solute carrier family 46 | 283537 | Channel or | Transporter |
| | member 3 | | transporter | |
| SLC47A1 | Solute carrier family 47 | 55244 | Channel or | Transporter |
| | member 1 | | transporter | |
| SLC47A2 | Solute carrier family 47 | 146802 | Channel or | Transporter |
| | member 2 | | transporter | |
| SLC48A1 | Solute carrier family 48 | 55652 | Channel or | Transporter |
| | member 1 | | transporter | |
| SLC4A1 | Solute carrier family 4 | 6521 | Channel or | Transporter |
| | member 1 | | transporter | |
| SLC4A10 | Solute carrier family 4 | 57282 | Channel or | Transporter |
| | member 10 | | transporter | |
| SLC4A11 | Solute carrier family 4 | 83959 | Channel or | Transporter |
| | member 11 | | transporter | |
| SLC4A2 | Solute carrier family 4 | 6522 | Channel or | Transporter |
| | member 2 | | transporter | |
| SLC4A3 | Solute carrier family 4 | 6508 | Channel or | Transporter |
| | member 3 | | transporter | |
| SLC4A4 | Solute carrier family 4 | 8671 | Channel or | Transporter |
| | member 4 | | transporter | |
| SLC4A5 | Solute carrier family 4 | 57835 | Channel or | Transporter |
| | member 5 | | transporter | |
| SLC4A7 | Solute carrier family 4 | 9497 | Channel or | Transporter |
| | member 7 | | transporter | |
| SLC4A8 | Solute carrier family 4 | 9498 | Channel or | Transporter |
| | member 8 | | transporter | |
| SLC4A9 | Solute carrier family 4 | 83697 | Channel or | Transporter |
| | member 9 | | transporter | |
| SLC50A1 | Solute carrier family 50 | 55974 | Channel or | Transporter |
| | member 1 | | transporter | |
| SLC51A | Solute carrier family 51 alpha | 200931 | Channel or | Transporter |
| | subunit | | transporter | |
| SLC51B | Solute carrier family 51 beta | 123264 | Channel or | Transporter |
| | subunit | | transporter | |
| SLC52A1 | Solute carrier family 52 | 55065 | Channel or | Transporter |
| | member 1 | | transporter | |
| SLC52A2 | Solute carrier family 52 | 79581 | Channel or | Transporter |
| | member 2 | | transporter | |
| SLC52A3 | Solute carrier family 52 | 113278 | Channel or | Transporter |
| | member 3 | | transporter | |
| SLC5A1 | Solute carrier family 5 | 6523 | Channel or | Transporter |
| | member 1 | | transporter | |
| SLC5A10 | Solute carrier family 5 | 125206 | Channel or | Transporter |
| | member 10 | | transporter | |
| SLC5A11 | Solute carrier family 5 | 115584 | Channel or | Transporter |
| | member 11 | | transporter | |
| SLC5A12 | Solute carrier family 5 | 159963 | Channel or | Transporter |
| | member 12 | | transporter | |
| SLC5A2 | Solute carrier family 5 | 6524 | Channel or | Transporter |
| | member 2 | | transporter | |
| SLC5A3 | Solute carrier family 5 | 6526 | Channel or | Transporter |
| | member 3 | | transporter | |
| SLC5A4 | Solute carrier family 5 | 6527 | Channel or | Transporter |
| | member 4 | | transporter | |
| SLC5A5 | Solute carrier family 5 | 6528 | Channel or | Transporter |
| | member 5 | | transporter | |
| SLC5A6 | Solute carrier family 5 | 8884 | Channel or | Transporter |
| | member 6 | | transporter | |
| SLC5A7 | Solute carrier family 5 | 60482 | Channel or | Transporter |
| | member 7 | | transporter | |
| SLC5A8 | Solute carrier family 5 | 160728 | Channel or | Transporter |
| | member 8 | | transporter | |
| SLC5A9 | Solute carrier family 5 | 200010 | Channel or | Transporter |
| | member 9 | | transporter | |
| SLC6A1 | Solute carrier family 6 | 6529 | Channel or | Transporter |
| | member 1 | | transporter | |
| SLC6A10P | Solute carrier family 6 | 386757 | Channel or | Transporter |
| | member 10, pseudogene | | transporter | |
| SLC6A10PB | Solute carrier family 6 | 653562 | Channel or | Transporter |
| | member 8 pseudogene | | transporter | |
| SLC6A11 | Solute carrier family 6 | 6538 | Channel or | Transporter |
| | member 11 | | transporter | |
| SLC6A12 | Solute carrier family 6 | 6539 | Channel or | Transporter |
| | member 12 | | transporter | |
| SLC6A13 | Solute carrier family 6 | 6540 | Channel or | Transporter |
| | member 13 | | transporter | |
| SLC6A14 | Solute carrier family 6 | 11254 | Channel or | Transporter |
| | member 14 | | transporter | |
| SLC6A15 | Solute carrier family 6 | 55117 | Channel or | Transporter |
| | member 15 | | transporter | |
| SLC6A16 | Solute carrier family 6 | 28968 | Channel or | Transporter |
| | member 16 | | transporter | |
| SLC6A17 | Solute carrier family 6 | 388662 | Channel or | Transporter |
| | member 17 | | transporter | |
| SLC6A18 | Solute carrier family 6 | 348932 | Channel or | Transporter |
| | member 18 | | transporter | |
| SLC6A19 | Solute carrier family 6 | 340024 | Channel or | Transporter |
| | member 19 | | transporter | |
| SLC6A2 | Solute carrier family 6 | 6530 | Channel or | Transporter |
| | member 2 | | transporter | |
| SLC6A20 | Solute carrier family 6 | 54716 | Channel or | Transporter |
| | member 20 | | transporter | |
| SLC6A21P | Solute carrier family 6 | 652969 | Channel or | Transporter |
| | member 21, pseudogene | | transporter | |
| SLC6A3 | Solute carrier family 6 | 6531 | Channel or | Transporter |
| | member 3 | | transporter | |
| SLC6A4 | Solute carrier family 6 | 6532 | Channel or | Transporter |
| | member 4 | | transporter | |
| SLC6A5 | Solute carrier family 6 | 9152 | Channel or | Transporter |
| | member 5 | | transporter | |
| SLC6A6 | Solute carrier family 6 | 6533 | Channel or | Transporter |
| | member 6 | | transporter | |
| SLC6A7 | Solute carrier family 6 | 6534 | Channel or | Transporter |
| | member 7 | | transporter | |
| SLC6A8 | Solute carrier family 6 | 6535 | Channel or | Transporter |
| | member 8 | | transporter | |
| SLC6A9 | Solute carrier family 6 | 6536 | Channel or | Transporter |
| | member 9 | | transporter | |
| SLC7A1 | Solute carrier family 7 | 6541 | Channel or | Transporter |
| | member 1 | | transporter | |
| SLC7A10 | Solute carrier family 7 | 56301 | Channel or | Transporter |
| | member 10 | | transporter | |
| SLC7A11 | Solute carrier family 7 | 23657 | Channel or | Transporter |
| | member 11 | | transporter | |
| SLC7A13 | Solute carrier family 7 | 157724 | Channel or | Transporter |
| | member 13 | | transporter | |
| SLC7A14 | Solute carrier family 7 | 57709 | Channel or | Transporter |
| | member 14 | | transporter | |
| SLC7A2 | Solute carrier family 7 | 6542 | Channel or | Transporter |
| | member 2 | | transporter | |
| SLC7A3 | Solute carrier family 7 | 84889 | Channel or | Transporter |
| | member 3 | | transporter | |
| SLC7A4 | Solute carrier family 7 | 6545 | Channel or | Transporter |
| | member 4 | | transporter | |
| SLC7A5 | Solute carrier family 7 | 8140 | Channel or | Transporter |
| | member 5 | | transporter | |
| SLC7A6 | Solute carrier family 7 | 9057 | Channel or | Transporter |
| | member 6 | | transporter | |
| SLC7A7 | Solute carrier family 7 | 9056 | Channel or | Transporter |
| | member 7 | | transporter | |
| SLC7A8 | Solute carrier family 7 | 23428 | Channel or | Transporter |
| | member 8 | | transporter | |
| SLC7A9 | Solute carrier family 7 | 11136 | Channel or | Transporter |
| | member 9 | | transporter | |
| SLC8A1 | Solute carrier family 8 | 6546 | Channel or | Transporter |
| | member A1 | | transporter | |
| SLC8A2 | Solute carrier family 8 | 6543 | Channel or | Transporter |
| | member A2 | | transporter | |
| SLC8A3 | Solute carrier family 8 | 6547 | Channel or | Transporter |
| | member A3 | | transporter | |
| SLC8B1 | Solute carrier family 8 | 80024 | Channel or | Transporter |
| | member B1 | | transporter | |
| SLC9A1 | Solute carrier family 9 | 6548 | Channel or | Transporter |
| | member A1 | | transporter | |
| SLC9A2 | Solute carrier family 9 | 6549 | Channel or | Transporter |
| | member A2 | | transporter | |
| SLC9A3 | Solute carrier family 9 | 6550 | Channel or | Transporter |
| | member A3 | | transporter | |
| SLC9A4 | Solute carrier family 9 | 389015 | Channel or | Transporter |
| | member A4 | | transporter | |
| SLC9A5 | Solute carrier family 9 | 6553 | Channel or | Transporter |
| | member A5 | | transporter | |
| SLC9A6 | Solute carrier family 9 | 10479 | Channel or | Transporter |
| | member A6 | | transporter | |
| SLC9A7 | Solute carrier family 9 | 84679 | Channel or | Transporter |
| | member A7 | | transporter | |
| SLC9A8 | Solute carrier family 9 | 23315 | Channel or | Transporter |
| | member A8 | | transporter | |
| SLC9A9 | Solute carrier family 9 | 285195 | Channel or | Transporter |
| | member A9 | | transporter | |
| SLC9B1 | Solute carrier family 9 | 150159 | Channel or | Transporter |
| | member B1 | | transporter | |
| SLC9B2 | Solute carrier family 9 | 133308 | Channel or | Transporter |
| | member B2 | | transporter | |
| SLC9C1 | Solute carrier family 9 | 285335 | Channel or | Transporter |
| | member C1 | | transporter | |
| SLC9C2 | Solute carrier family 9 | 284525 | Channel or | Transporter |
| | member C2 (putative) | | transporter | |
| SLCO1A2 | Solute carrier organic anion | 6579 | Channel or | Transporter |
| | transporter family member | | transporter | |
| | 1A2 | | | |
| SLCO1B1 | Solute carrier organic anion | 10599 | Channel or | Transporter |
| | transporter family member | | transporter | |
| | 1B1 | | | |
| SLCO1B3 | Solute carrier organic anion | 28234 | Channel or | Transporter |
| | transporter family member | | transporter | |
| | 1B3 | | | |
| SLCO1C1 | Solute carrier organic anion | 53919 | Channel or | Transporter |
| | transporter family member | | transporter | |
| | 1C1 | | | |
| SLCO2A1 | Solute carrier organic anion | 6578 | Channel or | Transporter |
| | transporter family member | | transporter | |
| | 2A1 | | | |
| SLCO2B1 | Solute carrier organic anion | 11309 | Channel or | Transporter |
| | transporter family member | | transporter | |
| | 2B1 | | | |
| SLCO3A1 | Solute carrier organic anion | 28232 | Channel or | Transporter |
| | transporter family member | | transporter | |
| | 3A1 | | | |
| SLCO4A1 | Solute carrier organic anion | 28231 | Channel or | Transporter |
| | transporter family member | | transporter | |
| | 4A1 | | | |
| SLCO4C1 | Solute carrier organic anion | 353189 | Channel or | Transporter |
| | transporter family member | | transporter | |
| | 4C1 | | | |
| SLCO5A1 | Solute carrier organic anion | 81796 | Channel or | Transporter |
| | transporter family member | | transporter | |
| | 5A1 | | | |
| SLCO6A1 | Solute carrier organic anion | 133482 | Channel or | Transporter |
| | transporter family member | | transporter | |
| | 6A1 | | | |
| SLURP1 | Secreted LY6/PLAUR domain | 57152 | Other | Miscelaneous |
| | containing 1 | | | |
| SMPD3 | Sphingomyelin | 55512 | Other | Miscelaneous |
| | phosphodiesterase 3 | | | |
| SNAP23 | Synaptosome associated | 8773 | Vesicular | Vesicles |
| | protein 23 | | | |
| SNAP25 | Synaptosome associated | 6616 | Vesicular | Vesicles |
| | protein 25 | | | |
| SNAP29 | Synaptosome associated | 9342 | Vesicular | Vesicles |
| | protein 29 | | | |
| SNAPIN | SNAP associated protein | 23557 | Signaling | Signaling |
| SNCA | Synuclein alpha | 6622 | Vesicular | Vesicles |
| SNCAIP | Synuclein alpha interacting | 9627 | Vesicular | Vesicles |
| | protein | | | |
| SNCB | Synuclein beta | 6620 | Vesicular | Vesicles |
| SNCG | Synuclein gamma | 6623 | Vesicular | Vesicles |
| SNPH | Syntaphilin | 9751 | Vesicular | Vesicles |
| SNTG1 | Syntrophin gamma 1 | 54212 | Neurotrophic | Biosynthesis |
| SNX13 | Sorting nexin 13 | 23161 | Neurotransmitter | Signaling |
| SOD2 | Superoxide dismutase 2, | 6648 | Other | Miscelaneous |
| | mitochondrial | | | |
| SORCS1 | Sortilin related VPS10 domain | 114815 | Neurotrophic | Receptor |
| | containing receptor 1 | | | |
| SORCS2 | Sortilin related VPS10 domain | 57537 | Neurotrophic | Receptor |
| | containing receptor 2 | | | |
| SORCS3 | Sortilin related VPS10 domain | 22986 | Neurotrophic | Receptor |
| | containing receptor 3 | | | |
| SORT1 | Sortilin 1 | 6272 | Neurotrophic | Receptor |
| SOS1 | SOS Ras/Rac guanine | 6654 | Signaling | Signaling |
| | nucleotide exchange factor 1 | | | |
| SPX | Spexin hormone | 80763 | Neuropeptide | Ligand |
| SRC | SRC proto-oncogene, non- | 6714 | Signaling | Signaling |
| | receptor tyrosine kinase | | | |
| SST | Somatostatin | 6750 | Neuropeptide | Ligand |
| SSTR1 | Somatostatin receptor 1 | 6751 | Neuropeptide | Receptor |
| SSTR2 | Somatostatin receptor 2 | 6752 | Neuropeptide | Receptor |
| SSTR3 | Somatostatin receptor 3 | 6753 | Neuropeptide | Receptor |
| SSTR4 | Somatostatin receptor 4 | 6754 | Neuropeptide | Receptor |
| SSTR5 | Somatostatin receptor 5 | 6755 | Neuropeptide | Receptor |
| STAC3 | SH3 and cysteine rich domain | 246329 | Other | Miscelaneous |
| | 3 | | | |
| STRN | Striatin | 6801 | Signaling | Signaling |
| STX10 | Syntaxin 10 | 8677 | Vesicular | Vesicles |
| STX11 | Syntaxin 11 | 8676 | Vesicular | Vesicles |
| STX16 | Syntaxin 16 | 8675 | Vesicular | Vesicles |
| STX19 | Syntaxin 19 | 415117 | Vesicular | Vesicles |
| STX1A | Syntaxin 1A | 6804 | Vesicular | Vesicles |
| STX1B | Syntaxin 1B | 112755 | Vesicular | Vesicles |
| STX2 | Syntaxin 2 | 2054 | Vesicular | Vesicles |
| STX3 | Syntaxin 3 | 6809 | Vesicular | Vesicles |
| STX4 | Syntaxin 4 | 6810 | Vesicular | Vesicles |
| STX6 | Syntaxin 6 | 10228 | Vesicular | Vesicles |
| STXBP1 | Syntaxin binding protein 1 | 6812 | Vesicular | Vesicles |
| STXBP5 | Syntaxin binding protein 5 | 134957 | Vesicular | Vesicles |
| SULF1 | Sulfatase 1 | 23213 | Neurotrophic | Signaling |
| SULF2 | Sulfatase 2 | 55959 | Neurotrophic | Signaling |
| SV2A | Synaptic vesicle glycoprotein | 9900 | Vesicular | Vesicles |
| | 2A | | | |
| SV2B | Synaptic vesicle glycoprotein | 9899 | Vesicular | Vesicles |
| | 2B | | | |
| SV2C | Synaptic vesicle glycoprotein | 22987 | Vesicular | Vesicles |
| | 2C | | | |
| SYN1 | Synapsin I | 6853 | Vesicular | Vesicles |
| SYN2 | Synapsin II | 6854 | Vesicular | Vesicles |
| SYN3 | Synapsin III | 8224 | Vesicular | Vesicles |
| SYNJ1 | Synaptojanin 1 | 8867 | Vesicular | Vesicles |
| SYT1 | Synaptotagmin 1 | 6857 | Vesicular | Vesicles |
| SYT10 | Synaptotagmin 10 | 341359 | Vesicular | Vesicles |
| SYT11 | Synaptotagmin 11 | 23208 | Vesicular | Vesicles |
| SYT12 | Synaptotagmin 12 | 91683 | Vesicular | Vesicles |
| SYT13 | Synaptotagmin 13 | 57586 | Vesicular | Vesicles |
| SYT14 | Synaptotagmin 14 | 255928 | Vesicular | Vesicles |
| SYT14P1 | Synaptotagmin 14 | 401135 | Vesicular | Vesicles |
| | pseudogene 1 | | | |
| SYT15 | Synaptotagmin 15 | 83849 | Vesicular | Vesicles |
| SYT16 | Synaptotagmin 16 | 83851 | Vesicular | Vesicles |
| SYT17 | Synaptotagmin 17 | 51760 | Vesicular | Vesicles |
| SYT2 | Synaptotagmin 2 | 127833 | Vesicular | Vesicles |
| SYT3 | Synaptotagmin 3 | 84258 | Vesicular | Vesicles |
| SYT4 | Synaptotagmin 4 | 6860 | Vesicular | Vesicles |
| SYT5 | Synaptotagmin 5 | 6861 | Vesicular | Vesicles |
| SYT6 | Synaptotagmin 6 | 148281 | Vesicular | Vesicles |
| SYT7 | Synaptotagmin 7 | 9066 | Vesicular | Vesicles |
| SYT8 | Synaptotagmin 8 | 90019 | Vesicular | Vesicles |
| SYT9 | Synaptotagmin 9 | 143425 | Vesicular | Vesicles |
| SYTL1 | Synaptotagmin like 1 | 84958 | Vesicular | Vesicles |
| SYTL2 | Synaptotagmin like 2 | 54843 | Vesicular | Vesicles |
| SYTL3 | Synaptotagmin like 3 | 94120 | Vesicular | Vesicles |
| SYTL4 | Synaptotagmin like 4 | 94121 | Vesicular | Vesicles |
| SYTL5 | Synaptotagmin like 5 | 94122 | Vesicular | Vesicles |
| TAAR5 | Trace amine associated | 9038 | Neurotransmitter | Receptor |
| | receptor 5 | | | |
| TAC1 | Tachykinin precursor 1 | 6863 | Neuropeptide | Ligand |
| TAC3 | Tachykinin 3 | 6866 | Neuropeptide | Ligand |
| TAC4 | Tachykinin 4 (hemokinin) | 255061 | Neuropeptide | Ligand |
| TACR1 | Tachykinin receptor 1 | 6869 | Neuropeptide | Receptor |
| TACR2 | Tachykinin receptor 2 | 6865 | Neuropeptide | Receptor |
| TACR3 | Tachykinin receptor 3 | 6870 | Neuropeptide | Receptor |
| TBXA2R | Thromboxane A2 receptor | 6915 | Neuropeptide | Receptor |
| TCIRG1 | T-cell immune regulator 1, | 10312 | Channel or | Transporter |
| | atpase H+ transporting V0 | | transporter | |
| | subunit a3 | | | |
| TENM1 | Teneurin transmembrane | 10178 | Other | Miscelaneous |
| | protein 1 | | | |
| TGM2 | Transglutaminase 2 | 7052 | Other | Miscelaneous |
| TH | Tyrosine hydroxylase | 7054 | Neurotransmitter | Biosynthesis |
| TMEM158 | Transmembrane protein 158 | 25907 | Neurotrophic | Receptor |
| | (gene/pseudogene) | | | |
| TMOD2 | Tropomodulin 2 | 29767 | Vesicular | Vesicles |
| TNF | Tumor necrosis factor | 7124 | Neurotrophic | Ligand |
| TNR | Tenascin R | 7143 | Other | Miscelaneous |
| TP73 | Tumor protein p73 | 7161 | Other | Miscelaneous |
| TPCN1 | Two pore segment channel 1 | 53373 | Channel or | Channel |
| | | | transporter | |
| TPCN2 | Two pore segment channel 2 | 219931 | Channel or | Channel |
| | | | transporter | |
| TPH1 | Tryptophan hydroxylase 1 | 7166 | Neurotransmitter | Biosynthesis |
| TPH2 | Tryptophan hydroxylase 2 | 121278 | Neurotransmitter | Biosynthesis |
| TPM3 | Tropomyosin 3 | 7170 | Neurotrophic | Receptor |
| TPR | Translocated promoter region, | 7175 | Other | Miscelaneous |
| | nuclear basket protein | | | |
| TRH | Thyrotropin releasing | 7200 | Neuropeptide | Ligand |
| | hormone | | | |
| TRHDE | Thyrotropin releasing | 29953 | Neurotransmitter | Biosynthesis |
| | hormone degrading enzyme | | | |
| TRHR | Thyrotropin releasing | 7201 | Neuropeptide | Receptor |
| | hormone receptor | | | |
| TRPA1 | Transient receptor potential | 8989 | Channel or | Channel |
| | cation channel subfamily A | | transporter | |
| | member 1 | | | |
| TRPC1 | Transient receptor potential | 7220 | Channel or | Channel |
| | cation channel subfamily C | | transporter | |
| | member 1 | | | |
| TRPC2 | Transient receptor potential | 7221 | Channel or | Channel |
| | cation channel subfamily C | | transporter | |
| | member 2, pseudogene | | | |
| TRPC3 | Transient receptor potential | 7222 | Channel or | Channel |
| | cation channel subfamily C | | transporter | |
| | member 3 | | | |
| TRPC4 | Transient receptor potential | 7223 | Channel or | Channel |
| | cation channel subfamily C | | transporter | |
| | member 4 | | | |
| TRPC5 | Transient receptor potential | 7224 | Channel or | Channel |
| | cation channel subfamily C | | transporter | |
| | member 5 | | | |
| TRPC6 | Transient receptor potential | 7225 | Channel or | Channel |
| | cation channel subfamily C | | transporter | |
| | member 6 | | | |
| TRPC7 | Transient receptor potential | 57113 | Channel or | Channel |
| | cation channel subfamily C | | transporter | |
| | member 7 | | | |
| TRPM1 | Transient receptor potential | 4308 | Channel or | Channel |
| | cation channel subfamily M | | transporter | |
| | member 1 | | | |
| TRPM2 | Transient receptor potential | 7226 | Channel or | Channel |
| | cation channel subfamily M | | transporter | |
| | member 2 | | | |
| TRPM3 | Transient receptor potential | 80036 | Channel or | Channel |
| | cation channel subfamily M | | transporter | |
| | member 3 | | | |
| TRPM4 | Transient receptor potential | 54795 | Channel or | Channel |
| | cation channel subfamily M | | transporter | |
| | member 4 | | | |
| TRPM5 | Transient receptor potential | 29850 | Channel or | Channel |
| | cation channel subfamily M | | transporter | |
| | member 5 | | | |
| TRPM6 | Transient receptor potential | 140803 | Channel or | Channel |
| | cation channel subfamily M | | transporter | |
| | member 6 | | | |
| TRPM7 | Transient receptor potential | 54822 | Channel or | Channel |
| | cation channel subfamily M | | transporter | |
| | member 7 | | | |
| TRPM8 | Transient receptor potential | 79054 | Channel or | Channel |
| | cation channel subfamily M | | transporter | |
| | member 8 | | | |
| TRPV1 | Transient receptor potential | 7442 | Channel or | Channel |
| | cation channel subfamily V | | transporter | |
| | member 1 | | | |
| TRPV2 | Transient receptor potential | 51393 | Channel or | Channel |
| | cation channel subfamily V | | transporter | |
| | member 2 | | | |
| TRPV3 | Transient receptor potential | 162514 | Channel or | Channel |
| | cation channel subfamily V | | transporter | |
| | member 3 | | | |
| TRPV4 | Transient receptor potential | 59341 | Channel or | Channel |
| | cation channel subfamily V | | transporter | |
| | member 4 | | | |
| TRPV5 | Transient receptor potential | 56302 | Channel or | Channel |
| | cation channel subfamily V | | transporter | |
| | member 5 | | | |
| TRPV6 | Transient receptor potential | 55503 | Channel or | Channel |
| | cation channel subfamily V | | transporter | |
| | member 6 | | | |
| TSHR | Thyroid stimulating hormone | 7253 | Neuropeptide | Receptor |
| | receptor | | | |
| TSPOAP1 | TSPO associated protein 1 | 9256 | Vesicular | Vesicles |
| UBL5 | Ubiquitin like 5 | 59286 | Other | Miscelaneous |
| UCN | Urocortin | 7349 | Neuropeptide | Ligand |
| UCN2 | Urocortin 2 | 90226 | Neuropeptide | Ligand |
| UCN3 | Urocortin 3 | 114131 | Neuropeptide | Ligand |
| UCP1 | Uncoupling protein 1 | 7350 | Channel or | Transporter |
| | | | transporter | |
| UCP2 | Uncoupling protein 2 | 7351 | Channel or | Transporter |
| | | | transporter | |
| UCP3 | Uncoupling protein 3 | 7352 | Channel or | Transporter |
| | | | transporter | |
| UNC119 | Unc-119 lipid binding | 9094 | Vesicular | Vesicles |
| | chaperone | | | |
| UNC13A | Unc-13 homolog A | 23025 | Vesicular | Vesicles |
| UNC13B | Unc-13 homolog B | 10497 | Vesicular | Vesicles |
| USP46 | Ubiquitin specific peptidase 46 | 64854 | Other | Miscelaneous |
| UTS2 | Urotensin 2 | 10911 | Neuropeptide | Ligand |
| UTS2B | Urotensin 2B | 257313 | Neuropeptide | Ligand |
| UTS2R | Urotensin 2 receptor | 2837 | Neuropeptide | Receptor |
| VAMP1 | Vesicle associated membrane | 6843 | Vesicular | Vesicles |
| | protein 1 | | | |
| VAMP2 | Vesicle associated membrane | 6844 | Vesicular | Vesicles |
| | protein 2 | | | |
| VAMP3 | Vesicle associated membrane | 9341 | Vesicular | Vesicles |
| | protein 3 | | | |
| VAMP8 | Vesicle associated membrane | 8673 | Vesicular | Vesicles |
| | protein 8 | | | |
| VDAC1 | Voltage dependent anion | 7416 | Channel or | Channel |
| | channel 1 | | transporter | |
| VEGFA | Vascular endothelial growth | 7422 | Neurotrophic | Ligand |
| | factor A | | | |
| VGF | VGF nerve growth factor | 7425 | Neurotrophic | Ligand |
| | inducible | | | |
| VIP | Vasoactive intestinal peptide | 7432 | Neuropeptide | Ligand |
| VIPR1 | Vasoactive intestinal peptide | 7433 | Neuropeptide | Receptor |
| | receptor 1 | | | |
| VIPR2 | Vasoactive intestinal peptide | 7434 | Neuropeptide | Receptor |
| | receptor 2 | | | |
| XCR1 | X-C motif chemokine receptor | 2829 | Neurotrophic | Receptor |
| | 1 | | | |
| ZACN | Zinc activated ion channel | 353174 | Channel or | Channel |
| | | | transporter | |
| ZN274 | Neurotrophin receptor- | 10782 | Neurotrophic | Signaling |
| | interacting factor homolog | | | |

TABLE 8
ION CHANNEL AND TRANSPORTER GENES

| | | | | | Ion |
|---|---|---|---|---|---|
| Approved | Approved | Entrez | Gene type/ | | channel |
| Symbol | name | Gene ID | family | Category | type |
| ASIC1 | Acid sensing ion channel | 41 | Channel or | Channel | Lgic |
| | subunit 1 | | transporter | | |
| ASIC2 | Acid sensing ion channel | 40 | Channel or | Channel | Lgic |
| | subunit 2 | | transporter | | |
| ASIC3 | Acid sensing ion channel | 9311 | Channel or | Channel | Lgic |
| | subunit 3 | | transporter | | |
| GABRA1 | Gamma-aminobutyric acid | 2554 | Neurotransmitter | Receptor | Lgic |
| | type A receptor alpha1 | | | | |
| | subunit | | | | |
| GABRA2 | Gamma-aminobutyric acid | 2555 | Neurotransmitter | Receptor | Lgic |
| | type A receptor alpha2 | | | | |
| | subunit | | | | |
| GABRA3 | Gamma-aminobutyric acid | 2556 | Neurotransmitter | Receptor | Lgic |
| | type A receptor alpha3 | | | | |
| | subunit | | | | |
| GABRA4 | Gamma-aminobutyric acid | 2557 | Neurotransmitter | Receptor | Lgic |
| | type A receptor alpha4 | | | | |
| | subunit | | | | |
| GABRA5 | Gamma-aminobutyric acid | 2558 | Neurotransmitter | Receptor | Lgic |
| | type A receptor alpha5 | | | | |
| | subunit | | | | |
| GABRA6 | Gamma-aminobutyric acid | 2559 | Neurotransmitter | Receptor | Lgic |
| | type A receptor alpha6 | | | | |
| | subunit | | | | |
| GABRB1 | Gamma-aminobutyric acid | 2560 | Neurotransmitter | Receptor | Lgic |
| | type A receptor beta1 | | | | |
| | subunit | | | | |
| GABRB2 | Gamma-aminobutyric acid | 2561 | Neurotransmitter | Receptor | Lgic |
| | type A receptor beta2 | | | | |
| | subunit | | | | |
| GABRB3 | Gamma-aminobutyric acid | 2562 | Neurotransmitter | Receptor | Lgic |
| | type A receptor beta3 | | | | |
| | subunit | | | | |
| GABRD | Gamma-aminobutyric acid | 2563 | Neurotransmitter | Receptor | Lgic |
| | type A receptor delta | | | | |
| | subunit | | | | |
| GABRE | Gamma-aminobutyric acid | 2564 | Neurotransmitter | Receptor | Lgic |
| | type A receptor epsilon | | | | |
| | subunit | | | | |
| GABRG1 | Gamma-aminobutyric acid | 2565 | Neurotransmitter | Receptor | Lgic |
| | type A receptor gamma1 | | | | |
| | subunit | | | | |
| GABRG2 | Gamma-aminobutyric acid | 2566 | Neurotransmitter | Receptor | Lgic |
| | type A receptor gamma2 | | | | |
| | subunit | | | | |
| GABRG3 | Gamma-aminobutyric acid | 2567 | Neurotransmitter | Receptor | Lgic |
| | type A receptor gamma3 | | | | |
| | subunit | | | | |
| GABRP | Gamma-aminobutyric acid | 2568 | Neurotransmitter | Receptor | Lgic |
| | type A receptor pi subunit | | | | |
| GABRQ | Gamma-aminobutyric acid | 55879 | Neurotransmitter | Receptor | Lgic |
| | type A receptor theta | | | | |
| | subunit | | | | |
| GABRR1 | Gamma-aminobutyric acid | 2569 | Neurotransmitter | Receptor | Lgic |
| | type A receptor rho1 subunit | | | | |
| GABRR2 | Gamma-aminobutyric acid | 2570 | Neurotransmitter | Receptor | Lgic |
| | type A receptor rho2 subunit | | | | |
| GABRR3 | Gamma-aminobutyric acid | 200959 | Neurotransmitter | Receptor | Lgic |
| | type A receptor rho3 subunit | | | | |
| | (gene/pseudogene) | | | | |
| GLRA1 | Glycine receptor alpha 1 | 2741 | Neurotransmitter | Receptor | Lgic |
| GLRA2 | Glycine receptor alpha 2 | 2742 | Neurotransmitter | Receptor | Lgic |
| GLRA3 | Glycine receptor alpha 3 | 8001 | Neurotransmitter | Receptor | Lgic |
| GLRA4 | Glycine receptor alpha 4 | 441509 | Neurotransmitter | Receptor | Lgic |
| GLRB | Glycine receptor beta | 2743 | Neurotransmitter | Receptor | Lgic |
| GRIA1 | Glutamate ionotropic | 2890 | Neurotransmitter | Receptor | Lgic |
| | receptor AMPA type subunit | | | | |
| | 1 | | | | |
| GRIA2 | Glutamate ionotropic | 2891 | Neurotransmitter | Receptor | Lgic |
| | receptor AMPA type subunit | | | | |
| | 2 | | | | |
| GRIA3 | Glutamate ionotropic | 2892 | Neurotransmitter | Receptor | Lgic |
| | receptor AMPA type subunit | | | | |
| | 3 | | | | |
| GRIA4 | Glutamate ionotropic | 2893 | Neurotransmitter | Receptor | Lgic |
| | receptor AMPA type subunit | | | | |
| | 4 | | | | |
| GRID1 | Glutamate ionotropic | 2894 | Neurotransmitter | Receptor | Lgic |
| | receptor delta type subunit | | | | |
| | 1 | | | | |
| GRID2 | Glutamate ionotropic | 2895 | Neurotransmitter | Receptor | Lgic |
| | receptor delta type subunit | | | | |
| | 2 | | | | |
| GRIK1 | Glutamate ionotropic | 2897 | Neurotransmitter | Receptor | Lgic |
| | receptor kainate type | | | | |
| | subunit 1 | | | | |
| GRIK2 | Glutamate ionotropic | 2898 | Neurotransmitter | Receptor | Lgic |
| | receptor kainate type | | | | |
| | subunit 2 | | | | |
| GRIK3 | Glutamate ionotropic | 2899 | Neurotransmitter | Receptor | Lgic |
| | receptor kainate type | | | | |
| | subunit 3 | | | | |
| GRIK4 | Glutamate ionotropic | 2900 | Neurotransmitter | Receptor | Lgic |
| | receptor kainate type | | | | |
| | subunit 4 | | | | |
| GRIK5 | Glutamate ionotropic | 2901 | Neurotransmitter | Receptor | Lgic |
| | receptor kainate type | | | | |
| | subunit 5 | | | | |
| GRIN1 | Glutamate ionotropic | 2902 | Neurotransmitter | Receptor | Lgic |
| | receptor NMDA type subunit | | | | |
| | 1 | | | | |
| GRIN2A | Glutamate ionotropic | 2903 | Neurotransmitter | Receptor | Lgic |
| | receptor NMDA type subunit | | | | |
| | 2A | | | | |
| GRIN2B | Glutamate ionotropic | 2904 | Neurotransmitter | Receptor | Lgic |
| | receptor NMDA type subunit | | | | |
| | 2B | | | | |
| GRIN2C | Glutamate ionotropic | 2905 | Neurotransmitter | Receptor | Lgic |
| | receptor NMDA type subunit | | | | |
| | 2C | | | | |
| GRIN2D | Glutamate ionotropic | 2906 | Neurotransmitter | Receptor | Lgic |
| | receptor NMDA type subunit | | | | |
| | 2D | | | | |
| GRIN3A | Glutamate ionotropic | 116443 | Neurotransmitter | Receptor | Lgic |
| | receptor NMDA type subunit | | | | |
| | 3A | | | | |
| GRIN3B | Glutamate ionotropic | 116444 | Neurotransmitter | Receptor | Lgic |
| | receptor NMDA type subunit | | | | |
| | 3B | | | | |
| HTR3A | 5-hydroxytryptamine | 3359 | Neurotransmitter | Receptor | Lgic |
| | receptor 3A | | | | |
| HTR3B | 5-hydroxytryptamine | 9177 | Neurotransmitter | Receptor | Lgic |
| | receptor 3B | | | | |
| HTR3C | 5-hydroxytryptamine | 170572 | Neurotransmitter | Receptor | Lgic |
| | receptor 3C | | | | |
| HTR3D | 5-hydroxytryptamine | 200909 | Neurotransmitter | Receptor | Lgic |
| | receptor 3D | | | | |
| HTR3E | 5-hydroxytryptamine | 285242 | Neurotransmitter | Receptor | Lgic |
| | receptor 3E | | | | |
| ITPR1 | Inositol 1,4,5-trisphosphate | 3708 | Neurotransmitter | Signaling | Lgic |
| | receptor type 1 | | | | |
| ITPR2 | Inositol 1,4,5-trisphosphate | 3709 | Neurotransmitter | Signaling | Lgic |
| | receptor type 2 | | | | |
| ITPR3 | Inositol 1,4,5-trisphosphate | 3710 | Neurotransmitter | Signaling | Lgic |
| | receptor type 3 | | | | |
| SCNN1A | Sodium channel epithelial 1 | 6337 | Channel or | Channel | Lgic |
| | alpha subunit | | transporter | | |
| SCNN1B | Sodium channel epithelial 1 | 6338 | Channel or | Channel | Lgic |
| | beta subunit | | transporter | | |
| SCNN1D | Sodium channel epithelial 1 | 6339 | Channel or | Channel | Lgic |
| | delta subunit | | transporter | | |
| SCNN1G | Sodium channel epithelial 1 | 6340 | Channel or | Channel | Lgic |
| | gamma subunit | | transporter | | |
| ZACN | Zinc activated ion channel | 353174 | Channel or | Channel | Lgic |
| | | | transporter | | |
| CLCN1 | Chloride voltage-gated | 1180 | Channel or | Channel | Other_ic |
| | channel 1 | | transporter | | |
| CLCN2 | Chloride voltage-gated | 1181 | Channel or | Channel | Other_ic |
| | channel 2 | | transporter | | |
| CLCN3 | Chloride voltage-gated | 1182 | Channel or | Channel | Other_ic |
| | channel 3 | | transporter | | |
| CLCN4 | Chloride voltage-gated | 1183 | Channel or | Channel | Other_ic |
| | channel 4 | | transporter | | |
| CLCN5 | Chloride voltage-gated | 1184 | Channel or | Channel | Other_ic |
| | channel 5 | | transporter | | |
| CLCN6 | Chloride voltage-gated | 1185 | Channel or | Channel | Other_ic |
| | channel 6 | | transporter | | |
| CLCN7 | Chloride voltage-gated | 1186 | Channel or | Channel | Other_ic |
| | channel 7 | | transporter | | |
| CLCNKA | Chloride voltage-gated | 1187 | Channel or | Channel | Other_ic |
| | channel Ka | | transporter | | |
| CLCNKB | Chloride voltage-gated | 1188 | Channel or | Channel | Other_ic |
| | channel Kb | | transporter | | |
| CLIC6 | Chloride intracellular | 54102 | Channel or | Channel | Other_ic |
| | channel 6 | | transporter | | |
| GJA1 | Gap junction protein alpha 1 | 2697 | Channel or | Channel | Other_ic |
| | | | transporter | | |
| GJA10 | Gap junction protein alpha | 84694 | Channel or | Channel | Other_ic |
| | 10 | | transporter | | |
| GJA3 | Gap junction protein alpha 3 | 2700 | Channel or | Channel | Other_ic |
| | | | transporter | | |
| GJA4 | Gap junction protein alpha 4 | 2701 | Channel or | Channel | Other_ic |
| | | | transporter | | |
| GJA5 | Gap junction protein alpha 5 | 2702 | Channel or | Channel | Other_ic |
| | | | transporter | | |
| GJA8 | Gap junction protein alpha 8 | 2703 | Channel or | Channel | Other_ic |
| | | | transporter | | |
| GJA9 | Gap junction protein alpha 9 | 81025 | Channel or | Channel | Other_ic |
| | | | transporter | | |
| GJB1 | Gap junction protein beta 1 | 2705 | Channel or | Channel | Other_ic |
| | | | transporter | | |
| GJB2 | Gap junction protein beta 2 | 2706 | Channel or | Channel | Other_ic |
| | | | transporter | | |
| GJB3 | Gap junction protein beta 3 | 2707 | Channel or | Channel | Other_ic |
| | | | transporter | | |
| GJB4 | Gap junction protein beta 4 | 127534 | Channel or | Channel | Other_ic |
| | | | transporter | | |
| GJB5 | Gap junction protein beta 5 | 2709 | Channel or | Channel | Other_ic |
| | | | transporter | | |
| GJB6 | Gap junction protein beta 6 | 10804 | Channel or | Channel | Other_ic |
| | | | transporter | | |
| GJB7 | Gap junction protein beta 7 | 375519 | Channel or | Channel | Other_ic |
| | | | transporter | | |
| GJC1 | Gap junction protein gamma | 10052 | Channel or | Channel | Other_ic |
| | 1 | | transporter | | |
| GJC2 | Gap junction protein gamma | 57165 | Channel or | Channel | Other_ic |
| | 2 | | transporter | | |
| GJC3 | Gap junction protein gamma | 349149 | Channel or | Channel | Other_ic |
| | 3 | | transporter | | |
| GJD2 | Gap junction protein delta 2 | 57369 | Channel or | Channel | Other_ic |
| | | | transporter | | |
| GJD3 | Gap junction protein delta 3 | 125111 | Channel or | Channel | Other_ic |
| | | | transporter | | |
| GJD4 | Gap junction protein delta 4 | 219770 | Channel or | Channel | Other_ic |
| | | | transporter | | |
| GJE1 | Gap junction protein epsilon | 100126572 | Channel or | Channel | Other_ic |
| | 1 | | transporter | | |
| KCNMB4 | Potassium calcium- | 27345 | Channel or | Channel | Other_ic |
| | activated channel subfamily | | transporter | | |
| | M regulatory beta subunit 4 | | | | |
| NALCN | Sodium leak channel, non- | 259232 | Channel or | Channel | Other_ic |
| | selective | | transporter | | |
| PANX1 | Pannexin 1 | 24145 | Channel or | Channel | Other_ic |
| | | | transporter | | |
| PANX2 | Pannexin 2 | 56666 | Channel or | Channel | Other_ic |
| | | | transporter | | |
| PANX3 | Pannexin 3 | 116337 | Channel or | Channel | Other_ic |
| | | | transporter | | |
| SHROOM1 | Shroom family member 1 | 134549 | Channel or | Channel | Other_ic |
| | | | transporter | | |
| SHROOM2 | Shroom family member 2 | 357 | Channel or | Channel | Other_ic |
| | | | transporter | | |
| SHROOM3 | Shroom family member 3 | 57619 | Channel or | Channel | Other_ic |
| | | | transporter | | |
| SHROOM4 | Shroom family member 4 | 57477 | Channel or | Channel | Other_ic |
| | | | transporter | | |
| ATP10A | Atpase phospholipid | 57194 | Channel or | Transporter | Transporter |
| | transporting 10A (putative) | | transporter | | |
| ATP10B | Atpase phospholipid | 23120 | Channel or | Transporter | Transporter |
| | transporting 10B (putative) | | transporter | | |
| ATP10D | Atpase phospholipid | 57205 | Channel or | Transporter | Transporter |
| | transporting 10D (putative) | | transporter | | |
| ATP11A | Atpase phospholipid | 23250 | Channel or | Transporter | Transporter |
| | transporting 11A | | transporter | | |
| ATP11B | Atpase phospholipid | 23200 | Channel or | Transporter | Transporter |
| | transporting 11B (putative) | | transporter | | |
| ATP11C | Atpase phospholipid | 286410 | Channel or | Transporter | Transporter |
| | transporting 11C | | transporter | | |
| ATP12A | Atpase H+/K+ transporting | 479 | Channel or | Transporter | Transporter |
| | non-gastric alpha2 subunit | | transporter | | |
| ATP1A1 | Atpase Na+/K+ transporting | 476 | Channel or | Transporter | Transporter |
| | subunit alpha 1 | | transporter | | |
| ATP1A2 | Atpase Na+/K+ transporting | 477 | Channel or | Transporter | Transporter |
| | subunit alpha 2 | | transporter | | |
| ATP1A3 | Atpase Na+/K+ transporting | 478 | Channel or | Transporter | Transporter |
| | subunit alpha 3 | | transporter | | |
| ATP1A4 | Atpase Na+/K+ transporting | 480 | Channel or | Transporter | Transporter |
| | subunit alpha 4 | | transporter | | |
| ATP1B1 | Atpase Na+/K+ transporting | 481 | Channel or | Transporter | Transporter |
| | subunit beta 1 | | transporter | | |
| ATP1B2 | Atpase Na+/K+ transporting | 482 | Channel or | Transporter | Transporter |
| | subunit beta 2 | | transporter | | |
| ATP1B3 | Atpase Na+/K+ transporting | 483 | Channel or | Transporter | Transporter |
| | subunit beta 3 | | transporter | | |
| ATP2A1 | Atpase | 487 | Channel or | Transporter | Transporter |
| | sarcoplasmic/endoplasmic | | transporter | | |
| | reticulum Ca2+ transporting | | | | |
| | 1 | | | | |
| ATP2A2 | Atpase | 488 | Channel or | Transporter | Transporter |
| | sarcoplasmic/endoplasmic | | transporter | | |
| | reticulum Ca2+ transporting | | | | |
| | 2 | | | | |
| ATP2A3 | Atpase | 489 | Channel or | Transporter | Transporter |
| | sarcoplasmic/endoplasmic | | transporter | | |
| | reticulum Ca2+ transporting | | | | |
| | 3 | | | | |
| ATP2B1 | Atpase plasma membrane | 490 | Channel or | Transporter | Transporter |
| | Ca2+ transporting 1 | | transporter | | |
| ATP2B2 | Atpase plasma membrane | 491 | Channel or | Transporter | Transporter |
| | Ca2+ transporting 2 | | transporter | | |
| ATP2B3 | Atpase plasma membrane | 492 | Channel or | Transporter | Transporter |
| | Ca2+ transporting 3 | | transporter | | |
| ATP2B4 | Atpase plasma membrane | 493 | Channel or | Transporter | Transporter |
| | Ca2+ transporting 4 | | transporter | | |
| ATP2C1 | Atpase secretory pathway | 27032 | Channel or | Transporter | Transporter |
| | Ca2+ transporting 1 | | transporter | | |
| ATP2C2 | Atpase secretory pathway | 9914 | Channel or | Transporter | Transporter |
| | Ca2+ transporting 2 | | transporter | | |
| ATP4A | Atpase H+/K+ transporting | 495 | Channel or | Transporter | Transporter |
| | alpha subunit | | transporter | | |
| ATP4B | Atpase H+/K+ transporting | 496 | Channel or | Transporter | Transporter |
| | beta subunit | | transporter | | |
| ATP5A1 | ATP synthase, H+ | 498 | Channel or | Transporter | Transporter |
| | transporting, mitochondrial | | transporter | | |
| | F1 complex, alpha subunit | | | | |
| | 1, cardiac muscle | | | | |
| ATP5B | ATP synthase, H+ | 506 | Channel or | Transporter | Transporter |
| | transporting, mitochondrial | | transporter | | |
| | F1 complex, beta | | | | |
| | polypeptide | | | | |
| ATP5C1 | ATP synthase, H+ | 509 | Channel or | Transporter | Transporter |
| | transporting, mitochondrial | | transporter | | |
| | F1 complex, gamma | | | | |
| | polypeptide 1 | | | | |
| ATP5D | ATP synthase, H+ | 513 | Channel or | Transporter | Transporter |
| | transporting, mitochondrial | | transporter | | |
| | F1 complex, delta subunit | | | | |
| ATP5E | ATP synthase, H+ | 514 | Channel or | Transporter | Transporter |
| | transporting, mitochondrial | | transporter | | |
| | F1 complex, epsilon subunit | | | | |
| ATP5F1 | ATP synthase, H+ | 515 | Channel or | Transporter | Transporter |
| | transporting, mitochondrial | | transporter | | |
| | Fo complex subunit B1 | | | | |
| ATP5H | ATP synthase, H+ | 10476 | Channel or | Transporter | Transporter |
| | transporting, mitochondrial | | transporter | | |
| | Fo complex subunit D | | | | |
| ATP5I | ATP synthase, H+ | 521 | Channel or | Transporter | Transporter |
| | transporting, mitochondrial | | transporter | | |
| | Fo complex subunit E | | | | |
| ATP5J | ATP synthase, H+ | 522 | Channel or | Transporter | Transporter |
| | transporting, mitochondrial | | transporter | | |
| | Fo complex subunit F6 | | | | |
| ATP5J2 | ATP synthase, H+ | 9551 | Channel or | Transporter | Transporter |
| | transporting, mitochondrial | | transporter | | |
| | Fo complex subunit F2 | | | | |
| ATP5L2 | ATP synthase, H+ | 267020 | Channel or | Transporter | Transporter |
| | transporting, mitochondrial | | transporter | | |
| | Fo complex subunit G2 | | | | |
| ATP6V0A1 | Atpase H+ transporting V0 | 535 | Channel or | Transporter | Transporter |
| | subunit a1 | | transporter | | |
| ATP6V0A2 | Atpase H+ transporting V0 | 23545 | Channel or | Transporter | Transporter |
| | subunit a2 | | transporter | | |
| ATP6V0A4 | Atpase H+ transporting V0 | 50617 | Channel or | Transporter | Transporter |
| | subunit a4 | | transporter | | |
| ATP6V0B | Atpase H+ transporting V0 | 533 | Channel or | Transporter | Transporter |
| | subunit b | | transporter | | |
| ATP6V0C | Atpase H+ transporting V0 | 527 | Channel or | Transporter | Transporter |
| | subunit c | | transporter | | |
| ATP6V0D1 | Atpase H+ transporting V0 | 9114 | Channel or | Transporter | Transporter |
| | subunit d1 | | transporter | | |
| ATP6V0D2 | Atpase H+ transporting V0 | 245972 | Channel or | Transporter | Transporter |
| | subunit d2 | | transporter | | |
| ATP6V0E1 | Atpase H+ transporting V0 | 8992 | Channel or | Transporter | Transporter |
| | subunit e1 | | transporter | | |
| ATP6V0E2 | Atpase H+ transporting V0 | 155066 | Channel or | Transporter | Transporter |
| | subunit e2 | | transporter | | |
| ATP6V1A | Atpase H+ transporting V1 | 523 | Channel or | Transporter | Transporter |
| | subunit A | | transporter | | |
| ATP6V1B1 | Atpase H+ transporting V1 | 525 | Channel or | Transporter | Transporter |
| | subunit B1 | | transporter | | |
| ATP6V1B2 | Atpase H+ transporting V1 | 526 | Channel or | Transporter | Transporter |
| | subunit B2 | | transporter | | |
| ATP6V1C1 | Atpase H+ transporting V1 | 528 | Channel or | Transporter | Transporter |
| | subunit C1 | | transporter | | |
| ATP6V1C2 | Atpase H+ transporting V1 | 245973 | Channel or | Transporter | Transporter |
| | subunit C2 | | transporter | | |
| ATP6V1D | Atpase H+ transporting V1 | 51382 | Channel or | Transporter | Transporter |
| | subunit D | | transporter | | |
| ATP6V1E1 | Atpase H+ transporting V1 | 529 | Channel or | Transporter | Transporter |
| | subunit E1 | | transporter | | |
| ATP6V1E2 | Atpase H+ transporting V1 | 90423 | Channel or | Transporter | Transporter |
| | subunit E2 | | transporter | | |
| ATP6V1F | Atpase H+ transporting V1 | 9296 | Channel or | Transporter | Transporter |
| | subunit F | | transporter | | |
| ATP6V1G1 | Atpase H+ transporting V1 | 9550 | Channel or | Transporter | Transporter |
| | subunit G1 | | transporter | | |
| ATP6V1G2 | Atpase H+ transporting V1 | 534 | Channel or | Transporter | Transporter |
| | subunit G2 | | transporter | | |
| ATP6V1G3 | Atpase H+ transporting V1 | 127124 | Channel or | Transporter | Transporter |
| | subunit G3 | | transporter | | |
| ATP6V1H | Atpase H+ transporting V1 | 51606 | Channel or | Transporter | Transporter |
| | subunit H | | transporter | | |
| ATP7A | Atpase copper transporting | 538 | Channel or | Transporter | Transporter |
| | alpha | | transporter | | |
| ATP7B | Atpase copper transporting | 540 | Channel or | Transporter | Transporter |
| | beta | | transporter | | |
| ATP8A1 | Atpase phospholipid | 10396 | Channel or | Transporter | Transporter |
| | transporting 8A1 | | transporter | | |
| ATP8A2 | Atpase phospholipid | 51761 | Channel or | Transporter | Transporter |
| | transporting 8A2 | | transporter | | |
| ATP8B1 | Atpase phospholipid | 5205 | Channel or | Transporter | Transporter |
| | transporting 8B1 | | transporter | | |
| ATP8B2 | Atpase phospholipid | 57198 | Channel or | Transporter | Transporter |
| | transporting 8B2 | | transporter | | |
| ATP8B3 | Atpase phospholipid | 148229 | Channel or | Transporter | Transporter |
| | transporting 8B3 | | transporter | | |
| ATP8B4 | Atpase phospholipid | 79895 | Channel or | Transporter | Transporter |
| | transporting 8B4 (putative) | | transporter | | |
| ATP9A | Atpase phospholipid | 10079 | Channel or | Transporter | Transporter |
| | transporting 9A (putative) | | transporter | | |
| ATP9B | Atpase phospholipid | 374868 | Channel or | Transporter | Transporter |
| | transporting 9B (putative) | | transporter | | |
| DIRC2 | Disrupted in renal | 84925 | Channel or | Transporter | Transporter |
| | carcinoma 2 | | transporter | | |
| FLVCR1 | Feline leukemia virus | 28982 | Channel or | Transporter | Transporter |
| | subgroup C cellular receptor | | transporter | | |
| | 1 | | | | |
| FLVCR2 | Feline leukemia virus | 55640 | Channel or | Transporter | Transporter |
| | subgroup C cellular receptor | | transporter | | |
| | family member 2 | | | | |
| FXYD2 | FXYD domain containing | 486 | Channel or | Transporter | Transporter |
| | ion transport regulator 2 | | transporter | | |
| HTL | High L-leucine transport | 3343 | Channel or | Transporter | Transporter |
| | | | transporter | | |
| MFSD7 | Major facilitator superfamily | 84179 | Channel or | Transporter | Transporter |
| | domain containing 7 | | transporter | | |
| MT-ATP6 | Mitochondrially encoded | 4508 | Channel or | Transporter | Transporter |
| | ATP synthase 6 | | transporter | | |
| MT-ATP8 | Mitochondrially encoded | 4509 | Channel or | Transporter | Transporter |
| | ATP synthase 8 | | transporter | | |
| MTCH1 | Mitochondrial carrier 1 | 23787 | Channel or | Transporter | Transporter |
| | | | transporter | | |
| MTCH2 | Mitochondrial carrier 2 | 23788 | Channel or | Transporter | Transporter |
| | | | transporter | | |
| NPC1L1 | NPC1 like intracellular | 29881 | Channel or | Transporter | Transporter |
| | cholesterol transporter 1 | | transporter | | |
| RHAG | Rh-associated glycoprotein | 6005 | Channel or | Transporter | Transporter |
| | | | transporter | | |
| RHBG | Rh family B glycoprotein | 57127 | Channel or | Transporter | Transporter |
| | (gene/pseudogene) | | transporter | | |
| RHCG | Rh family C glycoprotein | 51458 | Channel or | Transporter | Transporter |
| | | | transporter | | |
| SLC10A1 | Solute carrier family 10 | 6554 | Channel or | Transporter | Transporter |
| | member 1 | | transporter | | |
| SLC10A2 | Solute carrier family 10 | 6555 | Channel or | Transporter | Transporter |
| | member 2 | | transporter | | |
| SLC10A3 | Solute carrier family 10 | 8273 | Channel or | Transporter | Transporter |
| | member 3 | | transporter | | |
| SLC10A4 | Solute carrier family 10 | 201780 | Channel or | Transporter | Transporter |
| | member 4 | | transporter | | |
| SLC10A5 | Solute carrier family 10 | 347051 | Channel or | Transporter | Transporter |
| | member 5 | | transporter | | |
| SLC10A6 | Solute carrier family 10 | 345274 | Channel or | Transporter | Transporter |
| | member 6 | | transporter | | |
| SLC10A7 | Solute carrier family 10 | 84068 | Channel or | Transporter | Transporter |
| | member 7 | | transporter | | |
| SLC11A1 | Solute carrier family 11 | 6556 | Channel or | Transporter | Transporter |
| | member 1 | | transporter | | |
| SLC11A2 | Solute carrier family 11 | 4891 | Channel or | Transporter | Transporter |
| | member 2 | | transporter | | |
| SLC12A1 | Solute carrier family 12 | 6557 | Channel or | Transporter | Transporter |
| | member 1 | | transporter | | |
| SLC12A2 | Solute carrier family 12 | 6558 | Channel or | Transporter | Transporter |
| | member 2 | | transporter | | |
| SLC12A3 | Solute carrier family 12 | 6559 | Channel or | Transporter | Transporter |
| | member 3 | | transporter | | |
| SLC12A4 | Solute carrier family 12 | 6560 | Channel or | Transporter | Transporter |
| | member 4 | | transporter | | |
| SLC12A5 | Solute carrier family 12 | 57468 | Channel or | Transporter | Transporter |
| | member 5 | | transporter | | |
| SLC12A6 | Solute carrier family 12 | 9990 | Channel or | Transporter | Transporter |
| | member 6 | | transporter | | |
| SLC12A7 | Solute carrier family 12 | 10723 | Channel or | Transporter | Transporter |
| | member 7 | | transporter | | |
| SLC12A8 | Solute carrier family 12 | 84561 | Channel or | Transporter | Transporter |
| | member 8 | | transporter | | |
| SLC12A9 | Solute carrier family 12 | 56996 | Channel or | Transporter | Transporter |
| | member 9 | | transporter | | |
| SLC13A1 | Solute carrier family 13 | 6561 | Channel or | Transporter | Transporter |
| | member 1 | | transporter | | |
| SLC13A2 | Solute carrier family 13 | 9058 | Channel or | Transporter | Transporter |
| | member 2 | | transporter | | |
| SLC13A3 | Solute carrier family 13 | 64849 | Channel or | Transporter | Transporter |
| | member 3 | | transporter | | |
| SLC13A4 | Solute carrier family 13 | 26266 | Channel or | Transporter | Transporter |
| | member 4 | | transporter | | |
| SLC13A5 | Solute carrier family 13 | 284111 | Channel or | Transporter | Transporter |
| | member 5 | | transporter | | |
| SLC14A1 | Solute carrier family 14 | 6563 | Channel or | Transporter | Transporter |
| | member 1 (Kidd blood | | transporter | | |
| | group) | | | | |
| SLC14A2 | Solute carrier family 14 | 8170 | Channel or | Transporter | Transporter |
| | member 2 | | transporter | | |
| SLC15A1 | Solute carrier family 15 | 6564 | Channel or | Transporter | Transporter |
| | member 1 | | transporter | | |
| SLC15A2 | Solute carrier family 15 | 6565 | Channel or | Transporter | Transporter |
| | member 2 | | transporter | | |
| SLC15A3 | Solute carrier family 15 | 51296 | Channel or | Transporter | Transporter |
| | member 3 | | transporter | | |
| SLC15A4 | Solute carrier family 15 | 121260 | Channel or | Transporter | Transporter |
| | member 4 | | transporter | | |
| SLC16A1 | Solute carrier family 16 | 6566 | Channel or | Transporter | Transporter |
| | member 1 | | transporter | | |
| SLC16A10 | Solute carrier family 16 | 117247 | Channel or | Transporter | Transporter |
| | member 10 | | transporter | | |
| SLC16A11 | Solute carrier family 16 | 162515 | Channel or | Transporter | Transporter |
| | member 11 | | transporter | | |
| SLC16A12 | Solute carrier family 16 | 387700 | Channel or | Transporter | Transporter |
| | member 12 | | transporter | | |
| SLC16A13 | Solute carrier family 16 | 201232 | Channel or | Transporter | Transporter |
| | member 13 | | transporter | | |
| SLC16A14 | Solute carrier family 16 | 151473 | Channel or | Transporter | Transporter |
| | member 14 | | transporter | | |
| SLC16A2 | Solute carrier family 16 | 6567 | Channel or | Transporter | Transporter |
| | member 2 | | transporter | | |
| SLC16A3 | Solute carrier family 16 | 9123 | Channel or | Transporter | Transporter |
| | member 3 | | transporter | | |
| SLC16A4 | Solute carrier family 16 | 9122 | Channel or | Transporter | Transporter |
| | member 4 | | transporter | | |
| SLC16A5 | Solute carrier family 16 | 9121 | Channel or | Transporter | Transporter |
| | member 5 | | transporter | | |
| SLC16A6 | Solute carrier family 16 | 9120 | Channel or | Transporter | Transporter |
| | member 6 | | transporter | | |
| SLC16A7 | Solute carrier family 16 | 9194 | Channel or | Transporter | Transporter |
| | member 7 | | transporter | | |
| SLC16A8 | Solute carrier family 16 | 23539 | Channel or | Transporter | Transporter |
| | member 8 | | transporter | | |
| SLC16A9 | Solute carrier family 16 | 220963 | Channel or | Transporter | Transporter |
| | member 9 | | transporter | | |
| SLC17A1 | Solute carrier family 17 | 6568 | Channel or | Transporter | Transporter |
| | member 1 | | transporter | | |
| SLC17A2 | Solute carrier family 17 | 10246 | Channel or | Transporter | Transporter |
| | member 2 | | transporter | | |
| SLC17A3 | Solute carrier family 17 | 10786 | Channel or | Transporter | Transporter |
| | member 3 | | transporter | | |
| SLC17A4 | Solute carrier family 17 | 10050 | Channel or | Transporter | Transporter |
| | member 4 | | transporter | | |
| SLC17A5 | Solute carrier family 17 | 26503 | Channel or | Transporter | Transporter |
| | member 5 | | transporter | | |
| SLC17A6 | Solute carrier family 17 | 57084 | Channel or | Transporter | Transporter |
| | member 6 | | transporter | | |
| SLC17A7 | Solute carrier family 17 | 57030 | Channel or | Transporter | Transporter |
| | member 7 | | transporter | | |
| SLC17A8 | Solute carrier family 17 | 246213 | Channel or | Transporter | Transporter |
| | member 8 | | transporter | | |
| SLC17A9 | Solute carrier family 17 | 63910 | Channel or | Transporter | Transporter |
| | member 9 | | transporter | | |
| SLC18A1 | Solute carrier family 18 | 6570 | Channel or | Transporter | Transporter |
| | member A1 | | transporter | | |
| SLC18A2 | Solute carrier family 18 | 6571 | Channel or | Transporter | Transporter |
| | member A2 | | transporter | | |
| SLC18A3 | Solute carrier family 18 | 6572 | Channel or | Transporter | Transporter |
| | member A3 | | transporter | | |
| SLC18B1 | Solute carrier family 18 | 116843 | Channel or | Transporter | Transporter |
| | member B1 | | transporter | | |
| SLC19A1 | Solute carrier family 19 | 6573 | Channel or | Transporter | Transporter |
| | member 1 | | transporter | | |
| SLC19A2 | Solute carrier family 19 | 10560 | Channel or | Transporter | Transporter |
| | member 2 | | transporter | | |
| SLC19A3 | Solute carrier family 19 | 80704 | Channel or | Transporter | Transporter |
| | member 3 | | transporter | | |
| SLC1A1 | Solute carrier family 1 | 6505 | Channel or | Transporter | Transporter |
| | member 1 | | transporter | | |
| SLC1A2 | Solute carrier family 1 | 6506 | Channel or | Transporter | Transporter |
| | member 2 | | transporter | | |
| SLC1A3 | Solute carrier family 1 | 6507 | Channel or | Transporter | Transporter |
| | member 3 | | transporter | | |
| SLC1A6 | Solute carrier family 1 | 6511 | Channel or | Transporter | Transporter |
| | member 6 | | transporter | | |
| SLC1A7 | Solute carrier family 1 | 6512 | Channel or | Transporter | Transporter |
| | member 7 | | transporter | | |
| SLC20A1 | Solute carrier family 20 | 6574 | Channel or | Transporter | Transporter |
| | member 1 | | transporter | | |
| SLC20A2 | Solute carrier family 20 | 6575 | Channel or | Transporter | Transporter |
| | member 2 | | transporter | | |
| SLC22A1 | Solute carrier family 22 | 6580 | Channel or | Transporter | Transporter |
| | member 1 | | transporter | | |
| SLC22A10 | Solute carrier family 22 | 387775 | Channel or | Transporter | Transporter |
| | member 10 | | transporter | | |
| SLC22A11 | Solute carrier family 22 | 55867 | Channel or | Transporter | Transporter |
| | member 11 | | transporter | | |
| SLC22A12 | Solute carrier family 22 | 116085 | Channel or | Transporter | Transporter |
| | member 12 | | transporter | | |
| SLC22A13 | Solute carrier family 22 | 9390 | Channel or | Transporter | Transporter |
| | member 13 | | transporter | | |
| SLC22A14 | Solute carrier family 22 | 9389 | Channel or | Transporter | Transporter |
| | member 14 | | transporter | | |
| SLC22A15 | Solute carrier family 22 | 55356 | Channel or | Transporter | Transporter |
| | member 15 | | transporter | | |
| SLC22A16 | Solute carrier family 22 | 85413 | Channel or | Transporter | Transporter |
| | member 16 | | transporter | | |
| SLC22A17 | Solute carrier family 22 | 51310 | Channel or | Transporter | Transporter |
| | member 17 | | transporter | | |
| SLC22A18 | Solute carrier family 22 | 5002 | Channel or | Transporter | Transporter |
| | member 18 | | transporter | | |
| SLC22A2 | Solute carrier family 22 | 6582 | Channel or | Transporter | Transporter |
| | member 2 | | transporter | | |
| SLC22A20 | Solute carrier family 22 | 440044 | Channel or | Transporter | Transporter |
| | member 20 | | transporter | | |
| SLC22A23 | Solute carrier family 22 | 63027 | Channel or | Transporter | Transporter |
| | member 23 | | transporter | | |
| SLC22A24 | Solute carrier family 22 | 283238 | Channel or | Transporter | Transporter |
| | member 24 | | transporter | | |
| SLC22A25 | Solute carrier family 22 | 387601 | Channel or | Transporter | Transporter |
| | member 25 | | transporter | | |
| SLC22A3 | Solute carrier family 22 | 6581 | Channel or | Transporter | Transporter |
| | member 3 | | transporter | | |
| SLC22A31 | Solute carrier family 22 | 146429 | Channel or | Transporter | Transporter |
| | member 31 | | transporter | | |
| SLC22A4 | Solute carrier family 22 | 6583 | Channel or | Transporter | Transporter |
| | member 4 | | transporter | | |
| SLC22A5 | Solute carrier family 22 | 6584 | Channel or | Transporter | Transporter |
| | member 5 | | transporter | | |
| SLC22A6 | Solute carrier family 22 | 9356 | Channel or | Transporter | Transporter |
| | member 6 | | transporter | | |
| SLC22A7 | Solute carrier family 22 | 10864 | Channel or | Transporter | Transporter |
| | member 7 | | transporter | | |
| SLC22A8 | Solute carrier family 22 | 9376 | Channel or | Transporter | Transporter |
| | member 8 | | transporter | | |
| SLC22A9 | Solute carrier family 22 | 114571 | Channel or | Transporter | Transporter |
| | member 9 | | transporter | | |
| SLC23A1 | Solute carrier family 23 | 9963 | Channel or | Transporter | Transporter |
| | member 1 | | transporter | | |
| SLC23A2 | Solute carrier family 23 | 9962 | Channel or | Transporter | Transporter |
| | member 2 | | transporter | | |
| SLC23A3 | Solute carrier family 23 | 151295 | Channel or | Transporter | Transporter |
| | member 3 | | transporter | | |
| SLC23A4P | Solute carrier family 23 | 641842 | Channel or | Transporter | Transporter |
| | member 4, pseudogene | | transporter | | |
| SLC24A1 | Solute carrier family 24 | 9187 | Channel or | Transporter | Transporter |
| | member 1 | | transporter | | |
| SLC24A2 | Solute carrier family 24 | 25769 | Channel or | Transporter | Transporter |
| | member 2 | | transporter | | |
| SLC24A3 | Solute carrier family 24 | 57419 | Channel or | Transporter | Transporter |
| | member 3 | | transporter | | |
| SLC24A4 | Solute carrier family 24 | 123041 | Channel or | Transporter | Transporter |
| | member 4 | | transporter | | |
| SLC24A5 | Solute carrier family 24 | 283652 | Channel or | Transporter | Transporter |
| | member 5 | | transporter | | |
| SLC25A1 | Solute carrier family 25 | 6576 | Channel or | Transporter | Transporter |
| | member 1 | | transporter | | |
| SLC25A10 | Solute carrier family 25 | 1468 | Channel or | Transporter | Transporter |
| | member 10 | | transporter | | |
| SLC25A11 | Solute carrier family 25 | 8402 | Channel or | Transporter | Transporter |
| | member 11 | | transporter | | |
| SLC25A12 | Solute carrier family 25 | 8604 | Channel or | Transporter | Transporter |
| | member 12 | | transporter | | |
| SLC25A13 | Solute carrier family 25 | 10165 | Channel or | Transporter | Transporter |
| | member 13 | | transporter | | |
| SLC25A14 | Solute carrier family 25 | 9016 | Channel or | Transporter | Transporter |
| | member 14 | | transporter | | |
| SLC25A15 | Solute carrier family 25 | 10166 | Channel or | Transporter | Transporter |
| | member 15 | | transporter | | |
| SLC25A16 | Solute carrier family 25 | 8034 | Channel or | Transporter | Transporter |
| | member 16 | | transporter | | |
| SLC25A17 | Solute carrier family 25 | 10478 | Channel or | Transporter | Transporter |
| | member 17 | | transporter | | |
| SLC25A18 | Solute carrier family 25 | 83733 | Channel or | Transporter | Transporter |
| | member 18 | | transporter | | |
| SLC25A19 | Solute carrier family 25 | 60386 | Channel or | Transporter | Transporter |
| | member 19 | | transporter | | |
| SLC25A2 | Solute carrier family 25 | 83884 | Channel or | Transporter | Transporter |
| | member 2 | | transporter | | |
| SLC25A20 | Solute carrier family 25 | 788 | Channel or | Transporter | Transporter |
| | member 20 | | transporter | | |
| SLC25A21 | Solute carrier family 25 | 89874 | Channel or | Transporter | Transporter |
| | member 21 | | transporter | | |
| SLC25A22 | Solute carrier family 25 | 79751 | Channel or | Transporter | Transporter |
| | member 22 | | transporter | | |
| SLC25A23 | Solute carrier family 25 | 79085 | Channel or | Transporter | Transporter |
| | member 23 | | transporter | | |
| SLC25A24 | Solute carrier family 25 | 29957 | Channel or | Transporter | Transporter |
| | member 24 | | transporter | | |
| SLC25A25 | Solute carrier family 25 | 114789 | Channel or | Transporter | Transporter |
| | member 25 | | transporter | | |
| SLC25A26 | Solute carrier family 25 | 115286 | Channel or | Transporter | Transporter |
| | member 26 | | transporter | | |
| SLC25A27 | Solute carrier family 25 | 9481 | Channel or | Transporter | Transporter |
| | member 27 | | transporter | | |
| SLC25A28 | Solute carrier family 25 | 81894 | Channel or | Transporter | Transporter |
| | member 28 | | transporter | | |
| SLC25A29 | Solute carrier family 25 | 123096 | Channel or | Transporter | Transporter |
| | member 29 | | transporter | | |
| SLC25A3 | Solute carrier family 25 | 5250 | Channel or | Transporter | Transporter |
| | member 3 | | transporter | | |
| SLC25A30 | Solute carrier family 25 | 253512 | Channel or | Transporter | Transporter |
| | member 30 | | transporter | | |
| SLC25A31 | Solute carrier family 25 | 83447 | Channel or | Transporter | Transporter |
| | member 31 | | transporter | | |
| SLC25A32 | Solute carrier family 25 | 81034 | Channel or | Transporter | Transporter |
| | member 32 | | transporter | | |
| SLC25A33 | Solute carrier family 25 | 84275 | Channel or | Transporter | Transporter |
| | member 33 | | transporter | | |
| SLC25A34 | Solute carrier family 25 | 284723 | Channel or | Transporter | Transporter |
| | member 34 | | transporter | | |
| SLC25A35 | Solute carrier family 25 | 399512 | Channel or | Transporter | Transporter |
| | member 35 | | transporter | | |
| SLC25A36 | Solute carrier family 25 | 55186 | Channel or | Transporter | Transporter |
| | member 36 | | transporter | | |
| SLC25A37 | Solute carrier family 25 | 51312 | Channel or | Transporter | Transporter |
| | member 37 | | transporter | | |
| SLC25A38 | Solute carrier family 25 | 54977 | Channel or | Transporter | Transporter |
| | member 38 | | transporter | | |
| SLC25A39 | Solute carrier family 25 | 51629 | Channel or | Transporter | Transporter |
| | member 39 | | transporter | | |
| SLC25A4 | Solute carrier family 25 | 291 | Channel or | Transporter | Transporter |
| | member 4 | | transporter | | |
| SLC25A40 | Solute carrier family 25 | 55972 | Channel or | Transporter | Transporter |
| | member 40 | | transporter | | |
| SLC25A41 | Solute carrier family 25 | 284427 | Channel or | Transporter | Transporter |
| | member 41 | | transporter | | |
| SLC25A42 | Solute carrier family 25 | 284439 | Channel or | Transporter | Transporter |
| | member 42 | | transporter | | |
| SLC25A43 | Solute carrier family 25 | 203427 | Channel or | Transporter | Transporter |
| | member 43 | | transporter | | |
| SLC25A44 | Solute carrier family 25 | 9673 | Channel or | Transporter | Transporter |
| | member 44 | | transporter | | |
| SLC25A45 | Solute carrier family 25 | 283130 | Channel or | Transporter | Transporter |
| | member 45 | | transporter | | |
| SLC25A46 | Solute carrier family 25 | 91137 | Channel or | Transporter | Transporter |
| | member 46 | | transporter | | |
| SLC25A47 | Solute carrier family 25 | 283600 | Channel or | Transporter | T ransporter |
| | member 47 | | transporter | | |
| SLC25A48 | Solute carrier family 25 | 153328 | Channel or | Transporter | Transporter |
| | member 48 | | transporter | | |
| SLC25A5 | Solute carrier family 25 | 292 | Channel or | Transporter | Transporter |
| | member 5 | | transporter | | |
| SLC25A51 | Solute carrier family 25 | 92014 | Channel or | Transporter | Transporter |
| | member 51 | | transporter | | |
| SLC25A52 | Solute carrier family 25 | 147407 | Channel or | Transporter | Transporter |
| | member 52 | | transporter | | |
| SLC25A53 | Solute carrier family 25 | 401612 | Channel or | Transporter | Transporter |
| | member 53 | | transporter | | |
| SLC25A6 | Solute carrier family 25 | 293 | Channel or | Transporter | Transporter |
| | member 6 | | transporter | | |
| SLC26A1 | Solute carrier family 26 | 10861 | Channel or | Transporter | Transporter |
| | member 1 | | transporter | | |
| SLC26A10 | Solute carrier family 26 | 65012 | Channel or | Transporter | Transporter |
| | member 10 | | transporter | | |
| SLC26A11 | Solute carrier family 26 | 284129 | Channel or | Transporter | Transporter |
| | member 11 | | transporter | | |
| SLC26A2 | Solute carrier family 26 | 1836 | Channel or | Transporter | Transporter |
| | member 2 | | transporter | | |
| SLC26A3 | Solute carrier family 26 | 1811 | Channel or | Transporter | Transporter |
| | member 3 | | transporter | | |
| SLC26A4 | Solute carrier family 26 | 5172 | Channel or | Transporter | Transporter |
| | member 4 | | transporter | | |
| SLC26A5 | Solute carrier family 26 | 375611 | Channel or | Transporter | Transporter |
| | member 5 | | transporter | | |
| SLC26A6 | Solute carrier family 26 | 65010 | Channel or | Transporter | Transporter |
| | member 6 | | transporter | | |
| SLC26A7 | Solute carrier family 26 | 115111 | Channel or | Transporter | Transporter |
| | member 7 | | transporter | | |
| SLC26A8 | Solute carrier family 26 | 116369 | Channel or | Transporter | Transporter |
| | member 8 | | transporter | | |
| SLC26A9 | Solute carrier family 26 | 115019 | Channel or | Transporter | Transporter |
| | member 9 | | transporter | | |
| SLC27A1 | Solute carrier family 27 | 376497 | Channel or | Transporter | Transporter |
| | member 1 | | transporter | | |
| SLC27A2 | Solute carrier family 27 | 11001 | Channel or | Transporter | Transporter |
| | member 2 | | transporter | | |
| SLC27A3 | Solute carrier family 27 | 11000 | Channel or | Transporter | Transporter |
| | member 3 | | transporter | | |
| SLC27A4 | Solute carrier family 27 | 10999 | Channel or | Transporter | Transporter |
| | member 4 | | transporter | | |
| SLC27A5 | Solute carrier family 27 | 10998 | Channel or | Transporter | Transporter |
| | member 5 | | transporter | | |
| SLC27A6 | Solute carrier family 27 | 28965 | Channel or | Transporter | Transporter |
| | member 6 | | transporter | | |
| SLC28A1 | Solute carrier family 28 | 9154 | Channel or | Transporter | Transporter |
| | member 1 | | transporter | | |
| SLC28A2 | Solute carrier family 28 | 9153 | Channel or | Transporter | Transporter |
| | member 2 | | transporter | | |
| SLC28A3 | Solute carrier family 28 | 64078 | Channel or | Transporter | Transporter |
| | member 3 | | transporter | | |
| SLC29A1 | Solute carrier family 29 | 2030 | Channel or | Transporter | Transporter |
| | member 1 (Augustine blood | | transporter | | |
| | group) | | | | |
| SLC29A2 | Solute carrier family 29 | 3177 | Channel or | Transporter | Transporter |
| | member 2 | | transporter | | |
| SLC29A3 | Solute carrier family 29 | 55315 | Channel or | Transporter | Transporter |
| | member 3 | | transporter | | |
| SLC29A4 | Solute carrier family 29 | 222962 | Channel or | Transporter | Transporter |
| | member 4 | | transporter | | |
| SLC2A1 | Solute carrier family 2 | 6513 | Channel or | Transporter | Transporter |
| | member 1 | | transporter | | |
| SLC2A10 | Solute carrier family 2 | 81031 | Channel or | Transporter | Transporter |
| | member 10 | | transporter | | |
| SLC2A11 | Solute carrier family 2 | 66035 | Channel or | Transporter | Transporter |
| | member 11 | | transporter | | |
| SLC2A12 | Solute carrier family 2 | 154091 | Channel or | Transporter | Transporter |
| | member 12 | | transporter | | |
| SLC2A13 | Solute carrier family 2 | 114134 | Channel or | Transporter | Transporter |
| | member 13 | | transporter | | |
| SLC2A14 | Solute carrier family 2 | 144195 | Channel or | Transporter | Transporter |
| | member 14 | | transporter | | |
| SLC2A2 | Solute carrier family 2 | 6514 | Channel or | Transporter | Transporter |
| | member 2 | | transporter | | |
| SLC2A3 | Solute carrier family 2 | 6515 | Channel or | Transporter | Transporter |
| | member 3 | | transporter | | |
| SLC2A4 | Solute carrier family 2 | 6517 | Channel or | Transporter | Transporter |
| | member 4 | | transporter | | |
| SLC2A5 | Solute carrier family 2 | 6518 | Channel or | Transporter | Transporter |
| | member 5 | | transporter | | |
| SLC2A6 | Solute carrier family 2 | 11182 | Channel or | Transporter | Transporter |
| | member 6 | | transporter | | |
| SLC2A7 | Solute carrier family 2 | 155184 | Channel or | Transporter | Transporter |
| | member 7 | | transporter | | |
| SLC2A8 | Solute carrier family 2 | 29988 | Channel or | Transporter | Transporter |
| | member 8 | | transporter | | |
| SLC2A9 | Solute carrier family 2 | 56606 | Channel or | Transporter | Transporter |
| | member 9 | | transporter | | |
| SLC30A1 | Solute carrier family 30 | 7779 | Channel or | Transporter | Transporter |
| | member 1 | | transporter | | |
| SLC30A10 | Solute carrier family 30 | 55532 | Channel or | Transporter | Transporter |
| | member 10 | | transporter | | |
| SLC30A2 | Solute carrier family 30 | 7780 | Channel or | Transporter | Transporter |
| | member 2 | | transporter | | |
| SLC30A3 | Solute carrier family 30 | 7781 | Channel or | Transporter | Transporter |
| | member 3 | | transporter | | |
| SLC30A4 | Solute carrier family 30 | 7782 | Channel or | Transporter | Transporter |
| | member 4 | | transporter | | |
| SLC30A5 | Solute carrier family 30 | 64924 | Channel or | Transporter | Transporter |
| | member 5 | | transporter | | |
| SLC30A6 | Solute carrier family 30 | 55676 | Channel or | Transporter | Transporter |
| | member 6 | | transporter | | |
| SLC30A7 | Solute carrier family 30 | 148867 | Channel or | Transporter | Transporter |
| | member 7 | | transporter | | |
| SLC30A8 | Solute carrier family 30 | 169026 | Channel or | Transporter | Transporter |
| | member 8 | | transporter | | |
| SLC30A9 | Solute carrier family 30 | 10463 | Channel or | Transporter | Transporter |
| | member 9 | | transporter | | |
| SLC31A1 | Solute carrier family 31 | 1317 | Channel or | Transporter | Transporter |
| | member 1 | | transporter | | |
| SLC31A2 | Solute carrier family 31 | 1318 | Channel or | Transporter | Transporter |
| | member 2 | | transporter | | |
| SLC32A1 | Solute carrier family 32 | 140679 | Channel or | Transporter | Transporter |
| | member 1 | | transporter | | |
| SLC33A1 | Solute carrier family 33 | 9197 | Channel or | Transporter | Transporter |
| | member 1 | | transporter | | |
| SLC34A1 | Solute carrier family 34 | 6569 | Channel or | Transporter | Transporter |
| | member 1 | | transporter | | |
| SLC34A2 | Solute carrier family 34 | 10568 | Channel or | Transporter | Transporter |
| | member 2 | | transporter | | |
| SLC34A3 | Solute carrier family 34 | 142680 | Channel or | Transporter | Transporter |
| | member 3 | | transporter | | |
| SLC35A1 | Solute carrier family 35 | 10559 | Channel or | Transporter | Transporter |
| | member A1 | | transporter | | |
| SLC35A2 | Solute carrier family 35 | 7355 | Channel or | Transporter | Transporter |
| | member A2 | | transporter | | |
| SLC35A3 | Solute carrier family 35 | 23443 | Channel or | Transporter | Transporter |
| | member A3 | | transporter | | |
| SLC35A4 | Solute carrier family 35 | 113829 | Channel or | Transporter | Transporter |
| | member A4 | | transporter | | |
| SLC35A5 | Solute carrier family 35 | 55032 | Channel or | Transporter | Transporter |
| | member A5 | | transporter | | |
| SLC35B1 | Solute carrier family 35 | 10237 | Channel or | Transporter | Transporter |
| | member B1 | | transporter | | |
| SLC35B2 | Solute carrier family 35 | 347734 | Channel or | Transporter | Transporter |
| | member B2 | | transporter | | |
| SLC35B3 | Solute carrier family 35 | 51000 | Channel or | Transporter | Transporter |
| | member B3 | | transporter | | |
| SLC35B4 | Solute carrier family 35 | 84912 | Channel or | Transporter | Transporter |
| | member B4 | | transporter | | |
| SLC35C1 | Solute carrier family 35 | 55343 | Channel or | Transporter | Transporter |
| | member C1 | | transporter | | |
| SLC35C2 | Solute carrier family 35 | 51006 | Channel or | Transporter | Transporter |
| | member C2 | | transporter | | |
| SLC35D1 | Solute carrier family 35 | 23169 | Channel or | Transporter | Transporter |
| | member D1 | | transporter | | |
| SLC35D2 | Solute carrier family 35 | 11046 | Channel or | Transporter | Transporter |
| | member D2 | | transporter | | |
| SLC35D3 | Solute carrier family 35 | 340146 | Channel or | Transporter | Transporter |
| | member D3 | | transporter | | |
| SLC35E1 | Solute carrier family 35 | 79939 | Channel or | Transporter | Transporter |
| | member E1 | | transporter | | |
| SLC35E2 | Solute carrier family 35 | 9906 | Channel or | Transporter | Transporter |
| | member E2 | | transporter | | |
| SLC35E2B | Solute carrier family 35 | 728661 | Channel or | Transporter | Transporter |
| | member E2B | | transporter | | |
| SLC35E3 | Solute carrier family 35 | 55508 | Channel or | Transporter | Transporter |
| | member E3 | | transporter | | |
| SLC35E4 | Solute carrier family 35 | 339665 | Channel or | Transporter | Transporter |
| | member E4 | | transporter | | |
| SLC35F1 | Solute carrier family 35 | 222553 | Channel or | Transporter | Transporter |
| | member F1 | | transporter | | |
| SLC35F2 | Solute carrier family 35 | 54733 | Channel or | Transporter | Transporter |
| | member F2 | | transporter | | |
| SLC35F3 | Solute carrier family 35 | 148641 | Channel or | Transporter | Transporter |
| | member F3 | | transporter | | |
| SLC35F4 | Solute carrier family 35 | 341880 | Channel or | Transporter | Transporter |
| | member F4 | | transporter | | |
| SLC35F5 | Solute carrier family 35 | 80255 | Channel or | Transporter | Transporter |
| | member F5 | | transporter | | |
| SLC35F6 | Solute carrier family 35 | 54978 | Channel or | Transporter | Transporter |
| | member F6 | | transporter | | |
| SLC35G1 | Solute carrier family 35 | 159371 | Channel or | Transporter | Transporter |
| | member G1 | | transporter | | |
| SLC35G2 | Solute carrier family 35 | 80723 | Channel or | Transporter | Transporter |
| | member G2 | | transporter | | |
| SLC35G3 | Solute carrier family 35 | 146861 | Channel or | Transporter | Transporter |
| | member G3 | | transporter | | |
| SLC35G4 | Solute carrier family 35 | 646000 | Channel or | Transporter | Transporter |
| | member G4 | | transporter | | |
| SLC35G5 | Solute carrier family 35 | 83650 | Channel or | Transporter | Transporter |
| | member G5 | | transporter | | |
| SLC35G6 | Solute carrier family 35 | 643664 | Channel or | Transporter | Transporter |
| | member G6 | | transporter | | |
| SLC36A1 | Solute carrier family 36 | 206358 | Channel or | Transporter | Transporter |
| | member 1 | | transporter | | |
| SLC36A2 | Solute carrier family 36 | 153201 | Channel or | Transporter | Transporter |
| | member 2 | | transporter | | |
| SLC36A3 | Solute carrier family 36 | 285641 | Channel or | Transporter | Transporter |
| | member 3 | | transporter | | |
| SLC36A4 | Solute carrier family 36 | 120103 | Channel or | Transporter | Transporter |
| | member 4 | | transporter | | |
| SLC37A1 | Solute carrier family 37 | 54020 | Channel or | Transporter | Transporter |
| | member 1 | | transporter | | |
| SLC37A2 | Solute carrier family 37 | 219855 | Channel or | Transporter | Transporter |
| | member 2 | | transporter | | |
| SLC37A3 | Solute carrier family 37 | 84255 | Channel or | Transporter | Transporter |
| | member 3 | | transporter | | |
| SLC37A4 | Solute carrier family 37 | 2542 | Channel or | Transporter | Transporter |
| | member 4 | | transporter | | |
| SLC38A1 | Solute carrier family 38 | 81539 | Channel or | Transporter | Transporter |
| | member 1 | | transporter | | |
| SLC38A10 | Solute carrier family 38 | 124565 | Channel or | Transporter | Transporter |
| | member 10 | | transporter | | |
| SLC38A11 | Solute carrier family 38 | 151258 | Channel or | Transporter | Transporter |
| | member 11 | | transporter | | |
| SLC38A2 | Solute carrier family 38 | 54407 | Channel or | Transporter | Transporter |
| | member 2 | | transporter | | |
| SLC38A3 | Solute carrier family 38 | 10991 | Channel or | Transporter | Transporter |
| | member 3 | | transporter | | |
| SLC38A4 | Solute carrier family 38 | 55089 | Channel or | Transporter | Transporter |
| | member 4 | | transporter | | |
| SLC38A5 | Solute carrier family 38 | 92745 | Channel or | Transporter | Transporter |
| | member 5 | | transporter | | |
| SLC38A6 | Solute carrier family 38 | 145389 | Channel or | Transporter | Transporter |
| | member 6 | | transporter | | |
| SLC38A7 | Solute carrier family 38 | 55238 | Channel or | Transporter | Transporter |
| | member 7 | | transporter | | |
| SLC38A8 | Solute carrier family 38 | 146167 | Channel or | Transporter | Transporter |
| | member 8 | | transporter | | |
| SLC38A9 | Solute carrier family 38 | 153129 | Channel or | Transporter | Transporter |
| | member 9 | | transporter | | |
| SLC39A1 | Solute carrier family 39 | 27173 | Channel or | Transporter | Transporter |
| | member 1 | | transporter | | |
| SLC39A10 | Solute carrier family 39 | 57181 | Channel or | Transporter | Transporter |
| | member 10 | | transporter | | |
| SLC39A11 | Solute carrier family 39 | 201266 | Channel or | Transporter | Transporter |
| | member 11 | | transporter | | |
| SLC39A12 | Solute carrier family 39 | 221074 | Channel or | Transporter | Transporter |
| | member 12 | | transporter | | |
| SLC39A13 | Solute carrier family 39 | 91252 | Channel or | Transporter | Transporter |
| | member 13 | | transporter | | |
| SLC39A14 | Solute carrier family 39 | 23516 | Channel or | Transporter | Transporter |
| | member 14 | | transporter | | |
| SLC39A2 | Solute carrier family 39 | 29986 | Channel or | Transporter | Transporter |
| | member 2 | | transporter | | |
| SLC39A3 | Solute carrier family 39 | 29985 | Channel or | Transporter | Transporter |
| | member 3 | | transporter | | |
| SLC39A4 | Solute carrier family 39 | 55630 | Channel or | Transporter | Transporter |
| | member 4 | | transporter | | |
| SLC39A5 | Solute carrier family 39 | 283375 | Channel or | Transporter | Transporter |
| | member 5 | | transporter | | |
| SLC39A6 | Solute carrier family 39 | 25800 | Channel or | Transporter | Transporter |
| | member 6 | | transporter | | |
| SLC39A7 | Solute carrier family 39 | 7922 | Channel or | Transporter | Transporter |
| | member 7 | | transporter | | |
| SLC39A8 | Solute carrier family 39 | 64116 | Channel or | Transporter | Transporter |
| | member 8 | | transporter | | |
| SLC39A9 | Solute carrier family 39 | 55334 | Channel or | Transporter | Transporter |
| | member 9 | | transporter | | |
| SLC3A1 | Solute carrier family 3 | 6519 | Channel or | Transporter | Transporter |
| | member 1 | | transporter | | |
| SLC3A2 | Solute carrier family 3 | 6520 | Channel or | Transporter | Transporter |
| | member 2 | | transporter | | |
| SLC40A1 | Solute carrier family 40 | 30061 | Channel or | Transporter | Transporter |
| | member 1 | | transporter | | |
| SLC41A1 | Solute carrier family 41 | 254428 | Channel or | Transporter | Transporter |
| | member 1 | | transporter | | |
| SLC41A2 | Solute carrier family 41 | 84102 | Channel or | Transporter | Transporter |
| | member 2 | | transporter | | |
| SLC41A3 | Solute carrier family 41 | 54946 | Channel or | Transporter | Transporter |
| | member 3 | | transporter | | |
| SLC43A1 | Solute carrier family 43 | 8501 | Channel or | Transporter | Transporter |
| | member 1 | | transporter | | |
| SLC43A2 | Solute carrier family 43 | 124935 | Channel or | Transporter | Transporter |
| | member 2 | | transporter | | |
| SLC43A3 | Solute carrier family 43 | 29015 | Channel or | Transporter | Transporter |
| | member 3 | | transporter | | |
| SLC44A1 | Solute carrier family 44 | 23446 | Channel or | Transporter | Transporter |
| | member 1 | | transporter | | |
| SLC44A2 | Solute carrier family 44 | 57153 | Channel or | Transporter | Transporter |
| | member 2 | | transporter | | |
| SLC44A3 | Solute carrier family 44 | 126969 | Channel or | Transporter | Transporter |
| | member 3 | | transporter | | |
| SLC44A4 | Solute carrier family 44 | 80736 | Channel or | Transporter | Transporter |
| | member 4 | | transporter | | |
| SLC44A5 | Solute carrier family 44 | 204962 | Channel or | Transporter | Transporter |
| | member 5 | | transporter | | |
| SLC45A1 | Solute carrier family 45 | 50651 | Channel or | Transporter | Transporter |
| | member 1 | | transporter | | |
| SLC45A2 | Solute carrier family 45 | 51151 | Channel or | Transporter | Transporter |
| | member 2 | | transporter | | |
| SLC45A3 | Solute carrier family 45 | 85414 | Channel or | Transporter | Transporter |
| | member 3 | | transporter | | |
| SLC45A4 | Solute carrier family 45 | 57210 | Channel or | Transporter | Transporter |
| | member 4 | | transporter | | |
| SLC46A1 | Solute carrier family 46 | 113235 | Channel or | Transporter | Transporter |
| | member 1 | | transporter | | |
| SLC46A2 | Solute carrier family 46 | 57864 | Channel or | Transporter | Transporter |
| | member 2 | | transporter | | |
| SLC46A3 | Solute carrier family 46 | 283537 | Channel or | Transporter | Transporter |
| | member 3 | | transporter | | |
| SLC47A1 | Solute carrier family 47 | 55244 | Channel or | Transporter | Transporter |
| | member 1 | | transporter | | |
| SLC47A2 | Solute carrier family 47 | 146802 | Channel or | Transporter | Transporter |
| | member 2 | | transporter | | |
| SLC48A1 | Solute carrier family 48 | 55652 | Channel or | Transporter | Transporter |
| | member 1 | | transporter | | |
| SLC4A1 | Solute carrier family 4 | 6521 | Channel or | Transporter | Transporter |
| | member 1 | | transporter | | |
| SLC4A10 | Solute carrier family 4 | 57282 | Channel or | Transporter | Transporter |
| | member 10 | | transporter | | |
| SLC4A11 | Solute carrier family 4 | 83959 | Channel or | Transporter | Transporter |
| | member 11 | | transporter | | |
| SLC4A2 | Solute carrier family 4 | 6522 | Channel or | Transporter | Transporter |
| | member 2 | | transporter | | |
| SLC4A3 | Solute carrier family 4 | 6508 | Channel or | Transporter | Transporter |
| | member 3 | | transporter | | |
| SLC4A4 | Solute carrier family 4 | 8671 | Channel or | Transporter | Transporter |
| | member 4 | | transporter | | |
| SLC4A5 | Solute carrier family 4 | 57835 | Channel or | Transporter | Transporter |
| | member 5 | | transporter | | |
| SLC4A7 | Solute carrier family 4 | 9497 | Channel or | Transporter | Transporter |
| | member 7 | | transporter | | |
| SLC4A8 | Solute carrier family 4 | 9498 | Channel or | Transporter | Transporter |
| | member 8 | | transporter | | |
| SLC4A9 | Solute carrier family 4 | 83697 | Channel or | Transporter | Transporter |
| | member 9 | | transporter | | |
| SLC50A1 | Solute carrier family 50 | 55974 | Channel or | Transporter | Transporter |
| | member 1 | | transporter | | |
| SLC51A | Solute carrier family 51 | 200931 | Channel or | Transporter | Transporter |
| | alpha subunit | | transporter | | |
| SLC51B | Solute carrier family 51 beta | 123264 | Channel or | Transporter | Transporter |
| | subunit | | transporter | | |
| SLC52A1 | Solute carrier family 52 | 55065 | Channel or | Transporter | Transporter |
| | member 1 | | transporter | | |
| SLC52A2 | Solute carrier family 52 | 79581 | Channel or | Transporter | Transporter |
| | member 2 | | transporter | | |
| SLC52A3 | Solute carrier family 52 | 113278 | Channel or | Transporter | Transporter |
| | member 3 | | transporter | | |
| SLC5A1 | Solute carrier family 5 | 6523 | Channel or | Transporter | Transporter |
| | member 1 | | transporter | | |
| SLC5A10 | Solute carrier family 5 | 125206 | Channel or | Transporter | Transporter |
| | member 10 | | transporter | | |
| SLC5A11 | Solute carrier family 5 | 115584 | Channel or | Transporter | Transporter |
| | member 11 | | transporter | | |
| SLC5A12 | Solute carrier family 5 | 159963 | Channel or | Transporter | Transporter |
| | member 12 | | transporter | | |
| SLC5A2 | Solute carrier family 5 | 6524 | Channel or | Transporter | Transporter |
| | member 2 | | transporter | | |
| SLC5A3 | Solute carrier family 5 | 6526 | Channel or | Transporter | Transporter |
| | member 3 | | transporter | | |
| SLC5A4 | Solute carrier family 5 | 6527 | Channel or | Transporter | Transporter |
| | member 4 | | transporter | | |
| SLC5A5 | Solute carrier family 5 | 6528 | Channel or | Transporter | Transporter |
| | member 5 | | transporter | | |
| SLC5A6 | Solute carrier family 5 | 8884 | Channel or | Transporter | Transporter |
| | member 6 | | transporter | | |
| SLC5A7 | Solute carrier family 5 | 60482 | Channel or | Transporter | Transporter |
| | member 7 | | transporter | | |
| SLC5A8 | Solute carrier family 5 | 160728 | Channel or | Transporter | Transporter |
| | member 8 | | transporter | | |
| SLC5A9 | Solute carrier family 5 | 200010 | Channel or | Transporter | Transporter |
| | member 9 | | transporter | | |
| SLC6A1 | Solute carrier family 6 | 6529 | Channel or | Transporter | Transporter |
| | member 1 | | transporter | | |
| SLC6A10P | Solute carrier family 6 | 386757 | Channel or | Transporter | Transporter |
| | member 10, pseudogene | | transporter | | |
| SLC6A10PB | Solute carrier family 6 | 653562 | Channel or | Transporter | Transporter |
| | member 8 pseudogene | | transporter | | |
| SLC6A11 | Solute carrier family 6 | 6538 | Channel or | Transporter | Transporter |
| | member 11 | | transporter | | |
| SLC6A12 | Solute carrier family 6 | 6539 | Channel or | Transporter | Transporter |
| | member 12 | | transporter | | |
| SLC6A13 | Solute carrier family 6 | 6540 | Channel or | Transporter | Transporter |
| | member 13 | | transporter | | |
| SLC6A14 | Solute carrier family 6 | 11254 | Channel or | Transporter | Transporter |
| | member 14 | | transporter | | |
| SLC6A15 | Solute carrier family 6 | 55117 | Channel or | Transporter | Transporter |
| | member 15 | | transporter | | |
| SLC6A16 | Solute carrier family 6 | 28968 | Channel or | Transporter | Transporter |
| | member 16 | | transporter | | |
| SLC6A17 | Solute carrier family 6 | 388662 | Channel or | Transporter | Transporter |
| | member 17 | | transporter | | |
| SLC6A18 | Solute carrier family 6 | 348932 | Channel or | Transporter | Transporter |
| | member 18 | | transporter | | |
| SLC6A19 | Solute carrier family 6 | 340024 | Channel or | Transporter | Transporter |
| | member 19 | | transporter | | |
| SLC6A2 | Solute carrier family 6 | 6530 | Channel or | Transporter | Transporter |
| | member 2 | | transporter | | |
| SLC6A20 | Solute carrier family 6 | 54716 | Channel or | Transporter | Transporter |
| | member 20 | | transporter | | |
| SLC6A21P | Solute carrier family 6 | 652969 | Channel or | Transporter | Transporter |
| | member 21, pseudogene | | transporter | | |
| SLC6A3 | Solute carrier family 6 | 6531 | Channel or | Transporter | Transporter |
| | member 3 | | transporter | | |
| SLC6A4 | Solute carrier family 6 | 6532 | Channel or | Transporter | Transporter |
| | member 4 | | transporter | | |
| SLC6A5 | Solute carrier family 6 | 9152 | Channel or | Transporter | Transporter |
| | member 5 | | transporter | | |
| SLC6A6 | Solute carrier family 6 | 6533 | Channel or | Transporter | Transporter |
| | member 6 | | transporter | | |
| SLC6A7 | Solute carrier family 6 | 6534 | Channel or | Transporter | Transporter |
| | member 7 | | transporter | | |
| SLC6A8 | Solute carrier family 6 | 6535 | Channel or | Transporter | Transporter |
| | member 8 | | transporter | | |
| SLC6A9 | Solute carrier family 6 | 6536 | Channel or | Transporter | Transporter |
| | member 9 | | transporter | | |
| SLC7A1 | Solute carrier family 7 | 6541 | Channel or | Transporter | Transporter |
| | member 1 | | transporter | | |
| SLC7A10 | Solute carrier family 7 | 56301 | Channel or | Transporter | Transporter |
| | member 10 | | transporter | | |
| SLC7A11 | Solute carrier family 7 | 23657 | Channel or | Transporter | Transporter |
| | member 11 | | transporter | | |
| SLC7A13 | Solute carrier family 7 | 157724 | Channel or | Transporter | Transporter |
| | member 13 | | transporter | | |
| SLC7A14 | Solute carrier family 7 | 57709 | Channel or | Transporter | Transporter |
| | member 14 | | transporter | | |
| SLC7A2 | Solute carrier family 7 | 6542 | Channel or | Transporter | Transporter |
| | member 2 | | transporter | | |
| SLC7A3 | Solute carrier family 7 | 84889 | Channel or | Transporter | Transporter |
| | member 3 | | transporter | | |
| SLC7A4 | Solute carrier family 7 | 6545 | Channel or | Transporter | Transporter |
| | member 4 | | transporter | | |
| SLC7A5 | Solute carrier family 7 | 8140 | Channel or | Transporter | Transporter |
| | member 5 | | transporter | | |
| SLC7A6 | Solute carrier family 7 | 9057 | Channel or | Transporter | Transporter |
| | member 6 | | transporter | | |
| SLC7A7 | Solute carrier family 7 | 9056 | Channel or | Transporter | Transporter |
| | member 7 | | transporter | | |
| SLC7A8 | Solute carrier family 7 | 23428 | Channel or | Transporter | Transporter |
| | member 8 | | transporter | | |
| SLC7A9 | Solute carrier family 7 | 11136 | Channel or | Transporter | Transporter |
| | member 9 | | transporter | | |
| SLC8A1 | Solute carrier family 8 | 6546 | Channel or | Transporter | Transporter |
| | member A1 | | transporter | | |
| SLC8A2 | Solute carrier family 8 | 6543 | Channel or | Transporter | Transporter |
| | member A2 | | transporter | | |
| SLC8A3 | Solute carrier family 8 | 6547 | Channel or | Transporter | Transporter |
| | member A3 | | transporter | | |
| SLC8B1 | Solute carrier family 8 | 80024 | Channel or | Transporter | Transporter |
| | member B1 | | transporter | | |
| SLC9A1 | Solute carrier family 9 | 6548 | Channel or | Transporter | Transporter |
| | member A1 | | transporter | | |
| SLC9A2 | Solute carrier family 9 | 6549 | Channel or | Transporter | Transporter |
| | member A2 | | transporter | | |
| SLC9A3 | Solute carrier family 9 | 6550 | Channel or | Transporter | Transporter |
| | member A3 | | transporter | | |
| SLC9A4 | Solute carrier family 9 | 389015 | Channel or | Transporter | Transporter |
| | member A4 | | transporter | | |
| SLC9A5 | Solute carrier family 9 | 6553 | Channel or | Transporter | Transporter |
| | member A5 | | transporter | | |
| SLC9A6 | Solute carrier family 9 | 10479 | Channel or | Transporter | Transporter |
| | member A6 | | transporter | | |
| SLC9A7 | Solute carrier family 9 | 84679 | Channel or | Transporter | Transporter |
| | member A7 | | transporter | | |
| SLC9A8 | Solute carrier family 9 | 23315 | Channel or | Transporter | Transporter |
| | member A8 | | transporter | | |
| SLC9A9 | Solute carrier family 9 | 285195 | Channel or | Transporter | Transporter |
| | member A9 | | transporter | | |
| SLC9B1 | Solute carrier family 9 | 150159 | Channel or | Transporter | Transporter |
| | member B1 | | transporter | | |
| SLC9B2 | Solute carrier family 9 | 133308 | Channel or | Transporter | Transporter |
| | member B2 | | transporter | | |
| SLC9C1 | Solute carrier family 9 | 285335 | Channel or | Transporter | Transporter |
| | member C1 | | transporter | | |
| SLC9C2 | Solute carrier family 9 | 284525 | Channel or | Transporter | Transporter |
| | member C2 (putative) | | transporter | | |
| SLCO1A2 | Solute carrier organic anion | 6579 | Channel or | Transporter | Transporter |
| | transporter family member | | transporter | | |
| | 1A2 | | | | |
| SLCO1B1 | Solute carrier organic anion | 10599 | Channel or | Transporter | Transporter |
| | transporter family member | | transporter | | |
| | 1B1 | | | | |
| SLCO1B3 | Solute carrier organic anion | 28234 | Channel or | Transporter | Transporter |
| | transporter family member | | transporter | | |
| | 1B3 | | | | |
| SLCO1C1 | Solute carrier organic anion | 53919 | Channel or | Transporter | Transporter |
| | transporter family member | | transporter | | |
| | 1C1 | | | | |
| SLCO2A1 | Solute carrier organic anion | 6578 | Channel or | Transporter | Transporter |
| | transporter family member | | transporter | | |
| | 2A1 | | | | |
| SLCO2B1 | Solute carrier organic anion | 11309 | Channel or | Transporter | Transporter |
| | transporter family member | | transporter | | |
| | 2B1 | | | | |
| SLCO3A1 | Solute carrier organic anion | 28232 | Channel or | Transporter | Transporter |
| | transporter family member | | transporter | | |
| | 3A1 | | | | |
| SLCO4A1 | Solute carrier organic anion | 28231 | Channel or | Transporter | Transporter |
| | transporter family member | | transporter | | |
| | 4A1 | | | | |
| SLCO4C1 | Solute carrier organic anion | 353189 | Channel or | Transporter | Transporter |
| | transporter family member | | transporter | | |
| | 4C1 | | | | |
| SLCO5A1 | Solute carrier organic anion | 81796 | Channel or | Transporter | Transporter |
| | transporter family member | | transporter | | |
| | 5A1 | | | | |
| SLCO6A1 | Solute carrier organic anion | 133482 | Channel or | Transporter | Transporter |
| | transporter family member | | transporter | | |
| | 6A1 | | | | |
| TCIRG1 | T-cell immune regulator 1, | 10312 | Channel or | Transporter | Transporter |
| | atpase H+ transporting V0 | | transporter | | |
| | subunit a3 | | | | |
| UCP1 | Uncoupling protein 1 | 7350 | Channel or | Transporter | Transporter |
| | | | transporter | | |
| UCP2 | Uncoupling protein 2 | 7351 | Channel or | Transporter | Transporter |
| | | | transporter | | |
| UCP3 | Uncoupling protein 3 | 7352 | Channel or | Transporter | Transporter |
| | | | transporter | | |
| CACNA1A | Calcium voltage-gated | 773 | Channel or | Channel | Vgic |
| | channel subunit alpha1 A | | transporter | | |
| CACNA1B | Calcium voltage-gated | 774 | Channel or | Channel | Vgic |
| | channel subunit alpha1 B | | transporter | | |
| CACNA1C | Calcium voltage-gated | 775 | Channel or | Channel | Vgic |
| | channel subunit alpha1 C | | transporter | | |
| CACNA1D | Calcium voltage-gated | 776 | Channel or | Channel | Vgic |
| | channel subunit alpha1 D | | transporter | | |
| CACNA1E | Calcium voltage-gated | 777 | Channel or | Channel | Vgic |
| | channel subunit alpha1 E | | transporter | | |
| CACNA1F | Calcium voltage-gated | 778 | Channel or | Channel | Vgic |
| | channel subunit alpha1 F | | transporter | | |
| CACNA1G | Calcium voltage-gated | 8913 | Channel or | Channel | Vgic |
| | channel subunit alpha1 G | | transporter | | |
| CACNA1H | Calcium voltage-gated | 8912 | Channel or | Channel | Vgic |
| | channel subunit alpha1 H | | transporter | | |
| CACNA1I | Calcium voltage-gated | 8911 | Channel or | Channel | Vgic |
| | channel subunit alpha1 I | | transporter | | |
| CACNA1S | Calcium voltage-gated | 779 | Channel or | Channel | Vgic |
| | channel subunit alpha1 S | | transporter | | |
| CACNB1 | Calcium voltage-gated | 782 | Channel or | Channel | Vgic |
| | channel auxiliary subunit | | transporter | | |
| | beta 1 | | | | |
| CACNB2 | Calcium voltage-gated | 783 | Channel or | Channel | Vgic |
| | channel auxiliary subunit | | transporter | | |
| | beta 2 | | | | |
| CACNB4 | Calcium voltage-gated | 785 | Channel or | Channel | Vgic |
| | channel auxiliary subunit | | transporter | | |
| | beta 4 | | | | |
| CATSPER1 | Cation channel sperm | 117144 | Channel or | Channel | Vgic |
| | associated 1 | | transporter | | |
| CATSPER2 | Cation channel sperm | 117155 | Channel or | Channel | Vgic |
| | associated 2 | | transporter | | |
| CATSPER3 | Cation channel sperm | 347732 | Channel or | Channel | Vgic |
| | associated 3 | | transporter | | |
| CATSPER4 | Cation channel sperm | 378807 | Channel or | Channel | Vgic |
| | associated 4 | | transporter | | |
| CNGA1 | Cyclic nucleotide gated | 1259 | Channel or | Channel | Vgic |
| | channel alpha 1 | | transporter | | |
| CNGA2 | Cyclic nucleotide gated | 1260 | Channel or | Channel | Vgic |
| | channel alpha 2 | | transporter | | |
| CNGA3 | Cyclic nucleotide gated | 1261 | Channel or | Channel | Vgic |
| | channel alpha 3 | | transporter | | |
| CNGA4 | Cyclic nucleotide gated | 1262 | Channel or | Channel | Vgic |
| | channel alpha 4 | | transporter | | |
| CNGB1 | Cyclic nucleotide gated | 1258 | Channel or | Channel | Vgic |
| | channel beta 1 | | transporter | | |
| CNGB3 | Cyclic nucleotide gated | 54714 | Channel or | Channel | Vgic |
| | channel beta 3 | | transporter | | |
| HCN1 | Hyperpolarization activated | 348980 | Channel or | Channel | Vgic |
| | cyclic nucleotide gated | | transporter | | |
| | potassium channel 1 | | | | |
| HCN2 | Hyperpolarization activated | 610 | Channel or | Channel | Vgic |
| | cyclic nucleotide gated | | transporter | | |
| | potassium channel 2 | | | | |
| HCN3 | Hyperpolarization activated | 57657 | Channel or | Channel | Vgic |
| | cyclic nucleotide gated | | transporter | | |
| | potassium channel 3 | | | | |
| HCN4 | Hyperpolarization activated | 10021 | Channel or | Channel | Vgic |
| | cyclic nucleotide gated | | transporter | | |
| | potassium channel 4 | | | | |
| HVCN1 | Hydrogen voltage gated | 84329 | Channel or | Channel | Vgic |
| | channel 1 | | transporter | | |
| KCNA1 | Potassium voltage-gated | 3736 | Channel or | Channel | Vgic |
| | channel subfamily A | | transporter | | |
| | member 1 | | | | |
| KCNA10 | Potassium voltage-gated | 3744 | Channel or | Channel | Vgic |
| | channel subfamily A | | transporter | | |
| | member 10 | | | | |
| KCNA2 | Potassium voltage-gated | 3737 | Channel or | Channel | Vgic |
| | channel subfamily A | | transporter | | |
| | member 2 | | | | |
| KCNA3 | Potassium voltage-gated | 3738 | Channel or | Channel | Vgic |
| | channel subfamily A | | transporter | | |
| | member 3 | | | | |
| KCNA4 | Potassium voltage-gated | 3739 | Channel or | Channel | Vgic |
| | channel subfamily A | | transporter | | |
| | member 4 | | | | |
| KCNA5 | Potassium voltage-gated | 3741 | Channel or | Channel | Vgic |
| | channel subfamily A | | transporter | | |
| | member 5 | | | | |
| KCNA6 | Potassium voltage-gated | 3742 | Channel or | Channel | Vgic |
| | channel subfamily A | | transporter | | |
| | member 6 | | | | |
| KCNA7 | Potassium voltage-gated | 3743 | Channel or | Channel | Vgic |
| | channel subfamily A | | transporter | | |
| | member 7 | | | | |
| KCNAB1 | Potassium voltage-gated | 7881 | Channel or | Channel | Vgic |
| | channel subfamily A | | transporter | | |
| | member regulatory beta | | | | |
| | subunit 1 | | | | |
| KCNAB2 | Potassium voltage-gated | 8514 | Channel or | Channel | Vgic |
| | channel subfamily A | | transporter | | |
| | regulatory beta subunit 2 | | | | |
| KCNB1 | Potassium voltage-gated | 3745 | Channel or | Channel | Vgic |
| | channel subfamily B | | transporter | | |
| | member 1 | | | | |
| KCNB2 | Potassium voltage-gated | 9312 | Channel or | Channel | Vgic |
| | channel subfamily B | | transporter | | |
| | member 2 | | | | |
| KCNC1 | Potassium voltage-gated | 3746 | Channel or | Channel | Vgic |
| | channel subfamily C | | transporter | | |
| | member 1 | | | | |
| KCNC2 | Potassium voltage-gated | 3747 | Channel or | Channel | Vgic |
| | channel subfamily C | | transporter | | |
| | member 2 | | | | |
| KCNC3 | Potassium voltage-gated | 3748 | Channel or | Channel | Vgic |
| | channel subfamily C | | transporter | | |
| | member 3 | | | | |
| KCNC4 | Potassium voltage-gated | 3749 | Channel or | Channel | Vgic |
| | channel subfamily C | | transporter | | |
| | member 4 | | | | |
| KCND1 | Potassium voltage-gated | 3750 | Channel or | Channel | Vgic |
| | channel subfamily D | | transporter | | |
| | member 1 | | | | |
| KCND2 | Potassium voltage-gated | 3751 | Channel or | Channel | Vgic |
| | channel subfamily D | | transporter | | |
| | member 2 | | | | |
| KCND3 | Potassium voltage-gated | 3752 | Channel or | Channel | Vgic |
| | channel subfamily D | | transporter | | |
| | member 3 | | | | |
| KCNE2 | Potassium voltage-gated | 9992 | Channel or | Channel | Vgic |
| | channel subfamily E | | transporter | | |
| | regulatory subunit 2 | | | | |
| KCNE3 | Potassium voltage-gated | 10008 | Channel or | Channel | Vgic |
| | channel subfamily E | | transporter | | |
| | regulatory subunit 3 | | | | |
| KCNE4 | Potassium voltage-gated | 23704 | Channel or | Channel | Vgic |
| | channel subfamily E | | transporter | | |
| | regulatory subunit 4 | | | | |
| KCNF1 | Potassium voltage-gated | 3754 | Channel or | Channel | Vgic |
| | channel modifier subfamily | | transporter | | |
| | F member 1 | | | | |
| KCNG1 | Potassium voltage-gated | 3755 | Channel or | Channel | Vgic |
| | channel modifier subfamily | | transporter | | |
| | G member 1 | | | | |
| KCNG2 | Potassium voltage-gated | 26251 | Channel or | Channel | Vgic |
| | channel modifier subfamily | | transporter | | |
| | G member 2 | | | | |
| KCNG3 | Potassium voltage-gated | 170850 | Channel or | Channel | Vgic |
| | channel modifier subfamily | | transporter | | |
| | G member 3 | | | | |
| KCNG4 | Potassium voltage-gated | 93107 | Channel or | Channel | Vgic |
| | channel modifier subfamily | | transporter | | |
| | G member 4 | | | | |
| KCNH1 | Potassium voltage-gated | 3756 | Channel or | Channel | Vgic |
| | channel subfamily H | | transporter | | |
| | member 1 | | | | |
| KCNH2 | Potassium voltage-gated | 3757 | Channel or | Channel | Vgic |
| | channel subfamily H | | transporter | | |
| | member 2 | | | | |
| KCNH3 | Potassium voltage-gated | 23416 | Channel or | Channel | Vgic |
| | channel subfamily H | | transporter | | |
| | member 3 | | | | |
| KCNH4 | Potassium voltage-gated | 23415 | Channel or | Channel | Vgic |
| | channel subfamily H | | transporter | | |
| | member 4 | | | | |
| KCNH5 | Potassium voltage-gated | 27133 | Channel or | Channel | Vgic |
| | channel subfamily H | | transporter | | |
| | member 5 | | | | |
| KCNH6 | Potassium voltage-gated | 81033 | Channel or | Channel | Vgic |
| | channel subfamily H | | transporter | | |
| | member 6 | | | | |
| KCNH7 | Potassium voltage-gated | 90134 | Channel or | Channel | Vgic |
| | channel subfamily H | | transporter | | |
| | member 7 | | | | |
| KCNH8 | Potassium voltage-gated | 131096 | Channel or | Channel | Vgic |
| | channel subfamily H | | transporter | | |
| | member 8 | | | | |
| KCNJ1 | Potassium voltage-gated | 3758 | Channel or | Channel | Vgic |
| | channel subfamily J | | transporter | | |
| | member 1 | | | | |
| KCNJ10 | Potassium voltage-gated | 3766 | Channel or | Channel | Vgic |
| | channel subfamily J | | transporter | | |
| | member 10 | | | | |
| KCNJ11 | Potassium voltage-gated | 3767 | Channel or | Channel | Vgic |
| | channel subfamily J | | transporter | | |
| | member 11 | | | | |
| KCNJ12 | Potassium voltage-gated | 3768 | Channel or | Channel | Vgic |
| | channel subfamily J | | transporter | | |
| | member 12 | | | | |
| KCNJ13 | Potassium voltage-gated | 3769 | Channel or | Channel | Vgic |
| | channel subfamily J | | transporter | | |
| | member 13 | | | | |
| KCNJ14 | Potassium voltage-gated | 3770 | Channel or | Channel | Vgic |
| | channel subfamily J | | transporter | | |
| | member 14 | | | | |
| KCNJ15 | Potassium voltage-gated | 3772 | Channel or | Channel | Vgic |
| | channel subfamily J | | transporter | | |
| | member 15 | | | | |
| KCNJ16 | Potassium voltage-gated | 3773 | Channel or | Channel | Vgic |
| | channel subfamily J | | transporter | | |
| | member 16 | | | | |
| KCNJ2 | Potassium voltage-gated | 3759 | Channel or | Channel | Vgic |
| | channel subfamily J | | transporter | | |
| | member 2 | | | | |
| KCNJ3 | Potassium voltage-gated | 3760 | Channel or | Channel | Vgic |
| | channel subfamily J | | transporter | | |
| | member 3 | | | | |
| KCNJ4 | Potassium voltage-gated | 3761 | Channel or | Channel | Vgic |
| | channel subfamily J | | transporter | | |
| | member 4 | | | | |
| KCNJ5 | Potassium voltage-gated | 3762 | Channel or | Channel | Vgic |
| | channel subfamily J | | transporter | | |
| | member 5 | | | | |
| KCNJ6 | Potassium voltage-gated | 3763 | Channel or | Channel | Vgic |
| | channel subfamily J | | transporter | | |
| | member 6 | | | | |
| KCNJ8 | Potassium voltage-gated | 3764 | Channel or | Channel | Vgic |
| | channel subfamily J | | transporter | | |
| | member 8 | | | | |
| KCNJ9 | Potassium voltage-gated | 3765 | Channel or | Channel | Vgic |
| | channel subfamily J | | transporter | | |
| | member 9 | | | | |
| KCNK1 | Potassium two pore domain | 3775 | Channel or | Channel | Vgic |
| | channel subfamily K | | transporter | | |
| | member 1 | | | | |
| KCNK10 | Potassium two pore domain | 54207 | Channel or | Channel | Vgic |
| | channel subfamily K | | transporter | | |
| | member 10 | | | | |
| KCNK12 | Potassium two pore domain | 56660 | Channel or | Channel | Vgic |
| | channel subfamily K | | transporter | | |
| | member 12 | | | | |
| KCNK13 | Potassium two pore domain | 56659 | Channel or | Channel | Vgic |
| | channel subfamily K | | transporter | | |
| | member 13 | | | | |
| KCNK15 | Potassium two pore domain | 60598 | Channel or | Channel | Vgic |
| | channel subfamily K | | transporter | | |
| | member 15 | | | | |
| KCNK16 | Potassium two pore domain | 83795 | Channel or | Channel | Vgic |
| | channel subfamily K | | transporter | | |
| | member 16 | | | | |
| KCNK17 | Potassium two pore domain | 89822 | Channel or | Channel | Vgic |
| | channel subfamily K | | transporter | | |
| | member 17 | | | | |
| KCNK18 | Potassium two pore domain | 338567 | Channel or | Channel | Vgic |
| | channel subfamily K | | transporter | | |
| | member 18 | | | | |
| KCNK2 | Potassium two pore domain | 3776 | Channel or | Channel | Vgic |
| | channel subfamily K | | transporter | | |
| | member 2 | | | | |
| KCNK3 | Potassium two pore domain | 3777 | Channel or | Channel | Vgic |
| | channel subfamily K | | transporter | | |
| | member 3 | | | | |
| KCNK4 | Potassium two pore domain | 50801 | Channel or | Channel | Vgic |
| | channel subfamily K | | transporter | | |
| | member 4 | | | | |
| KCNK5 | Potassium two pore domain | 8645 | Channel or | Channel | Vgic |
| | channel subfamily K | | transporter | | |
| | member 5 | | | | |
| KCNK6 | Potassium two pore domain | 9424 | Channel or | Channel | Vgic |
| | channel subfamily K | | transporter | | |
| | member 6 | | | | |
| KCNK7 | Potassium two pore domain | 10089 | Channel or | Channel | Vgic |
| | channel subfamily K | | transporter | | |
| | member 7 | | | | |
| KCNK9 | Potassium two pore domain | 51305 | Channel or | Channel | Vgic |
| | channel subfamily K | | transporter | | |
| | member 9 | | | | |
| KCNMA1 | Potassium calcium- | 3778 | Channel or | Channel | Vgic |
| | activated channel subfamily | | transporter | | |
| | M alpha 1 | | | | |
| KCNN1 | Potassium calcium- | 3780 | Channel or | Channel | Vgic |
| | activated channel subfamily | | transporter | | |
| | N member 1 | | | | |
| KCNN2 | Potassium calcium- | 3781 | Channel or | Channel | Vgic |
| | activated channel subfamily | | transporter | | |
| | N member 2 | | | | |
| KCNN3 | Potassium calcium- | 3782 | Channel or | Channel | Vgic |
| | activated channel subfamily | | transporter | | |
| | N member 3 | | | | |
| KCNN4 | Potassium calcium- | 3783 | Channel or | Channel | Vgic |
| | activated channel subfamily | | transporter | | |
| | N member 4 | | | | |
| KCNQ1 | Potassium voltage-gated | 3784 | Channel or | Channel | Vgic |
| | channel subfamily Q | | transporter | | |
| | member 1 | | | | |
| KCNQ2 | Potassium voltage-gated | 3785 | Channel or | Channel | Vgic |
| | channel subfamily Q | | transporter | | |
| | member 2 | | | | |
| KCNQ3 | Potassium voltage-gated | 3786 | Channel or | Channel | Vgic |
| | channel subfamily Q | | transporter | | |
| | member 3 | | | | |
| KCNQ4 | Potassium voltage-gated | 9132 | Channel or | Channel | Vgic |
| | channel subfamily Q | | transporter | | |
| | member 4 | | | | |
| KCNQ5 | Potassium voltage-gated | 56479 | Channel or | Channel | Vgic |
| | channel subfamily Q | | transporter | | |
| | member 5 | | | | |
| KCNS1 | Potassium voltage-gated | 3787 | Channel or | Channel | Vgic |
| | channel modifier subfamily | | transporter | | |
| | S member 1 | | | | |
| KCNS2 | Potassium voltage-gated | 3788 | Channel or | Channel | Vgic |
| | channel modifier subfamily | | transporter | | |
| | S member 2 | | | | |
| KCNS3 | Potassium voltage-gated | 3790 | Channel or | Channel | Vgic |
| | channel modifier subfamily | | transporter | | |
| | S member 3 | | | | |
| KCNT1 | Potassium sodium-activated | 57582 | Channel or | Channel | Vgic |
| | channel subfamily T | | transporter | | |
| | member 1 | | | | |
| KCNT2 | Potassium sodium-activated | 343450 | Channel or | Channel | Vgic |
| | channel subfamily T | | transporter | | |
| | member 2 | | | | |
| KCNU1 | Potassium calcium- | 157855 | Channel or | Channel | Vgic |
| | activated channel subfamily | | transporter | | |
| | U member 1 | | | | |
| KCNV1 | Potassium voltage-gated | 27012 | Channel or | Channel | Vgic |
| | channel modifier subfamily | | transporter | | |
| | V member 1 | | | | |
| KCNV2 | Potassium voltage-gated | 169522 | Channel or | Channel | Vgic |
| | channel modifier subfamily | | transporter | | |
| | V member 2 | | | | |
| MCOLN1 | Mucolipin 1 | 57192 | Channel or | Channel | Vgic |
| | | | transporter | | |
| MCOLN2 | Mucolipin 2 | 255231 | Channel or | Channel | Vgic |
| | | | transporter | | |
| MCOLN3 | Mucolipin 3 | 55283 | Channel or | Channel | Vgic |
| | | | transporter | | |
| PKD2 | Polycystin 2, transient | 5311 | Channel or | Channel | Vgic |
| | receptor potential cation | | transporter | | |
| | channel | | | | |
| PKD2L1 | Polycystin 2 like 1, transient | 9033 | Channel or | Channel | Vgic |
| | receptor potential cation | | transporter | | |
| | channel | | | | |
| PKD2L2 | Polycystin 2 like 2, transient | 27039 | Channel or | Channel | Vgic |
| | receptor potential cation | | transporter | | |
| | channel | | | | |
| RYR1 | Ryanodine receptor 1 | 6261 | Channel or | Channel | Vgic |
| | | | transporter | | |
| RYR2 | Ryanodine receptor 2 | 6262 | Channel or | Channel | Vgic |
| | | | transporter | | |
| RYR3 | Ryanodine receptor 3 | 6263 | Channel or | Channel | Vgic |
| | | | transporter | | |
| SCN10A | Sodium voltage-gated | 6336 | Channel or | Channel | Vgic |
| | channel alpha subunit 10 | | transporter | | |
| SCN11A | Sodium voltage-gated | 11280 | Channel or | Channel | Vgic |
| | channel alpha subunit 11 | | transporter | | |
| SCN1A | Sodium voltage-gated | 6323 | Channel or | Channel | Vgic |
| | channel alpha subunit 1 | | transporter | | |
| SCN1B | Sodium voltage-gated | 6324 | Channel or | Channel | Vgic |
| | channel beta subunit 1 | | transporter | | |
| SCN2A | Sodium voltage-gated | 6326 | Channel or | Channel | Vgic |
| | channel alpha subunit 2 | | transporter | | |
| SCN2B | Sodium voltage-gated | 6327 | Channel or | Channel | Vgic |
| | channel beta subunit 2 | | transporter | | |
| SCN3A | Sodium voltage-gated | 6328 | Channel or | Channel | Vgic |
| | channel alpha subunit 3 | | transporter | | |
| SCN3B | Sodium voltage-gated | 55800 | Channel or | Channel | Vgic |
| | channel beta subunit 3 | | transporter | | |
| SCN4A | Sodium voltage-gated | 6329 | Channel or | Channel | Vgic |
| | channel alpha subunit 4 | | transporter | | |
| SCN4B | Sodium voltage-gated | 6330 | Channel or | Channel | Vgic |
| | channel beta subunit 4 | | transporter | | |
| SCN5A | Sodium voltage-gated | 6331 | Channel or | Channel | Vgic |
| | channel alpha subunit 5 | | transporter | | |
| SCN7A | Sodium voltage-gated | 6332 | Channel or | Channel | Vgic |
| | channel alpha subunit 7 | | transporter | | |
| SCN8A | Sodium voltage-gated | 6334 | Channel or | Channel | Vgic |
| | channel alpha subunit 8 | | transporter | | |
| SCN9A | Sodium voltage-gated | 6335 | Channel or | Channel | Vgic |
| | channel alpha subunit 9 | | transporter | | |
| TPCN1 | Two pore segment channel | 53373 | Channel or | Channel | Vgic |
| | 1 | | transporter | | |
| TPCN2 | Two pore segment channel | 219931 | Channel or | Channel | Vgic |
| | 2 | | transporter | | |
| TRPA1 | Transient receptor potential | 8989 | Channel or | Channel | Vgic |
| | cation channel subfamily A | | transporter | | |
| | member 1 | | | | |
| TRPC1 | Transient receptor potential | 7220 | Channel or | Channel | Vgic |
| | cation channel subfamily C | | transporter | | |
| | member 1 | | | | |
| TRPC2 | Transient receptor potential | 7221 | Channel or | Channel | Vgic |
| | cation channel subfamily C | | transporter | | |
| | member 2, pseudogene | | | | |
| TRPC3 | Transient receptor potential | 7222 | Channel or | Channel | Vgic |
| | cation channel subfamily C | | transporter | | |
| | member 3 | | | | |
| TRPC4 | Transient receptor potential | 7223 | Channel or | Channel | Vgic |
| | cation channel subfamily C | | transporter | | |
| | member 4 | | | | |
| TRPC5 | Transient receptor potential | 7224 | Channel or | Channel | Vgic |
| | cation channel subfamily C | | transporter | | |
| | member 5 | | | | |
| TRPC6 | Transient receptor potential | 7225 | Channel or | Channel | Vgic |
| | cation channel subfamily C | | transporter | | |
| | member 6 | | | | |
| TRPC7 | Transient receptor potential | 57113 | Channel or | Channel | Vgic |
| | cation channel subfamily C | | transporter | | |
| | member 7 | | | | |
| TRPM1 | Transient receptor potential | 4308 | Channel or | Channel | Vgic |
| | cation channel subfamily M | | transporter | | |
| | member 1 | | | | |
| TRPM2 | Transient receptor potential | 7226 | Channel or | Channel | Vgic |
| | cation channel subfamily M | | transporter | | |
| | member 2 | | | | |
| TRPM3 | Transient receptor potential | 80036 | Channel or | Channel | Vgic |
| | cation channel subfamily M | | transporter | | |
| | member 3 | | | | |
| TRPM4 | Transient receptor potential | 54795 | Channel or | Channel | Vgic |
| | cation channel subfamily M | | transporter | | |
| | member 4 | | | | |
| TRPM5 | Transient receptor potential | 29850 | Channel or | Channel | Vgic |
| | cation channel subfamily M | | transporter | | |
| | member 5 | | | | |
| TRPM6 | Transient receptor potential | 140803 | Channel or | Channel | Vgic |
| | cation channel subfamily M | | transporter | | |
| | member 6 | | | | |
| TRPM7 | Transient receptor potential | 54822 | Channel or | Channel | Vgic |
| | cation channel subfamily M | | transporter | | |
| | member 7 | | | | |
| TRPM8 | Transient receptor potential | 79054 | Channel or | Channel | Vgic |
| | cation channel subfamily M | | transporter | | |
| | member 8 | | | | |
| TRPV1 | Transient receptor potential | 7442 | Channel or | Channel | Vgic |
| | cation channel subfamily V | | transporter | | |
| | member 1 | | | | |
| TRPV2 | Transient receptor potential | 51393 | Channel or | Channel | Vgic |
| | cation channel subfamily V | | transporter | | |
| | member 2 | | | | |
| TRPV3 | Transient receptor potential | 162514 | Channel or | Channel | Vgic |
| | cation channel subfamily V | | transporter | | |
| | member 3 | | | | |
| TRPV4 | Transient receptor potential | 59341 | Channel or | Channel | Vgic |
| | cation channel subfamily V | | transporter | | |
| | member 4 | | | | |
| TRPV5 | Transient receptor potential | 56302 | Channel or | Channel | Vgic |
| | cation channel subfamily V | | transporter | | |
| | member 5 | | | | |
| TRPV6 | Transient receptor potential | 55503 | Channel or | Channel | Vgic |
| | cation channel subfamily V | | transporter | | |
| | member 6 | | | | |

Lgic = ligand-gated ion channel,
Vgic = voltage-gated ion channel,
Other_ic = other ion channel

Agent Modalities

A neuromodulating agent can be a number of different modalities. A neuromodulating agent can be a nucleic acid molecule (e.g., DNA molecule or RNA molecule, e.g., mRNA, guide RNA (gRNA), or inhibitory RNA molecule (e.g., siRNA, shRNA, or miRNA), or a hybrid DNA-RNA molecule), a small molecule (e.g., a neurotransmitter, an agonist, antagonist, or an epigenetic modifier), a peptide, or a polypeptide (e.g., an antibody molecule, e.g., an antibody or antigen binding fragment thereof, or a neuropeptide). A neuromodulating agent can also be a viral vector expressing a neurome gene or a cell infected with a viral vector. Any of these modalities can be a neuromodulating agent directed to target (e.g., to agonize or to inhibit) a gene or protein in a neurotransmitter, neuropeptide, neuronal growth factor, or neurome gene (e.g., biosynthesis, channel, transporter, ligand, receptor, signaling, synaptic, structural, or vesicular) pathway described herein (e.g., a gene or protein listed in Tables 1A-1C, Table 7, or Table 8).

The nucleic acid molecule, small molecule, peptide, polypeptide, or antibody molecule can be modified. For example, the modification can be a chemical modification, e.g., conjugation to a marker, e.g., fluorescent marker or a radioactive marker. In other examples, the modification can include conjugation to a molecule that enhances the stability or half-life of the neuromodulating agent. The modification can also include conjugation to an antibody to target the agent to a particular cell or tissue. Additionally, the modification can be a chemical modification, packaging modification (e.g., packaging within a nanoparticle or microparticle), or targeting modification to prevent the agent from crossing the blood brain barrier.

Small Molecules

Numerous small molecule neuromodulating agents useful in the methods of the invention are described herein and additional small molecule neuromodulating agents useful as therapies for cancer can also be screened based on their ability to modulate sympathetic and parasympathetic neural pathways. Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

In some embodiments, the neuromodulating agent is an agonist or antagonist listed in column 2 or column 3 of Table 2A or column 2 of Tables 2B-2L, which is directed to the corresponding neurotransmitter pathway member listed in column 1 of Tables 2A-2L. In some embodiments, the neuromodulating agent is a neurotransmitter or neuropeptide listed in Table 1A, 1B, or encoded by a gene in Table 7, or a neuronal growth factor listed in Table 1C or encoded by a gene in Table 7. Agonists and antagonists can be used to treat a disorder or condition described herein. A pharmaceutical composition comprising the agonist, antagonist, neurotransmitter, neuropeptide, or neuronal growth factor can be formulated for treatment of a cancer described herein. In some embodiments, a pharmaceutical composition that includes the agonist or antagonist is formulated for local administration, e.g., to the affected site in a subject.

Polypeptides

In embodiments, a neuromodulating agent described herein comprises a neuromodulating agent polypeptide or an analog thereof. For example, a neuromodulating agent described herein is a neuropeptide or an analog thereof.

The neuromodulating agent can be a neuropeptide listed in Table 1A or 1B, a neuronal growth factor listed in Table 1C, or a protein encoded by a neurome gene listed in Table 7 or Table 8 (e.g., a biosynthesis, channel, transporter, ligand, receptor, signaling, synaptic, structural, or vesicular protein), wherein the primary sequence of the neuromodulating agent is provided by reference to accession number or Entrez Gene ID. The agent can be a polypeptide having the sequence referenced by accession number or Entrez Gene ID of a neuropeptide listed in Table 1A or 1B, a neuronal growth factor listed in Table 1C, or a protein encoded by a neurome gene listed in Table 7, or an analog thereof, e.g., a sequence having at least 75%, 80%, 85%, 90%, 90%, 98%, 99% or 100% identity to the sequence referenced by accession number or Entrez Gene ID.

Percent identity in the context of two or more polypeptide sequences or nucleic acids, refers to two or more sequences that are the same. Two sequences are “substantially identical” if two sequences have a specified percentage of amino acid residues or nucleotides that are the same (e.g., at least 60% identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region, or, when not specified, over the entire sequence), when compared and aligned for maximum correspondence over a comparison window, or designated region as measured using a sequence comparison algorithms or by manual alignment and visual inspection. In some cases, the identity (or substantial identity) exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, Adv. Appl. Math. 2:482c, 1970, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389, 1977; and Altschul et al., J. Mol. Biol. 215:403, 1990, respectively. Software for performing BLAST analyses is publicly available through the National Center for Biotechnology Information.

The percent identity between two amino acid sequences can also be determined using the algorithm of E. Meyers and W. Miller, Comput. Appl. Biosci. 4:11, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Methods of making a therapeutic polypeptide are routine in the art. See, in general, Smales & James (Eds.), Therapeutic Proteins: Methods and Protocols (Methods in Molecular Biology), Humana Press 2005; and Crommelin, Sindelar & Meibohm (Eds.), Pharmaceutical Biotechnology: Fundamentals and Applications, Springer 2013.

Some methods for producing a neuromodulating agent polypeptide involve expression in mammalian cells, although recombinant proteins can also be produced using insect cells, yeast, bacteria, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press 2012.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer 2014.

Purification of protein therapeutics is known and is described, e.g., in Franks, Protein Biotechnology: Isolation, Characterization, and Stabilization, Humana Press 2013; and in Cutler, Protein Purification Protocols (Methods in Molecular Biology), Humana Press 2010.

Formulation of protein therapeutics is known and is described, e.g., in Meyer (Ed.), Therapeutic Protein Drug Products: Practical Approaches to formulation in the Laboratory, Manufacturing, and the Clinic, Woodhead Publishing Series 2012.

Antibodies

The neuromodulating agent can be an antibody or antigen binding fragment thereof. For example, a neuromodulating agent described herein is an antibody that blocks or potentiates activity and/or function of a receptor, neuropeptide, neurotransmitter or transporter listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene).

The making and use of therapeutic antibodies against a target antigen (e.g., against a protein in a neurotransmitter pathway described herein (e.g., a protein product of a gene listed in Table 1)) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Synthetic mRNA

In some embodiments, the neuromodulating agent is an mRNA molecule, e.g., a synthetic mRNA molecule encoding a protein listed in Tables 1A-1C, or a protein encoded by a gene in Table 7 or Table 8. The mRNA molecule may increase the level (e.g., protein and/or mRNA level) and/or activity or function of a neurotransmitter, neurotransmitter receptor, neuropeptide, neuropeptide receptor, neuronal growth factor, or neurome gene in Table 7 or Table 8 (e.g., a biosynthesis, channel, transporter, ligand, receptor, signaling, synaptic, structural, or vesicular gene), e.g., a positive regulator of function. The mRNA molecule can encode a neuromodulating agent or a fragment thereof. For example, the mRNA molecule encodes a polypeptide having at least 50% (e.g., at least 50%, 60%, 70%, 80%, 90%, 95%, 97%, 99%, or greater) identity to the amino acid sequence of a neuromodulating agent listed in Table 1A, a ligand listed in Table 1B, a neuronal growth factor listed in Table 1C, or neurome gene in Table 7 or Table 8 (e.g., a biosynthesis, channel, transporter, ligand, receptor, signaling, synaptic, structural, or vesicular gene), all with reference to accession number or Entrez Gene ID provided. In other examples, the mRNA molecule has at least 50% (e.g., at least 50%, 60%, 70%, 80%, 90%, 95%, 97%, 99%, or greater) identity to the nucleic acid sequence of a neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene). The mRNA molecule can encode an amino acid sequence differing by no more than 30 (e.g., no more than 30, 20, 10, 5, 4, 3, 2, or 1) amino acids to the amino acid sequence of a neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. The mRNA molecule can have a sequence encoding a fragment of a neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. For example, the fragment comprises 10-20, 20-40, 40-60, 60-80, 80-100, 100-120, 120-140, 140-160, 160-180, 180-200, 200-250, 250-300, 300-400, 400-500, 500-600, or more amino acids in length. In embodiments, the fragment is a functional fragment, e.g., having at least 20%, e.g., at least 20%, 30%, 40%, 50%, 60%, 70%, 80%, 90%, or greater, of an activity of a full length neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. In embodiments, the mRNA molecule increases the level and/or activity or function of or encodes a neuromodulating agent (or fragment thereof).

The synthetic mRNA molecule can be modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some embodiments, the modified RNA encoding a neuromodulating agent of interest described herein has modified nucleosides or nucleotides. Such modifications are known and are described, e.g., in WO2012019168. Additional modifications are described, e.g., in WO2015038892; WO2015038892; WO2015089511; WO2015196130; WO2015196118 and WO2015196128A2.

In some embodiments, the modified RNA encoding a polypeptide of interest described herein has one or more terminal modifications, e.g., a 5′ Cap structure and/or a poly-A tail (e.g., of between 100-200 nucleotides in length). The 5′ cap structure may be selected from the group consisting of CapO, CapI, ARCA, inosine, NI-methyl-guanosine, 2′ fluoro-guanosine, 7-deaza-guanosine, 8-oxo-guanosine, 2-amino-guanosine, LNA-guanosine, and 2-azido-guanosine. In some cases, the modified RNAs also contain a 5′ UTR comprising at least one Kozak sequence, and a 3′ UTR. Such modifications are known and are described, e.g., in WO2012135805 and WO2013052523. Additional terminal modifications are described, e.g., in WO2014164253 and WO2016011306. WO2012045075 and WO2014093924

Chimeric enzymes for synthesizing capped RNA molecules (e.g., modified mRNA) which may include at least one chemical modification are described in WO2014028429.

In some embodiments, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO2013151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO2013151666, WO2013151668, WO2013151663, WO2013151669, WO2013151670, WO2013151664, WO2013151665, WO2013151671, WO2013151672, WO2013151667 and WO2013151736.S Methods of purification include purifying an RNA transcript comprising a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO2014152031); using ion (e.g., anion) exchange chromatography that allows for separation of longer RNAs up to 10,000 nucleotides in length via a scalable method (WO2014144767); and subjecting a modified mRNA sample to DNAse treatment (WO2014152030).

Formulations of modified RNAs are known and are described, e.g., in WO2013090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO2013151666, WO2013151668, WO2013151663, WO2013151669, WO2013151670, WO2013151664, WO2013151665, and WO2013151736; Tables 6 and 7 of International Publication No. WO2013151672; Tables 6, 178 and 179 of International Publication No. WO2013151671; Tables 6, 185 and 186 of International Publication No. WO2013151667. Any of the foregoing may be synthesized as an IVT polynucleotide, chimeric polynucleotide or a circular polynucleotide, and each may comprise one or more modified nucleotides or terminal modifications.

Inhibitory RNA

In some embodiments, the neuromodulating agent is an inhibitory RNA molecule, e.g., that acts by way of the RNA interference (RNAi) pathway. An inhibitory RNA molecule can decrease the expression level (e.g., protein level or mRNA level) of a neurotransmitter, neuropeptide, receptor, neuronal growth factor, or neurome gene listed herein. For example, an inhibitory RNA molecule includes a short interfering RNA, short hairpin RNA, and/or a microRNA that targets a full length neuromodulating agent listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. A siRNA is a double-stranded RNA molecule that typically has a length of about 19-25 base pairs. A shRNA is a RNA molecule comprising a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. MiRNAs bind to target sites on mRNA molecules and silence the mRNA, e.g., by causing cleavage of the mRNA, destabilization of the mRNA, or inhibition of translation of the mRNA. In embodiments, the inhibitory RNA molecule decreases the level and/or activity of a negative regulator of function or a positive regulator of function. In other embodiments, the inhibitor RNA molecule decreases the level and/or activity of an inhibitor of a positive regulator of function.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of a neuromodulating agent. In embodiments, the inhibitory RNA molecule inhibits expression of a neuromodulating agent (e.g., inhibits translation to protein). In other embodiments, the inhibitor RNA molecule increases degradation of a neuromodulating agent and/or decreases the stability (i.e., half-life) of a neuromodulating agent. The inhibitory RNA molecule can be chemically synthesized or transcribed in vitro.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

Gene Editing

In some embodiments, the neuromodulating agent is a component of a gene editing system. For example, the neuromodulating agent introduces an alteration (e.g., insertion, deletion (e.g., knockout), translocation, inversion, single point mutation, or other mutation) in a gene related to a neurotransmitter pathway, e.g., a neuropeptide or receptor gene described in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al. Trends Biotechnol. 31.7 (2013):397-405.

CRISPR refers to a set of (or system comprising a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341: 833, 2013. In some examples, the CRISPR system includes the Cas9 protein, a nuclease that cuts on both strands of the DNA. See, e.g., i.d.

In some embodiments, in a CRISPR system for use described herein, e.g., in accordance with one or more methods described herein, the spacers of the CRISPR are derived from a target gene sequence, e.g., from a sequence (with reference to the accession number) of a neurotransmitter pathway gene, e.g., a neuropeptide or receptor gene listed in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

In some embodiments, the neuromodulating agent includes a guide RNA (gRNA) for use in a clustered regulatory interspaced short palindromic repeat (CRISPR) system for gene editing. In embodiments, the neuromodulating agent comprises a zinc finger nuclease (ZFN), or an mRNA encoding a ZFN, that targets (e.g., cleaves) a nucleic acid sequence (e.g., DNA sequence) of a gene related to a neurotransmitter pathway, e.g., a neuropeptide or receptor gene described in Table 1. In embodiments, the neuromodulating agent comprises a TALEN, or an mRNA encoding a TALEN, that targets (e.g., cleaves) a nucleic acid sequence (e.g., DNA sequence) in a gene related to a neurotransmitter pathway, e.g., a neuropeptide or receptor gene described in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

For example, the gRNA can be used in a CRISPR system to engineer an alteration in a gene (e.g., a gene related to a neurotransmitter pathway, e.g., a neuropeptide, neurotransmitter, neuronal growth factor or receptor gene described in Tables 1A, 1B, or 1C, or a neurome gene listed in Table 7 or Table 8 (e.g., a biosynthesis, channel, transporter, ligand, receptor, signaling, synaptic, structural, or vesicular gene)). In other examples, the ZFN and/or TALEN can be used to engineer an alteration in a gene (e.g., a gene related to a neurotransmitter pathway, e.g., a neuropeptide, neurotransmitter, neuronal growth factor, or receptor gene described in Tables 1A, 1B, or 1C, or a neurome gene listed in Table 7 or Table 8 (e.g., a biosynthesis, channel, transporter, ligand, receptor, signaling, synaptic, structural, or vesicular gene)). Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration increases the level and/or activity of a neuromodulator, e.g., the alteration is a positive regulator of function. In other examples, the alteration decreases the level and/or activity of (e.g., knocks down or knocks out) a neuromodulator, e.g., the alteration is a negative regulator of function. In yet another example, the alteration corrects a defect (e.g., a mutation causing a defect), in a gene related to a neurotransmitter pathway, e.g., a neuropeptide or receptor gene described in Table 1A, a ligand listed in Table 1B, a neuronal growth factor listed in Table 1C, or a neurome gene listed in Table 7 or Table 8 (e.g., a biosynthesis, channel, transporter, ligand, receptor, signaling, synaptic, structural, or vesicular gene), all with reference to accession number or Entrez Gene ID provided.

In certain embodiments, the CRISPR system is used to edit (e.g., to add or delete a base pair) a target gene, e.g., a neuromodulating agent, e.g., described herein. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a neuromodulator, e.g., described herein, for example, thereby blocking an RNA polymerase sterically.

In some embodiments, a CRISPR system can be generated to edit a neuromodulator (e.g., a gene related to a neurotransmitter pathway, e.g., a neuropeptide or receptor gene described in Table 1A-1C), using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., a gene encoding a neuromodulating agent (e.g., a neuropeptide, neurotransmitter, neuronal growth factor, neurome gene, or receptor described herein). In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-g RNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a neuromodulating agent (e.g., a neuropeptide, neurotransmitter, neuronal growth factor, neurome gene, or receptor described herein). In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5, 2016, incorporated herein by reference. In addition, dCas9-mediated epigenetic modifications and simultaneous activation and repression using CRISPR systems, as described in Dominguez et al., can be used to modulate a thymic function modulator or thymic function factor described herein.

Viral Vectors

The neuromodulating agent can be a viral vector (e.g., a viral vector expressing a neurome gene). Viral vectors can be used to express a transgene encoding a neurotransmitter, neuropeptide, receptor, or neuronal growth factor from Tables 1A-1C or a neurome gene in Table 7 or Table 8 (e.g., a biosynthesis, channel, transporter, ligand, receptor, signaling, synaptic, structural, or vesicular gene), all with reference to accession number or Entrez Gene ID provided. A viral vector may be administered to a cell or to a subject (e.g., a human subject or animal model) to increase expression of a neurotransmitter, neuropeptide, receptor, or neuronal growth factor from Tables 1A-1C or a neurome gene in Table 7 or Table 8 (e.g., a biosynthesis, channel, transporter, ligand, receptor, signaling, synaptic, structural, or vesicular gene). Viral vectors can also be used to express a neurotoxin from Table 3. A viral vector expressing a neurotoxin from Table 3 can be administered to a cell or to a subject (e.g., a human subject or animal model) to decrease neurotransmission. Viral vectors can be directly administered (e.g., injected) to a or tumor to treat cancer.

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Screening for New Agents

The invention also features a method of screening for an agent for the treatment of cancer. The method includes (a) providing a plurality of test agents, (b) evaluating the plurality of test agents for neuromodulating activity, and (c) selecting a test agent of the plurality as an anti-cancer if the test agent exhibits neuromodulating activity. The evaluation method can include introducing one or more test agents into a co-culture system containing at least one neuronal cell and at least one non-neuronal cell.

In certain embodiments, evaluating an agent for neuromodulating activity includes one or more of evaluating the agent for: ability to inhibit or potentiate a beta adrenergic pathway, ability to inhibit or potentiate a cholinergic pathway, ability to inhibit or potentiate a dopaminergic pathway, ability to inhibit or potentiate a serotonin pathway, ability of the agent to increase or decrease neurogenesis; ability to potentiate or inhibit the transmission of a nerve impulse; ability of the agent to increase or decrease neurome gene expression; ability of the agent to increase neurite (e.g., axon or dendrite) outgrowth; ability to increase or decrease synapse formation or maintenance; ability to increase or decrease neuropeptide signaling; or ability to increase or decrease innervation of a tissue or tumor. The method can include correlating the neuromodulating effect of an agent with a predicted effect of the agent on a mammal, e.g., a human, e.g., by providing (e.g., to the government, a health care provider, insurance company or patient) informational, marketing or instructional material, e.g., print material or computer readable material (e.g., a label, patient record or email), related to the agent or its use, identifying the agent as a possible or predicted treatment in a mammal, e.g., a human. The method can include identifying the agent as a treatment for, or lead compound for treatment of, cancer, e.g., a condition described herein. The identification can be in the form of informational, marketing or instructional material. In one embodiment, the methods include correlating a value for neuromodulation activity with ability to treat a cancer described herein by, e.g., generating a dataset of the correlation.

Evaluating the effect of the agent on neuromodulation can include administering the agent in-vivo to an experimental mammal, or in-vitro or ex-vivo to a nerve or nervous tissue of an animal and evaluating the effect of the agent on the mammal, nerve or nervous tissue. In some embodiments, the evaluation includes entering a value for the evaluation, e.g., into a database or other record. In some embodiments, the subject is an experimental animal, e.g., a wild-type or a transgenic experimental animal.

In some embodiments, the identifying step includes: (a) contacting the agent with a cell or tissue or non-human animal whose genome includes an exogenous nucleic acid that includes a regulatory region of a neuroactive protein, operably linked to a nucleotide sequence encoding a reporter polypeptide (e.g., a light based, e.g., a colorimetric (e.g., LacZ) or fluorescently detectable label, e.g., a fluorescent reporter polypeptide, e.g., GFP, EGFP, BFP, RFP); (b) evaluating the ability of a test agent to modulate the expression of the reporter polypeptide in the cell, tissue or non-human animal; and (c) selecting a test agent that modulates the expression of the reporter polypeptide as an agent that is useful in the treatment of cancer or an inflammatory or autoimmune condition described herein. In one embodiment, the cell or tissue is a nerve cell or tissue. In another embodiment, the non-human animal is a transgenic animal, e.g., a transgenic rodent, e.g., a mouse, rat or guinea pig, harboring the nucleic acid.

The test agents can be, e.g., nucleic acids (e.g., antisense RNA, ribozymes, modified mRNAs encoding an agent protein), polypeptides (antibodies or antigen-binding fragment thereof), peptide fragments, peptidomimetics, or small molecules (e.g., a small organic molecule with a molecular weight of less than 2000 daltons). In another embodiment, the test agent is a member of a combinatorial library, e.g., a peptide, antibody or organic combinatorial library, or a natural product library. In some embodiments, a plurality of test agents, e.g., library members, is tested. The test agents of the plurality, e.g., library, may share structural or functional characteristics. The test agent can also be a crude or semi-purified extract, e.g., a botanical extract such as a plant extract, or algal extract.

In one embodiment, the method includes two evaluating steps, e.g., the method includes a first step of evaluating the test agent in a first system, e.g., an in-vitro or cell-based or tissue system, and a second step of evaluating the test agent in a second system, e.g., a second cell or tissue system or in a non-human experimental animal (e.g., a rodent, a pig, a dog, a non-human primate). In other embodiments, the methods include two evaluating steps in the same type of system, e.g., the agent is re-evaluated in a non-human animal after a first evaluation in the same or a different non-human animal. The two evaluations can be separated by any length of time, e.g., days, weeks, months or years.

In some embodiments, the plurality of test agents are agents that do not cross the blood brain barrier. In some embodiments, the plurality of test agents is evaluated for ability to cross the blood brain barrier.

II. Blood Brain Barrier Permeability

In some embodiments, the neuromodulating agents for use in the present invention are agents that are not capable of crossing, or that do not cross, the blood brain barrier (BBB) of a mammalian subject. The BBB is a highly selective semipermeable membrane barrier that separates the circulating blood from the brain extracellular fluid (e.g., cerebrospinal fluid) in the central nervous system (CNS). The BBB is made up of high-density endothelial cells, which are connected by tight junctions. These cells prevent most molecular compounds in the bloodstream (e.g., large molecules and hydrophilic molecules) from entering the brain. Water, some gases (e.g., oxygen and carbon dioxide), and lipid-soluble molecules (e.g., hydrophobic molecules, such as steroid hormones) can cross the BBB by passive diffusion. Molecules that are needed for neural function, such as glucose and amino acids, are actively transported across the BBB.

A number of approaches can be used to render an agent BBB impermeable. These methods include modifications to increase an agent's size, polarity, or flexibility or reduce its lipophilicity, targeting approaches to direct an agent to another part of the body and away from the brain, and packaging approaches to deliver an agent in a form that does not freely diffuse across the BBB. These approaches can be used to render a BBB permeable neuromodulating agent impermeable, and they can also be used to improve the properties (e.g., cell-specific targeting) of a neuromodulating agent that does not cross the BBB. The methods that can be used to render an agent BBB impermeable are discussed in greater detail herein below.

Formulation of BBB-Permeable Agents for Enhanced Cell Targeting

One approach that can be used to render a neuromodulating agent BBB impermeable is to conjugate the agent to a targeting moiety that directs it somewhere other than the brain. The targeting moiety can be an antibody for a receptor expressed by the target cell (e.g., N-Acetylgalactosamine for liver transport; DGCR2, GBF1, GPR44 or SerpinB10 for pancreas transport; Secretoglobin, family 1A, member 1 for lung transport). The targeting moiety can also be a ligand of any receptor or other molecular identifier expressed on the target cell in the periphery. These targeting moieties can direct the neuromodulating agent of interest to its corresponding target cell, and can also prevent BBB crossing by directing the agent away from the BBB and increasing the size of the neuromodulating agent via conjugation of the targeting moiety.

Neuromodulating agents can also be rendered BBB impermeable through formulation in a particulate delivery system (e.g., a nanoparticle, liposome, or microparticle), such that the agent is not freely diffusible in blood and cannot cross the BBB. The particulate formulation used can be chosen based on the desired localization of the neuromodulating agent (e.g., a tumor), as particles of different sizes accumulate in different locations. For example, nanoparticles with a diameter of 45 nm or less enter the lymph node, while 100 nm nanoparticles exhibit poor lymph node trafficking. Some examples of the link between particle size and localization in vivo are described in Reddy et al., J Controlled Release 112:26 2006, and Reddy et al., Nature Biotechnology 25:1159 2007.

Neuromodulating agents can be tested after the addition of a targeting moiety or after formulation in a particulate delivery system to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A neuromodulating agent that exhibits BBB impermeability can be used in the methods described herein.

Modification of Existing Compounds to Render them BBB Impermeable

There are multiple parameters that have been empirically derived in the field of medicinal chemistry to predict whether a compound will cross the BBB. The most common numeric value for describing permeability across the BBB is the log BB, defined as the logarithmic ratio of the concentration of a compound in the brain and in the blood. Empirical rules of thumb have been developed to predict BBB permeability, including rules regarding molecular size, polar surface area, sum of oxygen and nitrogen atoms, lipophilicity (e.g., partition coefficient between apolar solvent and water), “lipoaffinity”, molecular flexibility, and number of rotable bonds (summarized in Muehlbacher et al., J Comput Aided Mol Des. 25: 1095 2011; and Geldenhuys et al., Ther Deliv. 6: 961 2015). Some preferred limits on various parameters for BBB permeability are listed in Table 1 of Ghose et al., ACS Chem Neurosci. 3: 50 2012, which is incorporated herein by reference. Based on the parameters shown in the table, one of skill in the art could modify an existing neuromodulating agent to render it BBB impermeable.

One method of modifying a neuromodulating agent to prevent BBB crossing is to add a molecular adduct that does not affect the target binding specificity, kinetics, or theromodynamics of the agent. Molecular adducts that can be used to render an agent BBB impermeable include polyethylene glycol (PEG), a carbohydrate monomer or polymer, a dendrimer, a polypeptide, a charged ion, a hydrophilic group, deuterium, and fluorine. Neuromodulating agents can be tested after the addition of one or more molecular adducts or after any other properties are altered to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A neuromodulating agent that exhibits BBB impermeability can be used in the methods described herein.

Screening for or Development of BBB Impermeable Agents

Another option for developing BBB impermeable agents is to find or develop new agents that do not cross the BBB. One method for finding new BBB impermeable agents is to screen for compounds that are BBB impermeable. Compound screening can be performed using in vitro models (e.g., monolayer models, co-culture models, dynamic models, multi-fluidic models, isolated brain microvessels), in vivo models, and computational models, as described in He et al., Stroke 45:2514 2014; Bickel, NeuroRx 2:15 2005; Wang et al., Int J Pharm 288:349 2005, and Czupalla et al., Methods Mol Biol 1135:415 2014. For example, the ability of a molecule to cross the blood brain barrier can be determined in vitro using a transwell BBB assay in which microvascular endothelial cells and pericytes are co-cultured separated by a thin macroporous membrane, see e.g., Naik et al., J Pharm Sci 101:1337 2012 and Hanada et al., Int J Mol Sci 15:1812 2014; or in vivo by tracking the brain uptake of the target molecule by histology or radio-detection. Compounds would be deemed appropriate for use as neuromodulating agents in the methods described herein if they do not display BBB permeability in the aforementioned models.

III. Cancer

The methods described herein can be used to treat cancer in a subject by administering to the subject an effective amount of a neuromodulating agent, e.g., a neuromodulating agent described herein. The method may include administering locally (e.g., intratumorally) to the subject a neuromodulating agent described herein in a dose (e.g., effective amount) and for a time sufficient to treat the cancer.

The neuromodulating agent may inhibit proliferation or disrupt the function of non-neural cells associated with the cancer, e.g., the method includes administering to the subject an effective amount of a neuromodulating agent for a time sufficient to inhibit proliferation or disrupt the function of non-neural cells associated with the cancer. Non-neural cells associated with the cancer include malignant cancer cells, malignant cancer cells in necrotic and hypoxic areas, adipocytes, pericytes, endothelial cells, cancer associated fibroblasts, fibroblasts, mesenchymal stem cells, red blood cells, or extracellular matrix. The proliferation of non-neural cells associated with the cancer may be decreased in the subject at least 1%, 2%, 5%, 10%, 15%, 20%, 25%, 30%, 35%, 40%, 50%, 60%, 70%, 80%, 90%, 95% or more, compared to before the administration. The proliferation of non-neural cells associated with the cancer can be decreased in the subject between 5-20%, between 5-50%, between 10-50%, between 20-80%, or between 20-70%.

The neuromodulating agent can be administered in an amount sufficient to treat cancer. For example, the stroma associated with the tumor, e.g., fibroblasts, is disrupted such that an essential function, e.g., the production of matrix metalloproteases, is altered to inhibit tumor survival or promote tumor control.

The neuromodulating agent can treat cancer by increasing cancer cell death in a subject (e.g., a human subject or animal model) or in a cancer cell culture (e.g., a culture generated from a patient tumor sample, a cancer cell line, or a repository of patient samples). A neuromodulating agent can increase cancer cell death by at least 1%, 2%, 5%, 10%, 15%, 20%, 25%, 30%, 35%, 40%, 50%, 60%, 70%, 80%, 90%, 95% or more compared to before administration to a subject or cancer cell culture. A neuromodulating agent can increase cancer cell death in a subject or cancer cell culture between 5-20%, between 5-50%, between 10-50%, between 20-80%, between 20-70%.

The neuromodulating agent can also act to inhibit cancer cell growth, proliferation, metastasis, or invasion, e.g., the method includes administering to the subject (e.g., a human subject or animal model) or a cancer cell culture (e.g., a culture generated from a patient tumor sample, a cancer cell line, or a repository of patient samples) a neuromodulating agent in an amount (e.g., an effective amount) and for a time sufficient to inhibit cancer cell growth, proliferation, metastasis, or invasion. Cancer cell growth, proliferation, metastasis, or invasion can be decreased in the subject or cancer cell culture at least 1%, 2%, 5%, 10%, 15%, 20%, 25%, 30%, 35%, 40%, 50%, 60%, 70%, 80%, 90%, 95% or more, compared to before the administration Cancer cell growth, proliferation, metastasis, or invasion can be decreased in the subject or cancer cell culture between 5-20%, between 5-50%, between 10-50%, between 20-80%, between 20-70%.

The neuromodulating agent can inhibit cancer cell invasion or metastasis along a nerve, e.g., the method includes administering to the subject (e.g., a human subject or animal model) a neuromodulating agent in an amount (e.g., an effective amount) and for a time sufficient to inhibit cancer cell invasion or metastasis along a nerve. For example, the neuromodulating agent is an antibody against a ligand selected from: Galanin; Semaphorin-4F; Caveolin-1; a chemokine such as CCL2, CCR2, CXCL12, and CXCR4; GDNF; GFRα1; NGF; neurotrophin-3 or -4; substance P; Neuropeptide Y; Peptide YY; Vasoactive intestinal peptide (VIP); or NCAM1. In other examples, the neuromodulating agent can be a receptor antagonist against the receptor for a ligand selected from: Galanin; Semaphorin-4F; Caveolin-1; a chemokine such as CCL2, CCR2, CXCL12, and CXCR4; GDNF; GFRα1; NGF; neurotrophin-3 or -4; substance P; Neuropeptide Y; Peptide YY; Vasoactive intestinal peptide (VIP); or NCAM1. The neuromodulating can decrease cancer cell invasion or metastasis along a nerve in the subject at least 1%, 2%, 5%, 10%, 15%, 20%, 25%, 30%, 35%, 40%, 50%, 60%, 70%, 80%, 90%, 95% or more, compared to before the administration. The neuromodulating agent can decrease cancer cell invasion or metastasis along a nerve in the subject between 5-20%, between 5-50%, between 10-50%, between 20-80%, between 20-70%.

The neuromodulating agent can also reduce the number of nerve fibers in the affected tissue or reduce the activity of peripheral nerve fibers in the affected tissue. For example, the method includes administering to the subject (e.g., a human subject or animal model) a neuromodulating agent in an amount (e.g., an effective amount) and for a time sufficient to reduce the number of nerve fibers in the affected tissue or reduce the activity of peripheral nerve fibers in the affected tissue. The affected tissue can be a tumor, a tumor micro-environment, or the bone marrow niche. The number of nerve fibers in the affected tissue or the activity of peripheral nerve fibers in the affected tissue can be decreased in the subject at least 1%, 2%, 5%, 10%, 15%, 20%, 25%, 30%, 35%, 40%, 50%, 60%, 70%, 80%, 90%, 95% or more, compared to before the administration. The number of nerve fibers in the affected tissue or the activity of peripheral nerve fibers in the affected tissue can be decreased in the subject between 5-20%, between 5-50%, between 10-50%, between 20-80%, or between 20-70%.

The neuromodulating agent can also increase the number of nerve fibers in the affected tissue or increase the activity of peripheral nerve fibers in the affected tissue. For example, the method includes administering to the subject (e.g., a human subject or animal model) a neuromodulating agent in an amount (e.g., an effective amount) and for a time sufficient to increase the number of nerve fibers in the affected tissue or increase the activity of peripheral nerve fibers in the affected tissue. The affected tissue can be a tumor, a tumor micro-environment, or the bone marrow niche. The number of nerve fibers in the affected tissue or the activity of peripheral nerve fibers in the affected tissue can be increased in the subject at least 1%, 2%, 5%, 10%, 15%, 20%, 25%, 30%, 35%, 40%, 50%, 60%, 70%, 80% or more, compared to before the administration. The number of nerve fibers in the affected tissue or the activity of peripheral nerve fibers in the affected tissue can be increased in the subject between 5-20%, between 5-50%, between 10-50%, between 20-80%, or between 20-70%.

The nerve fibers that are modulated can be part of the peripheral nervous system, e.g., a somatic nerve, an autonomic nerve, a sensory nerve, a cranial nerve, an optic nerve, an olfactory nerve, a sympathetic nerve, a parasympathetic nerve, a chemoreceptor, a photoreceptor, a mechanoreceptor, a thermoreceptor, a nociceptor, an efferent nerve fiber, or an afferent nerve fiber.

The methods described herein can also be used to treat a subject who has cancer by administering to a subject having a cancer listed in column 2 of Table 10 a neuromodulating agent that modulates the activity and/or function of a correspondingly listed gene in column 1 of Table 10, in an amount (e.g., an effective amount) and for a time sufficient to treat the subject. The neuromodulating agent can be a neuropeptide having the sequence referenced by accession number of a neuropeptide listed in column 3 of Table 10 for the corresponding gene, or an analog or fragment thereof, e.g., a sequence having at least 75%, 80%, 85%, 90%, 90%, 98%, or 99% identity to the sequence referenced by accession number.

The methods described herein can be used to treat a subject with neuro-dependent cancer (e.g., cancer that expresses one or more neurome genes listed in Table 7 or Table 8 (e.g., a biosynthesis, channel, transporter, ligand, receptor, signaling, synaptic, structural, or vesicular gene)). The neuromodulating agent can be a neurome gene expression modulator that decreases gene expression (e.g., an inhibitory RNA) in a subject with a neuro-dependent cancer that overexpresses one or more neurome genes. The neuromodulating agent can be a neurome gene expression modulator that increases gene expression (e.g., an mRNA or a viral vector encoding a neurome gene) in a subject with a neuro-dependent cancer that under-expresses one or more neurome genes. The neuromodulating agent can be an agent that increases neurotransmission or neuropeptide signaling (e.g., a neurotransmitter or neuropeptide receptor agonist) in a subject with a neuro-dependent cancer that under-expresses one or more genes needed for neurotransmission or neuropeptide signaling. The neuromodulating agent can be an agent that decreases neurotransmission or neuropeptide signaling (e.g., a neurotransmitter or neuropeptide receptor antagonist, or a neurotoxin) in a subject with a neuro-dependent cancer that overexpresses one or more genes needed for neurotransmission or neuropeptide signaling. If the subject has a neuro-dependent cancer that overexpresses a neuronal growth factor gene, the neuromodulating agent can be an agent that reduces neuronal growth factor signaling (e.g., a blocking antibody or growth factor receptor antagonist).

In the methods described herein relating to cancer, the cancer or neoplasm may be any solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Additional examples of cancers that can be treated according to the methods described herein include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other examples of cancers that can be treated according to the methods described herein include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastmic carcinoma, adrenocortical carcinoma, cholangiocarcinoma, Merkel cell carcinoma, ductal carcinoma in situ (DCIS), and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

In some embodiments, the cancer is a paraneoplastic cancer (e.g., a cancer that causes a paraneoplastic syndrome). Paraneoplastic syndromes are rare disorders that are triggered by an altered immune system response to a neoplasm, and are mediated by humoral factors such as hormones, cytokines, or auto-antibodies produced by the tumor. Symptoms of paraneoplastic syndrome may be endocrine, neuromuscular, or musculoskeletal, cardiovascular, cutaneous, hematologic, gastrointestinal, renal, or neurological. Paraneoplastic syndromes commonly present with lung, breast, and ovarian cancer and cancer of the lymphatic system (e.g., lymphoma). Paraneoplastic neurological disorders are disorders that affect the central or peripheral nervous system, and can include symptoms such as ataxia (difficulty with walking and balance), dizziness, nystagmus (rapid uncontrolled eye movements), difficulty swallowing, loss of muscle tone, loss of fine motor coordination, slurred speech memory loss, vision problems, sleep disturbances, dementia, seizures, or sensory loss in the limbs. Breast, ovarian, and lung cancers are most commonly associated with paraneoplastic neurological disorders. Other common types of paraneoplastic syndromes include paraneoplastic cerebellar degeneration, paraneoplastic pemphigus, paraneoplastic autonomic neuropathy, paraneoplastic encephalomyelitis, and cancer-associated autoimmune retinopathy.

Endocrine paraneoplastic syndromes include Cushing syndrome (caused by ectopic ACTH), which is most commonly caused by small cell lung cancer, pancreatic carcinoma, neural tumors, or thymoma; SIADH (caused by antidiuretic hormone), which is most commonly caused by small cell lung cancer and CNS malignancies; hypercalcemia (caused by PTHrp, TGFα, TNF, or IL-1), which is most commonly caused by lung cancer, breast carcinoma, renal and bladder carcinoma, multiple myeloma, adult T cell leukemia/lymphoma, ovarian carcinoma, and squamous cell carcinoma (e.g., lung, head, neck, or esophagus carcinoma); hyperglycemia (caused by insulin insulin-like substance, or “big” IGF-II), which is most commonly caused by fibrosarcoma, mesenchymal sarcomas, insulinoma, and hepatocellular carcinoma; carcinoid syndrome (caused by serotonin or bradykinin), which is most commonly caused by bronchial adenoma, pancreatic carcinoma, and gastric carcinoma; and hyperaldosteronism (caused by aldosterone), which is most commonly caused by adrenal adenoma/Conn's syndrome, non-Hodgkin's lymphoma, ovarian carcinoma, and pulmonary cancer.

Neurological paraneoplastic syndromes include Lambert-Eaton myasthenic syndrome (LEMS), which is most commonly caused by small cell lung cancer; paraneoplastic cerebellar degeneration, which is most commonly caused by lung cancer, ovarian cancer, breast carcinoma, and Hodgkin's lymphoma; encephalomyelitis; limbic encephalitis, which is most commonly caused by small cell lung carcinoma; myasthenia gravis, which is most commonly caused by thymoma; brainstem encephalitis; opsoclonus myoclonus ataxia (caused by autoimmune reaction against Nova-1), which is most commonly caused by breast carcinoma, ovarian carcinoma, small cell lung carcinoma, and neuroblastoma; anti-NMDA receptor encephalitis (caused by autoimmune reaction against NMDAR subunits), which is most commonly caused by teratoma; and polymyositis, which is most commonly caused by lung cancer, bladder cancer, and non-Hodgkin's lymphoma. Mucotaneous paraneoplastic syndromes include acanthosis nigricans, which is most commonly caused by gastric carcinoma, lung carcinoma, and uterine carcinoma; dermatomyositis, which is most commonly caused by bronchogenic carcinoma, breast carcinoma, ovarian cancer, pancreatic cancer, stomach cancer, colorectal cancer, and Non-Hodgkin's lymphoma; Leser-Trelat sign; necrolytic migratory erythema, which is most commonly caused by glucagonoma; Sweet's syndrome; florid cutaneous papillomatosis; pyoderma gangrenosum; and acquired generalized hypertrichosis.

Hematological syndromes include granulocytosis (caused by G-CSF); polycythemia (caused by erythropoietin), which is commonly caused by renal carcinoma, cerebellar hemangioma, and heptatocellular carcinoma; Trousseau sign (caused by mucins), which is commonly caused by pancreatic carcinoma and bronchogenic carcinoma; nonbacterial thrombotic endocarditis, which is caused by advanced cancers; and anemia, which is most commonly caused by thymic neoplasms. Other paraneoplastic syndromes include membranous glomerular nephritis; neoplastic fever; Staffer syndrome, which is caused by renal cell carcinoma; and tumor-induced osteomalacia (caused by FGF23), which is caused by hemangiopericytoma and phosphaturic mesenchymal tumor.

In some embodiments, a subject is identified as having cancer after presenting with symptoms of a paraneoplastic syndrome. A common symptom of paraneoplastic syndrome is fever. Auto-antibodies directed against nervous system proteins are also frequently observed in patients with paraneoplastic syndromes, including anti-Hu, anti-Yo, anti-Ri, anti-amphiphysin, anti-CV2, anti-Ma2, anti-recoverin, anti-transducin, anti-carbonic anhydrase II, anti-arrestin, anti-GCAP1, anti-GCAP2, anti-HSP27, anti-Rab6A, and anti-PNR. Other symptoms that can be used to identify a patient with paraneoplastic cancer include ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs. In some embodiments, a patient presents with symptoms of paraneoplastic syndrome and is then identified as having cancer based on imaging tests (e.g., CT, MRI, or PET scans).

The cancer may be highly innervated, metastatic, non-metastatic cancer, or benign (e.g., a benign tumor). The cancer may be a primary tumor or a metastasized tumor. The cancer may be neuro-dependent (e.g., a cancer cell or tumor that expresses one or more neurome genes listed in Table 7 or Table 8 (e.g., a biosynthesis, channel, transporter, ligand, receptor, signaling, synaptic, structural, or vesicular gene)). These genes include neurotransmitters, neurotransmitter receptors, neuropeptides, neuropeptide receptors, neurotransmitter transporters, neurotransmitter biogenesis or biosynthetic genes, ion channels, ion pumps, vesicular proteins, synaptic junction proteins, axonal guidance proteins, neurotrophic factors, and signaling molecules found downstream of neuronal cell surface receptors (see Table 7 for complete list). In some embodiments, the neurological gene signature (e.g., neurome gene expression profile) is unrelated to tumor type of origin. The one or more neurome genes expressed by the cancer cell or tumor can be used to determine patient treatment (e.g., the neuromodulating agent can be chosen based on the neurome gene(s) expressed by the cancer cell or tumor).

The neurome gene expression profile of a cancer cell or tumor can be determined using gene profiling. Profiling results can also be used to categorize (e.g., identify) a tumor as belonging to a neurological taxon (e.g., a neurotaxonomic group). In some embodiments, the neoplasm is placed into a group (e.g., neurotaxonomic group) based on profiling results. Placement within a neurotaxonomic group can be used to determine patient treatment (e.g., to determine which neuromodulating agent to administer). In some embodiments, the neuro-dependent tumor can be placed into one of four groups based on neurome gene expression: (family 1) HTR1 D, ADRA2A, CHRNA7; (family 2) ADRA2C, CHRM3; (family 3) GRM8, DRD2, CHRNB2, CHRM4; (family 4) ADRAB2, ADRA1B. In some embodiments, the neuro-dependent tumor overexpresses one or more neurome genes. For example, the cancer cell or tumor can be a cancer listed in Table 12 that overexpresses a corresponding gene in Table 12. In some embodiments, the neuro-dependent tumor under-expresses one or more neurome genes. For example, the cancer cell or tumor can be a cancer listed in Table 13 that under-expresses a corresponding gene in Table 13. In some embodiments, the neuro-dependent tumor co-expresses one or more genes encoding a neuropeptide or neurotransmitter, or a gene necessary for the biosynthesis or recycling of a neuropeptide or neurotransmitter, and genes encoding the cognate neurotransmitter or neuropeptide receptor. For example, the cancer cell or tumor can be a cancer listed in Tables 14A-14C that expresses the corresponding biosynthetic enzyme and cognate receptor shown in Tables 14A-14C. In some embodiments, the neuro-dependent tumor expresses one or more neurotrophic factors. Neuro-dependent tumors can be identified using quantitative RT-PCR, RNA sequencing, immunohistochemistry, or western blot analysis of tumor samples.

The methods described herein can include profiling a tumor sample to determine whether it expresses one or more neurome genes (e.g., a neurome gene in Table 7 or Table 8 (e.g., a biosynthesis, channel, transporter, ligand, receptor, signaling, synaptic, structural, or vesicular gene)). Profiling can be performed using RNA sequencing, microarray analysis, or serial analysis of gene expression (SAGE). Other techniques that can be used to assess gene expression include quantitative RT-PCR. Profiling results can be confirmed using methods such as immunohistochemistry, western blot analysis, or southern blot analysis. Profiling results can be used to determine which neuromodulating agent should be administered to treat the patient. In some embodiments, the methods described herein include selecting a neuromodulating agent based on the neurome gene expression profile of the tumor. For example, if a tumor expresses a neurotransmitter or neurotransmitter receptor, the neuromodulating agent can be a neurotransmission modulator or a neurome gene expression modulator. If a tumor expresses a neuropeptide or neuropeptide receptor, the neuromodulating agent can be a neuropeptide signaling modulator or a neurome gene expression modulator. If a tumor expresses a neuronal growth factor, the neuromodulating agent can be a neuronal growth factor modulator or a neurome gene expression modulator. Neuromodulating agents that decrease expression or activity can be used if the neurome gene is overexpressed, and neuromodulating agents that increase expression or activity can be used if the neurome gene is under-expressed.

Over- and under-expression can be determined by comparing expression of a neurome gene to a housekeeping gene. If the neurome gene is expressed at a higher level than the housekeeping gene (e.g., neurome gene expression is at least 1.5, 2, 2.5, 3, 4, 5, or 10 or more fold higher than housekeeping gene expression), the neurome gene is overexpressed. In some embodiments, the overexpressed neurome gene is a gene in Table 12 that is expressed in a corresponding cancer in Table 12. If the neurome gene is expressed at a lower level than the housekeeping gene (e.g., neurome gene expression is at least 1.5, 2, 2.5, 3, 4, 5, or 10 or more fold lower than housekeeping gene expression), the neurome gene is under-expressed. In some embodiments, the under-expressed neurome gene is a gene in Table 13 that is under-expressed in a corresponding cancer in Table 13.

Subjects who can be treated with the methods disclosed herein include subjects who have had one or more tumors resected, received chemotherapy or other pharmacological treatment for the cancer, received radiation therapy, and/or received other therapy for the cancer. Subjects who have not previously been treated for cancer can also be treated with the methods disclosed herein.

IV. Combination Therapies for Cancer

Combination Therapies for Cancer

A neuromodulating agent described herein can be administered in combination with a second therapeutic agent for treatment of cancer. In some embodiments, the neuromodulating agent is selected based on the neurome gene expression profile of the cancer. In some embodiments, the second therapeutic agent is selected based on tumor type, tumor tissue of origin, tumor stage, or mutations in non-neurome genes expressed by the tumor.

Checkpoint Inhibitors

One type of agent that can be administered in combination with a neuromodulating agent described herein is a checkpoint inhibitor. Checkpoint inhibitors can be broken down into at least 4 major categories: i) agents such as antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1 BB), iii) agents such as antibodies that block a suppressive pathway on immune cells or rely on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, or MUC1). Such agents described herein can be designed and produced, e.g., by conventional methods known in the art (e.g., Templeton, Gene and Cell Therapy, 2015; Green and Sambrook, Molecular Cloning, 2012).

Chemotherapy

A second type of therapeutic agent that can be administered in combination with a neuromodulating agent described herein is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed Engl. 33:183 1994); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc ASCO 18:233a, 1999, and Douillard et al., Lancet 355:1041, 2000.

Biologic Cancer Agents

Another type of therapeutic agent that can be administered in combination with a neuromodulating agent described herein is a therapeutic agent that is a biologic such a cytokine (e.g., interferon or an interleukin (e.g., IL-2)) used in cancer treatment. In other embodiments the biologic is an anti-angiogenic agent, such as an anti-VEGF agent, e.g., bevacizumab. In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituximab; Daclizumab; Basiliximab; Palivizumab; Infliximab; Trastuzumab; Gemtuzumab ozogamicin; Alemtuzumab; Ibritumomab tiuxetan; Adalimumab; Omalizumab; Tositumomab-I-131; Efalizumab; Cetuximab; Bevacizumab; Natalizumab; Tocilizumab; Panitumumab; Ranibizumab; Eculizumab; Certolizumab pegol; Golimumab; Canakinumab; Ustekinumab; Ofatumumab; Denosumab; Motavizumab; Raxibacumab; Belimumab; Ipilimumab; Brentuximab Vedotin; Pertuzumab; Ado-trastuzumab emtansine; and Obinutuzumab. Also included are antibody-drug conjugates. Examples of biologic cancer agents that can be used in combination with neuromodulating agents described herein are shown in Table 9 below.

TABLE 9
APPROVED CANCER ANTIBODIES

| Antibody | Company | Antigen | Indication |
|---|---|---|---|
| Ado-trastuzumab | Genentech | HER2 | Metastatic breast cancer |
| emtansine | | | |
| Alemtuzumab | Genzyme | CD52 | B-cell chronic lymphocytic leukemia |
| Atezolizumab | Genentech | PD-L1 | Urothelial carcinoma |
| Atezolizumab | Genentech | PD-L1 | Urothelial carcinoma |
| | | | Metastatic non-small cell lung cancer |
| Avelumab | EMD Serono | PD-L1 | Metastatic Merkel cell carcinoma |
| Bevacizumab | Genentech | VEGF | Metastatic colorectal cancer |
| Blinatumomab | Amgen | CD19 | Precursor B-cell acute lymphoblastic leukemia |
| Brentuximab | Seattle Genetics | CD30 | Hodgkin lymphoma |
| vedotin | | | Anaplastic large-cell lymphoma |
| Cetuximab | ImClone Systems | EGFR | Metastatic colorectal carcinoma |
| Daratumumab | Janssen Biotech | CD38 | Multiple myeloma |
| Dinutuximab | United Therapeutics | GD2 | Pediatric high-risk neuroblastoma |
| Durvalumab | AstraZeneca | PD-L1 | Urothelial carcinoma |
| Elotuzumab | Bristol-Myers | SLAMF7 | Multiple myeloma |
| | Squibb | | |
| Ibritumomab | Spectrum | CD20 | Relapsed or refractory low-grade, follicular, or |
| tiuxetan | Pharmaceuticals | | transformed B-cell non-Hodgkin's lymphoma |
| Ipilimumab | Bristol-Myers | CTLA-4 | Metastatic melanoma |
| | Squibb | | |
| Necitumumab | Eli Lilly | EGFR | Metastatic squamous non-small cell lung |
| | | | carcinoma |
| Nivolumab | Bristol-Myers | PD-1 | Metastatic melanoma |
| | Squibb | | |
| Nivolumab | Bristol-Myers | PD-1 | Metastatic squamous non-small cell lung |
| | Squibb | | carcinoma |
| Obinutuzumab | Genentech | CD20 | Chronic lymphocytic leukemia |
| Ofatumumab | Glaxo Grp | CD20 | Chronic lymphocytic leukemia |
| Olaratumab | Eli Lilly | PDGFRA | Soft tissue sarcoma |
| Panitumumab | Amgen | EGFR | Metastatic colorectal cancer |
| Pembrolizumab | Merck | PD-1 | Metastatic melanoma |
| Pertuzumab | Genentech | HER2 | Metastatic breast cancer |
| Ramucirumab | Eli Lilly | VEGFR2 | Gastric cancer |
| Rituximab | Genentech | CD20 | B-cell non-Hodgkin's lymphoma |
| Trastuzumab | Genentech | HER2 | Metastatic breast cancer |

Non-Drug Therapies

Another type of agent that can be administered in combination with a neuromodulating agent is a therapeutic agent that is a non-drug treatment. For example, the second therapeutic agent is radiation therapy, cryotherapy, hyperthermia and/or surgical excision of tumor tissue.

In any of the combination therapy approaches described herein, the first and second therapeutic agent (e.g., a neuromodulating agent described herein and the additional therapeutic agent) are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

V. Methods of Treatment

Administration

An effective amount of a neuromodulating agent described herein for treatment of cancer can be administered to a subject by standard methods. For example, the agent can be administered by any of a number of different routes including, e.g., intravenous, intradermal, subcutaneous, percutaneous injection, oral, transdermal (topical), or transmucosal. The neuromodulating agent can be administered orally or administered by injection, e.g., intramuscularly, or intravenously. The most suitable route for administration in any given case will depend on the particular agent administered, the patient, the particular disease or condition being treated, pharmaceutical formulation methods, administration methods (e.g., administration time and administration route), the patients age, body weight, sex, severity of the diseases being treated, the patient's diet, and the patient's excretion rate. The agent can be encapsulated or injected, e.g., in a viscous form, for delivery to a chosen site, e.g., a tumor. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The agent (e.g., peptide, neurotransmitter, small molecule, nucleic acid, protein such as an antibody) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a neuromodulating agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art.

Nucleic acid molecule agents described herein can be administered directly (e.g., therapeutic mRNAs) or inserted into vectors used as gene therapy vectors. Gene therapy vectors can be delivered to a subject by, for example, intravenous injection, local administration (see U.S. Pat. No. 5,328,470) or by stereotactic injection (see, e.g., Chen et al., PNAS 91:3054 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

The neuromodulating agents described herein may be administered in a unit dose form. For example, the methods described herein include administration of a unit dose form of a beta-adrenergic inhibitory agent. The unit dose can be less than or more than a unit dose of the beta blocker that is FDA approved for high blood pressure, a cardiac condition, angina, essential tremor, hypertrophic subaortic stenosis, migraine prophylaxis, myocardial infarction prophylaxis, pheochromocytoma, tachyarrhythmias, or thyrotoxicosis. The beta-adrenergic blocking agent can be selected from: acebutolol, atenolol, bisoprolol, metoprolol, nadolol, and propranolol. The agent can be formulated for parenteral administration, enteral administration (e.g., oral), or local administration (e.g., epicutaneous, inhalational, intra-articular, intrathecal, intravaginal, intravitreal, intrauterine, intra-lesional or intra-tumoral administration).

The unit dose form can be a unit dose of a cholinergic inhibitory agent. The unit dose can be less than or more than a unit dose of the cholinergic blocker that is FDA approved for Alzheimer's Disease, Cardiac and Respiratory Disorders, Atony and Neurogenic Bladder, motion sickness, Myasthenia gravis, Peptic ulcer, IBD, Glaucoma, Parkinson's Disease, reflex neurogenic bladder (spinal cord injury), or Incontinence-overactive bladder. The cholinergic blocking agent can be selected from: tacrine, galantamine, rivastigmine, donepezil. The unit dose can be configured for local administration, e.g., epicutaneous, inhalational, intra-articular, intrathecal, intravaginal, intravitreal, intrauterine, intra-lesional or intra-tumoral administration.

The unit dose form can be a unit dose of a dopaminergic inhibitory agent. The unit dose can be less than or more than a unit dose of the dopamine blocker that is FDA approved for schizophrenia, bipolar disorder, or nausea and vomiting. The dopamine blocking agent can be selected from: acepromazine, amisulpride, amoxapine, asenapine, azaperone, benperidol, Bromopride, butaclamol, chlorpromazine, chlorprothixene, clopenthixol, Domperidone, droperidol, eticlopride, flupenthixol, fluphenazine, fluspirilene, haloperidol, hydroxyzine, iodobenzamide, loxapine, mesoridazine, levomepromazine, metoclopramide, nafadotride, nemonapride, olanzapine, paliperidone, penfluridol, perazine, perphenazine, pimozide, prochlorperazine, promazine, quetiapine, raclopride, remoxipride, risperidone, spiperone, spiroxatrine, stepholidine, sulpiride, sultopride, tetrahydropalmatine, thiethylperazine, thioridazine, thiothixene, tiapride, trifluoperazine, trifluperidol, triflupromazine, and ziprasidone. The unit dose can be configured for local administration, e.g., epicutaneous, inhalational, intra-articular, intrathecal, intravaginal, intravitreal, intrauterine, intra-lesional or intra-tumoral administration.

The unit dose can be a unit dose of a serotonin inhibitory agent. The unit dose can be less than or more than a unit dose of the serotonin blocker that is FDA approved for treatment of a mood disorder, e.g., major depressive disorder (MDD), anxiety disorder, obsessive-compulsive disorder (OCD), attention deficit hyperactivity disorder (ADHD), chronic neuropathic pain, fibromyalgia syndrome (FMS), or for the relief of menopausal symptoms. The serotonin blocking agent can be selected from: Venlafaxine, Desvenlafaxine, Duloxetine, Milnacipran Levomilnacipran, Sibutramine, and Atomoxetine. The unit dose can be configured for local administration, e.g., epicutaneous, inhalational, intra-articular, intrathecal, intravaginal, intravitreal, intrauterine, intra-lesional or intra-tumoral administration.

Local Administration

The neuromodulating agents described herein can be administered locally, e.g., to the site of cancer in the subject. Examples of local administration include epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect. As an example, for the treatment of a cancer described herein, the neuromodulating agent may be administered locally (e.g., intratumorally) in a compound-impregnated substrate such as a wafer, microcassette, or resorbable sponge placed in direct contact with the affected tissue. Alternatively, the neuromodulating agent is infused into the brain or cerebrospinal fluid using standard methods. As yet another example, a pulmonary cancer described herein may be treated, for example, by administering the neuromodulating agent locally by inhalation, e.g., in the form of an aerosol spray from a pressured container or dispenser which contains a suitable propellant, e.g., a gas such as carbon dioxide or a nebulizer. A neuromodulating agent for use in the methods described herein can be administered or at the site of a tumor, e.g., intratumorally. In certain embodiments, the agent is administered to a mucous membrane of the subject.

Combination Therapy

The neuromodulating agents described herein may be administered in combination with one or more additional therapies (e.g., 1, 2, 3 or more additional therapeutic agents). The two or more agents can be administered at the same time (e.g., administration of all agents occurs within 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

For use in treating cancer, the second agent may be a checkpoint inhibitor, a chemotherapeutic drug, a biologic drug. In one embodiment, the inhibitor of checkpoint is an inhibitory antibody (e.g., a monospecific antibody such as a monoclonal antibody). The antibody may be, e.g., humanized or fully human. In other embodiments, the inhibitor of checkpoint is a fusion protein, e.g., an Fc-receptor fusion protein. In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein. In one embodiment, the inhibitor of checkpoint is an inhibitor (e.g., an inhibitory antibody or small molecule inhibitor) of CTLA-4 (e.g., an anti-CTLA4 antibody such as ipilimumab or tremelimumab). In one embodiment, the inhibitor of checkpoint is an inhibitor (e.g., an inhibitory antibody or small molecule inhibitor) of PD-1 (e.g., nivolumab; pembrolizumab; pidilizumab/CT-011). In one embodiment, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In one embodiment, the inhibitor of checkpoint is an inhibitor (e.g., an inhibitory antibody or Fc fusion or small molecule inhibitor) of PDL2 (e.g., a PDL2/Ig fusion protein such as AMP 224). In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. The second agent may also be an anti-angiogenic drug, e.g., an anti-VEGF antibody, or the second agent may be an oncolytic agent e.g., a chemotherapy, a drug that targets cancer metabolism, an antibody that marks a cancer cell surface for destruction, e.g., rituximab or trastuzumab an antibody-drug conjugate, e.g., trastuzumab emtansine, a cell therapy, or other commonly-used anti-neoplastic agent.

Dosing

Subjects that can be treated as described herein are subjects with cancer or at risk of developing cancer. The cancer may be a primary tumor or a metastasized tumor. Subjects who can be treated with the methods disclosed herein include subjects who have had one or more tumors resected, received chemotherapy or other pharmacological treatment for the cancer, received radiation therapy, and/or received other therapy for the cancer. Subjects who have never previously been treated for cancer can also be treated using the methods described herein.

In some embodiments, the agent is administered in an amount and for a time effective to result in one of (or more, e.g., 2 or more, 3 or more, 4 or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

The methods described herein may include a step of selecting a treatment for a patient. The method includes (a) identifying (e.g., diagnosing) a patient who has cancer or is at risk of developing cancer, and (b) selecting a neuromodulating agent, e.g., a neuromodulating agent described herein, to treat the condition in the patient. In some embodiments, the method includes administering the selected treatment to the subject. In some embodiments, a patient is identified as having cancer based on imaging (e.g., MRI, CT, or PET scan), biopsy, or blood sample (e.g., detection of blood antigen markers, circulating tumor DNA (e.g., by PCR). In some embodiments, a patient is identified as having cancer after presenting with one or more symptoms of a paraneoplastic syndrome (e.g., fever. auto-antibodies directed against nervous system proteins, ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs). In some embodiments, a patient presents with symptoms of paraneoplastic syndrome and is then identified as having cancer based on imaging (e.g., CT, MRI, or PET scans).

The method may also include (a) identifying (e.g., diagnosing) a patient who has a neoplasm, (b) optionally evaluating the neoplasm for innervation, and (c) selecting a neuromodulating agent (e.g., a neuromodulating agent described herein) to treat the patient if the neoplasm is highly innervated (e.g., if the level of innervation is at least 10% higher (e.g., at least 20%, 30%, 40%, 50%, 60%, 70%, 80% higher) than the level of innervation in control tissue, e.g., non-cancerous tissue of the same subject). Innervation may be measured by staining tissue sections for neural markers e.g., immuno-histochemical staining for tyrosine hydroxylase, vesicular acetylcholine transporter; NGF-Inducible Large External glycoprotein, choline acetyltransferase, parvalbumin, neurofilament protein, Synapsin, synaptophysin, NeuN, NSE, MAP2, Beta III tubulin, 160 kD Neurofilament medium/200 kD Neurofilament Heavy, NSE, PSD93/PSD95, Doublecortin (DCX), c-fos, PSA-NCAM, NeuroD or Beta2, Tau, Calbindin-D28k, Calretinin, Neurofilament Protein (NFP), Glial fibrillary acidic protein (GFAP), S100β, Vimentin and CNPase; or by staining tissue sections with cell-identifying stains, e.g., H&E stain, Nissl Stain, Cresyl violet, Neutral red, Thionine and Toluidine blue, Luxol Fast blue stain, Weigert's Chromium hematoxylin method, Page's iron-eriochrome cyanine R, Dextran Conjugates (Fluorescein, Tetramethylrhodamine, Texas Red, Rhodamine Green), Hydrazides & Biocytins, Isolectin GS-IB4 conjugates, Golgi silver stain, or myelin stain; or by imaging the nervous system, e.g., by MRI, CT, PET, EEG, EMG, Myelogram, or magnetoencephalography. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, prostate cancer, lung cancer, breast cancer, skin cancer (e.g., melanoma), renal cell carcinoma, or colorectal cancer. In some embodiments, the cancer is a cancer listed in Table 10. In some embodiments, the neoplasm is derived from a secretory tissue, glandular tissue, or endocrine or hormonal tissue.

In one embodiment, the method includes (a) identifying (e.g., diagnosing) a patient who has a neoplasm, (b) optionally evaluating the neoplasm for perineural invasion, and (c) selecting a neuromodulating agent to treat the patient if the neoplasm exhibits perineural invasion. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, and prostate cancer.

In one embodiment, the method includes (a) identifying (e.g., diagnosing) a patient who has a neoplasm, (b) optionally evaluating the subject for metastasis to brain or spinal cord, and (c) selecting a neuromodulating agent to treat the patient if the neoplasm exhibits metastasis to brain or spinal cord. In some embodiments, the neoplasm is a lung cancer, breast cancer, skin cancer (e.g., melanoma), lymphoma, renal cell carcinoma, GI tract cancer, prostate cancer, or colorectal cancer.

In some embodiments, the method includes administering the selected treatment to the subject.

The method may also include a step of assessing the subject for a parameter of cancer progression or remission, e.g., assessing the subject for one or more (e.g., 2 or more, 3 or more, 4 or more) of: primary tumor size (e.g., by imaging), number of metastases (e.g., by imaging or biopsy), cell death in situ (e.g., by biopsy), blood antigen markers (e.g., by ELISA), circulating tumor DNA (e.g., by PCR), or function of the affected organ (e.g., by a test of circulating enzymes for liver, albuminuria for kidney, lung capacity for lung, etc.).

The method can also include (a) identifying (e.g., diagnosing) a patient who has a neoplasm, (b) optionally evaluating (e.g., profiling) the neoplasm for neurome gene expression, and (c) identifying the patient as having neuro-dependent cancer if one or more neurome genes are expressed by the neoplasm. In some embodiments, the method includes selecting a neuromodulating agent to treat the patient based on neurome gene expression.

The method can also include (a) identifying (e.g., diagnosing) a patient who has a neoplasm, (b) optionally evaluating (e.g., profiling) the neoplasm for neurome gene expression, and (c) selecting a neuromodulating agent to treat the patient based on the neurome gene that is over- or under-expressed. In some embodiments, the neoplasm overexpresses one or more neurome genes. For example, the neoplasm can be a cancer listed in Table 12 that overexpresses a corresponding gene in Table 12. In some embodiments, the neoplasm underexpresses one or more neurome genes. For example, the neoplasm can be a cancer listed in Table 13 that under-expresses a corresponding gene in Table 13. In some embodiments, the neoplasm can be placed into a group (e.g., neurotaxonomic group) based on profiling results. In some embodiments, the neoplasm can be placed into one of four groups based on neurome gene expression: (family 1) HTR1 D, ADRA2A, CHRNA7; (family 2) ADRA2C, CHRM3; (family 3) GRM8, DRD2, CHRNB2, CHRM4; (family 4) ADRAB2, ADRA1B. In some embodiments, the neoplasm co-expresses one or more genes encoding a neuropeptide or neurotransmitter, or a gene necessary for the biosynthesis or recycling of a neuropeptide or neurotransmitter, and one or more genes encoding the cognate neurotransmitter or neuropeptide receptor. For example, the neoplasm can be a cancer listed in Tables 14A-14C that expresses the corresponding biosynthetic enzyme and cognate receptor shown in Tables 14A-14C. In some embodiments, the neuro-dependent tumor expresses one or more neurotrophic factors.

The methods described herein can include profiling a tumor sample to determine whether it expresses one or more neurome genes (e.g., a neurome gene in Table 7 or Table 8 (e.g., a biosynthesis, channel, transporter, ligand, receptor, signaling, synaptic, structural, or vesicular gene)). Profiling results can also be used to categorize (e.g., identify) a tumor as belonging to a neurological taxon (e.g., a neurotaxonomic group). In some embodiments, the neoplasm is placed into a group (e.g., neurotaxonomic group) based on profiling results. Placement within a neurotaxonomic group can be used to determine patient treatment (e.g., to determine which neuromodulating agent to administer to the patient). Profiling can be performed using RNA sequencing, microarray analysis, or serial analysis of gene expression (SAGE). Other techniques that can be used to assess gene expression include quantitative RT-PCR. Profiling results can be confirmed using methods such as immunohistochemistry, western blot analysis, or southern blot analysis. Profiling results can be used to determine which neuromodulating agent should be administered to treat the patient. In some embodiments, the methods described herein include selecting a neuromodulating agent based on the neurome gene expression profile of the tumor. For example, if a tumor expresses a neurotransmitter or neurotransmitter receptor, the neuromodulating agent can be a neurotransmission modulator or a neurome gene expression modulator. If a tumor expresses a neuropeptide or neuropeptide receptor, the neuromodulating agent can be a neuropeptide signaling modulator or a neurome gene expression modulator. If a tumor expresses a neuronal growth factor, the neuromodulating agent can be a neuronal growth factor modulator or a neurome gene expression modulator. Neuromodulating agents that decrease expression or activity can be used if the neurome gene is overexpressed, and neuromodulating agents that increase expression or activity can be used if the neurome gene is under-expressed.

Over- and under-expression can be determined by comparing expression of a neurome gene to a housekeeping gene. If the neurome gene is expressed at a higher level than the housekeeping gene (e.g., neurome gene expression is at least 1.5, 2, 2.5, 3, 4, 5, or 10 or more fold higher than housekeeping gene expression), the neurome gene is overexpressed. In some embodiments, the overexpressed neurome gene is a gene in Table 12 that is expressed in a corresponding cancer in Table 12. If the neurome gene is expressed at a lower level than the housekeeping gene (e.g., neurome gene expression is at least 1.5, 2, 2.5, 3, 4, 5, or 10 or more fold lower than housekeeping gene expression), the neurome gene is under-expressed. In some embodiments, the under-expressed neurome gene is a gene in Table 13 that is under-expressed in a corresponding cancer in Table 13.

In some embodiments, a neuro-dependent tumor is treated with a neuromodulating agent and a second therapeutic agent. The second therapeutic agent can be selected based on tumor type, tumor tissue of origin, tumor stage, or mutations in non-neurome genes expressed by the tumor.

A neuromodulating agent administered according to the methods described herein does not have a direct effect on the central nervous system (CNS) or gut. Any effect on the CNS or gut is reduced compared to the effect observed if the neuromodulating agent is administered directly to the CNS or gut. In some embodiments, direct effects on the CNS or gut are avoided by modifying the neuromodulating agent not to cross the BBB, as described herein above, or administering the agent locally to a subject.

Subjects with cancer or at risk of developing cancer are treated with an effective amount of a neuromodulating agent. The methods described herein also include contacting a tumor or cancer cell with an effective amount of a neuromodulating agent. In some embodiments, an effective amount of a neuromodulating agent is an amount sufficient to increase or decrease tumor innervation or nerve activity in a tumor. In some embodiments, an effective amount of a neuromodulating agent is an amount sufficient to treat the cancer or tumor, cause remission, reduce tumor growth, volume, metastasis, invasion, proliferation, or number, increase cancer cell death, increase time to recurrence, or improve survival.

The neuromodulating agents described herein are administered in an amount (e.g., an effective amount) and for a time sufficient to effect one of the outcomes described above. The neuromodulating agent may be administered once or more than once. The neuromodulating agent may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months or more following administration of a neuromodulating agent depending on the neuromodulating agent and route of administration used for treatment. Depending on the outcome of the evaluation, treatment may be continued or ceased, treatment frequency or dosage may change, or the patient may be treated with a different neuromodulating agent. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated.

### Kits

The invention also features a kit comprising (a) a unit dose described herein, and (b) instructions for administering the unit dose to treat cancer.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Screening Method for Serotonin Receptor Binders

High throughput methods for identifying compounds from libraries that bind to a target molecule have been described previously, see e.g., Janzen and Bernasconi (Eds.), High Throughput Screening: Methods and Protocols (Methods in Molecular Biology), Humana Press 2009. In brief, to identify compounds that bind to the serotonin receptor 5HT2C the following screening assay is performed:

Cell Culture & Membrane (Target Protein) Preparation:

AV12 cells are stably transfected with a eukaryotic expression vector containing the coding region for the human 5HT2C receptor (see e.g., Lucaites, V. L., et al., (1996) Life Sci. 59(13), 1081-1095). To prepare membrane protein preparations, using the technique of Bosworth and Towers, Nature 341, 167, 1989, cells are grown to a cell density of 2-3×10⁶ cells/mL, and 15 L are harvested on a daily basis by centrifugation, washed in phosphate-buffered saline (PBS), and stored as frozen cell pastes at −80° C. To loosen the frozen cell paste, 30 mL of 50 mM Tris-HCl, pH 7.4, at ambient temperature are added to 7.5 grams of pellet. The cell slurry is homogenized on ice in a 55-mL glass/teflon dounce, transferred to a 250-mL conical tube that is then filled to the neck with buffer, mixed, and centrifuged in a table top centrifuge at 200 g (1060 RPM, GH-3.7 rotor) at 4° C. for 15 min. The supernatant is collected and saved on ice. The pellet is resuspended and subjected to the homogenization and centrifugation procedure just described. The 200 g supernatant is again collected and combined with the first supernatant stored on ice. The combined supernatants are then centrifuged at 14,250 rpm in a Sorvall RC5 centrifuge (GSA SLA-1500 rotor) for 50 min at 4° C. The supernatant is gently removed and discarded, and the remaining membrane pellet is resuspended using the dounce homogenizer. The membrane protein concentration is determined (BCA kit) and aliquots of the membrane preparation are quick frozen in liquid nitrogen and stored at −80° C. The average yield is 1.2% of starting weight.

SPA-Format Receptor-Binding Assay:

Twenty microliter of test compound, unlabeled 5-HT control, or assay buffer is added to each well of a 96-well microtiter plate. Fifty microliter of 15-nM [3H]-5HT ligand (5-Hydroxy(3H)tryptamine trifluoroacetate (Code TRK1006 Amersham) at a final concentration of 5 nM/well) is then added to the wells followed by 80 μL (20 ug) of 5HT2C membranes as prepared above and the plates are shaken for 1 min. After a 30-min incubation at room temperature, 0.5 mg of Wheat Germ Agglutinin (WGA)-SPA beads (Amersham biotech) are added, plates are mixed by shaking every 30 min for 2 h and then counted in a MicroBeta Scintillation Counter (Perkin Elemer Wallac). The absence of binding of labeled 5HT ligand in a sample indicates that the test compound has successfully bound the target receptor. Test compounds that bind target receptor with greater than 100 nM EC50 (p<0.05 for at least 3 replicates) are selected for further testing.

### Example 2—Dose Finding Study for Neuromodulator Candidate

A lead candidate for treatment of a solid cancer is identified by the screening method of Example 1. Based on preclinical data from in vitro and in vivo testing of the identified lead compound, it is determined that 120 mg is a safe starting dose in humans.

A ‘3+3’ design of incremental escalation of dose in a cohort of subjects is employed to identify a Maximum Tolerated Dose (MTD) of the lead candidate. Dose escalation is determined using a Fibonacci sequence, whereby an additional 100% of the original dose is administered for the second time, 67% of the second dose for the third time, and so on, until the MTD is reached.

Three patients are given 120 mg of the identified lead compound. If none of the three patients report any dose limiting toxicity (DLT) of this first dose, then the dose is escalated for the next cohort of 3 subjects. If within any one particular cohort one of the patients reports a DLT, the study at that dose is repeated. If two of the patients report DLT, this dose is then regarded as the Maximum Tolerated Dose (MTD).

### Example 3—Inhibition of Cancer Cell Growth in 2D Culture

The agents selected as described in Example 1 are subjected to testing for cell proliferation.

A way to measure cell proliferation is to detect an antigen present in proliferating cells, but not nonproliferating cells, using a monoclonal antibody to the antigen. For example, in human cells, the antibody Ki-67 recognizes the protein of the same name, expressed during the S, G2 and M phases of the cell cycle but not during the G0 and G1 (nonproliferative) phases. Reagents and solutions can be purchased commercially, e.g., Muse system from Millipore. The following procedure is used:

Culture cells, including for positive and negative controls, in appropriate culture medium. Wash cell samples once with PBS, then transfer 5×10\hat{ }3-1×10\hat{ }5 cells per sample into each tube. Prepare 1× Fixation Solution (50 μL per test) and 1× Assay Buffer (500 μL per test). Add 50 μL of 1× Fixation Solution to each tube. Mix and incubate for 15 minutes at room temperature. Add 150 μL of 1× Assay Buffer, centrifuge, and remove supernatant. Add 100 μL of Permeabilization Solution to each tube. Mix and incubate for 15 minutes at room temperature. Add 100 μL of 1× Assay Buffer, centrifuge, and remove supernatant. Add 50 μL of 1× Assay Buffer to each tube. Mix and incubate for 15 minutes at room temperature. Add 10 μL of either Hu IgG1-PE isotype control or Hu Ki67-PE Antibody to each tube. Mix and incubate for 30 minutes at room temperature. Add 150 μL of 1× Assay Buffer to each tube and analyze by flow cytometry. Binding of anti-Ki67 detected by flow cytometry indicates that the agent causes proliferation.

### Example 4—Treatment of Prostate Cancer with a Neuromodulating Agent

A patient diagnosed with prostate cancer is treated locally with an antibody specific for ProNGF (nerve growth factor precursor). In a surgical approach, depending on the age and preference of the patient, either a general or local anesthetic is administered. If a general anesthetic is administered, the patient is intubated. The surgical approach may differ according to surgeon preference and patient characteristics, however it may include, but will not be limited to open retro-pubic (incision in lower abdomen), open perineal (incision in perineum) or laparoscopic approaches. After initial incision and dissection through the abdominal or perineal muscles, a surgical window is established, and the treatment involves administration of the antibody either intra-tumorally and/or towards the innervating structures of the prostate gland and/or tumor, including above and below the plexuses formed by the parasympathetic, visceral, efferent, and pre-ganglionic fibers that arise from the sacral levels (S2-S4) and the sympathetic fibers from the thoracolumbar levels (L1-L2). A drainage tube may be inserted into the bladder and kept in place for a period of up to 10 days following the surgery. Furthermore, a catheter may be placed inside the patient for continuous administration of the previously described compounds, for a time period after surgery. If intubated, patient is extubated and may be started on antibiotics.

A minimally invasive approach to local administration of drug may involve a transurethral approach, whereby, a catheter or other such device is passed through the urethra and placed in proximity to the prostate gland. The antibody is then administered through this catheter.

In a non-surgical approach, the antibody is administered intravenously. For intravenous administration, access to a vein is established through an intravenous cannula. If this is not possible, due to factors such as vasospasm of vessels or poorly accessible veins, a central venous catheter is employed. Once intravascular access is established, the previously described compound will be administered.

Following treatment, the patient's peripheral blood is specifically tested for Prostate Specific Antigen (PSA) levels. Active monitoring of PSA levels is carried out every 3 months for 2 years, after which, measurement of PSA levels may be extended to once every 6 months, depending on the discretion of the health care professional.

### Example 5—Identification of Novel Neurobiological Correlations with Cancer

To identify novel correlations of neurobiological signaling molecules and cancer cells, a list of neurotransmitter and neuropeptide genes and pathways was generated using a survey of published literature and UniProt (Table 1). These genes and pathways identified were used as inputs to publicly available cancer databases (e.g., Cancer Cell Line Encyclopedia). Through the bioinformatics analysis, novel correlations were found of overexpression by at least two-fold of certain neurobiological signaling genes of interest in certain individual cancer cell lines. Table 10 lists the neurobiological signaling molecules (column 1) that are targets for therapeutic intervention for such correlated cancers (column 2).

TABLE 10
CANCER TARGETS

| Gene | Cancer | Accession Number |
|---|---|---|
| Adra1d | Ewings Sarcoma | P25100 |
| Adrb1 | Ewings Sarcoma | P08588 |
| Adrb3 | Ewings Sarcoma | P13945 |
| Adrbk1 | Leuk. AML | P25098 |
| Adrbk1 | Leuk. B cell ALL | P25098 |
| Adrbk1 | Leuk. CML | P25098 |
| Adrbk1 | Leuk. T cell ALL | P25098 |
| Adrbk1 | Leukemia - other | P25098 |
| Adrbk1 | Lymphoma - Burkitt | P25098 |
| Adrbk1 | lymphoma - DLBCL | P25098 |
| Adrbk1 | Lymphoma -other | P25098 |
| Adrb2 | Pancreas | P07550 |
| Adrb2 | Prostate | P07550 |
| Adrb2 | upper aerodigestive | P07550 |
| Adrb2 | Urinary Tract | P07550 |
| Adrbk2 | Leuk. AML | P35626 |
| Adrbk2 | Leuk. B cell ALL | P35626 |
| Adrbk2 | Leuk. CML | P35626 |
| Adrbk2 | Leuk. T cell ALL | P35626 |
| Adrbk2 | neuroblastoma | P35626 |
| Adrbk2 | Lymphoma - Burkitt | P35626 |
| Adrbk2 | lymphoma - DLBCL | P35626 |
| Adrbk2 | Lymphoma -other | P35626 |
| Chrm3 | esophagus | P20309 |
| Chrm3 | multiple myeloma | P20309 |
| Chrna6 | Lymphoma - Burkitt | Q15825 |
| Chrna6 | Lymphoma - hodgkin | Q15825 |
| Chrna5 | neuroblastoma | P30532 |
| Chrna9 | Leuk. T cell ALL | Q9UGM1 |
| Chrna9 | Melanoma | Q9UGM1 |
| Chrnb1 | Leuk. B cell ALL | P11230 |
| Chrnb1 | Leuk. T cell ALL | P11230 |
| Chrnb4 | neuroblastoma | P30926 |
| Chrng | Meningioma | P07510 |
| ADCYAP1 R1 | neuroblastoma | P41586 |
| C4orf48 | Leukemia - other | Q5BLP8 |
| C4orf48 | Lymphoma - Burkitt | Q5BLP8 |
| CALCB | Ewings Sarcoma | P10092 |
| CALCB | lung - small cell | P10092 |
| CALCB | neuroblastoma | P10092 |
| CCK | Chondrosarcoma | P06307 |
| CCK | Ewings Sarcoma | P06307 |
| CPE | Leuk. AML | P16870 |
| CPE | Leuk. B cell ALL | P16870 |
| CPE | Leuk. CML | P16870 |
| CPE | Leuk. T cell ALL | P16870 |
| CPE | lung - small cell | P16870 |
| CPE | Lymphoma - Burkitt | P16870 |
| CPE | lymphoma - DLBCL | P16870 |
| CPE | Lymphoma - hodgkin | P16870 |
| CPE | Lymphoma -other | P16870 |
| CPE | multiple myeloma | P16870 |
| CPE | Prostate | P16870 |
| CPE | neuroblastoma | P16870 |
| CRHBP | lymphoma - DLBCL | P24387 |
| CYSLTR1 | Ewings Sarcoma | Q9Y271 |
| CYSLTR1 | Leuk. AML | Q9Y271 |
| CYSLTR1 | Leuk. B cell ALL | Q9Y271 |
| CYSLTR1 | Leuk. CML | Q9Y271 |
| CYSLTR1 | Leukemia - other | Q9Y271 |
| CYSLTR1 | Lymphoma - Burkitt | Q9Y271 |
| CYSLTR1 | lymphoma - DLBCL | Q9Y271 |
| CYSLTR1 | Lymphoma -other | Q9Y271 |
| CYSLTR1 | multiple myeloma | Q9Y271 |
| FOLH1 | Melanoma | Q04609 |
| FOLH1 | Prostate | Q04609 |
| GAL | Colorectal | P22466 |
| GAL | esophagus | P22466 |
| GAL | Leuk. CML | P22466 |
| GAL | mesothelioma | P22466 |
| GAL | Prostate | P22466 |
| GAL | neuroblastoma | P22466 |
| GALR3 | Meningioma | O60755 |
| GRP | Ewings Sarcoma | P11021 |
| GRP | lung - small cell | P11021 |
| KISS1R | Leuk. CML | Q969F8 |
| KISS1R | Leuk. CML | Q969F8 |
| KISS1R | Prostate | Q969F8 |
| KISS1R | multiple myeloma | Q969F8 |
| LTB4R | esophagus | Q15722 |
| LTB4R | Leuk. AML | Q15722 |
| LTB4R | Leuk. T cell ALL | Q15722 |
| LTB4R | Leukemia - other | Q15722 |
| LTB4R | Lymphoma - Burkitt | Q15722 |
| LTB4R | lymphoma - DLBCL | Q15722 |
| LTB4R | Lymphoma -other | Q15722 |
| MRGPRF | Meningioma | Q96AM1 |
| MRGPRF | mesothelioma | Q96AM1 |
| MRGPRF | osteosarcoma | Q96AM1 |
| NMU | Colorectal | P48645 |
| NMU | Endometrium | P48645 |
| NMU | Ewings Sarcoma | P48645 |
| NMU | Leuk. AML | P48645 |
| NMU | Leuk. B cell ALL | P48645 |
| NMU | Leuk. T cell ALL | P48645 |
| NMU | lung - small cell | P48645 |
| NMU | Lymphoma - Burkitt | P48645 |
| NMU | lymphoma - DLBCL | P48645 |
| NMU | Lymphoma -other | P48645 |
| NMU | Melanoma | P48645 |
| NMU | multiple myeloma | P48645 |
| NMU | osteosarcoma | P48645 |
| NMU | Ovary | P48645 |
| NMU | pancreas | P48645 |
| NMU | prostate | P48645 |
| NMU | Soft tissue | P48645 |
| NMU | upper aerodigestive | P48645 |
| NPB | Leukemia - other | Q8NG41 |
| NPBWR1 | Meningioma | P48145 |
| NPW | Ewings Sarcoma | Q8N729 |
| NPW | Leukemia - other | Q8N729 |
| NPW | Leukemia - other | Q8N729 |
| NPY | Ewings Sarcoma | P01303 |
| NPY | Leuk. B cell ALL | P01303 |
| NPY | neuroblastoma | P01303 |
| NPY5R | Ewings Sarcoma | Q15761 |
| NTS | esophagus | Q6FH20 |
| NTS | Ewings Sarcoma | Q6FH20 |
| NTS | lung - small cell | Q6FH20 |
| NTS | neuroblastoma | Q6FH20 |
| NTSR1 | Meningioma | P30989 |
| NXPH2 | Ewings Sarcoma | O95156 |
| PENK | Ewings Sarcoma | P01211 |
| PENK | osteosarcoma | P01211 |
| PNOC | Ewings Sarcoma | Q13519 |
| PNOC | Lymphoma - Burkitt | Q13519 |
| PNOC | lymphoma - DLBCL | Q13519 |
| PNOC | Lymphoma -other | Q13519 |
| PNOC | multiple myeloma | Q13519 |
| PROK2 | Ewings Sarcoma | Q9HC23 |
| PROK2 | Leuk. AML | Q9HC23 |
| PROK2 | Leuk. CML | Q9HC23 |
| PTH2R | Leuk. AML | Q9Y3E5 |
| PTH2R | Leuk. CML | Q9Y3E5 |
| PTH2R | neuroblastoma | Q9Y3E5 |
| RLN2 | lung - small cell | P04090 |
| RLN2 | Lymphoma - Burkitt | P04090 |
| RLN2 | pancreas | P04090 |
| RLN2 | prostate | P04090 |
| SCG2 | Chondrosarcoma | P13521 |
| SCG2 | Ewings Sarcoma | P13521 |
| SCG2 | Glioma | P13521 |
| SCG2 | lung - small cell | P13521 |
| SCG2 | Medulloblastoma | P13521 |
| SCG2 | Melanoma | P13521 |
| SCG2 | Soft Tissue | P13521 |
| SCG2 | Thyroid | P13521 |
| SCG2 | Meningioma | P13521 |
| SCG3 | lung - small cell | Q8WD2 |
| SCG3 | Medulloblastoma | Q8WD2 |
| SCG5 | Chondrosarcoma | P05408 |
| SCG5 | Glioma | P05408 |
| SCG5 | Kidney | P05408 |
| SCG5 | Thyroid | P05408 |
| SCG5 | lung - small cell | P05408 |
| SCG5 | mesothelioma | P05408 |
| SCG5 | neuroblastoma | P05408 |
| SORCS1 | Melanoma | Q8WY21 |
| SORCS2 | Chondrosarcoma | Q96PQ0 |
| SORCS2 | Upper aerodigestive | Q96PQ0 |
| SORCS2 | Medulloblastoma | Q96PQ0 |
| SSTR1 | Mesothelioma | P30872 |
| SSTR2 | Medulloblastoma | P30874 |
| SSTR2 | Neuroblastoma | P30874 |
| UCN2 | Melanoma | Q96RP3 |
| UTS2 | bile duct | O95399 |
| UTS2 | Ewings Sarcoma | O95399 |
| UTS2 | Leuk. AML | O95399 |
| VIP | Neuroblastoma | P01282 |
| VIPR1 | bile duct | P32241 |
| VIPR1 | Colorectal | P32241 |
| VIPR1 | Pancreas | P32241 |
| Htr2c | Esophagus | P28335 |

### Example 6—Neuromodulating Agents Affect Cancer Cell Growth

Cancer cell lines were grown in appropriate cell culture medium. On day 0, cells were plated in black organogenix nanocult 96-well plates with low-bind honeycomb pattern. Cells were plated in a low serum medium to increase the dynamic range observable for growth promotion. Within three days spheroids were visible.

On day 1 post plating, compounds were added at a range of doses to the cells, between mid-nanomolar and low micromolar, spanning at least a 100× dose range. On day 4 post plating, number of cells are quantified by staining with Cell Titer Glo and reading on a plate reader.

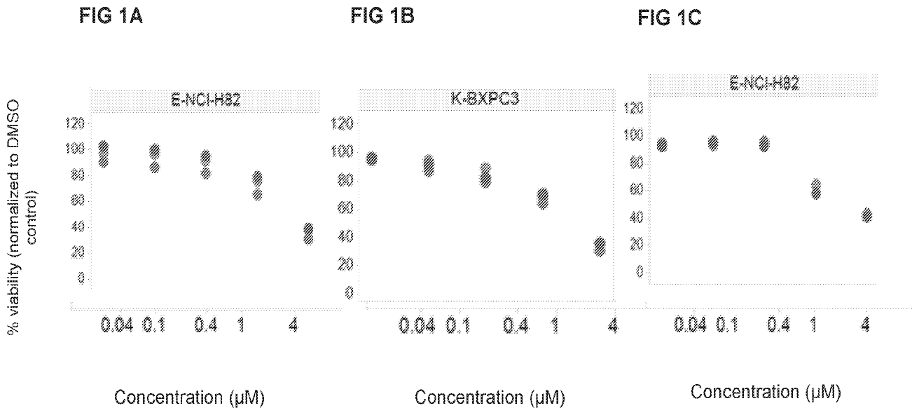

We observed that adenosine at 10-200 μM enhances the growth of multiple pancreatic cancer cell lines (CFPAC, PANC-1, PL45, ASPC-1, CAPAN-1, CAPAN-2) by at least 10% increased signal over 24 hours than untreated cells. Upon follow-up of the adenosine pathway, we performed a similar assay with chemical antagonists of adenosine receptors at a range of concentrations between 10 nM-10 uM. We found that the Adenosine Receptor 1 (A1R) antagonist KW3902 inhibited the growth of BXPC3 pancreatic cancer cells and NCI-H82 small cell lung cancer cells at low micromolar concentration (FIGS. 1A-1B). We also found that the Adenosine Receptor 3 (A3R) antagonist MRS-1220 inhibited the growth of NCI-H82 small cell lung cancer cells at low micromolar concentration (FIG. 1C).

We observed that histamine and the synthetic agonists 2-pyridylethylamine (H1 receptor), amthamine (H2 receptor), R-(−)-alpha-methylhistamine (H3 receptor), and VUF 10460 (H4 receptor) increase the growth rate of the pancreatic cancer cell lines PANC-1, PL45, ASPC-1, and CAPAN-2 by at least 5% compared to untreated control. These data suggest that inhibition of the histamine receptor signaling pathway and antagonism of the adenosine receptor signaling pathway inhibit cancer growth and can be a useful therapeutic for patients with pancreatic cancer, lung cancer, or other cancers that express these receptors.

### Example 7—Treatment of a Patient with Small Cell Lung Cancer with Adenosine Antagonist

According to the methods disclosed herein, a physician of skill in the art can treat a patient, such as a human patient with cancer (e.g., small cell lung cancer), so as to inhibit cancer growth, reduce tumor burden, or slow disease progression. To this end, a physician of skill in the art can administer to the human patient a neuromodulating agent that decreases adenosine signaling (e.g., an adenosine antagonist, such as MRS-1220, MRS-1334, MRS-1523, MRS-3777, MRE3008F20, PSB-10, PSB-11, and VUF-5574). MRS-1220 is administered orally to decrease cancer growth. MRS-1220 is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, MRS-1220 is administered bimonthly, once a month, once every two weeks, or at least once a week or more (e.g., 1, 2, 3, 4, 5, 6, or 7 times a week or more). MRS-1220 is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of MRS-1220 can be compared to evaluate the efficacy of the treatment, and the rate of disease progression can be assessed by comparison to the patient's medical history prior to administration of MRS-1220. A finding of a reduction in the total number of tumors, number of primary tumors, volume of tumors, growth of tumors, or rate of disease progression indicates that MRS-1220 has successfully treated the cancer.

### Example 8—Treatment of a Patient with Pancreatic Cancer with Histamine Antagonist

According to the methods disclosed herein, a physician of skill in the art can treat a patient, such as a human patient with cancer (e.g., pancreatic cancer), so as to inhibit cancer growth, reduce tumor burden, or slow disease progression. To this end, a physician of skill in the art can administer to the human patient a neuromodulating agent that decreases histamine signaling (e.g., a histamine H₁ receptor antagonist, such as acrivastine, azelastine, astemizole, bilastine, bromodiphenhydramine, brompheniramine, buclizine, carbinoxamine, and cetirizine). The histamine receptor antagonist is administered locally (e.g., injected intratumorally) to decrease cancer growth. The histamine receptor antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, The histamine receptor antagonist is administered bimonthly, once a month, once every two weeks, or at least once a week or more (e.g., 1, 2, 3, 4, 5, 6, or 7 times a week or more). The histamine receptor antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the histamine receptor antagonist can be compared to evaluate the efficacy of the treatment, and the rate of disease progression can be assessed by comparison to the patient's medical history prior to administration of the histamine receptor antagonist. A finding of a reduction in the total number of tumors, number of primary tumors, volume of tumors, growth of tumors, or rate of disease progression indicates that the histamine receptor antagonist has successfully treated the cancer.

### Example 9—Orthotopic MIAPACA2 Pancreatic Cancer Treated with Haloperidol

### Cell Culture

MIAPaCa2 Fluc cells were grown in DMEM tissue culture media with 10% fetal calf serum, 2.5% Horse Serum, 1% penicillin/streptomycin and Puromycin (20 ug/mL). On the day of implantation, cells were washed in PBS, trypsinized and resuspended at a density of 2×10⁶ cells/ml in a Matrigel solution prior to being implanted into the pancreas a volume of 0.05 mL using a 25 G needle.

### Mice

Nude mice (Nu/Nu) were anesthetized and the pancreas exposed via peritoneal incision and physical displacement of the spleen. Cells were injected into the pancreas in a total volume of 0.05 mL and the surgical wound closed with VetBond. Bioluminescence was assessed by administration of D-luciferin and imaging on an IVIS system. Mice with leakage of bioluminescent signal immediately after surgery were removed from study. Mice were randomized into groups of equal mean bioluminescence approximately 14 days post-implant.

### Drug Dosing

Haloperidol, an agent that blocks a variety of dopamine receptors and is used in the treatment of psychiatric disorders in humans, was administered daily IP in a solution of 0.85% lactic acid and 0.5% Cremophor EL. One group of mice received haloperidol alone, a second group received gemcitabine chemotherapy (the standard of care for human pancreatic cancer treatment), and a third group received a combination of haloperidol and gemcitabine. For groups also receiving gemcitabine, gemcitabine was dosed 50 mg/kg IP daily. Tumor growth was determined bi-weekly with bioluminescent measurement.

### End of Study

The study was terminated three weeks after initiation of drug dosing. At termination, mice were sacrificed and pancreas collected and weighed.

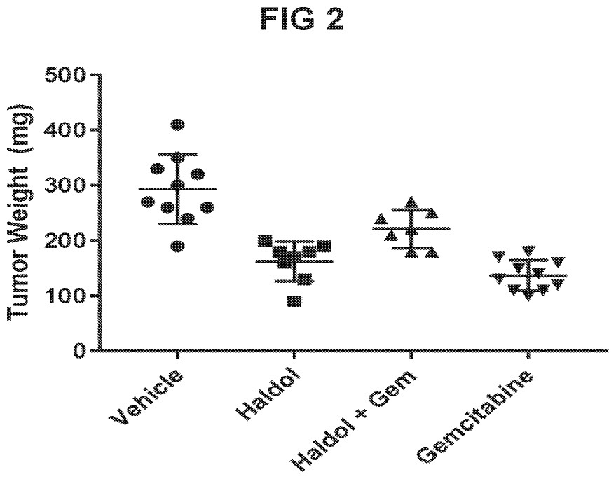

As shown in FIG. 2, mice that received Haldol saw a ˜50% reduction in tumor weight at the end of study (150 mg vs 300 mg for the vehicle-treated group). This reduction was equivalent to the reduction observed in the gemcitabine treated group. The combination treated group showed a reduction of tumor weight by about 67% compared to the vehicle-treated control group (200 mg vs 300 mg).

These data suggest that patients with pancreatic cancer that express dopamine receptor may be candidates for treatment with a dopaminergic antagonist, and that this pathway antagonism may result in reduced tumor growth and better outcomes. The dopaminergic antagonist may act by an orthogonal mechanism to standard chemotherapy, thus opening up this treatment to patients who have been previously treated with chemotherapy.

### Example 10—Pancreatic Cancer Treatment Using a Dopamine Antagonist

According to the methods disclosed herein, a physician of skill in the art can treat a patient, such as a human patient with cancer (e.g., pancreatic cancer), so as to inhibit cancer growth, reduce tumor burden, or slow disease progression. To this end, a physician of skill in the art can administer to the human patient a neuromodulating agent that decreases dopamine signaling (e.g., a dopamine antagonist, such as haloperidol, paliperidone, clozapine, risperidone, olanzapine, quetiapine, ziprasidone, amoxapine, clomipramine, and trimipramine). The dopamine antagonist can be administered at a dose lower or higher than that administered to a patient with a neuropsychiatric disorder, or it can be chemically modified (e.g., PEGylated), delivered in a particulate formulation (e.g., a nanoparticle or microparticle), or injected into the pancreatic tumor so that it does not cross the blood brain barrier. The dopamine antagonist is administered locally (e.g., injected intratumorally) to decrease cancer growth. The dopamine antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, The dopamine antagonist is administered bimonthly, once a month, once every two weeks, or at least once a week or more (e.g., 1, 2, 3, 4, 5, 6, or 7 times a week or more). The dopamine antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the dopamine antagonist can be compared to evaluate the efficacy of the treatment, and the rate of disease progression can be assessed by comparison to the patient's medical history prior to administration of the dopamine antagonist. A finding of a reduction in the total number of tumors, number of primary tumors, volume of tumors, growth of tumors, or rate of disease progression indicates that the dopamine antagonist has successfully treated the cancer.

### Example 11—Treatment of a Subcutaneous Tumor (SCLC PDX) Using an Implanted Microcassette

Small cell lung cancer cells derived from a human donor (CrownBio, San Diego Calif.) were injected subcutaneously into the flanks of nude mice. Tumors were grown to a size of approximately 6-10 mm before device implantation.

Microdose drug delivery devices were manufactured as described in Jonas et al., Sci. Transl. Med. 7, 284ra57 (2015). Briefly, cylindrical, micro-scale devices with 0.82 (diameter)×4 mm (length) were manufactured from medical-grade Delrin acetyl resin blocks (DuPont) by micromachining (CNC Micromachining Center, Cameron). Circular reservoirs were shaped on the outer surface of devices in dimensions of 230 um (diameter)×250 um (depth). Drug-polymer mixtures were packed into device reservoirs using a tapered, metal needle (Electron Microscopy Sciences) until the reservoirs were completely filled.

Devices were implanted directly into the mouse tumor using a 19-gauge spinal biopsy needle (Angiotech) and a retractable needle obturator to push the device into the tissue. Devices containing the drugs remained in situ for 24 hours.

The flank tumor was excised and the tissue containing the device was fixed for 24 hours in 10% formalin and perfused with paraffin. This specimen was sectioned using a standard microtome and tissue sections were collected from each reservoir. Sections were antibody-stained by standard immunohistochemistry using antibodies against cleaved-caspase-3 (CC3), KI-67, and phospho-AKT (Cell Signaling) and scored using an ImageJ image (v1.48) analysis algorithm in a blinded manner.

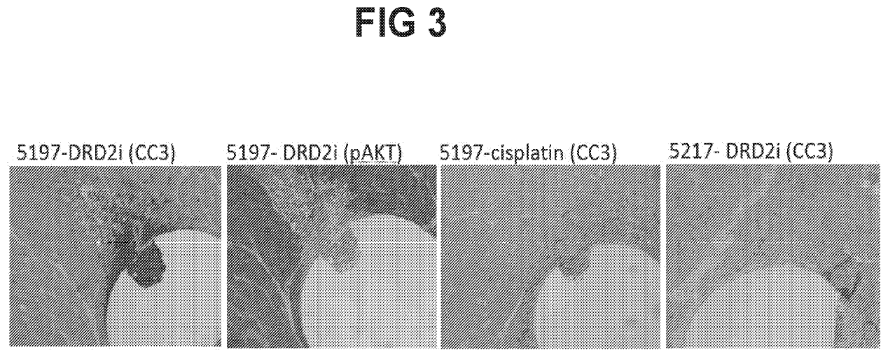

When the tumors were analyzed (FIG. 3), patient-derived xenograft tumors from patient 5197 that had been exposed to the reservoir containing the dopaminergic receptor DRD2 antagonist L-741,626, showed a dose-dependent killing as measured by CC3 positive tissue with a clear gradient away from the reservoir. The same sample showed a negative gradient for phospho-AKT indicating that the L-741,626 was inhibiting signaling via dopamine receptor, as AKT is downstream of DRD2. The same tumor exposed to cisplatin chemotherapy showed minimal cell killing, suggesting that dopaminergic antagonism is killing the cells by an independent mechanism. Interestingly, patient-derived xenograft tumors from patient 5217 that expresses ˜10× lower levels of DRD2 mRNA compared to patient 5197 were not impacted at all by treatment with the L-741,626 dopaminergic antagonist, suggesting again that the effect is mediated by signaling through dopamine receptor.

These data suggest that a patient whose tumor expresses a neurological gene signature with modulators of the activated neurological pathway may be a good candidate for treatment with a drug that targets the active neurological pathway in the tumor.

### Example 12—Treatment of SCLC Using a Dopamine Antagonist

According to the methods disclosed herein, a physician of skill in the art can treat a patient, such as a human patient with cancer (e.g., small cell lung cancer), so as to inhibit cancer growth, reduce tumor burden, or slow disease progression. The method of treatment can include diagnosing or identifying a patient as a candidate for treatment with a neuromodulating agent based on neurome gene expression in a biopsy. For example, a biopsy can be collected from a patient with small cell lung cancer and assayed for expression of a dopamine receptor using quantitative RT-PCR (qPCR), western blot analysis, immunohistochemistry, or ELISA. The expression of the dopamine receptor can be compared to the expression of a housekeeping gene, and a tumor with a higher relative expression of a dopamine receptor (e.g., 1.5, 2, 2.5, 3, 4, 5, or 10 or more fold higher relative expression of a dopamine receptor compared to a housekeeping gene) will be considered positive for expression of a dopamine receptor. A patient with such a tumor can be treated with a dopamine receptor antagonist. To this end, a physician of skill in the art can administer to the human patient a neuromodulating agent that decreases dopamine signaling (e.g., a dopamine antagonist, such as haloperidol, L-741,626, paliperidone, clozapine, risperidone, olanzapine, quetiapine, ziprasidone, amoxapine, clomipramine, and trimipramine). The dopamine antagonist can be administered at a dose lower or higher than that administered to a patient with a neuropsychiatric disorder, or it can be chemically modified (e.g., PEGylated), delivered in a particulate formulation (e.g., a nanoparticle or microparticle), conjugated to a targeting moiety, or administered locally so that it does not cross the blood brain barrier.

The dopamine antagonist is administered locally (e.g., injected intratumorally) to decrease cancer growth. The dopamine antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, The dopamine antagonist is administered bimonthly, once a month, once every two weeks, or at least once a week or more (e.g., 1, 2, 3, 4, 5, 6, or 7 times a week or more). The dopamine antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the dopamine antagonist can be compared to evaluate the efficacy of the treatment, and the rate of disease progression can be assessed by comparison to the patient's medical history prior to administration of the dopamine antagonist. A finding of a reduction in the total number of tumors, number of primary tumors, volume of tumors, growth of tumors, or rate of disease progression indicates that the dopamine antagonist has successfully treated the cancer.

### Example 13—Intratumoral Injection of Botox to Prevent Gastric Tumor Initiation

According to the methods disclosed herein, a physician of skill in the art can treat a patient, such as a human patient with cancer or at risk of developing cancer (e.g., gastric cancer), so as to inhibit cancer growth, reduce tumor burden, or prevent tumor initiation. The method of treatment can include diagnosing or identifying a patient as a candidate for treatment based on a genetic predisposition for developing cancer (e.g., mutations in CDH1, CTNNA1, DOT1L, FBXO24, PRSS1, MAP3K6, MSR1, or INSR for gastric cancer, see Petrovchich et al., Semin oncol 43:554 (2016)). To treat the patient, a physician of skill in the art can administer to the human patient a neuromodulating agent that decreases neurotransmission (e.g., a neurotoxin such as botulinum toxin, tetanus toxin, tetrodotoxin, or conotoxin). The neurotoxin (e.g., botulinum toxin A (Botox)) is administered locally (e.g., injected into the wall of the stomach). The dose of Botox administered is 200 U in a 2 mL volume, with higher or lower doses administered depending on tumor burden (e.g., higher doses can be administered to patients with larger or later stage tumors, while lower doses can be administered to prevent tumor initiation in patients with genetic mutations who have not yet developed cancer). In some embodiments, Botox is administered, twice a year, once every four months, bimonthly, once a month, once every two weeks, or at least once a week or more (e.g., 1, 2, 3, 4, 5, 6, or 7 times a week or more).

Botox is administered to the patient in an amount sufficient to decrease tumor growth decrease tumor burden, prevent tumor initiation, or decrease neurotransmission by 10% or more (e.g., 10%, 20%, 30%, 40%, 50%, 60%, 70%, 80%, 90%, 95% or more). Botox efficacy can be assessed by evaluating a biopsy sample of the patient's stomach wall for a reduction in electrical activity (e.g., using in vitro electrophysiological recording), or by evaluating the presence of cleaved SNARE proteins (the molecular target of Botox) using ELISA. Tumor growth and tumor burden can be assessed using standard imaging methods (e.g., digital radiography, positron emission tomography (PET) scan, computed tomography (CT) scan, or magnetic resonance imaging (MRI) scan). In patients at risk for developing cancer, gastric tissue biopsies can be compared before and after Botox administration to determine if the epithelial layers of the stomach are less neoplastic by histology. Images from before and after administration of Botox can be compared to evaluate the efficacy of the treatment, and the rate of disease progression can be assessed by comparison to the patient's medical history prior to administration of Botox. A finding of a reduction in the total number of tumors, number of primary tumors, volume of tumors, or growth of tumors, or stomach tissue that appears less neoplastic indicates that Botox has successfully treated the cancer or reduced the likelihood or prolonged the development of cancer.

### Example 14—Targeting Neurons in Pancreatic Cancer Using a Viral Vector Encoding a Neurotoxin

According to the methods disclosed herein, a physician of skill in the art can treat a patient, such as a human patient with cancer (e.g., pancreatic cancer), so as to inhibit cancer growth, reduce tumor burden, or prevent tumor initiation. The method of treatment can include diagnosing or identifying a patient as a candidate for treatment based on tumor innervation (e.g., based on a histological analysis of a tumor biopsy from the patient using neuron-specific antibodies such as anti-Neurofilament and anti-PGP9.5 to detect the presence of nerves). To treat a patient, a physician of skill in the art can administer to the human patient a neuromodulating agent that decreases neurotransmission (e.g., a neurotoxin such as botulinum toxin, tetanus toxin, tetrodotoxin, or conotoxin). A gene encoding tetanus toxin (e.g., TeNT, UniProt P04958) can be encoded in a replication-deficient herpes virus, as described in Burton et al., DNA & Cell Biology 21:915 (2002). For additional specificity, TeNT gene expression can be driven by a neuron-specific promoter, such as the synapsin promoter. The genetically modified herpes viral vector can be prepared for injection in bolus doses of 1×10\hat{ }11 particles per dose. The viral vector encoding a neurotoxin can be administered in combination with other cancer treatments, such as chemotherapeutic agents.

The patient can be started on a standard treatment of Gemcitabine intravenously at a dose of 1000 mg/m²+nab-paclitaxel 125 mg/m² as per hospital standards. One cycle will be one dose of Gemcitabine+nab-paclitaxel given on days 1, 8, and 15 of a 28 day cycle. The TeNT-encoding herpes viral vector can be administered by intratumoral injection every other week for 6 doses starting on day 15 of the first cycle of chemotherapy. The most common route of injection is ultrasound-guided percutaneous injection, but endoscopic ultrasound-guidance can be used for some patients as appropriate. All patients can receive anti-anxiety medication or sedation as needed for comfort during the procedure.

The viral vector infects tumor-resident neurons and travels to the dorsal root ganglion where it begins to produce recombinant TeNT. The toxin inhibits neurotransmitter release in the virally-transduced neurons, thus depriving the cancer of functional nerves essential for its survival. Neurotoxin efficacy can be assessed by evaluating a biopsy sample of the patient's tumor for a reduction in electrical activity (e.g., using in vitro electrophysiological recording), or by evaluating the presence of nerves using PGP9.5 or Neurofilament immunohistochemical staining in biopsy tissue sections. Electrical activity or tumor innervation can be reduced by 10% or more (e.g., 10%, 20%, 30%, 40%, 50%, 60%, 70%, 80%, 90%, 95% or more) compared to biopsy samples prior to viral vector administration, or compared to biopsy samples from patients treated with chemotherapy alone. Tumor growth and tumor burden can be assessed using standard imaging methods (e.g., digital radiography, positron emission tomography (PET) scan, computed tomography (CT) scan, or magnetic resonance imaging (MRI) scan). Images from before and after treatment can be compared to evaluate the efficacy of the treatment, and the rate of disease progression can be assessed by comparison to the patient's medical history prior to administration of the TeNT-encoding viral vector. Outcomes can also be compared between patients receiving chemotherapy alone and patients receiving combination therapy with the TeNT-encoding viral vector. A finding of a reduction in the total number of tumors, number of primary tumors, volume of tumors, or growth of tumors, or innervation of tumors indicates that the TeNT-encoding viral vector has improved cancer outcomes.

### Example 15—Administration of a Neurotrophic Factor Blocking Antibody to Treat Cancer

According to the methods disclosed herein, a physician of skill in the art can treat a patient, such as a human patient, so as to inhibit tumor growth (e.g., osteosarcoma growth). To this end, a physician of skill in the art can administer to the human patient a neuromodulating agent that inhibits neurogenic/axonogenic signals (e.g., a blocking or inhibitory antibody against a neurotrophic factor, such as NGF, BDNF, ProNGF, Sortilin, TGFβ family ligands, GFRα family ligands, CNTF, LIF, neurturin, artemin, persephin, neurotrophin, chemokines, cytokines, and others listed in Table 1C).

One exemplary antibody that can be used is an anti-NGF sequestering antibody (e.g., mAb 911, Rinat/Pfizer), the CDRs for which are described in Hongo et al., Hybridoma 19:215 (2000) and Cattaneo, Curr Opin Mol Ther 12:94 (2010). The anti-NGF antibody can be administered to the patient, for example, by parenteral administration (e.g., intramuscular or intravenous administration) or intratumorally, to inhibit tumor growth. The anti-NGF antibody is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the blocking or inhibitory antibody is administered at least once a week or more (e.g., 1, 2, 3, 4, 5, 6, or 7 times a week or more). The anti-NGF antibody can be administered to the patient in an amount sufficient to inhibit tumor growth or reduce tumor volume. Tumor growth can be assessed using imaging methods (e.g., digital radiography, computed tomography (CT) scan, or magnetic resonance imaging (MRI) scan). For bone cancer such as osteosarcoma, bone density can be assessed using digital radiography to monitor tumor growth. Radiograph images of the affected bones are used to evaluate tumor-induced bone destruction by assigning scores of 0-4: 0, normal bone with no signs of destruction; 1, small radiolucent lesions indicative of bone destruction (1-3 lesions); 2, increase in the number of lesions (3-6 lesions) and loss of medullary bone; 3, loss of medullary bone and erosion of cortical bone; 4, full-thickness unicortical bone loss. The patient can be evaluated, for instance, 1 month, 2 months, 3 months, 4 months, 5 months, 6 months, or more following administration of the anti-NGF antibody. A finding of the same or a reduced score following administration of the anti-NGF antibody provides an indication that the treatment has successfully inhibited tumor growth.

### Example 16—Colony Formation Assay to Test DRD2 Inhibition in Glioblastoma Cell Lines

Glioblastoma lines A172, LN-18, T98G, LN-229, U87 MG, and U118 MG were passaged in culture per manufacturer's recommendations.

On day 0 of the experiment, cells were plated in 24 well plates at a density of 500 cells per well in triplicate. On day 1, DRD2 antagonists haloperidol and L741,626 were added to the wells at a range of concentrations between 1 μM and 20 μM. Media was changed every three days. On day 9 of the experiment, cells were fixed with formalin and stained with Cyto60 (a nucleic acid stain). Fluorescence was read on a Licor-Odyssey imager to assess the number of viable cells in the assay.

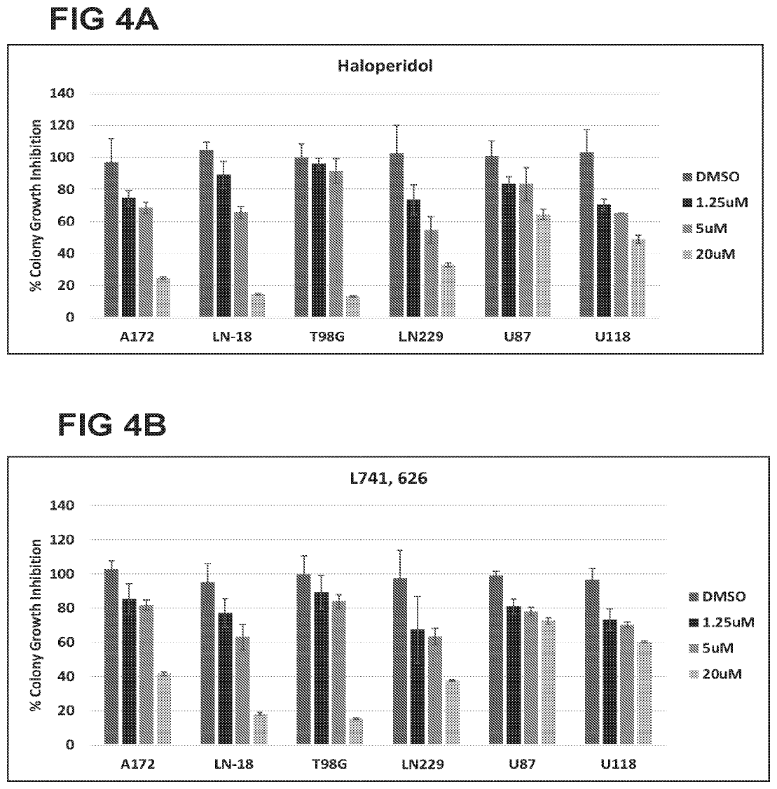

As shown FIGS. 4A-4B, haloperidol and L741,626 were able to reduce the number of glioblastoma cells growing in culture compared to DMSO control in a dose-dependent fashion in the cell lines. The effect was strongest for both agents in A172, LN-18, LN 229, and U118 cell lines.

These data suggest that dopaminergic antagonism is sufficient to limit the growth of glioma cell lines in culture, suggest that dopamine antagonists could be used to treat patients with glioma.

### Example 17—Treatment of Glioma Using a Dopamine Antagonist

According to the methods disclosed herein, a physician of skill in the art can treat a patient, such as a human patient with cancer (e.g., glioma), so as to inhibit cancer growth, reduce tumor burden, or slow disease progression. To this end, a physician of skill in the art can administer to the human patient a neuromodulating agent that decreases dopamine signaling (e.g., a dopamine antagonist, such as haloperidol, paliperidone, clozapine, risperidone, olanzapine, quetiapine, ziprasidone, amoxapine, clomipramine, L-741,626, and trimipramine). The dopamine antagonist is administered locally (e.g., injected intratumorally) to decrease cancer growth. The dopamine antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, The dopamine antagonist is administered bimonthly, once a month, once every two weeks, or at least once a week or more (e.g., 1, 2, 3, 4, 5, 6, or 7 times a week or more). The dopamine antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the dopamine antagonist can be compared to evaluate the efficacy of the treatment, and the rate of disease progression can be assessed by comparison to the patient's medical history prior to administration of the dopamine antagonist. A finding of a reduction in the total number of tumors, number of primary tumors, volume of tumors, growth of tumors, or rate of disease progression indicates that the dopamine antagonist has successfully treated the cancer.

### Example 18—Neuro-Dependent Cancer is a Novel Cancer Taxonomy

We observed that certain cancers express a gene signature that can be characterized as highly neurological, as defined by genes whose essential function is in the process of neurotransmission (e.g., neurome genes). These genes include neurotransmitter receptors, neuropeptide receptors, neurotransmitter transporters, neurotransporter biogenesis or biosynthetic genes, ion channels, ion pumps, vesicular proteins, synaptic junction proteins, axonal guidance proteins, neurotrophic factors, structural proteins, transporters, and signaling molecules found downstream of neuronal cell surface receptors (See Complete List in Table 7 and Table 8).

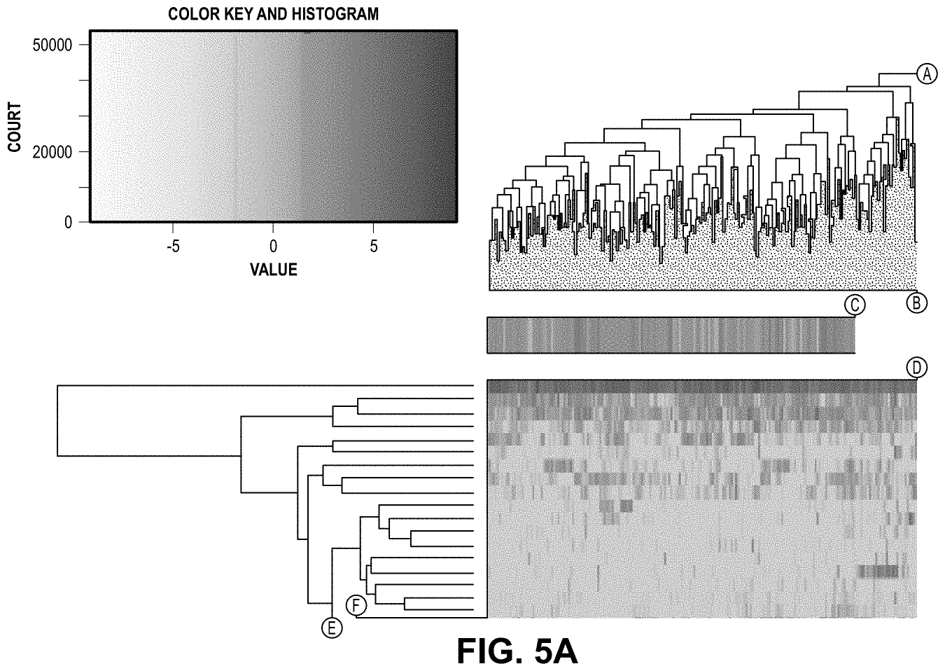

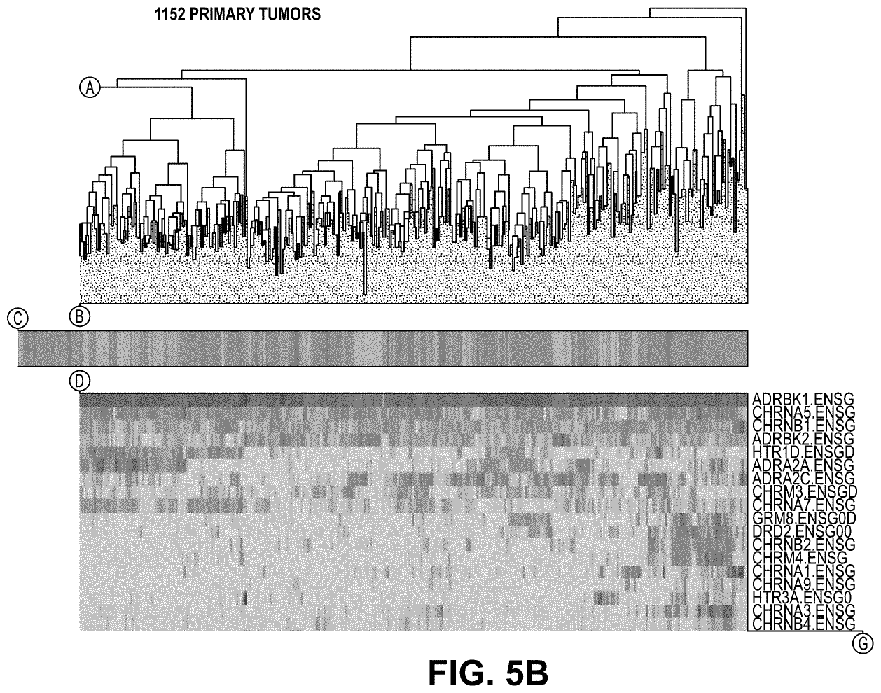

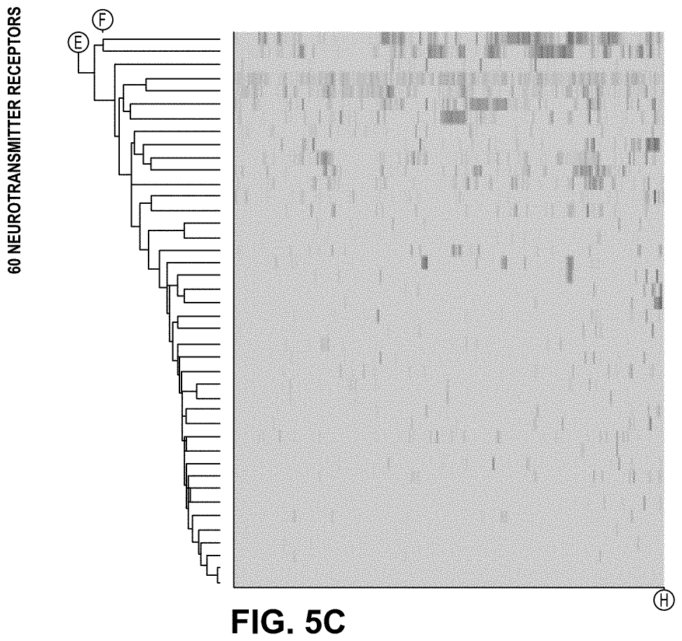

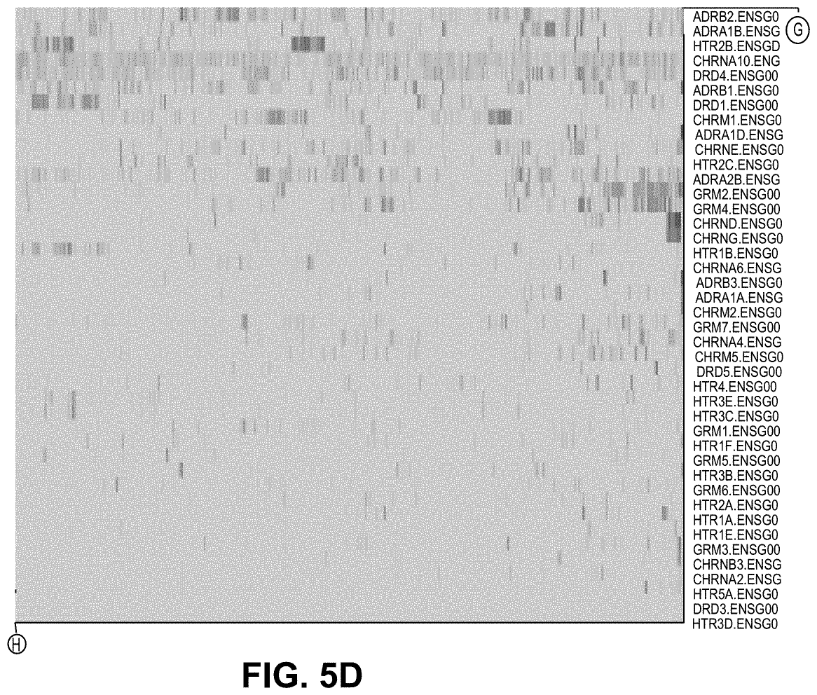

Surprisingly, the neurological gene signature of a tumor did not always correspond with the tissue type of origin. For example, in the heatmap (FIG. 5), 1152 primary tumors (columns) from human subjects were classified by their RNA expression of 60 different neurotransmitter receptors (rows). The tissue of origin of the tumors is denoted by the colored identifier bar at the top of each column. From this analysis, several neuro-defined groups emerged based on high expression of one or more of the following neurotransmitter receptor genes: (family 1) HTR1 D, ADRA2A, CHRNA7; (family 2) ADRA2C, CHRM3; (family 3) GRM8, DRD2, CHRNB2, CHRM4; (family 4) ADRAB2, ADRA1B.

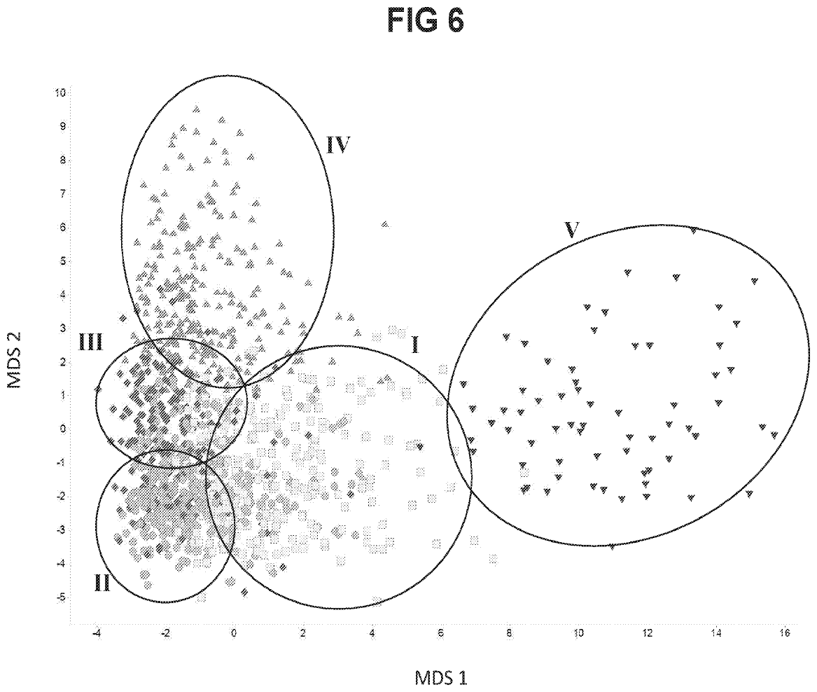

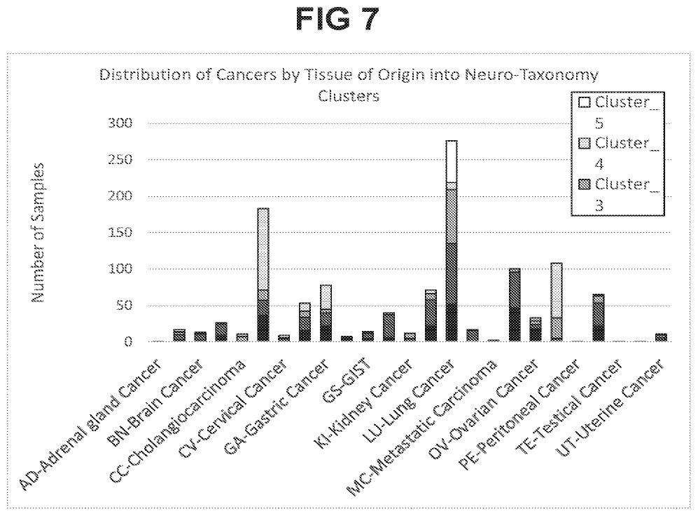

This phenomenon is amplified for visual effect by performing a principal components analysis, shown in the plot (FIG. 6). Essentially, neurotransmitter gene expression enabled the clustering of tumors into five distinct taxa, as shown in the left dot-plot where each dot represents a tumor type and the shape of the dot corresponds to the neurological taxon. In FIG. 7, the same data is represented but the tumors are displayed according to the tissue of origin. As is evident by inspection, the neurological taxonomy does not correspond to the tissue of origin and thus represents a new taxonomy for classifying tumors.

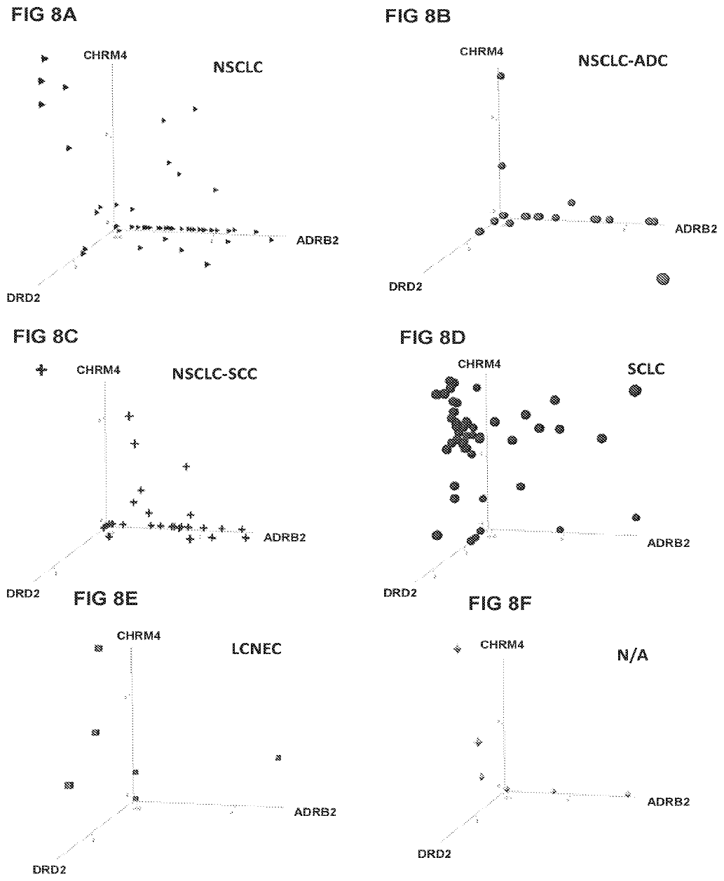

Deeper analysis of additional data sets supports this novel neurological classification. For example, as shown in FIGS. 8A-8F, our analysis of 276 primary lung cancers by RNA sequencing identified distinct clusters of tumor types based on their (1) co-expression of muscarinic receptor CHRM4 and dopamine receptor DRD2, (2) expression of adrenergic receptor ADRB2, or (3) low expression of all three. These graphs show that the neurome gene expression clustering does not correspond to common histological taxonomy. Most small cell lung cancer (SCLC) appear to be CHRM4 and DRD2 high (Group 1, corresponding to family 3 mentioned above. FIG. 8D), though others are negative (Group 3). Non-small cell lung cancer (NSCLC) samples are distributed into all three groups (FIGS. 8A-8C), with no correlation between squamous cell (SCC) and adenocarcinoma (ADC) subtypes.

This suggests that neurological diversity exists even within tumors that are homogeneously classified as “Lung cancer” according to tissue of origin, histology, or common oncogenic markers.

The implications for this novel neuro-taxonomy are that tumors that display an expression signature of neurome genes may be susceptible to particular interventions targeting these pathways or may be highly dependent on nerve-derived signals and thus responsive to neuro-ablative or neuromodulatory therapies.

### Example 19—Neurome Gene Overexpression in Cancer

One form of cancer neuro-dependence is marked by the overexpression of neurome genes, like neurotransmitter or neuropeptide receptors, synaptic junction proteins, neuronal growth factors, channels, transporter, signaling proteins, or others as listed in Table 7 or Table 8. Tumors that overexpress neurome genes may be more dependent on the signaling or molecular cues that are transmitted by these genes and proteins, and as such, these genes and proteins represent potential vulnerabilities for therapeutic intervention.

Shown in Tables 11A-11D below are gene expression signatures for four different neurotransmitter receptors across a range of human cancers: Dopamine receptor DRD2, muscarinic receptor CHRM3, adrenergic receptor ADRB2, and adrenergic receptor ADRB3. Each table displays RNAseq data for a particular neurotransmitter in tumor samples from patients with different types of cancer. Gene expression assessed using RNAseq was scored by log 2(FPKM). Values >0 are deemed to be “overexpressed” in this assay. The table shows the percentage of the total number of tumors analyzed from each cancer type (N) that overexpress the receptor. What is clear from this data is that multiple cancers across different tissue types express these neurotransmitter receptors. The ADRB3 data shows that this receptor is essentially not expressed across any cancer types, indicating that the patterns of expression for the other receptors is not a random fluctuation.

Table 12 shows the results of an analysis of neurome gene expression data (450 genes) from The Cancer Genome Atlas (TCGA), highlighting genes that were overexpressed in particular cancers present in the TCGA collection. Overexpressed gene-study pairs are selected if a gene was overexpressed in >20% of patients in a given study (frequently overexpressed) or if the mean Z score for patients with overexpression was >10 SD above the mean (abundantly overexpressed).

TABLE 11A
ADRB2 EXPRESSION IN CANCER

| Cancer type | | FKPM | FKPM | FKPM |
|---|---|---|---|---|
| ADRB2 | N | <0 | 0-2 | >2 |
| BL-Bladder Cancer | 10 | 90.0% | 0.0% | 10.0% |
| BN-Brain Cancer | 6 | 66.7% | 16.7% | 16.7% |
| BR-Breast Cancer | 16 | 87.5% | 12.5% | 0.0% |
| CR-Colorectal Cancer | 47 | 93.6% | 6.4% | 0.0% |
| ES-Esophageal Cancer | 20 | 80.0% | 15.0% | 5.0% |
| GS-GIST | 14 | 92.9% | 7.1% | 0.0% |
| HN-Head and Neck Cancer | 10 | 10.0% | 20.0% | 70.0% |
| KI-Kidney Cancer | 7 | 85.7% | 14.3% | 0.0% |
| LU-Lung Cancer | 194 | 71.6% | 20.1% | 8.2% |
| ME-Melanoma | 95 | 73.7% | 20.0% | 6.3% |
| OV-Ovarian Cancer | 19 | 94.7% | 5.3% | 0.0% |
| PA-Pancreatic Cancer | 24 | 75.0% | 20.8% | 4.2% |
| SA-Sarcoma | 57 | 56.1% | 28.1% | 15.8% |
| UT-Uterine Cancer | 11 | 90.9% | 9.1% | 0.0% |

TABLE 11B
ADRB3 EXPRESSION IN CANCER

| Cancer type | | FKPM | FKPM | FKPM |
|---|---|---|---|---|
| ADRB3 | N | <0 | 0-2 | >2 |
| BL-Bladder Cancer | 10 | 100.0% | 0.0% | 0.0% |
| BN-Brain Cancer | 6 | 100.0% | 0.0% | 0.0% |
| BR-Breast Cancer | 16 | 100.0% | 0.0% | 0.0% |
| CR-Colorectal Cancer | 47 | 100.0% | 0.0% | 0.0% |
| ES-Esophageal Cancer | 20 | 100.0% | 0.0% | 0.0% |
| GS-GIST | 14 | 78.6% | 7.1% | 14.3% |
| HN-Head and Neck Cancer | 10 | 80.0% | 10.0% | 10.0% |
| KI-Kidney Cancer | 7 | 100.0% | 0.0% | 0.0% |
| LU-Lung Cancer | 194 | 100.0% | 0.0% | 0.0% |
| ME-Melanoma | 95 | 100.0% | 0.0% | 0.0% |
| OV-Ovarian Cancer | 19 | 100.0% | 0.0% | 0.0% |
| PA-Pancreatic Cancer | 24 | 100.0% | 0.0% | 0.0% |
| SA-Sarcoma | 57 | 84.2% | 5.3% | 10.5% |
| UT-Uterine Cancer | 11 | 100.0% | 0.0% | 0.0% |

TABLE 11C
CHRM3 EXPRESSION IN CANCER

| Cancer type | | FKPM | FKPM | FKPM |
|---|---|---|---|---|
| CHRM3 | N | <0 | 0-2 | >2 |
| BL-Bladder Cancer | 10 | 70.0% | 30.0% | 0.0% |
| BN-Brain Cancer | 6 | 16.7% | 33.3% | 50.0% |
| BR-Breast Cancer | 16 | 62.5% | 18.8% | 18.8% |
| CR-Colorectal Cancer | 47 | 25.5% | 34.0% | 40.4% |
| ES-Esophageal Cancer | 20 | 75.0% | 20.0% | 5.0% |
| GS-GIST | 14 | 50.0% | 14.3% | 35.7% |
| HN-Head and Neck Cancer | 10 | 50.0% | 40.0% | 10.0% |
| KI-Kidney Cancer | 7 | 71.4% | 14.3% | 14.3% |
| LU-Lung Cancer | 194 | 60.8% | 29.9% | 9.3% |
| ME-Melanoma | 95 | 69.5% | 20.0% | 10.5% |
| OV-Ovarian Cancer | 19 | 52.6% | 42.1% | 5.3% |
| PA-Pancreatic Cancer | 24 | 79.2% | 20.8% | 0.0% |
| SA-Sarcoma | 57 | 78.9% | 10.5% | 10.5% |
| UT-Uterine Cancer | 11 | 54.5% | 27.3% | 18.2% |

TABLE 11D
DRD2 EXPRESSION IN CANCER

| Cancer type | | FKPM | FKPM | FKPM |
|---|---|---|---|---|
| DRD2 | N | <0 | 0-2 | >2 |
| BL-Bladder Cancer | 10 | 70.0% | 0.0% | 30.0% |
| BN-Brain Cancer | 6 | 66.7% | 16.7% | 16.7% |
| BR-Breast Cancer | 16 | 68.8% | 31.3% | 0.0% |
| CR-Colorectal Cancer | 47 | 63.8% | 27.7% | 8.5% |
| ES-Esophageal Cancer | 20 | 95.0% | 5.0% | 0.0% |
| GS-GIST | 14 | 100.0% | 0.0% | 0.0% |
| HN-Head and Neck Cancer | 10 | 100.0% | 0.0% | 0.0% |
| KI-Kidney Cancer | 7 | 100.0% | 0.0% | 0.0% |
| LU-Lung Cancer | 194 | 67.0% | 9.3% | 23.7% |
| ME-Melanoma | 95 | 95.8% | 3.2% | 1.1% |
| OV-Ovarian Cancer | 19 | 68.4% | 21.1% | 10.5% |
| PA-Pancreatic Cancer | 24 | 100.0% | 0.0% | 0.0% |
| SA-Sarcoma | 57 | 71.9% | 12.3% | 15.8% |
| UT-Uterine Cancer | 11 | 90.9% | 9.1% | 0.0% |

TABLE 12
OVEREXPRESSION OF NEUROME GENES IN CANCER

ADCY5
Kidney Renal Clear Cell Carcinoma (TCGA, Nature 2013)
ADCY6
Papillary Thyroid Carcinoma (TCGA, Cell 2014)
Thyroid Carcinoma (TCGA, Provisional)
ADCY7
Uterine Carcinosarcoma (TCGA, Provisional)
ADK
Kidney Chromophobe (TCGA, Cancer Cell 2014)
Kidney Chromophobe (TCGA, Provisional)
Lymphoid Neoplasm Diffuse Large B-cell Lymphoma (TCGA, Provisional)
ADRBK1
Glioblastoma Multiforme (TCGA, Provisional)
Lung Adenocarcinoma (TCGA, Nature 2014)
Lymphoid Neoplasm Diffuse Large B-cell Lymphoma (TCGA, Provisional)
ADRBK2
Uterine Carcinosarcoma (TCGA, Provisional)
AKAP10
Acute Myeloid Leukemia (TCGA, NEJM 2013)
Acute Myeloid Leukemia (TCGA, Provisional)
Bladder Urothelial Carcinoma (TCGA, Provisional)
Brain Lower Grade Glioma (TCGA, Provisional)
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Cholangiocarcinoma (TCGA, Provisional)
Colorectal Adenocarcinoma (TCGA, Provisional)
Esophageal Carcinoma (TCGA, Provisional)
Head and Neck Squamous Cell Carcinoma (TCGA, Nature 2015)
Head and Neck Squamous Cell Carcinoma (TCGA, Provisional)
Kidney Renal Clear Cell Carcinoma (TCGA, Nature 2013)
Kidney Renal Clear Cell Carcinoma (TCGA, Provisional)
Kidney Renal Papillary Cell Carcinoma (TCGA, Provisional)
Lung Adenocarcinoma (TCGA, Nature 2014)
Mesothelioma (TCGA, Provisional)
Ovarian Serous Cystadenocarcinoma (TCGA, Provisional)
Pancreatic Adenocarcinoma (TCGA, Provisional)
Papillary Thyroid Carcinoma (TCGA, Cell 2014)
Pheochromocytoma and Paraganglioma (TCGA, Provisional)
Prostate Adenocarcinoma (TCGA, Provisional)
Skin Cutaneous Melanoma (TCGA, Provisional)
Stomach Adenocarcinoma (TCGA, Provisional)
Testicular Germ Cell Cancer (TCGA, Provisional)
Thyroid Carcinoma (TCGA, Provisional)
Uterine Carcinosarcoma (TCGA, Provisional)
AKAP11
Adrenocortical Carcinoma (TCGA, Provisional)
Cervical Squamous Cell Carcinoma and Endocervical Adenocarcinoma (TCGA, Provisional)
Cholangiocarcinoma (TCGA, Provisional)
Esophageal Carcinoma (TCGA, Provisional)
Glioblastoma (TCGA, Cell 2013)
Glioblastoma Multiforme (TCGA, Provisional)
Lung Adenocarcinoma (TCGA, Nature 2014)
Lung Adenocarcinoma (TCGA, Provisional)
Lung Squamous Cell Carcinoma (TCGA, Provisional)
Ovarian Serous Cystadenocarcinoma (TCGA, Provisional)
AKAP6
Brain Lower Grade Glioma (TCGA, Provisional)
Kidney Renal Clear Cell Carcinoma (TCGA, Nature 2013)
AKAP7
Mesothelioma (TCGA, Provisional)
Skin Cutaneous Melanoma (TCGA, Provisional)
Thymoma (TCGA, Provisional)
Uveal Melanoma (TCGA, Provisional)
AKAP8
Bladder Urothelial Carcinoma (TCGA, Provisional)
Cervical Squamous Cell Carcinoma and Endocervical Adenocarcinoma (TCGA, Provisional)
Cholangiocarcinoma (TCGA, Provisional)
Mesothelioma (TCGA, Provisional)
Thymoma (TCGA, Provisional)
Uterine Carcinosarcoma (TCGA, Provisional)
ALDH5A1
Brain Lower Grade Glioma (TCGA, Provisional)
Kidney Chromophobe (TCGA, Cancer Cell 2014)
Kidney Chromophobe (TCGA, Provisional)
ALDH9A1
Bladder Urothelial Carcinoma (TCGA, Nature 2014)
ATP1A1
Acute Myeloid Leukemia (TCGA, NEJM 2013)
Acute Myeloid Leukemia (TCGA, Provisional)
Cholangiocarcinoma (TCGA, Provisional)
Testicular Germ Cell Cancer (TCGA, Provisional)
Uveal Melanoma (TCGA, Provisional)
ATP2A2
Cholangiocarcinoma (TCGA, Provisional)
Ovarian Serous Cystadenocarcinoma (TCGA, Provisional)
BACE1
Cholangiocarcinoma (TCGA, Provisional)
Kidney Chromophobe (TCGA, Cancer Cell 2014)
Kidney Chromophobe (TCGA, Provisional)
Pheochromocytoma and Paraganglioma (TCGA, Provisional)
Testicular Germ Cell Cancer (TCGA, Provisional)
Uterine Carcinosarcoma (TCGA, Provisional)
CASK
Uterine Carcinosarcoma (TCGA, Provisional)
CDK5
Esophageal Carcinoma (TCGA, Provisional)
CDK5R1
Uterine Carcinosarcoma (TCGA, Provisional)
CHRNB1
Thymoma (TCGA, Provisional)
Uterine Carcinosarcoma (TCGA, Provisional)
COMT
Esophageal Carcinoma (TCGA, Provisional)
Glioblastoma (TCGA, Cell 2013)
Glioblastoma Multiforme (TCGA, Provisional)
Mesothelioma (TCGA, Provisional)
Ovarian Serous Cystadenocarcinoma (TCGA, Provisional)
CRCP
Acute Myeloid Leukemia (TCGA, NEJM 2013)
Acute Myeloid Leukemia (TCGA, Provisional)
Bladder Urothelial Carcinoma (TCGA, Nature 2014)
Brain Lower Grade Glioma (TCGA, Provisional)
Cervical Squamous Cell Carcinoma and Endocervical Adenocarcinoma (TCGA, Provisional)
Glioblastoma (TCGA, Cell 2013)
Kidney Chromophobe (TCGA, Cancer Cell 2014)
Kidney Chromophobe (TCGA, Provisional)
Lymphoid Neoplasm Diffuse Large B-cell Lymphoma (TCGA, Provisional)
Ovarian Serous Cystadenocarcinoma (TCGA, Provisional)
Testicular Germ Cell Cancer (TCGA, Provisional)
Thymoma (TCGA, Provisional)
Uterine Carcinosarcoma (TCGA, Provisional)
CSK
Colorectal Adenocarcinoma (TCGA, Provisional)
Kidney Renal Clear Cell Carcinoma (TCGA, Nature 2013)
Lymphoid Neoplasm Diffuse Large B-cell Lymphoma (TCGA, Provisional)
Ovarian Serous Cystadenocarcinoma (TCGA, Provisional)
Uterine Carcinosarcoma (TCGA, Provisional)
CSNK1E
Brain Lower Grade Glioma (TCGA, Provisional)
Breast Invasive Carcinoma (TCGA, Cell 2015)
Breast Invasive Carcinoma (TCGA, Provisional)
Ovarian Serous Cystadenocarcinoma (TCGA, Provisional)
Pheochromocytoma and Paraganglioma (TCGA, Provisional)
Uterine Corpus Endometrial Carcinoma (TCGA, Provisional)
CYSLTR2
Uterine Carcinosarcoma (TCGA, Provisional)
DAGLA
Uterine Carcinosarcoma (TCGA, Provisional)
DAGLB
Lymphoid Neoplasm Diffuse Large B-cell Lymphoma (TCGA, Provisional)
Ovarian Serous Cystadenocarcinoma (TCGA, Provisional)
Stomach Adenocarcinoma (TCGA, Nature 2014)
DDR1
Brain Lower Grade Glioma (TCGA, Provisional)
Kidney Chromophobe (TCGA, Cancer Cell 2014)
Kidney Chromophobe (TCGA, Provisional)
Thyroid Carcinoma (TCGA, Provisional)
DPYSL2
Kidney Renal Clear Cell Carcinoma (TCGA, Nature 2013)
Kidney Renal Clear Cell Carcinoma (TCGA, Provisional)
Uterine Carcinosarcoma (TCGA, Provisional)
EEF2K
Kidney Renal Clear Cell Carcinoma (TCGA, Nature 2013)
Kidney Renal Clear Cell Carcinoma (TCGA, Provisional)
Ovarian Serous Cystadenocarcinoma (TCGA, Provisional)
ERBB2
Kidney Chromophobe (TCGA, Cancer Cell 2014)
Kidney Chromophobe (TCGA, Provisional)
Ovarian Serous Cystadenocarcinoma (TCGA, Provisional)
F2RL1
Kidney Chromophobe (TCGA, Cancer Cell 2014)
Kidney Chromophobe (TCGA, Provisional)
FNTA
Acute Myeloid Leukemia (TCGA, NEJM 2013)
Acute Myeloid Leukemia (TCGA, Provisional)
Bladder Urothelial Carcinoma (TCGA, Provisional)
Cervical Squamous Cell Carcinoma and Endocervical Adenocarcinoma (TCGA, Provisional)
Cholangiocarcinoma (TCGA, Provisional)
Head and Neck Squamous Cell Carcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Liver Hepatocellular Carcinoma (TCGA, Provisional)
Lymphoid Neoplasm Diffuse Large B-cell Lymphoma (TCGA, Provisional)
Ovarian Serous Cystadenocarcinoma (TCGA, Provisional)
Pancreatic Adenocarcinoma (TCGA, Provisional)
Papillary Thyroid Carcinoma (TCGA, Cell 2014)
Pheochromocytoma and Paraganglioma (TCGA, Provisional)
Prostate Adenocarcinoma (TCGA, Provisional)
Sarcoma (TCGA, Provisional)
Skin Cutaneous Melanoma (TCGA, Provisional)
Stomach Adenocarcinoma (TCGA, Provisional)
Thymoma (TCGA, Provisional)
Thyroid Carcinoma (TCGA, Provisional)
Uterine Corpus Endometrial Carcinoma (TCGA, Nature 2013)
GABARAP
Colorectal Adenocarcinoma (TCGA, Provisional)
Lung Adenocarcinoma (TCGA, Nature 2014)
Uterine Carcinosarcoma (TCGA, Provisional)
Uterine Corpus Endometrial Carcinoma (TCGA, Nature 2013)
GNA11
Brain Lower Grade Glioma (TCGA, Provisional)
Breast Invasive Carcinoma (TCGA, Provisional)
Cervical Squamous Cell Carcinoma and Endocervical Adenocarcinoma (TCGA, Provisional)
Cholangiocarcinoma (TCGA, Provisional)
Head and Neck Squamous Cell Carcinoma (TCGA, Nature 2015)
Kidney Chromophobe (TCGA, Cancer Cell 2014)
Kidney Chromophobe (TCGA, Provisional)
Liver Hepatocellular Carcinoma (TCGA, Provisional)
Ovarian Serous Cystadenocarcinoma (TCGA, Provisional)
Pheochromocytoma and Paraganglioma (TCGA, Provisional)
Prostate Adenocarcinoma (TCGA, Provisional)
Thyroid Carcinoma (TCGA, Provisional)
GNAI2
Bladder Urothelial Carcinoma (TCGA, Nature 2014)
Glioblastoma (TCGA, Cell 2013)
Head and Neck Squamous Cell Carcinoma (TCGA, Nature 2015)
Head and Neck Squamous Cell Carcinoma (TCGA, Provisional)
Lung Squamous Cell Carcinoma (TCGA, Provisional)
Lymphoid Neoplasm Diffuse Large B-cell Lymphoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
Thymoma (TCGA, Provisional)
Uterine Carcinosarcoma (TCGA, Provisional)
Uveal Melanoma (TCGA, Provisional)
GNAI3
Acute Myeloid Leukemia (TCGA, NEJM 2013)
Acute Myeloid Leukemia (TCGA, Provisional)
Brain Lower Grade Glioma (TCGA, Provisional)
Cholangiocarcinoma (TCGA, Provisional)
Colorectal Adenocarcinoma (TCGA, Provisional)
Head and Neck Squamous Cell Carcinoma (TCGA, Nature 2015)
Head and Neck Squamous Cell Carcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Lung Adenocarcinoma (TCGA, Nature 2014)
Lung Squamous Cell Carcinoma (TCGA, Provisional)
Lymphoid Neoplasm Diffuse Large B-cell Lymphoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
Pheochromocytoma and Paraganglioma (TCGA, Provisional)
Prostate Adenocarcinoma (TCGA, Provisional)
Sarcoma (TCGA, Provisional)
Thymoma (TCGA, Provisional)
Uterine Carcinosarcoma (TCGA, Provisional)
Uveal Melanoma (TCGA, Provisional)
GNAO1
Brain Lower Grade Glioma (TCGA, Provisional)
Pheochromocytoma and Paraganglioma (TCGA, Provisional)
GNAQ
Adrenocortical Carcinoma (TCGA, Provisional)
Cholangiocarcinoma (TCGA, Provisional)
Colorectal Adenocarcinoma (TCGA, Provisional)
Lung Adenocarcinoma (TCGA, Nature 2014)
Lung Adenocarcinoma (TCGA, Provisional)
Stomach Adenocarcinoma (TCGA, Nature 2014)
Uterine Carcinosarcoma (TCGA, Provisional)
GNAS
Acute Myeloid Leukemia (TCGA, NEJM 2013)
Acute Myeloid Leukemia (TCGA, Provisional)
GNB1
Bladder Urothelial Carcinoma (TCGA, Nature 2014)
Brain Lower Grade Glioma (TCGA, Provisional)
Colorectal Adenocarcinoma (TCGA, Provisional)
Glioblastoma (TCGA, Cell 2013)
Glioblastoma Multiforme (TCGA, Provisional)
Head and Neck Squamous Cell Carcinoma (TCGA, Nature 2015)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Kidney Renal Papillary Cell Carcinoma (TCGA, Provisional)
Liver Hepatocellular Carcinoma (TCGA, Provisional)
Lung Squamous Cell Carcinoma (TCGA, Provisional)
Lymphoid Neoplasm Diffuse Large B-cell Lymphoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
Pheochromocytoma and Paraganglioma (TCGA, Provisional)
Prostate Adenocarcinoma (TCGA, Provisional)
Sarcoma (TCGA, Provisional)
Thymoma (TCGA, Provisional)
Uterine Carcinosarcoma (TCGA, Provisional)
Uterine Corpus Endometrial Carcinoma (TCGA, Provisional)
GNB2
Kidney Renal Clear Cell Carcinoma (TCGA, Nature 2013)
Kidney Renal Clear Cell Carcinoma (TCGA, Provisional)
Kidney Renal Papillary Cell Carcinoma (TCGA, Provisional)
Lung Adenocarcinoma (TCGA, Nature 2014)
Lung Adenocarcinoma (TCGA, Provisional)
GNB4
Uterine Carcinosarcoma (TCGA, Provisional)
GNB5
Papillary Thyroid Carcinoma (TCGA, Cell 2014)
Pheochromocytoma and Paraganglioma (TCGA, Provisional)
Thyroid Carcinoma (TCGA, Provisional)
GNG10
Kidney Chromophobe (TCGA, Cancer Cell 2014)
Kidney Chromophobe (TCGA, Provisional)
Ovarian Serous Cystadenocarcinoma (TCGA, Provisional)
Uterine Carcinosarcoma (TCGA, Provisional)
GNG12
Liver Hepatocellular Carcinoma (TCGA, Provisional)
Pancreatic Adenocarcinoma (TCGA, Provisional)
Pheochromocytoma and Paraganglioma (TCGA, Provisional)
GNG5
Adrenocortical Carcinoma (TCGA, Provisional)
Brain Lower Grade Glioma (TCGA, Provisional)
Esophageal Carcinoma (TCGA, Provisional)
Head and Neck Squamous Cell Carcinoma (TCGA, Nature 2015)
Mesothelioma (TCGA, Provisional)
Uterine Carcinosarcoma (TCGA, Provisional)
GPI
Kidney Chromophobe (TCGA, Cancer Cell 2014)
Kidney Chromophobe (TCGA, Provisional)
GRB2
Bladder Urothelial Carcinoma (TCGA, Nature 2014)
Colorectal Adenocarcinoma (TCGA, Provisional)
Glioblastoma Multiforme (TCGA, Provisional)
Head and Neck Squamous Cell Carcinoma (TCGA, Provisional)
Lymphoid Neoplasm Diffuse Large B-cell Lymphoma (TCGA, Provisional)
Ovarian Serous Cystadenocarcinoma (TCGA, Provisional)
Pancreatic Adenocarcinoma (TCGA, Provisional)
Pheochromocytoma and Paraganglioma (TCGA, Provisional)
Prostate Adenocarcinoma (TCGA, Provisional)
Thymoma (TCGA, Provisional)
Uterine Carcinosarcoma (TCGA, Provisional)
GRID1
Brain Lower Grade Glioma (TCGA, Provisional)
GRIK5
Brain Lower Grade Glioma (TCGA, Provisional)
GRK4
Mesothelioma (TCGA, Provisional)
GRK5
Glioblastoma Multiforme (TCGA, Provisional)
HMOX2
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Ovarian Serous Cystadenocarcinoma (TCGA, Provisional)
Prostate Adenocarcinoma (TCGA, Provisional)
HNMT
Pancreatic Adenocarcinoma (TCGA, Provisional)
Prostate Adenocarcinoma (TCGA, Provisional)
IL6R
Kidney Chromophobe (TCGA, Cancer Cell 2014)
Kidney Chromophobe (TCGA, Provisional)
KRAS
Cervical Squamous Cell Carcinoma and Endocervical Adenocarcinoma (TCGA, Provisional)
Cholangiocarcinoma (TCGA, Provisional)
LIFR
Kidney Chromophobe (TCGA, Cancer Cell 2014)
Kidney Chromophobe (TCGA, Provisional)
LRTOMT
Adrenocortical Carcinoma (TCGA, Provisional)
Pheochromocytoma and Paraganglioma (TCGA, Provisional)
MAOB
Kidney Renal Clear Cell Carcinoma (TCGA, Nature 2013)
Kidney Renal Clear Cell Carcinoma (TCGA, Provisional)
MAP2K1
Colorectal Adenocarcinoma (TCGA, Provisional)
Ovarian Serous Cystadenocarcinoma (TCGA, Provisional)
Pancreatic Adenocarcinoma (TCGA, Provisional)
Testicular Germ Cell Cancer (TCGA, Provisional)
MAP2K2
Cholangiocarcinoma (TCGA, Provisional)
Esophageal Carcinoma (TCGA, Provisional)
Kidney Renal Clear Cell Carcinoma (TCGA, Nature 2013)
Kidney Renal Clear Cell Carcinoma (TCGA, Provisional)
Lung Squamous Cell Carcinoma (TCGA, Provisional)
MAP3K1
Head and Neck Squamous Cell Carcinoma (TCGA, Nature 2015)
Uterine Carcinosarcoma (TCGA, Provisional)
MAPK1
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Cholangiocarcinoma (TCGA, Provisional)
Colorectal Adenocarcinoma (TCGA, Provisional)
Esophageal Carcinoma (TCGA, Provisional)
Glioblastoma Multiforme (TCGA, Provisional)
Head and Neck Squamous Cell Carcinoma (TCGA, Nature 2015)
Kidney Chromophobe (TCGA, Cancer Cell 2014)
Kidney Chromophobe (TCGA, Provisional)
Kidney Renal Papillary Cell Carcinoma (TCGA, Provisional)
Lung Adenocarcinoma (TCGA, Nature 2014)
Lung Squamous Cell Carcinoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
Papillary Thyroid Carcinoma (TCGA, Cell 2014)
Pheochromocytoma and Paraganglioma (TCGA, Provisional)
Stomach Adenocarcinoma (TCGA, Provisional)
Thyroid Carcinoma (TCGA, Provisional)
Uterine Carcinosarcoma (TCGA, Provisional)
Uterine Corpus Endometrial Carcinoma (TCGA, Provisional)
MAPK14
Brain Lower Grade Glioma (TCGA, Provisional)
Cholangiocarcinoma (TCGA, Provisional)
Glioblastoma (TCGA, Cell 2013)
Kidney Chromophobe (TCGA, Cancer Cell 2014)
Kidney Chromophobe (TCGA, Provisional)
Kidney Renal Clear Cell Carcinoma (TCGA, Nature 2013)
Lung Squamous Cell Carcinoma (TCGA, Provisional)
Ovarian Serous Cystadenocarcinoma (TCGA, Provisional)
Pancreatic Adenocarcinoma (TCGA, Provisional)
Thymoma (TCGA, Provisional)
MAPK3
Kidney Renal Clear Cell Carcinoma (TCGA, Nature 2013)
Kidney Renal Clear Cell Carcinoma (TCGA, Provisional)
Ovarian Serous Cystadenocarcinoma (TCGA, Provisional)
Uterine Carcinosarcoma (TCGA, Provisional)
NMUR1
Kidney Chromophobe (TCGA, Cancer Cell 2014)
Kidney Chromophobe (TCGA, Provisional)
NSF
Acute Myeloid Leukemia (TCGA, NEJM 2013)
Acute Myeloid Leukemia (TCGA, Provisional)
Kidney Chromophobe (TCGA, Cancer Cell 2014)
Kidney Chromophobe (TCGA, Provisional)
Ovarian Serous Cystadenocarcinoma (TCGA, Provisional)
Pheochromocytoma and Paraganglioma (TCGA, Provisional)
OGFR
Glioblastoma Multiforme (TCGA, Provisional)
PARK2
Adrenocortical Carcinoma (TCGA, Provisional)
Glioblastoma Multiforme (TCGA, Provisional)
Mesothelioma (TCGA, Provisional)
Uveal Melanoma (TCGA, Provisional)
PARK7
Brain Lower Grade Glioma (TCGA, Provisional)
Glioblastoma (TCGA, Cell 2013)
Lung Squamous Cell Carcinoma (TCGA, Provisional)
Pheochromocytoma and Paraganglioma (TCGA, Provisional)
Uveal Melanoma (TCGA, Provisional)
PDE4A
Kidney Renal Clear Cell Carcinoma (TCGA, Nature 2013)
Kidney Renal Clear Cell Carcinoma (TCGA, Provisional)
Lung Adenocarcinoma (TCGA, Provisional)
Pancreatic Adenocarcinoma (TCGA, Provisional)
PDE4D
Kidney Chromophobe (TCGA, Cancer Cell 2014)
Kidney Chromophobe (TCGA, Provisional)
PDPK1
Glioblastoma (TCGA, Cell 2013)
Head and Neck Squamous Cell Carcinoma (TCGA, Nature 2015)
Kidney Chromophobe (TCGA, Cancer Cell 2014)
PINK1
Brain Lower Grade Glioma (TCGA, Provisional)
Colorectal Adenocarcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
PKD2
Papillary Thyroid Carcinoma (TCGA, Cell 2014)
PLCB1
Kidney Renal Clear Cell Carcinoma (TCGA, Nature 2013)
PLCD1
Uveal Melanoma (TCGA, Provisional)
PLCG1
Head and Neck Squamous Cell Carcinoma (TCGA, Nature 2015)
PLCL2
Thymoma (TCGA, Provisional)
PPP1CA
Kidney Renal Clear Cell Carcinoma (TCGA, Nature 2013)
Kidney Renal Clear Cell Carcinoma (TCGA, Provisional)
PPP1R12A
Cholangiocarcinoma (TCGA, Provisional)
Head and Neck Squamous Cell Carcinoma (TCGA, Provisional)
Kidney Chromophobe (TCGA, Cancer Cell 2014)
Kidney Chromophobe (TCGA, Provisional)
Ovarian Serous Cystadenocarcinoma (TCGA, Provisional)
Papillary Thyroid Carcinoma (TCGA, Cell 2014)
Skin Cutaneous Melanoma (TCGA, Provisional)
Thyroid Carcinoma (TCGA, Provisional)
Uterine Carcinosarcoma (TCGA, Provisional)
PPP1R9A
Brain Lower Grade Glioma (TCGA, Provisional)
Colorectal Adenocarcinoma (TCGA, Provisional)
Kidney Chromophobe (TCGA, Cancer Cell 2014)
Kidney Chromophobe (TCGA, Provisional)
PPP2CA
Acute Myeloid Leukemia (TCGA, NEJM 2013)
Acute Myeloid Leukemia (TCGA, Provisional)
Bladder Urothelial Carcinoma (TCGA, Nature 2014)
Bladder Urothelial Carcinoma (TCGA, Provisional)
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Colorectal Adenocarcinoma (TCGA, Provisional)
Esophageal Carcinoma (TCGA, Provisional)
Glioblastoma (TCGA, Cell 2013)
Glioblastoma Multiforme (TCGA, Provisional)
Head and Neck Squamous Cell Carcinoma (TCGA, Nature 2015)
Head and Neck Squamous Cell Carcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Liver Hepatocellular Carcinoma (TCGA, Provisional)
Lung Adenocarcinoma (TCGA, Nature 2014)
Lung Squamous Cell Carcinoma (TCGA, Provisional)
Ovarian Serous Cystadenocarcinoma (TCGA, Provisional)
Pancreatic Adenocarcinoma (TCGA, Provisional)
Pheochromocytoma and Paraganglioma (TCGA, Provisional)
Prostate Adenocarcinoma (TCGA, Provisional)
Sarcoma (TCGA, Provisional)
Skin Cutaneous Melanoma (TCGA, Provisional)
Stomach Adenocarcinoma (TCGA, Provisional)
Testicular Germ Cell Cancer (TCGA, Provisional)
Uterine Carcinosarcoma (TCGA, Provisional)
Uterine Corpus Endometrial Carcinoma (TCGA, Provisional)
Uveal Melanoma (TCGA, Provisional)
PPP3CA
Lung Adenocarcinoma (TCGA, Nature 2014)
Lung Squamous Cell Carcinoma (TCGA, Provisional)
Ovarian Serous Cystadenocarcinoma (TCGA, Provisional)
PPP3CB
Bladder Urothelial Carcinoma (TCGA, Nature 2014)
Bladder Urothelial Carcinoma (TCGA, Provisional)
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Cholangiocarcinoma (TCGA, Provisional)
Glioblastoma (TCGA, Cell 2013)
Head and Neck Squamous Cell Carcinoma (TCGA, Nature 2015)
Kidney Renal Papillary Cell Carcinoma (TCGA, Provisional)
Liver Hepatocellular Carcinoma (TCGA, Provisional)
Lymphoid Neoplasm Diffuse Large B-cell Lymphoma (TCGA, Provisional)
Mesothelioma (TCGA, Provisional)
Papillary Thyroid Carcinoma (TCGA, Cell 2014)
Prostate Adenocarcinoma (TCGA, Provisional)
Sarcoma (TCGA, Provisional)
Skin Cutaneous Melanoma (TCGA, Provisional)
Thymoma (TCGA, Provisional)
Thyroid Carcinoma (TCGA, Provisional)
Uterine Carcinosarcoma (TCGA, Provisional)
PPP3CC
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Colorectal Adenocarcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
Liver Hepatocellular Carcinoma (TCGA, Provisional)
Lung Adenocarcinoma (TCGA, Nature 2014)
Lung Adenocarcinoma (TCGA, Provisional)
Lung Squamous Cell Carcinoma (TCGA, Provisional)
Pancreatic Adenocarcinoma (TCGA, Provisional)
Pheochromocytoma and Paraganglioma (TCGA, Provisional)
Prostate Adenocarcinoma (TCGA, Provisional)
Skin Cutaneous Melanoma (TCGA, Provisional)
Stomach Adenocarcinoma (TCGA, Provisional)
PPP3R1
Cervical Squamous Cell Carcinoma and Endocervical Adenocarcinoma (TCGA, Provisional)
Cholangiocarcinoma (TCGA, Provisional)
Colorectal Adenocarcinoma (TCGA, Provisional)
Head and Neck Squamous Cell Carcinoma (TCGA, Provisional)
Lymphoid Neoplasm Diffuse Large B-cell Lymphoma (TCGA, Provisional)
Mesothelioma (TCGA, Provisional)
Pancreatic Adenocarcinoma (TCGA, Provisional)
Papillary Thyroid Carcinoma (TCGA, Cell 2014)
Pheochromocytoma and Paraganglioma (TCGA, Provisional)
Skin Cutaneous Melanoma (TCGA, Provisional)
Testicular Germ Cell Cancer (TCGA, Provisional)
Thyroid Carcinoma (TCGA, Provisional)
Uterine Corpus Endometrial Carcinoma (TCGA, Provisional)
PPT1
Brain Lower Grade Glioma (TCGA, Provisional)
Cholangiocarcinoma (TCGA, Provisional)
Kidney Renal Clear Cell Carcinoma (TCGA, Nature 2013)
Kidney Renal Clear Cell Carcinoma (TCGA, Provisional)
Pheochromocytoma and Paraganglioma (TCGA, Provisional)
Uveal Melanoma (TCGA, Provisional)
PRKACA
Glioblastoma (TCGA, Cell 2013)
Glioblastoma Multiforme (TCGA, Provisional)
Kidney Renal Clear Cell Carcinoma (TCGA, Nature 2013)
Kidney Renal Clear Cell Carcinoma (TCGA, Provisional)
Lung Adenocarcinoma (TCGA, Provisional)
PRKAR1A
Ovarian Serous Cystadenocarcinoma (TCGA, Provisional)
Testicular Germ Cell Cancer (TCGA, Provisional)
PRKAR2A
Esophageal Carcinoma (TCGA, Provisional)
Head and Neck Squamous Cell Carcinoma (TCGA, Nature 2015)
Kidney Renal Clear Cell Carcinoma (TCGA, Provisional)
Mesothelioma (TCGA, Provisional)
PTEN
Bladder Urothelial Carcinoma (TCGA, Nature 2014)
Bladder Urothelial Carcinoma (TCGA, Provisional)
Brain Lower Grade Glioma (TCGA, Provisional)
Breast Invasive Carcinoma (TCGA, Provisional)
Cervical Squamous Cell Carcinoma and Endocervical Adenocarcinoma (TCGA, Provisional)
Colorectal Adenocarcinoma (TCGA, Provisional)
Esophageal Carcinoma (TCGA, Provisional)
Glioblastoma Multiforme (TCGA, Provisional)
Head and Neck Squamous Cell Carcinoma (TCGA, Nature 2015)
Head and Neck Squamous Cell Carcinoma (TCGA, Provisional)
Kidney Chromophobe (TCGA, Cancer Cell 2014)
Kidney Chromophobe (TCGA, Provisional)
Liver Hepatocellular Carcinoma (TCGA, Provisional)
Lung Squamous Cell Carcinoma (TCGA, Provisional)
Lymphoid Neoplasm Diffuse Large B-cell Lymphoma (TCGA, Provisional)
Mesothelioma (TCGA, Provisional)
Ovarian Serous Cystadenocarcinoma (TCGA, Provisional)
Papillary Thyroid Carcinoma (TCGA, Cell 2014)
Prostate Adenocarcinoma (TCGA, Provisional)
Skin Cutaneous Melanoma (TCGA, Provisional)
Stomach Adenocarcinoma (TCGA, Nature 2014)
Stomach Adenocarcinoma (TCGA, Provisional)
Testicular Germ Cell Cancer (TCGA, Provisional)
Thymoma (TCGA, Provisional)
Thyroid Carcinoma (TCGA, Provisional)
Uterine Carcinosarcoma (TCGA, Provisional)
Uterine Corpus Endometrial Carcinoma (TCGA, Provisional)
PTGER3
Kidney Chromophobe (TCGA, Cancer Cell 2014)
Kidney Chromophobe (TCGA, Provisional)
PTK2
Breast Invasive Carcinoma (TCGA, Cell 2015)
Breast Invasive Carcinoma (TCGA, Provisional)
Colorectal Adenocarcinoma (TCGA, Provisional)
Kidney Chromophobe (TCGA, Cancer Cell 2014)
Kidney Chromophobe (TCGA, Provisional)
Ovarian Serous Cystadenocarcinoma (TCGA, Provisional)
Papillary Thyroid Carcinoma (TCGA, Cell 2014)
Pheochromocytoma and Paraganglioma (TCGA, Provisional)
Prostate Adenocarcinoma (TCGA, Provisional)
Stomach Adenocarcinoma (TCGA, Provisional)
Thyroid Carcinoma (TCGA, Provisional)
Uveal Melanoma (TCGA, Provisional)
PTK2B
Colorectal Adenocarcinoma (TCGA, Provisional)
Lung Adenocarcinoma (TCGA, Nature 2014)
Ovarian Serous Cystadenocarcinoma (TCGA, Provisional)
Prostate Adenocarcinoma (TCGA, Provisional)
PXK
Head and Neck Squamous Cell Carcinoma (TCGA, Nature 2015)
Pheochromocytoma and Paraganglioma (TCGA, Provisional)
RAB3GAP1
Brain Lower Grade Glioma (TCGA, Provisional)
Cholangiocarcinoma (TCGA, Provisional)
Colorectal Adenocarcinoma (TCGA, Provisional)
Glioblastoma (TCGA, Cell 2013)
Head and Neck Squamous Cell Carcinoma (TCGA, Nature 2015)
Head and Neck Squamous Cell Carcinoma (TCGA, Provisional)
Kidney Renal Clear Cell Carcinoma (TCGA, Nature 2013)
Kidney Renal Clear Cell Carcinoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
Papillary Thyroid Carcinoma (TCGA, Cell 2014)
Pheochromocytoma and Paraganglioma (TCGA, Provisional)
Prostate Adenocarcinoma (TCGA, Provisional)
Sarcoma (TCGA, Provisional)
Skin Cutaneous Melanoma (TCGA, Provisional)
Thyroid Carcinoma (TCGA, Provisional)
RIC3
Kidney Chromophobe (TCGA, Cancer Cell 2014)
Kidney Chromophobe (TCGA, Provisional)
Pheochromocytoma and Paraganglioma (TCGA, Provisional)
RNLS
Cervical Squamous Cell Carcinoma and Endocervical Adenocarcinoma (TCGA, Provisional)
Lymphoid Neoplasm Diffuse Large B-cell Lymphoma (TCGA, Provisional)
Ovarian Serous Cystadenocarcinoma (TCGA, Provisional)
Prostate Adenocarcinoma (TCGA, Provisional)
Uveal Melanoma (TCGA, Provisional)
SORCS3
Brain Lower Grade Glioma (TCGA, Provisional)
SRC
Adrenocortical Carcinoma (TCGA, Provisional)
Mesothelioma (TCGA, Provisional)
Uterine Carcinosarcoma (TCGA, Provisional)
TRPV1
Liver Hepatocellular Carcinoma (TCGA, Provisional)
VIPR2
Thymoma (TCGA, Provisional)

According to the methods disclosed herein, a physician of skill in the art can treat a patient, such as a human patient with cancer (e.g., colorectal cancer), so as to inhibit cancer growth, reduce tumor burden, or slow disease progression. The method of treatment can include diagnosing or identifying a patient as a candidate for treatment with a neuromodulating agent based on neurome gene expression in a biopsy. For example, a biopsy can be collected from a patient's colorectal cancer tumor, and the tumor can be analyzed for CHRM3 RNA content by qPCR or RNAseq, and analyzed for CHRM3 protein content by ELISA or Western Blot analysis. The expression of CHRM3 can be compared to the expression of a housekeeping gene, and if the tumor has a higher relative expression of CHRM3 (e.g., 1.5, 2, 2.5, 3, 4, 5, or 10 or more fold higher relative expression of CHRM3 compared to a housekeeping gene), it will be identified as overexpressing CHRM3. A patient with a tumor that overexpresses CHRM3 is a good candidate for treatment with a CHRM3 antagonist (e.g., darifenacin, atropine, dicycloverine, hyoscyamine, alcidium bromide, 4-DAMP, darifenacin, DAU-5884, oxybutynin, tiotropium, zamifenacin, and tolterodine). To this end, a physician of skill in the art can administer a patient with a tumor that overexpresses CHRM3 a selective CHRM3 antagonist (e.g., Darifenacin). The CHRM3 antagonist can be administered locally (e.g., injected into the CHRM3 overexpressing tumor) to decrease tumor growth or volume. The CHRM3 antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, The CHRM3 antagonist is administered bimonthly, once a month, once every two weeks, or at least once a week or more (e.g., 1, 2, 3, 4, 5, 6, or 7 times a week or more).

The CHRM3 antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the CHRM3 antagonist can be compared to evaluate the efficacy of the treatment, and the rate of disease progression can be assessed by comparison to the patient's medical history prior to administration of the CHRM3 antagonist. A finding of a reduction in the total number of tumors, number of primary tumors, volume of tumors, growth of tumors, or rate of disease progression indicates that the CHRM3 antagonist has successfully treated the cancer.

### Example 20—Neurome Gene Under-Expression in Cancer

One form of cancer neuro-dependence is marked by the under-expression of neurome genes, like neurotransmitter or neuropeptide receptors, synaptic junction proteins, neuronal growth factors, structural proteins, channels, transporters, signaling proteins, or others as listed in Table 7 or Table 8. Tumors that under-express neurome genes may be suppressing their expression in order to evade mechanisms of cell growth control and hyper-proliferate. These under-expressed genes and proteins represent potential targets for therapeutic intervention, either by re-introducing the protein by a means of overexpression, such as mRNA or gene therapy, or by identifying alternate signaling paths to recover the growth-suppressive signals.

Table 13 shows the results of an analysis of neurome gene expression data (450 genes) from The Cancer Genome Atlas (TCGA), highlighting genes that were under-expressed in particular cancers present in the TCGA collection. Under-expressed gene-study pairs are selected if a gene was under-expressed in >10% of patients in a given study (frequently under-expressed) or if the mean Z score for patients with under-expression was <2.0 SD below the mean (abundantly under-expressed).

TABLE 13
UNDER-EXPRESSION OF NEUROME GENES IN CANCER

ABAT
Adrenocortical Carcinoma (TCGA, Provisional)
Breast Invasive Carcinoma (TCGA, Cell 2015)
ACHE
Glioblastoma (TCGA, Cell 2013)
Glioblastoma Multiforme (TCGA, Provisional)
Kidney Renal Papillary Cell Carcinoma (TCGA, Provisional)
ADCY1
Lymphoid Neoplasm Diffuse Large B-cell Lymphoma (TCGA, Provisional)
Testicular Germ Cell Cancer (TCGA, Provisional)
ADCY10
Cholangiocarcinoma (TCGA, Provisional)
Liver Hepatocellular Carcinoma (TCGA, Provisional)
ADCY2
Cholangiocarcinoma (TCGA, Provisional)
ADCY3
Uterine Carcinosarcoma (TCGA, Provisional)
ADCY4
Uterine Carcinosarcoma (TCGA, Provisional)
ADCY5
Lung Adenocarcinoma (TCGA, Nature 2014)
Uterine Carcinosarcoma (TCGA, Provisional)
ADCY8
Bladder Urothelial Carcinoma (TCGA, Provisional)
Uterine Corpus Endometrial Carcinoma (TCGA, Provisional)
ADCY9
Kidney Renal Papillary Cell Carcinoma (TCGA, Provisional)
ADCYAP1
Cervical Squamous Cell Carcinoma and Endocervical Adenocarcinoma (TCGA, Provisional)
ADRA1A
Bladder Urothelial Carcinoma (TCGA, Provisional)
ADRA1D
Uterine Carcinosarcoma (TCGA, Provisional)
ADRA2A
Glioblastoma (TCGA, Cell 2013)
Glioblastoma Multiforme (TCGA, Provisional)
ADRB1
Kidney Renal Clear Cell Carcinoma (TCGA, Nature 2013)
AGTR1
Uterine Carcinosarcoma (TCGA, Provisional)
AKAP1
Kidney Renal Papillary Cell Carcinoma (TCGA, Provisional)
AKAP10
Sarcoma (TCGA, Provisional)
AKAP11
Colorectal Adenocarcinoma (TCGA, Provisional)
AKAP14
Testicular Germ Cell Cancer (TCGA, Provisional)
AKAP8
Adrenocortical Carcinoma (TCGA, Provisional)
Ovarian Serous Cystadenocarcinoma (TCGA, Provisional)
Uterine Carcinosarcoma (TCGA, Provisional)
Uterine Corpus Endometrial Carcinoma (TCGA, Provisional)
AKAP9
Adrenocortical Carcinoma (TCGA, Provisional)
Esophageal Carcinoma (TCGA, Provisional)
Testicular Germ Cell Cancer (TCGA, Provisional)
ALDH5A1
Uterine Carcinosarcoma (TCGA, Provisional)
Uveal Melanoma (TCGA, Provisional)
ALDH9A1
Bladder Urothelial Carcinoma (TCGA, Nature 2014)
Bladder Urothelial Carcinoma (TCGA, Provisional)
Breast Invasive Carcinoma (TCGA, Cell 2015)
Breast Invasive Carcinoma (TCGA, Provisional)
Lung Adenocarcinoma (TCGA, Nature 2014)
Lung Adenocarcinoma (TCGA, Provisional)
Uterine Corpus Endometrial Carcinoma (TCGA, Nature 2013)
Uterine Corpus Endometrial Carcinoma (TCGA, Provisional)
ALOX15
Cholangiocarcinoma (TCGA, Provisional)
ATP1A2
Skin Cutaneous Melanoma (TCGA, Provisional)
AVPR1B
Uterine Carcinosarcoma (TCGA, Provisional)
BCHE
Lung Squamous Cell Carcinoma (TCGA, Provisional)
CALCR
Cholangiocarcinoma (TCGA, Provisional)
Esophageal Carcinoma (TCGA, Provisional)
Lung Squamous Cell Carcinoma (TCGA, Provisional)
Stomach Adenocarcinoma (TCGA, Provisional)
Uterine Carcinosarcoma (TCGA, Provisional)
CAMK2A
Adrenocortical Carcinoma (TCGA, Provisional)
CARTPT
Cholangiocarcinoma (TCGA, Provisional)
Mesothelioma (TCGA, Provisional)
Pancreatic Adenocarcinoma (TCGA, Provisional)
CASK
Uterine Carcinosarcoma (TCGA, Provisional)
CCKAR
Cervical Squamous Cell Carcinoma and Endocervical Adenocarcinoma (TCGA, Provisional)
Pancreatic Adenocarcinoma (TCGA, Provisional)
Uterine Carcinosarcoma (TCGA, Provisional)
CCKBR
Lymphoid Neoplasm Diffuse Large B-cell Lymphoma (TCGA, Provisional)
CDK5
Adrenocortical Carcinoma (TCGA, Provisional)
Kidney Chromophobe (TCGA, Cancer Cell 2014)
Kidney Chromophobe (TCGA, Provisional)
Kidney Renal Papillary Cell Carcinoma (TCGA, Provisional)
Skin Cutaneous Melanoma (TCGA, Provisional)
Testicular Germ Cell Cancer (TCGA, Provisional)
CDK5R1
Kidney Renal Papillary Cell Carcinoma (TCGA, Provisional)
Uterine Carcinosarcoma (TCGA, Provisional)
CHRNB1
Kidney Renal Papillary Cell Carcinoma (TCGA, Provisional)
Uterine Carcinosarcoma (TCGA, Provisional)
CHRNB2
Kidney Chromophobe (TCGA, Cancer Cell 2014)
Kidney Chromophobe (TCGA, Provisional)
Thymoma (TCGA, Provisional)
CHRNB3
Adrenocortical Carcinoma (TCGA, Provisional)
Mesothelioma (TCGA, Provisional)
Uterine Corpus Endometrial Carcinoma (TCGA, Provisional)
Uveal Melanoma (TCGA, Provisional)
CPA4
Adrenocortical Carcinoma (TCGA, Provisional)
Kidney Chromophobe (TCGA, Cancer Cell 2014)
Kidney Chromophobe (TCGA, Provisional)
CRCP
Adrenocortical Carcinoma (TCGA, Provisional)
Bladder Urothelial Carcinoma (TCGA, Provisional)
Colorectal Adenocarcinoma (TCGA, Provisional)
Esophageal Carcinoma (TCGA, Provisional)
Glioblastoma (TCGA, Cell 2013)
Glioblastoma Multiforme (TCGA, Provisional)
Kidney Renal Clear Cell Carcinoma (TCGA, Nature 2013)
Kidney Renal Papillary Cell Carcinoma (TCGA, Provisional)
Lung Adenocarcinoma (TCGA, Nature 2014)
Lung Adenocarcinoma (TCGA, Provisional)
Lymphoid Neoplasm Diffuse Large B-cell Lymphoma (TCGA, Provisional)
Mesothelioma (TCGA, Provisional)
Skin Cutaneous Melanoma (TCGA, Provisional)
Stomach Adenocarcinoma (TCGA, Provisional)
Uterine Carcinosarcoma (TCGA, Provisional)
CRHR1
Bladder Urothelial Carcinoma (TCGA, Nature 2014)
CRLF1
Mesothelioma (TCGA, Provisional)
CSNK1E
Testicular Germ Cell Cancer (TCGA, Provisional)
CYSLTR2
Uterine Carcinosarcoma (TCGA, Provisional)
DAGLB
Colorectal Adenocarcinoma (TCGA, Provisional)
Esophageal Carcinoma (TCGA, Provisional)
Head and Neck Squamous Cell Carcinoma (TCGA, Nature 2015)
Kidney Renal Papillary Cell Carcinoma (TCGA, Provisional)
Lung Adenocarcinoma (TCGA, Nature 2014)
Lung Adenocarcinoma (TCGA, Provisional)
Stomach Adenocarcinoma (TCGA, Nature 2014)
Stomach Adenocarcinoma (TCGA, Provisional)
Testicular Germ Cell Cancer (TCGA, Provisional)
DCLK1
Uterine Carcinosarcoma (TCGA, Provisional)
DDC
Colorectal Adenocarcinoma (TCGA, Provisional)
Esophageal Carcinoma (TCGA, Provisional)
DDR1
Kidney Chromophobe (TCGA, Cancer Cell 2014)
Kidney Chromophobe (TCGA, Provisional)
DGKI
Adrenocortical Carcinoma (TCGA, Provisional)
Uterine Corpus Endometrial Carcinoma (TCGA, Provisional)
DRD2
Cholangiocarcinoma (TCGA, Provisional)
Kidney Renal Clear Cell Carcinoma (TCGA, Nature 2013)
Skin Cutaneous Melanoma (TCGA, Provisional)
DRD3
Uterine Carcinosarcoma (TCGA, Provisional)
DRD4
Uterine Carcinosarcoma (TCGA, Provisional)
DRD5
Uterine Corpus Endometrial Carcinoma (TCGA, Provisional)
EEF2K
Adrenocortical Carcinoma (TCGA, Provisional)
Breast Invasive Carcinoma (TCGA, Cell 2015)
Breast Invasive Carcinoma (TCGA, Provisional)
ERBB2
Breast Invasive Carcinoma (TCGA, Cell 2015)
Breast Invasive Carcinoma (TCGA, Provisional)
Cervical Squamous Cell Carcinoma and Endocervical Adenocarcinoma (TCGA, Provisional)
Colorectal Adenocarcinoma (TCGA, Provisional)
Esophageal Carcinoma (TCGA, Provisional)
Lung Adenocarcinoma (TCGA, Nature 2014)
Ovarian Serous Cystadenocarcinoma (TCGA, Provisional)
Stomach Adenocarcinoma (TCGA, Nature 2014)
Stomach Adenocarcinoma (TCGA, Provisional)
Uterine Carcinosarcoma (TCGA, Provisional)
Uterine Corpus Endometrial Carcinoma (TCGA, Nature 2013)
Uterine Corpus Endometrial Carcinoma (TCGA, Provisional)
FNTA
Adrenocortical Carcinoma (TCGA, Provisional)
Breast Invasive Carcinoma (TCGA, Cell 2015)
Breast Invasive Carcinoma (TCGA, Provisional)
Colorectal Adenocarcinoma (TCGA, Provisional)
Stomach Adenocarcinoma (TCGA, Nature 2014)
Stomach Adenocarcinoma (TCGA, Provisional)
Testicular Germ Cell Cancer (TCGA, Provisional)
Uterine Carcinosarcoma (TCGA, Provisional)
Uterine Corpus Endometrial Carcinoma (TCGA, Provisional)
FRS2
Bladder Urothelial Carcinoma (TCGA, Nature 2014)
Bladder Urothelial Carcinoma (TCGA, Provisional)
Glioblastoma (TCGA, Cell 2013)
Glioblastoma Multiforme (TCGA, Provisional)
Lung Adenocarcinoma (TCGA, Nature 2014)
Sarcoma (TCGA, Provisional)
FRS3
Cholangiocarcinoma (TCGA, Provisional)
Uveal Melanoma (TCGA, Provisional)
FSHR
Kidney Chromophobe (TCGA, Cancer Cell 2014)
Kidney Chromophobe (TCGA, Provisional)
GABRA1
Acute Myeloid Leukemia (TCGA, NEJM 2013)
Acute Myeloid Leukemia (TCGA, Provisional)
Kidney Renal Clear Cell Carcinoma (TCGA, Nature 2013)
Kidney Renal Clear Cell Carcinoma (TCGA, Provisional)
Lymphoid Neoplasm Diffuse Large B-cell Lymphoma (TCGA, Provisional)
Pancreatic Adenocarcinoma (TCGA, Provisional)
GABRA2
Adrenocortical Carcinoma (TCGA, Provisional)
GABRA3
Adrenocortical Carcinoma (TCGA, Provisional)
Kidney Chromophobe (TCGA, Cancer Cell 2014)
Kidney Chromophobe (TCGA, Provisional)
GABRA4
Adrenocortical Carcinoma (TCGA, Provisional)
Liver Hepatocellular Carcinoma (TCGA, Provisional)
Ovarian Serous Cystadenocarcinoma (TCGA, Provisional)
Uterine Carcinosarcoma (TCGA, Provisional)
GABRA5
Kidney Chromophobe (TCGA, Cancer Cell 2014)
Kidney Chromophobe (TCGA, Provisional)
Uterine Carcinosarcoma (TCGA, Provisional)
GABRA6
Sarcoma (TCGA, Provisional)
GABRB2
Adrenocortical Carcinoma (TCGA, Provisional)
GABRD
Kidney Chromophobe (TCGA, Cancer Cell 2014)
Kidney Chromophobe (TCGA, Provisional)
Uterine Carcinosarcoma (TCGA, Provisional)
GABRG2
Cholangiocarcinoma (TCGA, Provisional)
Mesothelioma (TCGA, Provisional)
Uterine Corpus Endometrial Carcinoma (TCGA, Provisional)
GABRR1
Adrenocortical Carcinoma (TCGA, Provisional)
GABRR3
Uveal Melanoma (TCGA, Provisional)
GAD2
Kidney Renal Clear Cell Carcinoma (TCGA, Nature 2013)
GAL
Lung Adenocarcinoma (TCGA, Nature 2014)
GALR3
Mesothelioma (TCGA, Provisional)
GCGR
Lung Adenocarcinoma (TCGA, Nature 2014)
Stomach Adenocarcinoma (TCGA, Nature 2014)
Stomach Adenocarcinoma (TCGA, Provisional)
GCHFR
Kidney Chromophobe (TCGA, Cancer Cell 2014)
Kidney Chromophobe (TCGA, Provisional)
GFRA3
Mesothelioma (TCGA, Provisional)
Ovarian Serous Cystadenocarcinoma (TCGA, Provisional)
Uterine Carcinosarcoma (TCGA, Provisional)
GFRA4
Thymoma (TCGA, Provisional)
GHRHR
Glioblastoma (TCGA, Cell 2013)
Glioblastoma Multiforme (TCGA, Provisional)
Pancreatic Adenocarcinoma (TCGA, Provisional)
GIP
Kidney Renal Clear Cell Carcinoma (TCGA, Nature 2013)
Uterine Carcinosarcoma (TCGA, Provisional)
GLRA1
Adrenocortical Carcinoma (TCGA, Provisional)
GLRA4
Kidney Chromophobe (TCGA, Cancer Cell 2014)
Kidney Chromophobe (TCGA, Provisional)
GLUL
Breast Invasive Carcinoma (TCGA, Cell 2015)
Uterine Corpus Endometrial Carcinoma (TCGA, Provisional)
GNA11
Glioblastoma Multiforme (TCGA, Provisional)
GNAI1
Adrenocortical Carcinoma (TCGA, Provisional)
GNAI3
Uterine Carcinosarcoma (TCGA, Provisional)
GNAL
Uterine Carcinosarcoma (TCGA, Provisional)
GNAO1
Adrenocortical Carcinoma (TCGA, Provisional)
Uterine Carcinosarcoma (TCGA, Provisional)
GNAS
Colorectal Adenocarcinoma (TCGA, Provisional)
Ovarian Serous Cystadenocarcinoma (TCGA, Provisional)
Stomach Adenocarcinoma (TCGA, Nature 2014)
Stomach Adenocarcinoma (TCGA, Provisional)
GNB2
Esophageal Carcinoma (TCGA, Provisional)
Glioblastoma (TCGA, Cell 2013)
Glioblastoma Multiforme (TCGA, Provisional)
Kidney Chromophobe (TCGA, Cancer Cell 2014)
Kidney Chromophobe (TCGA, Provisional)
Kidney Renal Papillary Cell Carcinoma (TCGA, Provisional)
Lung Squamous Cell Carcinoma (TCGA, Provisional)
Testicular Germ Cell Cancer (TCGA, Provisional)
GNB4
Cervical Squamous Cell Carcinoma and Endocervical Adenocarcinoma (TCGA, Provisional)
Esophageal Carcinoma (TCGA, Provisional)
Lung Squamous Cell Carcinoma (TCGA, Provisional)
Uterine Carcinosarcoma (TCGA, Provisional)
GNG4
Kidney Chromophobe (TCGA, Cancer Cell 2014)
Kidney Chromophobe (TCGA, Provisional)
GNG5
Uterine Carcinosarcoma (TCGA, Provisional)
GNG8
Adrenocortical Carcinoma (TCGA, Provisional)
GNMT
Esophageal Carcinoma (TCGA, Provisional)
Kidney Chromophobe (TCGA, Cancer Cell 2014)
Kidney Chromophobe (TCGA, Provisional)
Kidney Renal Clear Cell Carcinoma (TCGA, Nature 2013)
Kidney Renal Clear Cell Carcinoma (TCGA, Provisional)
GPER1
Uterine Carcinosarcoma (TCGA, Provisional)
GPI
Adrenocortical Carcinoma (TCGA, Provisional)
GPR139
Esophageal Carcinoma (TCGA, Provisional)
GPR149
Esophageal Carcinoma (TCGA, Provisional)
Head and Neck Squamous Cell Carcinoma (TCGA, Nature 2015)
Pancreatic Adenocarcinoma (TCGA, Provisional)
GPR21
Uterine Carcinosarcoma (TCGA, Provisional)
GPR78
Mesothelioma (TCGA, Provisional)
GPR83
Uterine Carcinosarcoma (TCGA, Provisional)
GRB2
Kidney Renal Papillary Cell Carcinoma (TCGA, Provisional)
Lung Adenocarcinoma (TCGA, Nature 2014)
Uterine Carcinosarcoma (TCGA, Provisional)
GRIA1
Adrenocortical Carcinoma (TCGA, Provisional)
GRIA2
Esophageal Carcinoma (TCGA, Provisional)
Kidney Chromophobe (TCGA, Cancer Cell 2014)
Kidney Chromophobe (TCGA, Provisional)
GRIA4
Lung Adenocarcinoma (TCGA, Provisional)
Lymphoid Neoplasm Diffuse Large B-cell Lymphoma (TCGA, Provisional)
Uterine Carcinosarcoma (TCGA, Provisional)
GRIK1
Adrenocortical Carcinoma (TCGA, Provisional)
Kidney Chromophobe (TCGA, Cancer Cell 2014)
Kidney Chromophobe (TCGA, Provisional)
GRIK5
Uterine Carcinosarcoma (TCGA, Provisional)
GRIN2A
Adrenocortical Carcinoma (TCGA, Provisional)
Uterine Carcinosarcoma (TCGA, Provisional)
GRIN2B
Uterine Carcinosarcoma (TCGA, Provisional)
GRIN2B.1
Uterine Carcinosarcoma (TCGA, Provisional)
GRIN2C
Pancreatic Adenocarcinoma (TCGA, Provisional)
GRIN2D
Bladder Urothelial Carcinoma (TCGA, Nature 2014)
GRIN3A
Sarcoma (TCGA, Provisional)
GRM5
Bladder Urothelial Carcinoma (TCGA, Nature 2014)
GRM7
Uveal Melanoma (TCGA, Provisional)
GRPR
Kidney Chromophobe (TCGA, Cancer Cell 2014)
Kidney Chromophobe (TCGA, Provisional)
HMOX1
Uterine Corpus Endometrial Carcinoma (TCGA, Nature 2013)
HMOX2
Kidney Renal Papillary Cell Carcinoma (TCGA, Provisional)
HRH3
Bladder Urothelial Carcinoma (TCGA, Nature 2014)
Breast Invasive Carcinoma (TCGA, Cell 2015)
Cholangiocarcinoma (TCGA, Provisional)
Colorectal Adenocarcinoma (TCGA, Provisional)
Kidney Chromophobe (TCGA, Cancer Cell 2014)
Kidney Chromophobe (TCGA, Provisional)
Lymphoid Neoplasm Diffuse Large B-cell Lymphoma (TCGA, Provisional)
Stomach Adenocarcinoma (TCGA, Nature 2014)
Stomach Adenocarcinoma (TCGA, Provisional)
HTR1A
Adrenocortical Carcinoma (TCGA, Provisional)
HTR1D
Kidney Chromophobe (TCGA, Cancer Cell 2014)
Kidney Chromophobe (TCGA, Provisional)
HTR1E
Kidney Chromophobe (TCGA, Cancer Cell 2014)
Kidney Chromophobe (TCGA, Provisional)
HTR1F
Cholangiocarcinoma (TCGA, Provisional)
HTR2C
Brain Lower Grade Glioma (TCGA, Provisional)
Liver Hepatocellular Carcinoma (TCGA, Provisional)
Uterine Carcinosarcoma (TCGA, Provisional)
HTR3C
Esophageal Carcinoma (TCGA, Provisional)
Uterine Corpus Endometrial Carcinoma (TCGA, Provisional)
HTR3D
Esophageal Carcinoma (TCGA, Provisional)
Lung Adenocarcinoma (TCGA, Nature 2014)
Lung Squamous Cell Carcinoma (TCGA, Provisional)
Stomach Adenocarcinoma (TCGA, Nature 2014)
Stomach Adenocarcinoma (TCGA, Provisional)
HTR3E
Bladder Urothelial Carcinoma (TCGA, Nature 2014)
Cervical Squamous Cell Carcinoma and Endocervical Adenocarcinoma (TCGA, Provisional)
Lung Squamous Cell Carcinoma (TCGA, Provisional)
Uterine Carcinosarcoma (TCGA, Provisional)
Uterine Corpus Endometrial Carcinoma (TCGA, Provisional)
HTR5A
Adrenocortical Carcinoma (TCGA, Provisional)
Mesothelioma (TCGA, Provisional)
Ovarian Serous Cystadenocarcinoma (TCGA, Provisional)
Pancreatic Adenocarcinoma (TCGA, Provisional)
HTR6
Kidney Chromophobe (TCGA, Cancer Cell 2014)
Kidney Chromophobe (TCGA, Provisional)
HTR7
Glioblastoma (TCGA, Cell 2013)
Glioblastoma Multiforme (TCGA, Provisional)
Uterine Carcinosarcoma (TCGA, Provisional)
KISS1R
Thymoma (TCGA, Provisional)
KRAS
Esophageal Carcinoma (TCGA, Provisional)
Lung Squamous Cell Carcinoma (TCGA, Provisional)
Ovarian Serous Cystadenocarcinoma (TCGA, Provisional)
Stomach Adenocarcinoma (TCGA, Provisional)
Uterine Carcinosarcoma (TCGA, Provisional)
LHCGR
Skin Cutaneous Melanoma (TCGA, Provisional)
LPAR3
Kidney Renal Clear Cell Carcinoma (TCGA, Nature 2013)
Kidney Renal Clear Cell Carcinoma (TCGA, Provisional)
LRRK2
Kidney Renal Papillary Cell Carcinoma (TCGA, Provisional)
LRTOMT
Esophageal Carcinoma (TCGA, Provisional)
MAP2K2
Glioblastoma Multiforme (TCGA, Provisional)
Kidney Chromophobe (TCGA, Cancer Cell 2014)
Kidney Chromophobe (TCGA, Provisional)
MAP3K1
Adrenocortical Carcinoma (TCGA, Provisional)
MAPK1
Lung Squamous Cell Carcinoma (TCGA, Provisional)
MAPK14
Liver Hepatocellular Carcinoma (TCGA, Provisional)
Skin Cutaneous Melanoma (TCGA, Provisional)
Uveal Melanoma (TCGA, Provisional)
MC1R
Adrenocortical Carcinoma (TCGA, Provisional)
MC2R
Uterine Carcinosarcoma (TCGA, Provisional)
MC3R
Bladder Urothelial Carcinoma (TCGA, Nature 2014)
Colorectal Adenocarcinoma (TCGA, Provisional)
Thymoma (TCGA, Provisional)
MC4R
Glioblastoma (TCGA, Cell 2013)
Glioblastoma Multiforme (TCGA, Provisional)
Prostate Adenocarcinoma (TCGA, Provisional)
MC5R
Cholangiocarcinoma (TCGA, Provisional)
MCHR1
Bladder Urothelial Carcinoma (TCGA, Nature 2014)
Prostate Adenocarcinoma (TCGA, Provisional)
MCHR2
Prostate Adenocarcinoma (TCGA, Provisional)
MME
Cholangiocarcinoma (TCGA, Provisional)
Uterine Carcinosarcoma (TCGA, Provisional)
MMP13
Cholangiocarcinoma (TCGA, Provisional)
Kidney Chromophobe (TCGA, Cancer Cell 2014)
Kidney Chromophobe (TCGA, Provisional)
MRAP
Uterine Carcinosarcoma (TCGA, Provisional)
NAALAD2
Esophageal Carcinoma (TCGA, Provisional)
NAMPT
Glioblastoma (TCGA, Cell 2013)
Glioblastoma Multiforme (TCGA, Provisional)
Testicular Germ Cell Cancer (TCGA, Provisional)
NGFR
Kidney Chromophobe (TCGA, Cancer Cell 2014)
Kidney Chromophobe (TCGA, Provisional)
Uterine Carcinosarcoma (TCGA, Provisional)
NMUR2
Kidney Chromophobe (TCGA, Cancer Cell 2014)
Kidney Chromophobe (TCGA, Provisional)
Liver Hepatocellular Carcinoma (TCGA, Provisional)
NOS1
Adrenocortical Carcinoma (TCGA, Provisional)
Cholangiocarcinoma (TCGA, Provisional)
Kidney Chromophobe (TCGA, Cancer Cell 2014)
Kidney Chromophobe (TCGA, Provisional)
NPBWR1
Testicular Germ Cell Cancer (TCGA, Provisional)
Uterine Corpus Endometrial Carcinoma (TCGA, Provisional)
NPBWR2
Adrenocortical Carcinoma (TCGA, Provisional)
NPFFR2
Ovarian Serous Cystadenocarcinoma (TCGA, Provisional)
Uterine Carcinosarcoma (TCGA, Provisional)
Uterine Corpus Endometrial Carcinoma (TCGA, Provisional)
NPSR1
Cholangiocarcinoma (TCGA, Provisional)
Testicular Germ Cell Cancer (TCGA, Provisional)
NPY2R
Adrenocortical Carcinoma (TCGA, Provisional)
NPY6R
Uterine Carcinosarcoma (TCGA, Provisional)
NQO1
Testicular Germ Cell Cancer (TCGA, Provisional)
NR4A2
Kidney Chromophobe (TCGA, Cancer Cell 2014)
Kidney Chromophobe (TCGA, Provisional)
NSF
Kidney Renal Papillary Cell Carcinoma (TCGA, Provisional)
Lung Adenocarcinoma (TCGA, Nature 2014)
Lung Adenocarcinoma (TCGA, Provisional)
NTRK1
Glioblastoma (TCGA, Cell 2013)
Glioblastoma Multiforme (TCGA, Provisional)
Pancreatic Adenocarcinoma (TCGA, Provisional)
Skin Cutaneous Melanoma (TCGA, Provisional)
Uterine Carcinosarcoma (TCGA, Provisional)
NTRK2
Adrenocortical Carcinoma (TCGA, Provisional)
NTSR1
Mesothelioma (TCGA, Provisional)
NTSR2
Lymphoid Neoplasm Diffuse Large B-cell Lymphoma (TCGA, Provisional)
OGFR
Adrenocortical Carcinoma (TCGA, Provisional)
Stomach Adenocarcinoma (TCGA, Provisional)
OXT
Adrenocortical Carcinoma (TCGA, Provisional)
Uterine Carcinosarcoma (TCGA, Provisional)
P2RX3
Bladder Urothelial Carcinoma (TCGA, Nature 2014)
Bladder Urothelial Carcinoma (TCGA, Provisional)
P2RY1
Cervical Squamous Cell Carcinoma and Endocervical Adenocarcinoma (TCGA, Provisional)
Head and Neck Squamous Cell Carcinoma (TCGA, Nature 2015)
P2RY11
Adrenocortical Carcinoma (TCGA, Provisional)
P2RY13
Uveal Melanoma (TCGA, Provisional)
P2RY6
Cholangiocarcinoma (TCGA, Provisional)
PAH
Esophageal Carcinoma (TCGA, Provisional)
PARK7
Uterine Carcinosarcoma (TCGA, Provisional)
PCSK1
Esophageal Carcinoma (TCGA, Provisional)
Pancreatic Adenocarcinoma (TCGA, Provisional)
PCSK1N
Esophageal Carcinoma (TCGA, Provisional)
Uterine Carcinosarcoma (TCGA, Provisional)
PDE4D
Cholangiocarcinoma (TCGA, Provisional)
PLCG1
Bladder Urothelial Carcinoma (TCGA, Nature 2014)
Bladder Urothelial Carcinoma (TCGA, Provisional)
Colorectal Adenocarcinoma (TCGA, Provisional)
Kidney Renal Papillary Cell Carcinoma (TCGA, Provisional)
Ovarian Serous Cystadenocarcinoma (TCGA, Provisional)
Skin Cutaneous Melanoma (TCGA, Provisional)
Stomach Adenocarcinoma (TCGA, Nature 2014)
Stomach Adenocarcinoma (TCGA, Provisional)
PLCL2
Uveal Melanoma (TCGA, Provisional)
PPP1R12A
Adrenocortical Carcinoma (TCGA, Provisional)
Lymphoid Neoplasm Diffuse Large B-cell Lymphoma (TCGA, Provisional)
PPP1R1B
Breast Invasive Carcinoma (TCGA, Cell 2015)
Breast Invasive Carcinoma (TCGA, Provisional)
Esophageal Carcinoma (TCGA, Provisional)
PPP1R9A
Kidney Renal Papillary Cell Carcinoma (TCGA, Provisional)
PPP2CA
Adrenocortical Carcinoma (TCGA, Provisional)
Kidney Renal Clear Cell Carcinoma (TCGA, Nature 2013)
PPP3CB
Uterine Carcinosarcoma (TCGA, Provisional)
PPT1
Ovarian Serous Cystadenocarcinoma (TCGA, Provisional)
PPY
Cholangiocarcinoma (TCGA, Provisional)
PRKACA
Ovarian Serous Cystadenocarcinoma (TCGA, Provisional)
PRKACB
Kidney Chromophobe (TCGA, Cancer Cell 2014)
Kidney Chromophobe (TCGA, Provisional)
PRKAR1A
Bladder Urothelial Carcinoma (TCGA, Nature 2014)
PRLHR
Pancreatic Adenocarcinoma (TCGA, Provisional)
PRLR
Cholangiocarcinoma (TCGA, Provisional)
Uterine Carcinosarcoma (TCGA, Provisional)
PROKR1
Lung Adenocarcinoma (TCGA, Nature 2014)
PROKR2
Adrenocortical Carcinoma (TCGA, Provisional)
PTEN
Adrenocortical Carcinoma (TCGA, Provisional)
PTGER3
Uterine Carcinosarcoma (TCGA, Provisional)
PTGS2
Sarcoma (TCGA, Provisional)
PTK2
Adrenocortical Carcinoma (TCGA, Provisional)
Bladder Urothelial Carcinoma (TCGA, Nature 2014)
Breast Invasive Carcinoma (TCGA, Cell 2015)
Breast Invasive Carcinoma (TCGA, Provisional)
Cholangiocarcinoma (TCGA, Provisional)
Colorectal Adenocarcinoma (TCGA, Provisional)
Esophageal Carcinoma (TCGA, Provisional)
Head and Neck Squamous Cell Carcinoma (TCGA, Nature 2015)
Head and Neck Squamous Cell Carcinoma (TCGA, Provisional)
Liver Hepatocellular Carcinoma (TCGA, Provisional)
Lung Adenocarcinoma (TCGA, Nature 2014)
Lung Adenocarcinoma (TCGA, Provisional)
Lung Squamous Cell Carcinoma (TCGA, Provisional)
Ovarian Serous Cystadenocarcinoma (TCGA, Provisional)
Pancreatic Adenocarcinoma (TCGA, Provisional)
Skin Cutaneous Melanoma (TCGA, Provisional)
Stomach Adenocarcinoma (TCGA, Nature 2014)
Stomach Adenocarcinoma (TCGA, Provisional)
Testicular Germ Cell Cancer (TCGA, Provisional)
Uterine Carcinosarcoma (TCGA, Provisional)
Uterine Corpus Endometrial Carcinoma (TCGA, Provisional)
Uveal Melanoma (TCGA, Provisional)
PTPRN2
Glioblastoma (TCGA, Cell 2013)
Glioblastoma Multiforme (TCGA, Provisional)
PXK
Kidney Renal Clear Cell Carcinoma (TCGA, Nature 2013)
QRFPR
Lung Adenocarcinoma (TCGA, Nature 2014)
Lung Adenocarcinoma (TCGA, Provisional)
Mesothelioma (TCGA, Provisional)
Uterine Carcinosarcoma (TCGA, Provisional)
RAMP3
Glioblastoma (TCGA, Cell 2013)
Glioblastoma Multiforme (TCGA, Provisional)
Uterine Carcinosarcoma (TCGA, Provisional)
RXFP1
Mesothelioma (TCGA, Provisional)
RXFP4
Glioblastoma (TCGA, Cell 2013)
Glioblastoma Multiforme (TCGA, Provisional)
Liver Hepatocellular Carcinoma (TCGA, Provisional)
Thymoma (TCGA, Provisional)
SORCS1
Kidney Chromophobe (TCGA, Cancer Cell 2014)
Kidney Chromophobe (TCGA, Provisional)
SORCS3
Head and Neck Squamous Cell Carcinoma (TCGA, Provisional)
SRC
Adrenocortical Carcinoma (TCGA, Provisional)
Colorectal Adenocarcinoma (TCGA, Provisional)
Esophageal Carcinoma (TCGA, Provisional)
Head and Neck Squamous Cell Carcinoma (TCGA, Nature 2015)
Ovarian Serous Cystadenocarcinoma (TCGA, Provisional)
Stomach Adenocarcinoma (TCGA, Provisional)
Uterine Carcinosarcoma (TCGA, Provisional)
SSTR3
Mesothelioma (TCGA, Provisional)
SSTR4
Uterine Corpus Endometrial Carcinoma (TCGA, Provisional)
SSTR5
Kidney Chromophobe (TCGA, Cancer Cell 2014)
Kidney Chromophobe (TCGA, Provisional)
SULF2
Cholangiocarcinoma (TCGA, Provisional)
TACR2
Kidney Chromophobe (TCGA, Cancer Cell 2014)
Kidney Chromophobe (TCGA, Provisional)
Testicular Germ Cell Cancer (TCGA, Provisional)
TACR3
Uterine Carcinosarcoma (TCGA, Provisional)
Uterine Corpus Endometrial Carcinoma (TCGA, Provisional)
TGM2
Kidney Chromophobe (TCGA, Cancer Cell 2014)
Kidney Chromophobe (TCGA, Provisional)
TPH1
Lung Adenocarcinoma (TCGA, Nature 2014)
TPH2
Sarcoma (TCGA, Provisional)
TRHR
Lung Adenocarcinoma (TCGA, Nature 2014)
Lung Adenocarcinoma (TCGA, Provisional)
TRPC4
Cholangiocarcinoma (TCGA, Provisional)
USP46
Adrenocortical Carcinoma (TCGA, Provisional)
UTS2R
Uterine Corpus Endometrial Carcinoma (TCGA, Provisional)
VIPR2
Uterine Carcinosarcoma (TCGA, Provisional)
XCR1
Uveal Melanoma (TCGA, Provisional)

According to the methods disclosed herein, a physician of skill in the art can treat a patient, such as a human patient with cancer (e.g., rhabdomyosarcoma), so as to inhibit cancer growth, reduce tumor burden, or slow disease progression. The method of treatment can include diagnosing or identifying a patient as a candidate for treatment with a neuromodulating agent based on neurome gene expression in a biopsy. For example, a tissue sample can be collected from a patient's rhabdomyosarcoma, and the sample can be analyzed for acetylcholinesterase (ACHE) expression by qPCR or RNAseq. The expression of ACHE can be compared to the expression of a housekeeping gene, and if the tumor has a lower relative expression of ACHE (e.g., 1.5, 2, 2.5, 3, 4, 5, or 10 or more fold lower relative expression of ACHE compared to a housekeeping gene), it will be identified as under-expressing ACHR. A patient with ACHE under-expression in a cancer can be treated by restoring acetylcholine signaling. To this end, a physician of skill in the art can administer a patient with a tumor that under-expresses ACHE acetylcholine or a muscarinic or nicotinic receptor agonist (e.g., AF102B, AF267B, pilocarpine, cevimeline, bethanechol, carbachol, and methacholine). The nicotinic receptor agonist can be administered locally (e.g., injected into an ACHE under-expressing tumor) to decrease tumor growth or volume. The nicotinic receptor agonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the nicotinic receptor agonist is administered bimonthly, once a month, once every two weeks, or at least once a week or more (e.g., 1, 2, 3, 4, 5, 6, or 7 times a week or more).

The nicotinic receptor agonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the nicotinic receptor agonist can be compared to evaluate the efficacy of the treatment, and the rate of disease progression can be assessed by comparison to the patient's medical history prior to administration of the nicotinic receptor agonist. A finding of a reduction in the total number of tumors, number of primary tumors, volume of tumors, growth of tumors, or rate of disease progression indicates that the nicotinic receptor agonist has successfully treated the cancer.

### Example 21—Neurome Gene Co-Expression with Biosynthetic Pathway Gene

One form of cancer neuro-dependence is the co-expression within a tumor of the genes necessary for the enzymatic biosynthesis or recycling of a neurotransmitter or the genes encoding a neuropeptide, and the genes for the cognate neurotransmitter or neuropeptide receptor. This co-expression suggests that cancer cells may produce their own neurotransmitters, and then capture/detect them in a feed-forward autocrine or paracrine loop to promote growth and proliferation in a nerve-independent fashion. Neural signaling pathways may not require nerves to be present and active.

In such a case, the biosynthetic enzymes are potential drug targets for cancers in which these autocrine loops are active—especially since these autocrine loops may result in a higher local concentration of ligand at the site, as well as enhanced expression of the cognate receptor, which may help in localizing a therapy to the proper site for activity; the biosynthetic enzymes (or the presence of enzyme & receptor) can be a “Companion diagnostic” to assess whether a patient is a good candidate for treatment. A patient with a tumor that expresses both a gene necessary for the enzymatic biosynthesis or recycling of a neurotransmitter or a gene encoding a neuropeptide, and a gene for the cognate neurotransmitter or neuropeptide receptor can be treated by blocking the receptor (e.g., with a neurotransmitter or neuropeptide antagonist), disrupting the enzyme (e.g., targeting the synthetic enzyme with an inhibitory RNA), or disrupting the neurotransmitter or neuropeptide (e.g., using an antibody to block or sequester the neurotransmitter or neuropeptide).

The tables below show the results of our analysis of gene expression data in the Cancer Cell Line Encyclopedia (CCLE) for cancer cell lines that co-express the rate-limiting enzyme for the biosynthesis of norepinephrine, acetylcholine, and serotonin, along with at least one of the receptors for these neurotransmitters and are thus candidates for therapy along this axis.

TABLE 14A
Cell lines that express tyrosine hydroxylase
(TH) and any beta adrenergic receptor (ADRB)

| CELL LINE NAME | CANCER TYPE |
|---|---|
| CHP126 | AUTONOMIC GANGLIA |
| CHP212 | AUTONOMIC GANGLIA |
| KELLY | AUTONOMIC GANGLIA |
| KPNRTBM1 | AUTONOMIC GANGLIA |
| KPNYN | AUTONOMIC GANGLIA |
| NH6 | AUTONOMIC GANGLIA |
| SIMA | AUTONOMIC GANGLIA |
| SKNBE2 | AUTONOMIC GANGLIA |
| SAOS2 | BONE |
| AU565 | BREAST |
| BT20 | BREAST |
| DU4475 | BREAST |
| EFM192A | BREAST |
| HCC1419 | BREAST |
| MDAMB134VI | BREAST |
| MDAMB361 | BREAST |
| MDAMB415 | BREAST |
| SKBR3 | BREAST |
| UACC893 | BREAST |
| 42MGBA | CENTRAL NERVOUS SYSTEM |
| KS1 | CENTRAL NERVOUS SYSTEM |
| YH13 | CENTRAL NERVOUS SYSTEM |
| RL952 | ENDOMETRIUM |
| CL14 | LARGE INTESTINE |
| CL40 | LARGE INTESTINE |
| COLO201 | LARGE INTESTINE |
| LS1034 | LARGE INTESTINE |
| LS123 | LARGE INTESTINE |
| SNU1033 | LARGE INTESTINE |
| SNU1197 | LARGE INTESTINE |
| SW1417 | LARGE INTESTINE |
| SW480 | LARGE INTESTINE |
| SW620 | LARGE INTESTINE |
| T84 | LARGE INTESTINE |
| HCC1833 | LUNG |
| NCIH1915 | LUNG |
| NCIH2126 | LUNG |
| NCIH2347 | LUNG |
| NCIH292 | LUNG |
| NCIH358 | LUNG |
| NCIH854 | LUNG |
| PC14 | LUNG |
| JHESOAD1 | OESOPHAGUS |
| OVCAR8 | OVARY |
| BXPC3 | PANCREAS |
| DANG | PANCREAS |
| HS766T | PANCREAS |
| PATU8902 | PANCREAS |
| PK59 | PANCREAS |
| T3M4 | PANCREAS |
| YAPC | PANCREAS |
| PC3 | PROSTATE |
| A253 | SALIVARY GLAND |
| ECC10 | STOMACH |
| GSS | STOMACH |
| KATOIII | STOMACH |
| OCUM1 | STOMACH |
| SNU216 | STOMACH |
| SNU719 | STOMACH |
| CAL62 | THYROID |
| SCC4 | UPPER AERODIGESTIVE TRACT |
| 5637 | URINARY TRACT |

TABLE 14B
Cell lines that express acetylcholinesterase
(ACHE) and any cholinergic receptor (CHRM/N)

| CELL LINE NAME | CANCER TYPE |
|---|---|
| IMR32 | AUTONOMIC GANGLIA |
| EWS502 | BONE |
| RDES | BONE |
| TC32 | BONE |
| HCC1569 | BREAST |
| BV173 | HAEMATOPOIETIC AND LYMPHOID TISSUE |
| GA10 | HAEMATOPOIETIC AND LYMPHOID TISSUE |
| KASUMI1 | HAEMATOPOIETIC AND LYMPHOID TISSUE |
| NB4 | HAEMATOPOIETIC AND LYMPHOID TISSUE |
| P3HR1 | HAEMATOPOIETIC AND LYMPHOID TISSUE |
| SLR20 | KIDNEY |
| UMRC6 | KIDNEY |
| CORL311 | LUNG |
| NCIH211 | LUNG |
| NCIH292 | LUNG |
| NCIH82 | LUNG |
| RERFLCAD2 | LUNG |
| T3M4 | PANCREAS |

TABLE 14C
Cell lines that express tryptophan hydroxylase
(TPH1) and any serotonin receptor (HTR)

| CELL LINE NAME | CANCER TYPE |
|---|---|
| COLO684 | ENDOMETRIUM |
| CORL95 | LUNG |
| DMS153 | LUNG |
| NCIH2029 | LUNG |
| NCIH2196 | LUNG |
| NCIH292 | LUNG |
| NCIH69 | LUNG |
| NCIH82 | LUNG |
| COLO704 | OVARY |
| QGP1 | PANCREAS |
| ECC10 | STOMACH |

According to the methods disclosed herein, a physician of skill in the art can treat a patient, such as a human patient with cancer (e.g., lung cancer), so as to inhibit cancer growth, reduce tumor burden, or slow disease progression. The method of treatment can include diagnosing or identifying a patient as a candidate for treatment with a neuromodulating agent based on neurome gene expression in a biopsy. For example, a tissue sample can be collected from a patient's lung cancer and analyzed for RNA expression by qPCR or RNAseq analysis, and the lung cancer can be found to express both tryptophan hydroxylase and serotonin receptor HTR2A. This neurome gene expression suggests that the patient's tumor may have co-opted an autocrine serotonin signaling loop to drive growth. To reduce the autocrine signaling, a physician of skill in the art can administer a serotonin blocking antibody, an inhibitory RNA that targets tryptophan hydroxylate, or an HTR2A antagonist (e.g., trazodone, mirtazapine, nefazodone, pizotifen, and hydroxyzine). The HTR2A antagonist can be administered locally (e.g., injected into the lung cancer) to decrease tumor growth or volume. The HTR2A antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the HTR2A antagonist is administered bimonthly, once a month, once every two weeks, or at least once a week or more (e.g., 1, 2, 3, 4, 5, 6, or 7 times a week or more).

The HTR2A antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the HTR2A antagonist can be compared to evaluate the efficacy of the treatment, and the rate of disease progression can be assessed by comparison to the patient's medical history prior to administration of the HTR2A antagonist. A finding of a reduction in the total number of tumors, number of primary tumors, volume of tumors, growth of tumors, or rate of disease progression indicates that the HTR2A antagonist has successfully treated the cancer.

### Example 22—Neurotrophic Factor Overexpression in Cancer

One form of cancer neuro-dependence is the expression by the cancer cells of neurotrophic factors that promote the growth of neurons into the tumor and the surrounding microenvironment. These neurons may then provide growth-promoting signals to the tumor. As such, inhibition of neurotrophic factors in tumors that express them may prevent the ingrowth of neurons and thus induce a growth-inhibitory effect on the tumor.

Tables 15A and 15B below show gene expression assessed by RNAseq for two common neurotrophic factors NGF and BDNF (a complete list of neurotrophic factors is provided in Table 7). Each table displays RNAseq data for a particular neurotrophic factor in tumor samples from patients with different types of cancer. Gene expression assessed using RNAseq was scored by log 2(FPKM). Values >0 are deemed to be “overexpressed” in this assay. The table shows the percentage of the total number of tumors analyzed from each cancer type (N) that overexpress the neurotrophic factor.

TABLE 15A
NGF EXPRESSION IN CANCER

| Cancer type | | | | |
|---|---|---|---|---|
| NGF | N | FKPM <0 | FKPM 0-2 | FKPM >2 |
| BL-Bladder Cancer | 21 | 71.4% | 14.3% | 14.3% |
| BN-Brain Cancer | 15 | 86.7% | 6.7% | 6.7% |
| BR-Breast Cancer | 43 | 88.4% | 9.3% | 2.3% |
| CC-Cholangiocarcinoma | 12 | 100.0% | 0.0% | 0.0% |
| CR-Colorectal Cancer | 194 | 99.5% | 0.5% | 0.0% |
| CV-Cervical Cancer | 11 | 90.9% | 9.1% | 0.0% |
| ES-Esophageal Cancer | 54 | 77.8% | 11.1% | 11.1% |
| GA-Gastric Cancer | 80 | 97.5% | 1.3% | 1.3% |
| GL-Gallbladder Cancer | 8 | 100.0% | 0.0% | 0.0% |
| GS-GIST | 14 | 85.7% | 0.0% | 14.3% |
| HN-Head and Neck Cancer | 56 | 53.6% | 19.6% | 26.8% |
| KI-Kidney Cancer | 16 | 93.8% | 6.3% | 0.0% |
| LI-Liver Cancer | 74 | 85.1% | 12.2% | 2.7% |
| LU-Lung Cancer | 281 | 59.8% | 22.8% | 17.4% |
| LY-Lymphoma | 18 | 100.0% | 0.0% | 0.0% |
| MC-Metastatic Carcinoma | 5 | 100.0% | 0.0% | 0.0% |
| ME-Melanoma | 135 | 95.6% | 2.2% | 2.2% |
| OV-Ovarian Cancer | 41 | 92.7% | 2.4% | 4.9% |
| PA-Pancreatic Cancer | 108 | 97.2% | 1.9% | 0.9% |
| SA-Sarcoma | 71 | 49.3% | 29.6% | 21.1% |
| UT-Uterine Cancer | 11 | 100.0% | 0.0% | 0.0% |

TABLE 15B
BDNF EXPRESSION IN CANCER

| Cancer type | | | | |
|---|---|---|---|---|
| BDNF | N | FKPM <0 | FKPM 0-2 | FKPM >2 |
| BL-Bladder Cancer | 21 | 85.7% | 9.5% | 4.8% |
| BN-Brain Cancer | 15 | 93.3% | 6.7% | 0.0% |
| BR-Breast Cancer | 43 | 95.3% | 4.7% | 0.0% |
| CC-Cholangiocarcinoma | 12 | 83.3% | 8.3% | 8.3% |
| CR-Colorectal Cancer | 194 | 95.4% | 4.1% | 0.5% |
| CV-Cervical Cancer | 11 | 81.8% | 9.1% | 9.1% |
| ES-Esophageal Cancer | 54 | 59.3% | 37.0% | 3.7% |
| GA-Gastric Cancer | 80 | 90.0% | 6.3% | 3.8% |
| GL-Galbladder Cancer | 8 | 75.0% | 25.0% | 0.0% |
| GS-GIST | 14 | 71.4% | 28.6% | 0.0% |
| HN-Head and Neck Cancer | 56 | 51.8% | 35.7% | 12.5% |
| KI-Kidney Cancer | 16 | 68.8% | 12.5% | 18.8% |
| LI-Liver Cancer | 74 | 87.8% | 5.4% | 6.8% |
| LU-Lung Cancer | 281 | 59.4% | 29.2% | 11.4% |
| LY-Lymphoma | 18 | 77.8% | 11.1% | 11.1% |
| MC-Metastatic Carcinoma | 5 | 40.0% | 60.0% | 0.0% |
| ME-Melanoma | 135 | 80.7% | 11.9% | 7.4% |
| OV-Ovarian Cancer | 41 | 75.6% | 14.6% | 9.8% |
| PA-Pancreatic Cancer | 108 | 76.9% | 18.5% | 4.6% |
| SA-Sarcoma | 71 | 60.6% | 25.4% | 14.1% |
| UT-Uterine Cancer | 11 | 90.9% | 9.1% | 0.0% |

### Example 23—Chrna6 Correlates with 5 Year Survival in Cancer Patients

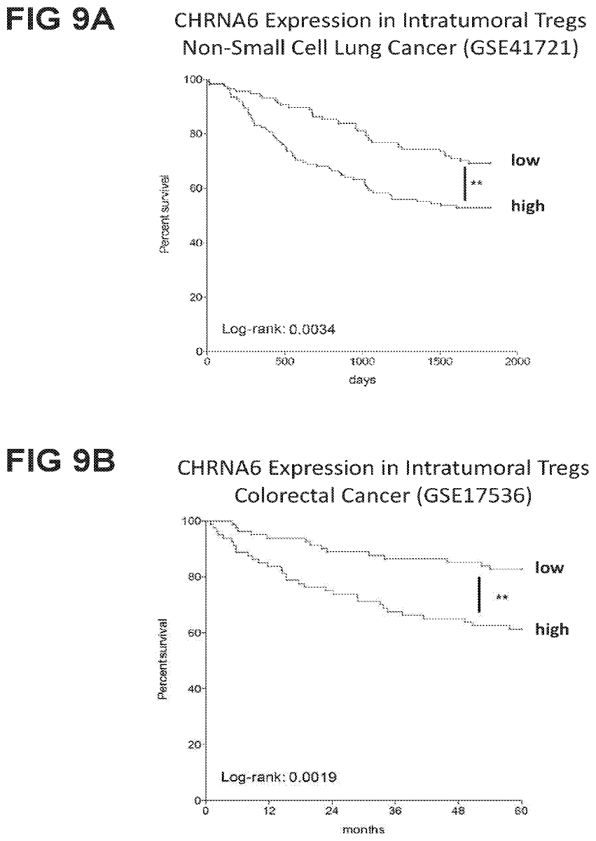

We evaluated the expression of nicotinic receptor Chrna6 in the TCGA GSE41721 (non-small cell lung cancer) and GSE17536 (colorectal cancer) data sets to determine whether there was a correlation between Chrna6 expression level and survival. We analyzed data from 275 non-small cell lung cancer (NSCLC) patients and 177 colorectal cancer (CRC) patients. Expression was normalized to CD3G in order to account for differential immune infiltration. We observed a significant improvement in 5 year survival in both NSCLC and CRC patients whose cancer expressed low levels of Chrna6 compared to NSCLC and CRC patients whose cancer expressed high levels of Chrna6 (FIGS. 9A-9B). These data suggest that high levels of Chrna6 lead to worse outcomes for patients with cancer.

### Example 24—Treatment of a Patient with NSLC with a Chrna6 Antagonist

According to the methods disclosed herein, a physician of skill in the art can treat a patient, such as a human patient with cancer (e.g., non-small cell lung cancer), so as to inhibit cancer growth, reduce tumor burden, or slow disease progression. The method of treatment can include diagnosing or identifying a patient as a candidate for treatment with a neuromodulating agent based on neurome gene expression in a biopsy. For example, a tissue sample can be collected from a patient's non-small cell lung cancer and analyzed for RNA expression by qPCR or RNAseq analysis, and the lung cancer can be found to express high levels of Chrna6. To treat the patient, a physician of skill in the art can administer a neuromodulating agent that decreases Chrna6 expression or function (e.g., an siRNA directed to Chrna6 or a Chrna6 antagonist, e.g., mecamylamine). The Chrna6 antagonist can be administered locally (e.g., injected into the non-small cell lung cancer or formulated for inhalation) to decrease tumor growth or volume. The Chrna6 antagonist is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the Chrna6 antagonist is administered bimonthly, once a month, once every two weeks, or at least once a week or more (e.g., 1, 2, 3, 4, 5, 6, or 7 times a week or more).

The Chrna6 antagonist is administered to the patient in an amount sufficient to decrease tumor growth decrease tumor burden, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, positron emission tomography (PET) scan, computed tomography (CT) scan, or magnetic resonance imaging (MRI) scan). Images from before and after administration of the Chrna6 antagonist can be compared to evaluate the efficacy of the treatment, and the rate of disease progression can be assessed by comparison to the patient's medical history prior to administration of the Chrna6 antagonist. A finding of a reduction in the total number of tumors, number of primary tumors, volume of tumors, growth of tumors, or rate of disease progression indicates that the Chrna6 antagonist has successfully treated the cancer.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1-6. (canceled)

7. A method comprising administering to a subject in need thereof an adenosine receptor antagonist in an amount effective to inhibit growth of cancer cells in the subject, relative to a control.

8. The method of claim 7, wherein the cancer cells are pancreatic cancer cells.

9. The method of claim 7, wherein the cancer cells are lung cancer cells.

10. The method of claim 7, wherein the adenosine receptor antagonist is selected from KW3902, MRS-1220, MRS-1334, MRS-1523, MRS-3777, MRE3008F20, PSB-10, PSB-11, and VUF-5574.

11. A method comprising administering to a subject in need thereof a histamine receptor antagonist in an amount effective to inhibit growth of cancer cells in the subject, relative to a control.

12. The method of claim 11, wherein the cancer cells are pancreatic cancer cells.

13. The method of claim 11, wherein the histamine receptor antagonist is selected from acrivastine, azelastine, astemizole, bilastine, bromodiphenhydramine, brompheniramine, buclizine, carbinoxamine, and cetirizine.

14. A method comprising administering to a subject in need thereof a dopamine antagonist in an amount effective to inhibit growth of cancer cells in the subject, relative to a control.

15. The method of claim 14, wherein the cancer cells are pancreatic cancer cells.

16. The method of claim 14, wherein the cancer cells are glioblastoma cells.

17. The method of claim 14, wherein the dopamine antagonist is selected from haloperidol, paliperidone, clozapine, risperidone, olanzapine, quetiapine, ziprasidone, amoxapine, clomipramine, and trimipramine.

18. The method of claim 14, wherein the amount effective to inhibit growth of cancer cells is also effective to reduce tumor weight by at least 50%, relative to a control.

19. A method comprising culturing cancer cells with a neuromodulating agent and measuring growth of the cancer cells.

20. A method comprising contacting cancer cells with a neuromodulating agent in an amount effective to inhibit growth of the cancer cells.
